(19) United States
(12) Patent Application Publication
(10) Pub. No.: US 2024/0247259 A1
(43) Pub. Date: Jul. 25, 2024

(54) COMPOSITIONS AND METHODS FOR MODULATING GENE EXPRESSION

(71) Applicant: ENTRADA THERAPEUTICS, INC., Boston, MA (US)

(72) Inventors: Xiulong SHEN, Waltham, MA (US); Xiang LI, Belmont, MA (US); Ziqing QIAN, Wellesley, MA (US); Natarajan SETHURAMAN, Hanover, NH (US); Haoming LIU, Canton, MA (US)

(21) Appl. No.: 18/289,944

(22) PCT Filed: May 9, 2022
(86) PCT No.: PCT/US2022/028354

§ 371 Date: Nov. 8, 2023

Related U.S. Application Data
(60) Provisional application No. 63/186,664, filed on May 10, 2021.
(60) Provisional application No. 63/210,866, filed on Jun. 15, 2021.
(60) Provisional application No. 63/210,876, filed on Jun. 15, 2021.
(60) Provisional application No. 63/239,671, filed on Sep. 1, 2021.
(60) Provisional application No. 63/290,817, filed on Dec. 17, 2021.
(60) Provisional application No. 63/298,587, filed on Jan. 11, 2022.
(60) Provisional application No. 63/318,201, filed on Mar. 9, 2022.
(60) Provisional application No. 63/321,918, filed on Mar. 21, 2022.
(60) Provisional application No. 63/362,295, filed on Mar. 31, 2022.

(51) Int. Cl. C12N 15/113; A61K 47/64; A61P 21/00; C07K 7/64

(52) U.S. Cl. CPC C12N 15/113; A61K 47/6455; A61P 21/00; C07K 7/64; C12N 2310/11; C12N 2310/3233; C12N 2310/341; C12N 2310/3513

(57) ABSTRACT

Compounds that include a cyclic cell penetrating peptide and a therapeutic moiety that modulates polyadenylation of a gene transcript. The therapeutic moiety may be an antisense compound (AC) that binds a target gene transcript. The AC may bind to at least a portion of a polyadenylation signal element (PSE) or may bind in sufficiently close proximity to the PSE to modulate polyadenylation of the target gene transcript. Methods include administering the aforementioned compounds to cells or subjects to modulate diseases or conditions.

### CROSS REFERENCE TO RELATED APPLICATIONS

This application claims the benefit of U.S. Provisional Application Ser. No. 63/186,664, filed on May 10, 2021; 63/210,876, filed on Jun. 15, 2021; 63/290,817, filed on Dec. 17, 2021; 63/321,918, filed on Mar. 21, 2022; 63/362,295, filed on Mar. 31, 2022; 63/239,671, filed on Sep. 1, 2021; 63/210,866, filed on Jun. 15, 2021; 63/298,587, filed on Jan. 11, 2022, and 63/318,201, filed on Mar. 9, 2022, which provisional applications are incorporated by reference herein in their respective entireties to the extent that they do not conflict with the disclosure presented herein.

### FIELD OF THE INVENTION

Provided herein are compositions and methods for modulating the expression of a gene, in particular, compositions and methods are provided for targeting one or more polyadenylation sequence elements of a gene transcript.

### BACKGROUND

The poly(A) or polyadenosine tail is a long chain of adenosines that is added to an RNA transcript during RNA processing. The poly(A) tail makes the RNA molecule more stable, prevents its degradation, and allows the mature mRNA molecule to be exported from the nucleus and translated into a protein by ribosomes in the cytoplasm (Kantor, et al., Advances in Genetics (87): 125-197). Altering the ability of a cell to add a poly(A) tail to a gene transcript, such as a pre-mRNA, may alter the stability, rate of degradation, degree of cellular transport, or degree of translation of the gene transcript. For example, interfering with the ability of a cell to add a poly(A) tail to a gene transcript may result in decreased amounts of pre-mRNA, mRNA, and protein associated with the gene. As such, interfering with the ability of a cell to add a poly(A) tail to a gene transcript may serve as a useful therapeutic strategy when one or more of the pre-mRNA, mRNA, or protein are associated with a disease. Such a strategy may be useful regardless of the underlying cause of the disease.

Aberrant gene transcription, splicing and/or translation of a multitude of genes can lead to host of human diseases. For instance, Type I interferons (IFNs) are suggested to play a key role in disease and increased expression of IFN-induced genes is associated with autoimmune diseases. For example, IRF-5 upregulation and polymorphisms have been implicated in the development of numerous inflammatory and autoimmune diseases, including, but not limited to, Rheumatoid Arthritis (RA), systemic sclerosis, multiple sclerosis (MS), inflammatory bowel disease (IBD), systemic lupus erythematosus (SLE) and Sjögrens syndrome (Kristjansdottir et al. J. Med. Genet. (2008), 45:362-369; Thompson et al. Front. Immunol. (2018), doi.org/10.3389/fimmu.2018.02622; Almuttaqi and Udalova, FEBS J. 2019, 286:1624-1637). Similarly, at the RNA level, the accumulation of mutant ribonucleic acid (RNA) molecules can be toxic to a cell, causing human disease through trans-acting dominant mechanisms that share unifying pathogenic events mediated by toxic RNA accumulation and disruption of RNA-binding proteins (Sicot and Gomes-Pereira, Biochimica et Biophysica Acta 1832 (2013) 1390-1409).

Abnormal expansion of trinucleotide repeat sequences has been identified as the cause of human diseases in fragile-X syndrome, spinobulbar muscular atrophy (SBMA) or Kennedy disease, and in a number of human inherited diseases associated with the expansion of trinucleotide repeats at specific chromosomal loci, such as myotonic dystrophy type 1 (DM1), Huntington disease (HD), Friedreich ataxia (FRDA), dentatorubral-pallidoluysian atrophy (DRPLA) and many spinocerebellar ataxias (SCAs) (Oberle et al., Science (1991) 252, 1097-1102; Kremer et al., Science (1991), 252, 1711-1714; Yu et al., Science (1991), 252, 1179-1181; Verkerk et al., Cell (1991), 65, 905-914; La Spada et al., Nature (1991), 352, 77-79; Pearson et al., Nat. Rev. Genet. (2005), 6, 729-742; Gomes-Pereira and Monckton, Mutat. Res. (2006), 598, 15-34; Gatchel and Zoghbi, Nat. Rev. Genet. (2005), 6, 743-755).

Despite the similar genetic defect shared by these conditions, the downstream pathophysiology can be mediated by multiple pathways, which may be partially due to the different locations of the expanded repeat within a variety of otherwise unrelated genes and can manifest through either gain of function or loss of function mechanisms. The expanded repeat tract can be located in protein-coding sequences, and therefore affect the final gene product of the mutant gene, such as in CAG expansion disorders (e.g.: HD, SBMA, DRPLA, SCA1, SCA2, SAC3/MJD, SCA7 and SCA17), whose molecular pathogenesis appears to be primarily mediated by a deleterious gain of function of the polyglutamine tract encoded by the expanded trinucleotide sequence (Takahashi et al., J. Mol. Cell Biol. (2010), 2, 180-191). Alternatively, although not changing the final protein sequence, non-coding triplet repeats and loss of function mutations can also be pathogenic, as the untranslated disease-associated repeat expansions can map in the 5′ or 3′ untranslated regions (UTRs), promoters or introns of the affected gene (Sakamoto et al., Mol. Cell (1999), 3, 465-475; Pieretti et al., Cell (1991), 66, 817-822).

Current understanding suggests that disease-causing RNA likely mediates the molecular pathogenesis of other conditions, many of them caused by the expansion of non-coding repeat sequences, such as DM type 2 (DM2), fragile X tremor ataxia syndrome (FXTAS), SCA type 8 (SCA 8), SCA 10, SCA 12, SCA 31, SCA 36, Huntington disease-like 2 (HDL2) and amyotrophic lateral sclerosis (ALS) (Liquori et al., Science (2001), 293, 864-867; Hagerman and Hagerman, Am. J. Hum. Genet. (2004), 74, 805-816; Daughters et al., PLoS Genet. (2009), 5, e1000600; White et al., PLoS Genet. (2010), 6 e1000984; Holmes et al., Brain Res. Bull. (2001), 56, 397-403; Sato et al., Am. J. Hum. Genet. (2009), 85, 544-557; Kobayashi et al., Am. J. Hum. Genet. (2011), 89, 121-130; Rudnicki et al., Ann. Neurol. (2007), 67, 272-282; DeJesus-Hernandez et al., Neuron (2011), 72, 245-256). Toxic RNAs have also been implicated in polyglutamine expansion disorders primarily mediated by proteotoxicity, suggesting that the contribution of RNA toxicity to disease might have wider implications than previously thought, and participate in multiple human conditions (Wojciechowska and Krzyzosiak, RNA Biol. 8 (2011) 565-571).

Therapeutic compounds, such as antisense compounds, that modulate polyadenylation of a gene transcript may be effective at treating diseases regardless of their underlying genetic mechanism. For example, such compounds may be useful for treating diseases associated with genetic mutations, with aberrant gene transcription, splicing, translation, trinucleotide repeats, and combinations thereof. The therapeutic applications of antisense compounds are extremely broad, since these compounds can be synthesized with any nucleotide sequence directed against virtually any target gene, gene transcript, or genomic segment.

Major problems for the use of antisense compounds in therapeutics include their limited ability to gain access to the intracellular compartment when administered systemically, their limited ability to achieve wide or specifically targeted tissue distribution, and the challenge of obtaining sufficient specificity for the targeted RNA to minimize off-target effects. Intracellular delivery of antisense compounds can be facilitated by use of carrier systems such as polymers, cationic liposomes or by chemical modification of the construct, for example by the covalent attachment of cholesterol molecules. However, intracellular delivery efficiency is low and tissue distribution can be narrow. In addition, existing technologies remain hampered by off-target interactions. As a consequence, improved delivery systems are still required to increase the effectiveness of these antisense approaches, and their remains an unmet need for effective compositions to deliver antisense compounds to intracellular compartments broadly to all effected tissue types to specially target a given gene product, so as to treat diseases caused by, e.g., aberrant gene transcription, splicing and/or translation, trinucleotide repeats, or the like, and combinations thereof.

### SUMMARY

This disclosure generally relates to compounds, compositions, and methods for modulating expression of genes, such as genes associated with diseases. In embodiments, this disclosure relates to compounds and compositions that include a therapeutic moiety (TM) and a cell penetrating peptide (CPP), such as a cyclic CPP (cCPP), to modulate gene expression, and methods for using such compounds and compositions. In embodiments, the TM is capable of modulating polyadenylation of an RNA transcript, which may modulate levels of pre-mRNA, mRNA, and protein associated with the transcript. In embodiments, the TM is an antisense compound (AC).

The compounds may comprise a CPP conjugated to or chemically linked to the TM. The CPP may be a cyclic CPP (cCPP). The compounds may comprise an endosomal escape vehicle (EEV). The EEV may be conjugated to or chemically linked to the TM. The EEV may comprise a cCPP.

Described herein are methods in which the compounds or compositions described herein are used to treat a disease. In embodiments, the disease is a genetic disease. In embodiments, the compounds or compositions are used to treat the genetic disease by modulating expression of a gene associated with the disease. In embodiments, the compounds or compositions treat the genetic disease by modulating polyadenylation of a gene transcript associated with the disease. In embodiments, the methods comprise administering the compound or compositions described herein to a subject in need thereof. In embodiments, the subject in need thereof is a patient having, or at risk of having, the genetic disease. In embodiments, the method comprises administering a therapeutically effective amount of the compound or compositions described herein to the subject in need thereof. In embodiments, the genetic disease is a disease associated with aberrant expression of IRF-5, DPMK, or DUX4, or a genetic variant thereof.

The CPP may enhance intracellular deliver of the AC to enhance the effectiveness of the AC to modulate polyadenylation of the target transcript. The CPP can be a cyclic CPP (cCPP).

The compounds described herein may comprise an endosomal escape vehicle (EEV) configured to allow compounds, or moieties thereof, that are internalized into the cell in endosomes to escape the endosomes and enter the cytosol or cellular compartment to allow the AC act on the target transcript and modulate polyadenylation. In embodiments, the EEV comprises the CPP, such as the cCPP.

In embodiments, the cCPP is of Formula (A):

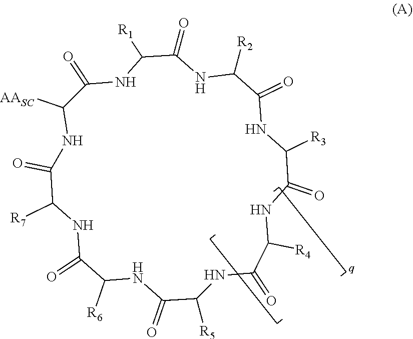

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, β-(1-piperidinyl)alanine;
AA_{SC} is an amino acid side chain; and
q is 1, 2, 3 or 4.

In embodiments, the cCPP is of Formula (A) is of Formula (I):

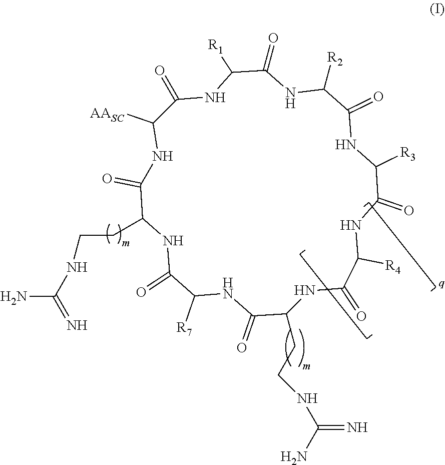

or a protonated form or salt thereof,
wherein each m is independently an integer from 0-3.

In embodiments, the cCPP is of Formula (A) is of Formula (I-1):

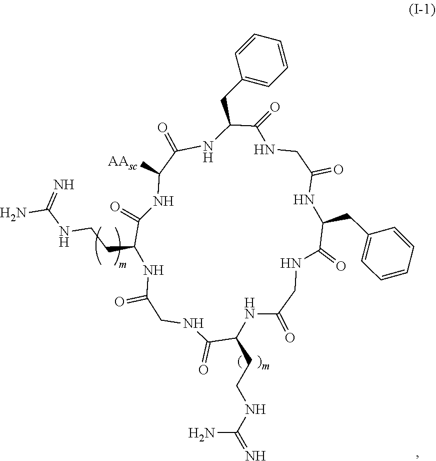

or a protonated form or salt thereof.

In embodiments, the cCPP is of Formula (A) is of Formula (I-2):

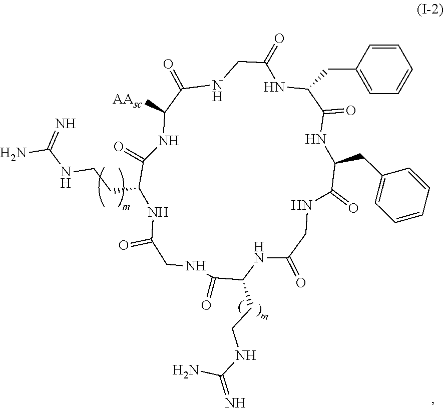

or a protonated form or salt thereof.

In embodiments, the cCPP is of Formula (A) is of Formula (I-3):

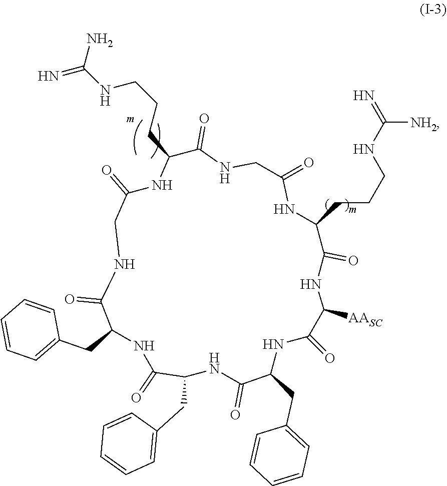

or a protonated form or salt thereof.

In embodiments, the cCPP is of Formula (A) is of Formula (I4):

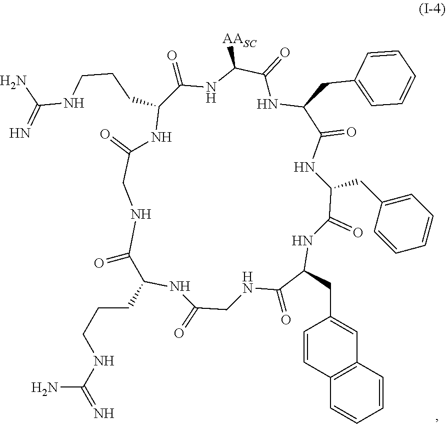

or a protonated form or salt thereof.

In embodiments, the cCPP is of Formula (A) is of Formula (I-5):

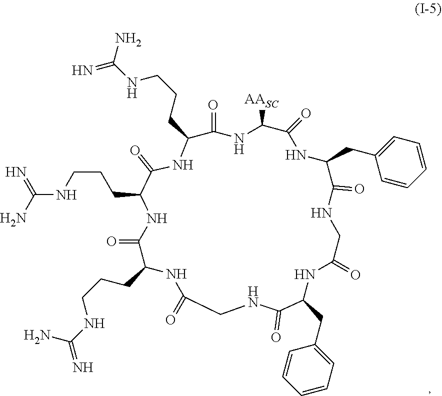

or a protonated form or salt thereof.

In embodiments, the cCPP is of Formula (A) is of Formula (I-6):

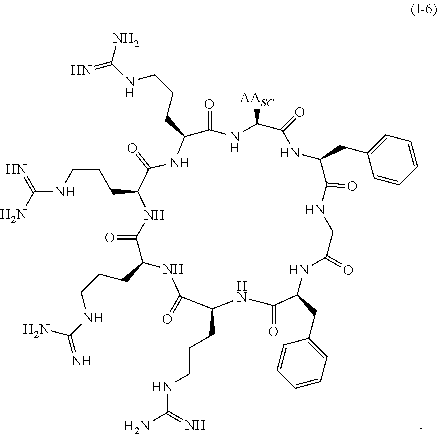

or a protonated form or salt thereof.

In embodiments, the cCPP is of Formula (II):

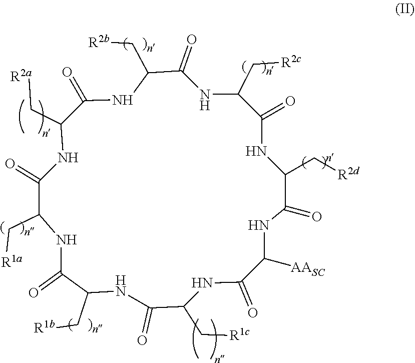

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R², R^{2b}, R^{2c} and R^{2d} is

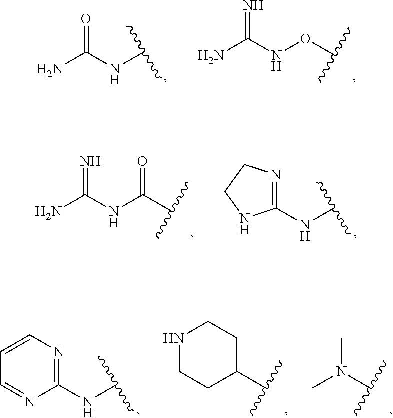

or a protonated form or salt thereof;
at least one of R^{2a}, R^{2b}, R^{2c} and R^{2d} is guanidine or a protonated form or salt thereof;
each n″ is independently an integer from 0 to 5;
each n′ is independently an integer from 0 to 3; and
if n′ is 0 then R^{2a}, R^{2b}, R^{2b} or R^{d} is absent.

In embodiments, the cCPP of Formula (II) is of Formula (II-1):

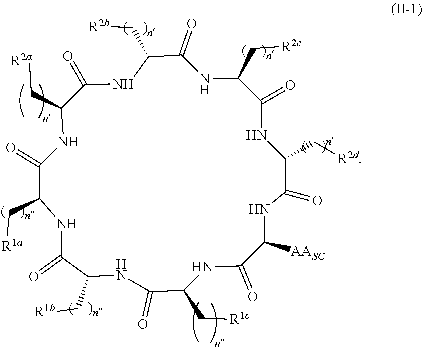

In embodiments, the cCPP of Formula (II) is of Formula (IIa):

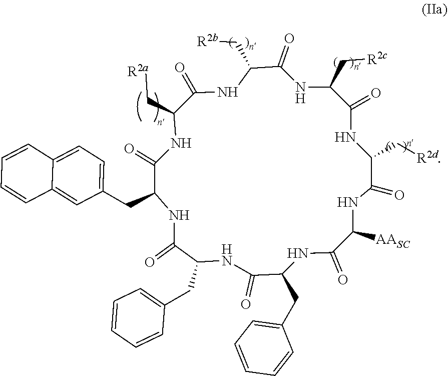

In embodiments, the cCPP of Formula (II) is of Formula (IIb):

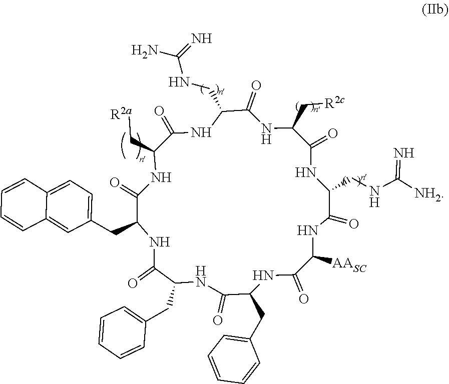

In embodiments, the cCPP of Formula (II) is of Formula (IIc):

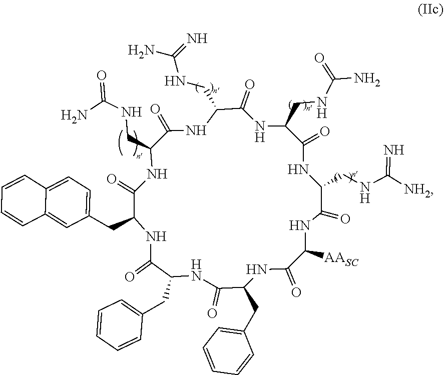

or a protonated form or salt thereof.

In embodiments, the cCPP has the structure:

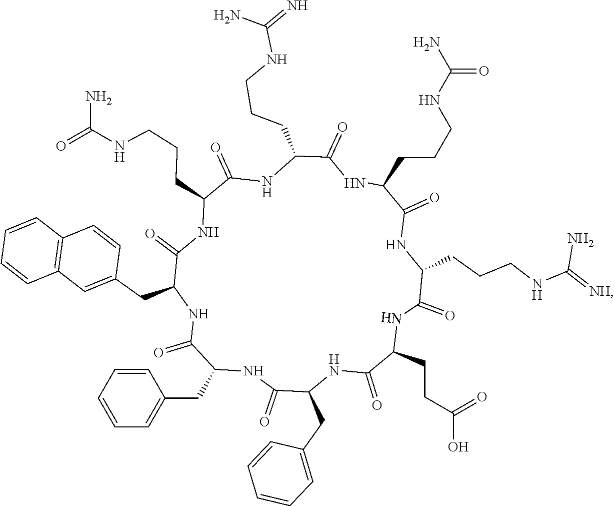 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the cCPP has the structure:

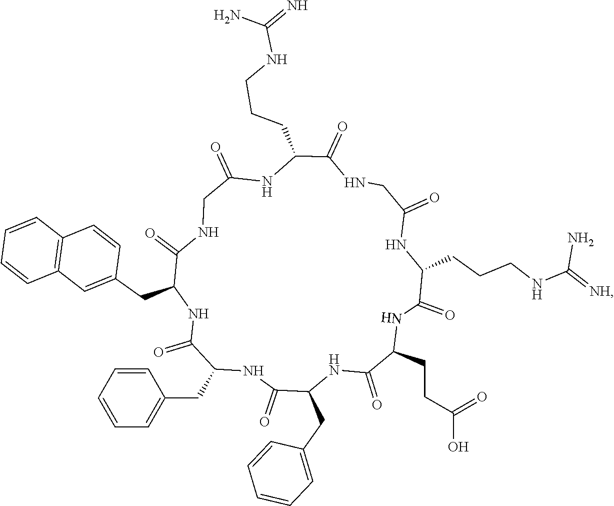 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the compound comprises an exocyclic peptide (EP). In embodiments, the EP comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine.

In embodiments, the compound is of Formula (C):

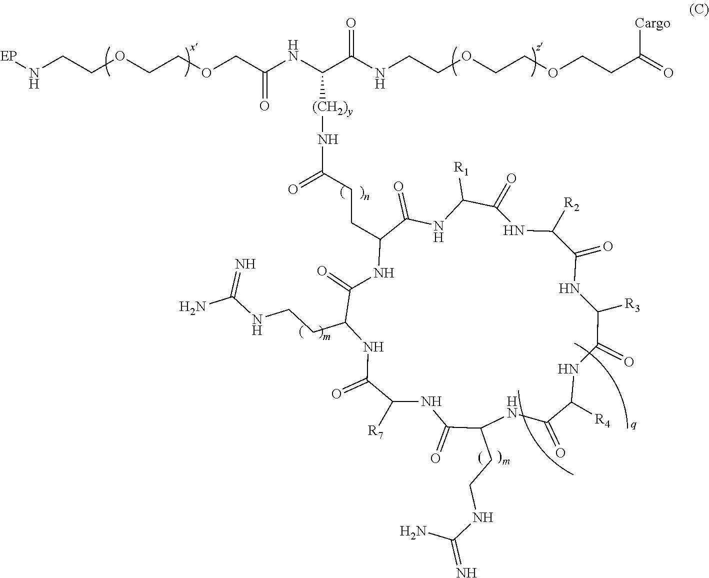

or a protonated form or salt thereof, wherein:
R₁, R₂, and R₃ are each independently H or a side chain comprising an aryl or heteroaryl group, wherein at least one of R₁, R₂, and R₃ is a side chain comprising an aryl or heteroaryl group;
R₄ and R₇ are independently H or an amino acid side chain;
EP is an exocyclic peptide;
each m is independently an integer from 0-3;
n is an integer from 0-2;
x′ is an integer from 1-23;
y is an integer from 1-5;
q is an integer from 1-4;
z′ is an integer from 1-23, and
Cargo is the therapeutic moiety.

In embodiments, the compound comprises the structure of Formula (C-1), (C-2), (C-3), or (C-4):

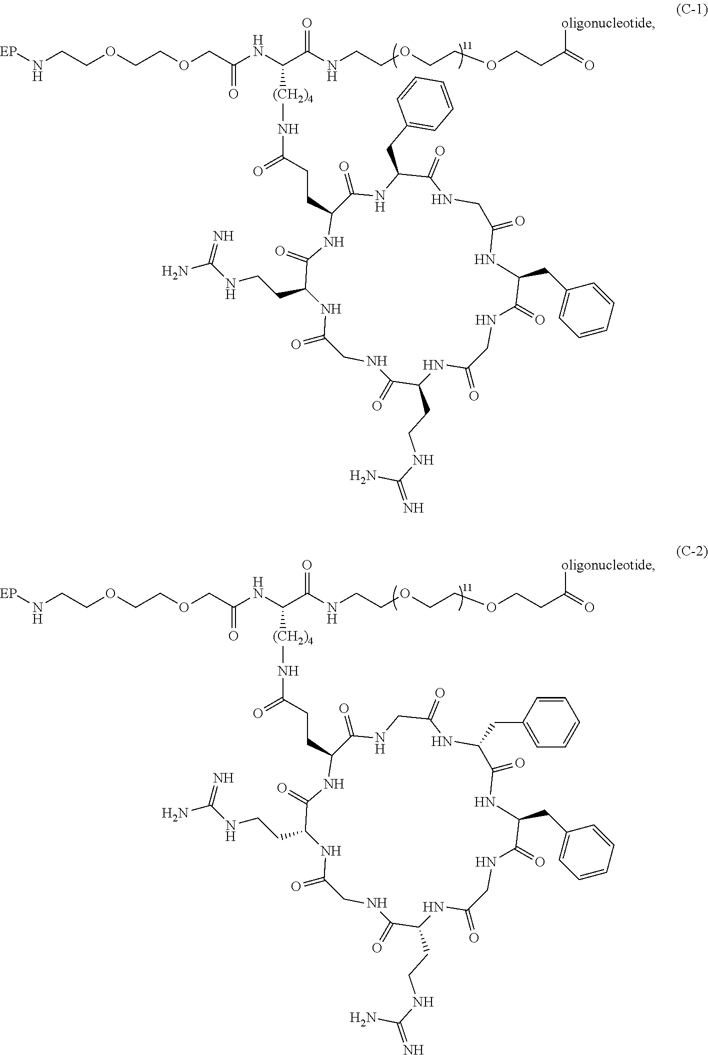

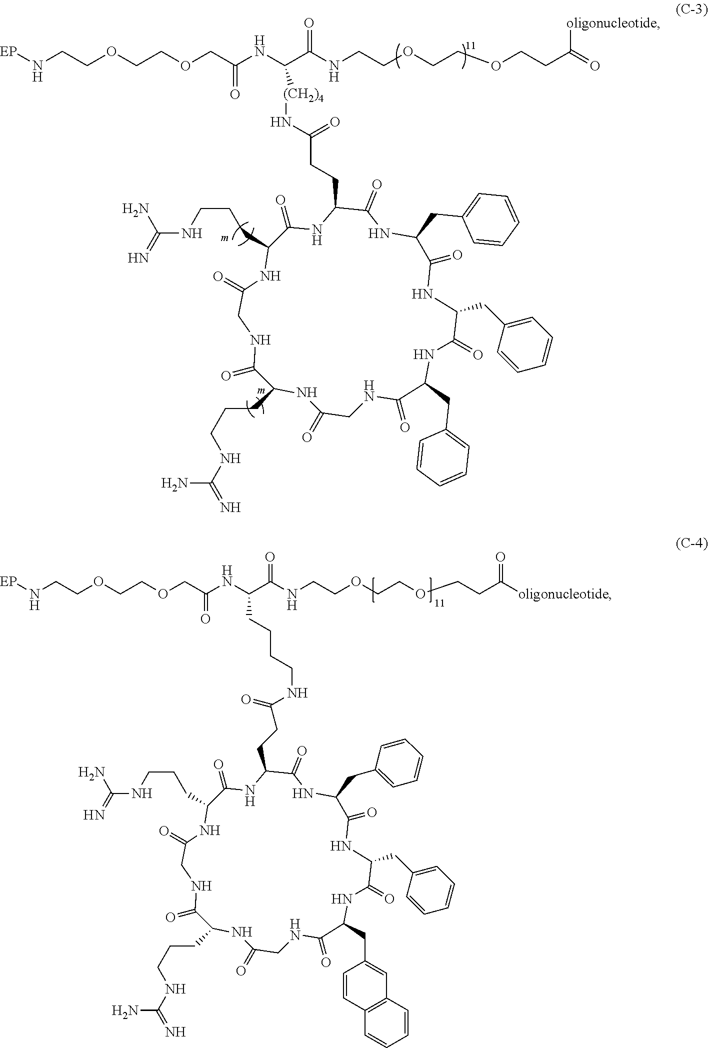

or a protonated form or salt thereof, wherein EP is an exocyclic peptide, and oligonucleotide is the therapeutic moiety.

### BRIEF DESCRIPTION OF THE DRAWINGS

FIG. 1 is a schematic representation of RNA before and after cleavage and addition of the poly(A) tail showing the location of polyadenylation sequence element (PSEs), such as the polyadenylation signal (PAS), the cleavage site (CS), and the downstream element (DSE) and the intervening sequence (IS) between the PAS and the CS.

FIG. 2 shows modified nucleotides used in antisense oligonucleotides (ASOs) described herein. Structures 1-3 (1=Phosphorothioate; 2=(S_{C5}-Rₚ)-α,β-CAN; 3=PMO) are phosphate backbone modifications; 4 (2-thio-dT) is a base modification; 5-8 (5=2′-OMe-RNA; 6=2′O-MOE-RNA; 7=2′F-RNA; 8=2′F-ANA) are 2′ sugar modifications; 9-11 are constrained nucleotides; 12-14 (9=LNA; 10=(S)-cET, 11=tcDNA; 12=FHNA; 13=(S)5′-C-methyl; 14=UNA) are additional sugar modification; and 15-18 (15=E-VP; 16=Methyl phosphonate, 17=5′ phosphorothioate; 18=(S)-5′-C-methyl with phosphate) are 5′ phosphate stabilization modifications; 19 is a morpholino sugar. Reformatted from Khvorova, A., et al., Nat. Biotechnol. 2017 March; 35(3): 238-248.

FIGS. 3A-3D provide structures of the adenine (A), cytosine (B), guanine (C), and thymine (D) morpholino subunit monomers used in synthesizing phosphorodiamidate-linked morpholino oligomers (PMOs).

FIGS. 4A-D illustrate conjugation chemistries for connecting an antisense compound (AC) to a cyclic cell penetrating peptide (cCPP). FIG. 4A shows the amide bond formation between peptides with carboxylic acid group or with TFP activated ester and primary amine residues at the 5′ end of AC. FIG. 4B shows the conjugation of secondary amine or primary amine modified AC at 3′ and peptide-TFP ester through amide bond formation. FIG. 4C shows the conjugation of peptide-azide to the 5′ cyclooctyne modified AC via copper-free azide-alkyne cycloaddition. FIG. 4D demonstrates another conjugation between 3′ modified cyclooctyne ACs or 3′ modified azide ACs and cCPP containing linker-azide or linker-alkyne/cyclooctyne moiety, via a copper-free azide-alkyne cycloaddition or copper catalyzed azide-alkyne cycloaddition, respectively (click reaction). In FIGS. 4B-D, B refers to a nucleobase.

FIG. 5 shows conjugation chemistry for connecting an AC and a CPP with an additional linker modality containing a polyethylene glycol (PEG) moiety.

FIG. 6 shows the IRF-5 expression levels in THP1 cells transfected with various phosphorodiamidate morpholino oligomers (PMOs) targeting the polyadenylation sequence (PAS) of IRF-5. NT is no nucleofection and not treated (no nucleic acid), and nucleofection is nucleofected but not treated.

FIG. 7 shows the result of the transfection of DM1 patient-derived fibroblasts with various ACs that have a target nucleotide sequence that includes the polyadenylation signal (PAS) of a DMPK1 transcript.

FIGS. 8A-D show mRNA levels of (A) MBD3L3, (B) ZSCAN4 (C), TRIM43 (D), and DUX4-3′UTR relative to RPL19 after a FSHD cell line (GM16283) and two undiseased cell lines (WT-1 and WT-2; GM16281 and GM16275) were treated with varying concentrations of the PMO-EEV construct PAS-EEV (127-777). n=3, *p<0.05, **p<0.01, ***p<0.001 relative to FSHD no treatment by student's t-test.

FIGS. 9A-D show mRNA levels of DUX4 3′UTR (A), MBD3L3 (B), ZSCAN4 (C), and TRIM43 (E) after a FSHD cell line (GM16283; NT) and two undiseased cell lines (GM16281 and GM16275) were treated with the PMOs in Table 13 via nucleofection transfection. n=3, *p<0.05, **p<0.01, ***p<0.001 relative to FSHD no treatment by student's t-test.

FIGS. 10A-D show mRNA levels of DUX4 3′UTR (A), MBD3L3 (B), ZSCAN4 (C), and TRIM43 (E) after a FSHD cell line (GM16283; NT) and two undiseased cell lines (GM16281 and GM16275) were treated with the PMOs in Table 13 via endoporter transfection. n=3, *p<0.05, **p<0.01, ***p<0.001 relative to FSHD no treatment by student's t-test.

FIG. 11 shows the chemical structure of a compound comprising EEV777, which includes Ac-PKKKRKV-Lys(cyclo(Ff-Nal-RrRrQ) (Ac-SEQ ID NO:42-Lys(cyclo(SEQ ID NO:67)), linked to an AC.

FIGS. 12A-12B show the relative transcription of ZSCAN4 (A) and TRIM43 (B) in FSHD patient-derived muscle cells treated with EEV-PMO (127-777). N=3 biological replicates Data are mean±SD.

### DETAILED DESCRIPTION

### Polyadenylation

Although most cells in multicellular organisms contain the same genome, spatial and temporal diversity in gene transcripts is observed. The diversity is due to the multiple levels of processing during the stages of the transcribing the gene into RNA and translating the RNA into a protein. In many cases the gene is transcribed into pre-messenger RNA (pre-mRNA) and processed into a mature mRNA, which is then translated into a protein.

Pre-mRNA is primary transcript and the immediate product of transcription. The pre-mRNA is processed into mature mRNA, which may be translated into a protein. Mature mRNA contains the coding sequence (e.g., no introns) of a gene. Pre-mRNA is processed to give functional mature mRNA. In eukaryotes, processing pre-mRNA into functional mature mRNA generally includes adding a cap on the 5′ untranslated region, adding a polyadenosine (poly(A)) tail on the 3′ untranslated region, and splicing.

The poly(A) tails of processed mRNA in eukaryotes influence the stability of the mRNA, the translation or translation efficiency, and/or the transport of the mRNA from the nucleus to the cytoplasm and thereby ultimately governs the production of a protein. More specifically, the poly(A) tail allows for the transport of the RNA molecule from the nucleus to the cytoplasm, enhances translation efficiency, and controls RNA degradation (see, Nourse et al. (2020), Biomolecules 10(915) doi:10.3390/biom10060915). Formation of the poly(A) tail is also connected to other transcriptional and post-transcriptional processes, including for example splicing and transcriptional termination.

The process of adding the poly(A) tail is termed polyadenylation. The polyadenylation process is generally a two-step process involving a cleavage reaction followed by the addition of the poly(A) tail. The cleavage reaction involves endonucleolytic cleavage. Following cleavage, in almost all cases, multiple adenosines (e.g., about 50 to about 300) are enzymatically added to the resulting 3′ cleaved end to generate the poly(A) tail (Tian et al., (2005) Nuc. Acid. Res. 33(1):201-212 and Neve et al., (2017) RNA Biology, 14(7):865-890).

The polyadenylation process is governed by more than 80 RNA-binding proteins; however, fewer than 20 factors make up the core of the polyadenylation protein complex needed to mediate cleavage and polyadenylation in vitro (Marsollier et al., (2018), Int. J. Mol. Sci. 19, 1347, doi:10.3390/ijms19051347). These 20 factors are distributed in eight complexes: cleavage and polyadenylation specific factor (CPSF); cleavage stimulation factor (CstF); Symplekin; Mammalian cleavage factor I (CFIm); Mammalian cleavage factor II (CFIIm); Poly(A) polymerase (PAP); RNA polymerase II (PolII); and PolII C-Terminal Domain (CTD) (Ibid).

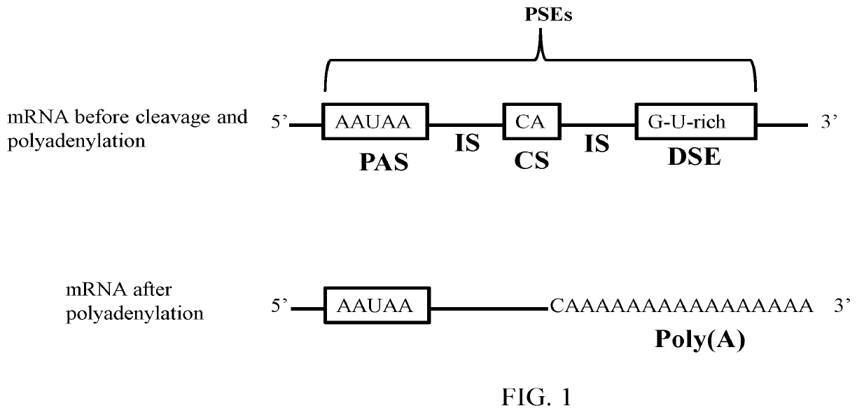

The factors interact with (e.g., bind to) a polyadenylation sequence element (PSE) during the polyadenylation process. Generally, the polyadenylation sequence elements include a polyadenylation signal (PAS), a cleavage site (CS), and a GU-rich downstream element (DSE) (FIG. 1). In some cases, the polyadenylation sequence elements may also include one or more of an auxiliary upstream element (USE), a G-rich sequence (GRS) auxiliary downstream element (AUX DSE), and/or a sequence downstream of a core U-rich element (URE)(not depicted in FIG. 1; Chen and Wilusz (1998) Nuc. Acid. Rec. 1998 26(12):2891-2898). Each PSE may be separated by intervening nucleotide sequence (IS) (FIG. 1). Each intervening IS may be a PSE in and of itself (See, Venkataraman et al., (2005) Genes and Dev. 19:1315-1327).

The PAS is an adenosine-rich hexamer sequence that includes a canonical AATAAA hexamer or a variant differing by a single nucleotide (e.g., AAUAAA, AUUAAA, UAUAAA, AGUAAA, AAGAAA, AAUAUA, AAUACA, CAUAAA, GAUAAA, CAUAAA, GAUAAA, AAUGAA, UUUAAA, ACUAAA, AAUAGA, AAAAAG, AAAACA, GGGGCU; Marsollier et al. Int. J. Mol. Sci., (2018), 19, 1347, doi:10.3390/ijms19051347; Beaudoing, et al. Genome Res. (2000), 10, 1001-1010; and Tian, B. et al., Nucleic Acids Res. (2005), 33, 201-212). The hexamer sequences occur with varying frequencies, with AAUAAA and AUUAAA being the most frequent (see, Ibid). The PAS is typically found upstream of the CS. The hexamer sequence of the PAS serves as the binding site for a cleavage and polyadenylation specific factor (CPSF). The PAS can also be determined by the presence of other auxiliary elements, such as upstream U-rich elements (USE) (see, Tian et al., Nuc. Acid. Res. (2005), 33(1):201-212 and Neve et al. (2017) RNA Biology, 2017, 14(7):865-890).

The DSE is a U-rich or U/G-rich element that serves as the binding site for a cleavage stimulatory factor (CstF). The DSE is typically found downstream of the CS. The DSE may be followed by a stretch of three or more uracil bases present downstream of the CS, often within 20 to 40 nucleotides of the CS. In mammals, CA and UA are the most frequent dinucleotides that precede the cleavage site (CS), although the actual cleavage site is known to be heterogeneous.

CPSF and CstF, two multi-subunit complexes, cooperate with each other and two additional factors (cleavage factors I and II) to cleave the mRNA sequence. Poly(A) polymerase (PAP), a single-subunit enzyme is also involved in cleavage of most pre-mRNAs, as is RNA polymerase II. CPSF and PAP together with a poly(A) binding protein II and cleavage stimulating factor (CstF) are involved in the addition of the poly(A) tail (Takagaki and Manley, Mol Cell Biol. (2000), 20(5):1515-1525).

Methods for identifying polyadenylation sequence elements are known and can include but are not limited to, for example, the methodologies described by: Tian et al., Nuc. Acid. Res. (2005) 33(1):201-212; Beaudoing, et al., Genome Res. (2000), 10, 1001-1010; Marsollier et al., Int. J. Mol. Sci. (2018), 19, 1347, doi:10.3390/ijms19051347; Chen, Molec. Therapy (2016), 24(8) 1405-1411; Venkataraman et al. Genes and Dev. (2005) 19:1315-1327; Nourse et al. Biomolecules (2000), 10(915) doi:10.3390/biom10060915; and Vickers et al. Nucleic Acids Research (2001) 29(6) 1293-1299.

### Compounds

Disclosed herein, are compounds that modulate the expression of a gene of interest. In embodiments, the compounds modulate polyadenylation and/or expression of a gene transcript of interest. In embodiments, the compounds inhibit polyadenylation of a gene transcript of interest. In embodiments, the compound includes at least one cell penetrating peptide (CPP; discussed in detail herein) and at least one therapeutic moiety (TM) that modulates polyadenylation of the gene transcript.

Described herein, among other things, are compounds and compositions for modulating expression of a gene. The compounds and compositions may modulate expression of a gene by modulating polyadenylation of a gene transcript. The compounds and compositions may comprise a therapeutic moiety (TM) that targets one or more polyadenylation sequence elements of a gene transcript. The TM may comprise an antisense compound (AC).

The compounds may comprise a cell penetrating peptide (CPP). The CPP may be conjugated to or chemically linked to the TM. The CPP may be a cyclic CPP (cCPP).

The compounds may comprise an endosomal escape vehicle (EEV). The EEV may be conjugated to or chemically linked to the TM. The EEV may comprise a cCPP.

### Therapeutic Moiety

In embodiments, the compounds include one or more therapeutic moieties (TM) that are capable of modulating polyadenylation a transcript of interest from a gene of interest. In embodiments, the TM inhibits polyadenylation of the gene transcript. As used herein, a “gene of interest” or “target gene” are used interchangeably and refer to a gene for which modulation of expression is desired or intended. A gene of interest may be a gene associated with a disease, such as an Interferon Regulatory Factor 5 (IRF-5) gene, a myotonic dystrophy protein kinase or DM1 protein kinase (DMPK) gene, a double homeobox 4 (DUX4) gene, or combinations thereof. In embodiments, modulation of the expression of the gene of interest may treat the disease.

In embodiments, the TM binds to (e.g., hybridizes with) a target nucleotide sequence. As used herein, the terms “target nucleotide sequence,” “target nucleic acid,” and “target nucleic acid sequence” refer to the specific nucleotide sequence with which the TM directly interacts (e.g., binds to). The target nucleotide sequence is generally contained within a gene transcript, such as pre-mRNA. A gene transcript that contains the target nucleotide sequence is referred to herein as a “target transcript” or “target gene transcript.”

The TM may be any suitable compound that may modulate gene expression or modulate polyadenylation of a target gene transcript. In embodiments, the TM is an antisense compound (AC), one or more of the elements associated with clustered regularly interspaced short palindromic repeats (CRISPR) gene editing machinery, a polypeptide, a detectable moiety, or combinations thereof.

In embodiments, the TM binds to the target transcript and alters polyadenylation, translation, processing, translocation from the nucleus to the cytoplasm, degradation of the target transcript, or combinations thereof. In embodiments, the TM binds to a target nucleotide sequence, for example, a portion of transcript of interest, at a position proximate to and/or including one or more polyadenylation sequence elements.

### Antisense Compound (AC)

In embodiments, the therapeutic moiety includes an antisense compound (AC) that can alter the expression of a target gene. In embodiments, the AC includes an oligonucleotide having DNA bases, modified DNA bases, RNA bases, modified RNA bases, traditional internucleoside linkages, modified internucleoside linkages, traditional DNA sugars, modified DNA sugars, traditional RNA sugars, modified RNA sugars, of combinations thereof. In embodiments, the AC includes a nucleotide sequence that is complementary to a target nucleotide sequence found within a target transcript. In embodiments, the AC includes a nucleotide sequence that is complementary to a target nucleotide sequence that is proximate to and/or includes at least a portion of a polyadenylation sequence element (PSE) of pre-mRNA transcript of interest. Binding of the AC to a target nucleotide sequence within the target transcript that is proximate to and/or includes at least a portion of a polyadenylation sequence element may inhibit the ability of one or more proteins associated with polyadenylation from binding to one or more sequence elements, which may inhibit polyadenylation of the target transcript. Inhibition of polyadenylation of the target transcript may decrease the stability of the transcript (and increase the rate of degradation), may inhibit translocation of the transcript from the nucleus to the cytoplasm, or the like, or combinations thereof. The resulting effects of binding of the AC to the target nucleotide sequence of the target transcript may include reduced cellular concentration of the target transcript pre-mRNA, the processed mature mRNA of the target transcript, the protein translated from the processed mature mRNA of the target transcript, or combinations thereof. Accordingly, an AC that binds a target transcript at a location that is proximate to and/or includes at least a portion of a polyadenylation sequence element may be effective for treating a disease for which decreased cellular concentrations of the target transcript pre-mRNA, the processed mature mRNA target transcript, or the translated protein of the target transcript is desired. Such ACs may be effectively employed regardless of the mechanism that results in the disease, such as aberrant splicing, trinucleotide repeats, or the like.

The ACs described herein may contain one or more asymmetric centers and thus give rise to enantiomers, diastereomers, and other stereoisomeric configurations that may be defined, in terms of absolute stereochemistry, as (R) or (S); α or β; or as (D) or (L). Included in the antisense compounds provided herein are all such possible isomers, as well as their racemic and optically pure forms.

In embodiments, the AC hybridizes with a target nucleotide sequence that is from about 5 to about 50 nucleic acids in length. In embodiments, the AC is the same length as the target nucleotide sequence. In embodiments, the AC is a different length than the target nucleotide sequence. In embodiments, the AC is longer than the target nucleotide sequence.

In embodiments, the AC is 5 or more, 10 or more, 15 or more, 20 or more, 25 or more, 30 or more, 35 or more, 40 or more, or 45 or more nucleic acids in length. In embodiments, the AC is 50 or less, 45 or less, 40 or less, 35 or less, 30 or less, 25 or less, 20 or less, 15 or less, or 10 or less nucleic acids in length. In embodiments, the AC is 5 to 50, 5 to 45, 5 to 40, 5 to 35, 5 to 30, 5 to 25, 5 to 20, 5 to 15, or 5 to 10 nucleic acids in length. In embodiments, the AC is 10 to 50, 10 to 45, 10 to 40, 10 to 35, 10 to 30, 10 to 25, 10 to 20, or 10 to 15 nucleic acids in length. In embodiments, the AC is 15 to 50, 15 to 45, 15 to 40, 15 to 35, 15 to 30, 15 to 25, or 15 to 20 nucleic acids in length. In embodiments, the AC is 20 to 50, 20 to 45, 20 to 40, 20 to 35, 20 to 30, or 20 to 25 nucleic acids in length. In embodiments, the AC is 25 to 50, 25 to 45, 25 to 40, 25 to 35, or 25 to 30 nucleic acids in length. In embodiments, the AC is 30 to 50, 30 to 45, 30 to 40, or 30 to 35 nucleic acids in length. In embodiments, the AC is 35 to 50, 35 to 45, or 35 to 40 nucleic acids in length. In embodiments, the AC is 40 to 50 or 40 to 45 nucleic acids in length. In embodiments, the AC is 45 to 50 nucleic acids in length. In embodiments, the AC is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acids in length.

In embodiments, the AC has 100% complementarity to a target nucleotide sequence. In embodiments, the AC does not have 100% complementarity to a target nucleotide sequence. As used herein, the term “percent complementarity” refers to the number of nucleobases of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the antisense compound.

In embodiments, the AC includes 20% or less, 15% or less, 10% or less, 5% or less, or zero mismatches to the target nucleotide sequence. In some embodiments, the AC includes 5% or more, 10% or more, or 15% or more mismatches to the target nucleotide sequence. In embodiments, the AC includes zero to 5%, zero to 10%, zero to 15%, or zero to 20% mismatches to the target nucleotide sequence. In embodiments, the AC includes 5% to 10%, 5% to 15%, or 5% to 20% mismatches to the target nucleotide sequence. In embodiments, the AC includes 10% to 15% or 10% to 20% mismatches to the target nucleotide sequence. In embodiments, the AC includes 10% to 20% mismatches to the target nucleotide sequence.

In embodiments, the AC has 80% or greater, 85% or greater, 90% or greater, 95% or greater, 96% or greater, 97% or greater, 98% or greater, or 99% or greater complementarity to a target nucleotide sequence. In embodiments, the AC has 100% or less, 99% or less, 98% or less, 97% or less 96% or less 95% or less, 90% or less, 85% or less complementarity to a target nucleotide sequence. In embodiments, the AC has 80% to 100%, 80% to 99%, 80% to 98%, 80% to 97% 80% to 96%, 80% to 95%, 80% to 90% or 80% to 85% complementarity to a target nucleotide sequence. In embodiments, the AC has 85% to 100%, 85% to 99%, 85% to 98%, 85% to 97% 85% to 96%, 85% to 95%, or 85% to 90% complementarity to a target nucleotide sequence. In embodiments, the AC has 90% to 100%, 90% to 99%, 90% to 98%, 90% to 97%, 90% to 96%, or 90% to 95 complementarity to a target nucleotide sequence. In embodiments, the AC has 95% to 100%, 95% to 99%, 95% to 98%, 95% to 97%, or 95% to 96% complementarity to a target nucleotide sequence. In embodiments, the AC has 96% to 100%, 96% to 99%, 96% to 98%, or 96% to 97% complementarity to a target nucleotide sequence. In embodiments, the AC has 97% to 100%, 97% to 99%, or 97% to 98 complementarity to a target nucleotide sequence. In embodiments, the AC has 98% to 100% or 98% to 99% complementarity to a target nucleotide sequence. In embodiments, the AC has 99% to 100% complementarity to a target nucleotide sequence. Percent complementarity of an oligonucleotide is calculated by dividing the number of complementarity nucleobases by the total number of nucleobases of the oligonucleotide.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between an AC and a target nucleotide sequence, such as by determining the thermal melting temperature (Tm). Tm or ΔTm can be calculated by techniques that are familiar to one of ordinary skill in the art. For example, techniques described in Freier et al. (Nucleic Acids Research (1997) 25, 22: 4429-4443) allow one of ordinary skill in the art to evaluate nucleotide modifications for their ability to increase the melting temperature of an RNA:DNA duplex.

In embodiments, the target nucleotide sequence is within an IRF-5, DUX4, or a DMPK target transcript. In embodiments, the AC has 100% or less complementarity (e.g., as described elsewhere herein) to a target nucleotide sequence is within an IRF-5, DUX4, or a DMPK target transcript. In embodiments, the AC has 100% or less complementarity (e.g., as described elsewhere herein) to a target nucleotide sequence is within an IRF-5, DUX4, or a DMPK target transcript and has a length of 5 to 50 nucleotides (e.g., as described elsewhere herein).

### AC Targeting Target Nucleotide Sequences and Mechanisms

In embodiments, the AC includes a nucleotide sequence that is at least partially complementary to a target nucleotide sequence of a target transcript/gene that encodes a portion of a disease-causing RNA transcript. In embodiments, the disease-causing RNA transcript is an IRF-5 pre-mRNA transcript. In embodiments, the disease-causing RNA transcript is a DMPK pre-mRNA transcript. In embodiments, the disease-causing RNA transcript is a DUX4 pre-mRNA transcript.

In embodiments, the AC binds to a target nucleotide sequence that includes at least a portion of at least one polyadenylation sequence element (PSE) of a target transcript. In embodiments, the target nucleotide sequence includes the entire PSE of a target transcript. In embodiments, the target nucleotide sequence includes the entire PSE and one or more flanking sequences that are upstream and/or downstream of the PSE of a target transcript. In embodiments, the target nucleotide sequence includes a portion, but not the entirety, of the PSE of a target transcript. In embodiments, the target nucleotide sequence includes a portion, but not the entirety, of the PSE and one or more flanking sequences that are upstream and/or downstream of the PSE of a target transcript.

In embodiments, the AC binds to a target nucleotide sequence that includes at least a portion of one or more specific PSEs of a target transcript. In embodiments, the target nucleotide sequence includes at least a portion of the consensus hexamer sequence of the PAS of a target transcript. In embodiments, the target nucleotide sequence includes at least a portion of the CS of a target transcript. In embodiments, the target nucleotide sequence includes at least a portion of the DSE of a target transcript. In embodiments, the target nucleotide sequence includes at least a portion of a USE of a target transcript. In embodiments, the target nucleotide sequence includes at least a portion of a G-rich sequence (GRS) auxiliary downstream element (AUX DSE) of a target transcript. In embodiments, the target nucleotide sequence includes at least a portion of an element core U-rich element (URE) of a target transcript. In embodiments, the target nucleotide sequence includes at least a portion of an intervening sequence (IS) of a target transcript. In embodiments, the target nucleotide includes at least a portion of more than one PSE of a target transcript. For example, in embodiments, the target nucleotide sequence includes at least a portion of the PAS and at least a portion of the CS of a target transcript. In embodiments, the target nucleotide sequence includes at least a portion of the PAS and at least a portion of the IS between the PAS and the CS of a target transcript. In embodiments, the target nucleotide sequence includes at least a portion of the PAS, at least a portion of the CS, and at least a portion of the IS between the PAS and the CS of a target transcript.

In embodiments, the AC binds to a target nucleotide sequence that includes at least a portion of one or more PSEs and one or more sequences that flank the one or more PSEs of a target transcript. In embodiments, the flanking sequences is a sequence that is upstream of the PSE. In embodiments, the flanking sequence is a sequence that is downstream of the PSE. In embodiments, the AC binds to a target nucleotide sequence that includes at least a portion of a PSE, at least a portion of a flanking sequence that is downstream of the PSE, and at least a portion of a flanking sequence that is upstream of the PSE of a target transcript.

In embodiments, the flanking sequence includes 1 or more, 2 or more, 3 or more, 4 or more, 5 or more, 10 or more, 15 or more, or 20 or more bases on one or both sides of a PSE of a target transcript. In embodiments, the flanking sequence includes 25 or less, 20 or less, 15 or less, 10 or less, 5 or less, 4 or less, 3 or less, or 2 or less bases on one or both sides of a PSE of a target transcript. In embodiments, the flanking sequence includes 1 to 25, 1 to 20, 1 to 15, 1 to 10, 1 to 5, 1 to 4, 1 to 3 or 1 to 2 bases on one or both sides of the PSE of a target transcript. In embodiments, the flanking sequence includes 2 to 25, 2 to 20, 2 to 15, 2 to 10, 2 to 5, 2 to 4, or 2 to 3 bases on one or both sides of a PSE of a target transcript. In embodiments, the flanking sequence includes 3 to 25, 3 to 20, 3 to 15, 3 to 10, 3 to 5, or 3 to 4 bases on one or both sides of a PSE of a target transcript. In embodiments, the flanking sequence includes 4 to 25, 4 to 20, 4 to 15, 4 to 10, or 4 to 5 bases on one or both sides of a PSE of a target transcript. In embodiments, the flanking sequence includes 5 to 25, 5 to 20, 5 to 15, or 5 to 10 bases on one or both sides of a PSE of a target transcript. In embodiments, the flanking sequence includes 10 to 25, 10 to 20, or 10 to 15 bases on one or both sides of a PSE of a target transcript. In embodiments, the flanking sequence includes 15 to 25 or 15 to 20 bases on one or both sides of a PSE. In embodiments, the flanking sequence includes 20 to 25 bases on one or both sides of a PSE of a target transcript.

In embodiments, the AC includes a nucleotide sequence that binds to a target nucleotide sequence of a PSE or a portion of a PSE of a target transcript encoding one or more isoforms of Interferon Regulatory Factor-5. (IRF-5). In embodiments, the AC includes a nucleotide sequence binds to a target nucleotide sequence of a PSE or a portion of a PSE of a target DMPK transcript encoding myotonic dystrophy (DM1) protein kinase. In embodiments, the AC includes a nucleotide sequence that binds to a target nucleotide sequence of a PSE or a portion of a PSE of a DUX4 target transcript that encodes double homeobox 4 (DUX4).

In embodiments, the AC binds to a target nucleotide sequence that does not include a PSE or a portion thereof of target transcript. In embodiments, the AC binds to a target nucleotide sequence that is in sufficiently close proximity to a PSE to inhibit cleavage and/or addition of a poly(A) tail to the RNA transcript of interest.

In embodiments, the AC binds to a target nucleotide sequence with a 3′ end and/or a 5′ end that is 1 or more, 2 or more, 3 or more, 4 or more, 5 or more, 10 or more, 15 or more, or 20 or more nucleotides from the 5′ end and/or 3′ end of a PSE of a target transcript. In embodiments, the AC binds to a target nucleotide sequence with a 3′ end and/or a 5′ end that is 25 or less, 20 or less, 15 or less, 10 or less, 5 or less, 4 or less, 3 or less, or 2 or less nucleotides from the 5′ end and/or 3′ end of a PSE of a target transcript. In embodiments, the AC binds to a target nucleotide sequence with a 3′ end and/or a 5′ end that is 1 to 25, 1 to 20, 1 to 15, 1 to 10, 1 to 5, 1 to 4, 1 to 3, or 1 to 2 nucleotides form the 5′ end and/or 3′ end of a PSE of a target transcript. In embodiments, the AC binds to a target nucleotide sequence with a 3′ end and/or a 5′ end that is 2 to 25, 2 to 20, 2 to 15, 2 to 10, 2 to 5, 2 to 4, or 2 to 3 nucleotides form the 5′ end and/or 3′ end of a PSE of a target transcript. In embodiments, the AC binds to a target nucleotide sequence with a 3′ end and/or a 5′ end that is 3 to 25, 3 to 20, 3 to 15, 3 to 10, 3 to 5, or 3 to 4 nucleotides form the 5′ end and/or 3′ end of a PSE of a target transcript. In embodiments, the AC binds to a target nucleotide sequence with a 3′ end and/or a 5′ end that is 4 to 25, 4 to 20, 4 to 15, 4 to 10, or 4 to 5 nucleotides form the 5′ end and/or 3′ end of a PSE of a target transcript. In embodiments, the AC binds to a target nucleotide sequence with a 3′ end and/or a 5′ end that is 5 to 25, 5 to 20, 5 to 15, or 5 to 10 nucleotides form the 5′ end and/or 3′ end of a PSE of a target transcript. In embodiments, the AC binds to a target nucleotide sequence with a 3′ end and/or a 5′ end that is 10 to 25 or 10 to 20 nucleotides form the 5′ end and/or 3′ end of a PSE of a target transcript. In embodiments, the AC binds to a target nucleotide sequence with a 3′ end and/or a 5′ end that is 20 to 25 nucleotides form the 5′ end and/or 3′ end of a PSE of a target transcript.

In embodiments, the AC binds to a target nucleotide sequence that includes at least a portion of at least one PSE of an IRF-5, DUX4, or DMPK target transcript. The AC may bind to a portion or the entirety of one or more PSE and a flanking sequence (e.g., as described elsewhere herein) of an IRF-5, DUX4, or DMPK target transcript.

The antisense mechanism functions via hybridization of an AC with a target nucleotide sequence. Hybridizing of an AC to a target nucleotide sequence that includes at least a portion of a of a transcript of interest, may have a number of different effects. In embodiments, the AC hybridizing to its target nucleotide sequence downregulates expression of the target transcript/gene expression product, such as a protein. In embodiments, the AC hybridizing to its target nucleotide sequence downregulates expression of one or more protein isomers encoded by the target transcript/gene. In embodiments, the AC hybridizing to its target sequence upregulates the expression of the protein encoded by the target transcript/gene. In embodiments, the AC hybridizing to its target nucleotide sequence increases expression of one or more protein isomers encoded by the target transcript/gene. In embodiments, modulation of cellular concentrations of pre-mRNA, mature mRNA, and/or protein product of the target transcript/gene modulates expression of one or more genes other than the target gene, such as downstream genes. In embodiments, the AC hybridizing to its target nucleotide sequence downregulates the expression of one or more proteins that are affected by the expression of the target transcript/gene. In embodiments, the AC hybridizing to its target nucleotide sequence upregulates expression of one or more proteins that are affected by the expression of the target transcript/gene. In embodiments, the AC hybridizes to the target nucleotide sequence of the transcript of interest thereby increasing stability of the mRNA transcript. In embodiments, the AC hybridizes to the target nucleotide sequence of the transcript of interest thereby decreasing stability of the mRNA transcript. In embodiments, the AC hybridizes to the target nucleotide sequence of the transcript of interest thereby resulting in degradation of the mRNA transcript. For example, in embodiments, the AC hybridizes to the target nucleotide sequence of the transcript of interest thereby resulting in degradation of the polyadenylation sequence element based on a RNase H-mediated mechanism. In embodiments, the AC hybridizes to the target nucleotide sequence of the transcript of interest does not result in the degradation of the PSE of the mRNA transcript.

In embodiments, the AC hybridizing to a target IRF-5, DUX4, or DMPK transcript results in upregulated or downregulated expression (e.g., as described elsewhere herein) of the IRF-5, DUX4, or DMPK transcript.

In embodiments, the hybridization of an AC to a target transcript regulates transcription, processing, translocation, and/or translation of a target transcript through steric blocking. In embodiments, the AC hybridizes to the target nucleotide sequence of the transcript of interest thereby sterically blocking the binding of one or more proteins to the mRNA transcript. In embodiments, the AC regulates RNA processing through steric blocking of machinery needed for the polyadenylation of a transcript of interest (Roberts et al., Nature Reviews Drug Discovery (2020) 19: 673-694). In embodiments, the AC regulates translation and/or protein expression by preventing one or more components of the polyadenylation protein complex from binding to one or more polyadenylation sequence elements of a target transcript. In embodiments, the AC hybridizes to the target nucleotide sequence of the transcript of interest thereby sterically blocking the binding of CPSF to the mRNA transcript. In embodiments, the AC hybridizes to the target nucleotide sequence of the transcript of interest thereby sterically blocking the binding of CstF to the mRNA transcript.

In embodiments, the AC hybridizing to a target IRF-5, DUX4, or DMPK transcript regulates transcription, processing, translocation, and/or translation of the IRF-5, DUX4, or DMPK transcript through steric blocking (e.g., as described elsewhere herein).

RNA transcripts can include more than one location at which a poly(A) tail may be added. Targeting a PSE that directs addition of the poly(A) tail a particular location may be used to differentially affect the formation and or prevalence of alternative mRNA transcripts. In embodiments, binding of the AC to, or in proximity to, a PSE redirects binding of the polyadenylation complex to another PSE on the mRNA transcript, resulting in the formation of an alternative transcript. In embodiments, the alternative transcript contains fewer destabilization sequences, such that binding of the AC to, or in proximity to, the PSE results in an increase in mRNA stability. In embodiments, the alternative transcript contains more destabilization sequences, such that binding of the AC to the PSE results in decreased mRNA stability (Vickers et al., Nucleic Acids Res. (2001), 29(6):1293-1299). In embodiments, hybridization of the AC to a target nucleotide sequence that includes a PSE results in steric blockage of the polyadenylation sequence element and preferential cleavage at a cleavage site that is not blocked by the AC. In embodiments, the target transcript includes multiple PSEs and the AC hybridizes to a target nucleotide sequence that includes a PSE of the first (or most 5′) PSE. In embodiments, the gene or RNA transcript includes multiple cleavage sites, and the AC hybridizes a target nucleotide sequence that includes the last (or most 3′) cleavage site.

In embodiments, binding of the AC to, or in proximity to, a PSE redirects binding of the polyadenylation complex to another PSE on an IRF-5, DUX4, and/or DMPK target transcript, resulting in the formation of an alternative transcript (e.g., as described elsewhere herein).

The efficacy of the ACs may be assessed by evaluating the antisense activity effected by their administration. As used herein, the term “antisense activity” refers to any detectable and/or measurable activity attributable to the hybridization of an AC to its target nucleotide sequence. Such detection and/or measuring may be direct or indirect. In embodiments, antisense activity is assessed by detecting and or measuring the amount of the protein expressed from the transcript of interest. In embodiments, antisense activity is assessed by detecting and/or measuring the amount of the transcript of interest. In some embodiments, antisense activity is assessed by detecting and/or measuring the amount of alternative polyadenylation isoforms (APA) of the transcript of interest. In embodiments, antisense activity is assessed by detecting and/or measuring the amount of a downstream transcript and/or protein that is regulated by the gene of interest.

### Antisense Compound Design

Design of ACs will depend upon the target transcript/gene. Targeting an AC to a particular target nucleotide sequence can be a multistep process. The process usually begins with the identification of gene of interest. In some embodiments, the gene of interest is IRF-5, DM1, or DUX4. The transcript of the gene of interest is analyzed and a target nucleotide sequence may be identified. In embodiments, the target nucleotide sequence includes at least a portion of at least one PSE of the target transcript or is in sufficient proximity to (e.g., adjacent or within 1 to 20 nucleotides) of a PSE to sterically block binding of one or more proteins of machinery needed for the polyadenylation of a transcript of interest. In embodiments, the target nucleotide sequence includes at least a portion of at least one PSE or is in sufficient proximity to a PSE of an IRF-5 mRNA transcript. In embodiments, the target nucleotide sequence includes at least a portion of at least one PSE or is in sufficient proximity to a PSE of a DM1 transcript. In embodiments, the target nucleotide sequence includes at least a portion of at least one PSE or is in sufficient proximity to a PSE of a DUX4 transcript.

One of skill in the art will be able to design, synthesize, and screen of different nucleobase sequences to identify a sequence that results in antisense activity. For example, an AC can be designed that inhibits expression of a target transcript/gene. Methods for designing, synthesizing, and screening ACs for antisense activity against a preselected target transcript/gene can be found, for example in “Antisense Drug Technology, Principles, Strategies, and Applications” Edited by Stanley T. Crooke, CRC Press, Boca Raton, Florida, which is incorporated by reference in its entirety for any purpose.

### AC Structure

The AC includes an oligonucleotide and/or an oligonucleoside. Oligonucleotides and/or oligonucleosides are nucleosides linked through internucleoside linkages. Nucleosides include a pentose sugar (e.g., ribose or deoxyribose) and a nitrogenous base covalently attached to sugar. The naturally occurring (traditional) bases found in DNA and/or RNA are adenine (A), guanine (G), thymine (T), cytosine (C), and uracil (U). The naturally occurring (traditional) sugars found in DNA and/or RNA deoxyribose (DNA) and ribose (RNA). The naturally occurring (traditional) nucleoside linkage is a phosphodiester bond. In embodiments, the ACs of the present disclosure may have all natural sugars, bases, and internucleoside linkages.

Chemically modified nucleosides are routinely used for incorporation into antisense compounds to enhance one or more properties, such as nuclease resistance, pharmacokinetics or affinity for a target RNA. In embodiments, the ACs of the present disclosure may have one or more modified nucleosides. In embodiments, the ACs of the present disclosure may have one or more modified sugars. In embodiments, the ACs of the present disclosure may have one or more modified bases. In embodiments, the ACs of the present disclosure may have one or more modified internucleoside linkages.

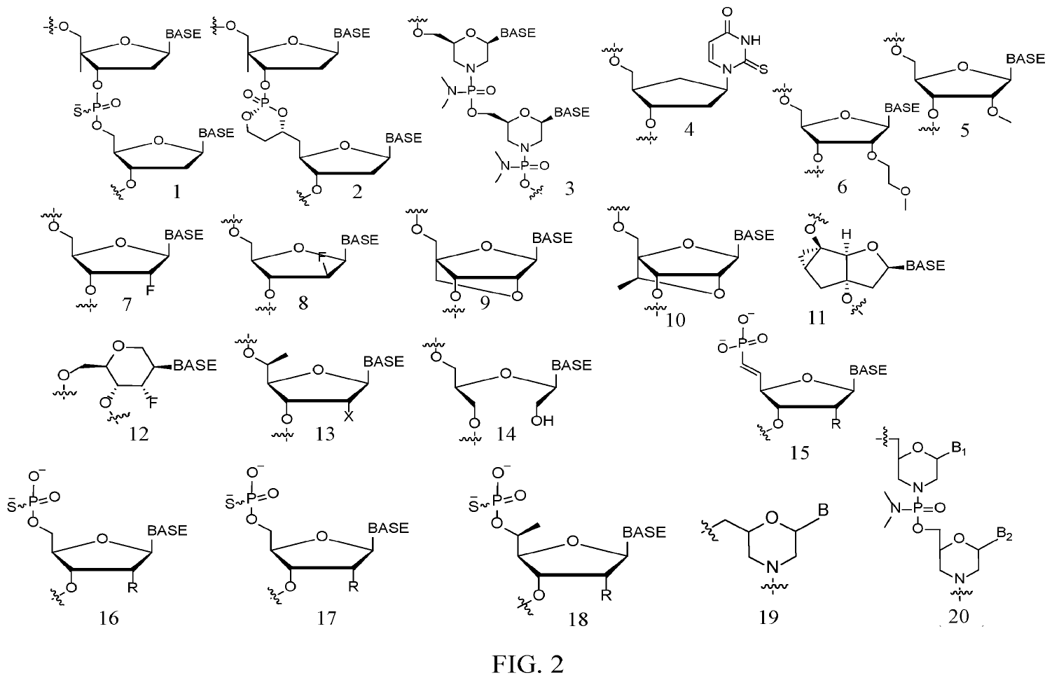

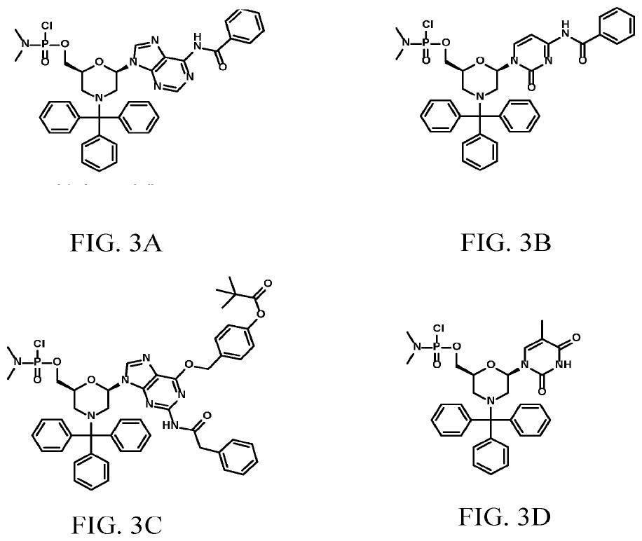

In general, a nucleobase is any group that contains one or more atom or groups of atoms capable of hydrogen bonding to a base of another nucleic acid. In addition to “unmodified” or “natural” nucleobases (A, G, T, C, and U) many modified nucleobases or nucleobase mimetics are known to those skilled in the art are amenable with the compounds described herein. Generally, a modified nucleobase refers to a nucleobase that is fairly similar in structure to the parent nucleobase, such as for example a 7-deaza purine, a 5-methyl cytosine, 2-thio-dT (FIG. 2) or a G-clamp. Generally, a nucleobase mimetic is a nucleobase that includes a structure that is more complicated than a modified nucleobase, such as for example a tricyclic phenoxazine nucleobase mimetic. Methods for preparation of the above noted modified nucleobases are well known to those skilled in the art.

In embodiments, the AC may include one or more nucleosides having a modified sugar moiety. In embodiments, the furanosyl sugar of a natural nucleoside may have a 2′ modification, modifications to make a constrained nucleoside, and others (see FIG. 2). For example, in embodiments, the furanosyl sugar ring of a natural nucleoside can be modified in a number of ways including, but not limited to, addition of a substituent group; bridging of two non-geminal ring atoms to form a bicyclic nucleic acid (BNA) or a locked nucleic acid; exchanging the oxygen of the furanosyl ring with C or N; and/or substitution of an atom or group (see FIG. 2). Modified sugars are well known and can be used to increase or decrease the affinity of the AC for its target nucleotide sequence. Modified sugars may also be used to increase the AC nuclease resistance. Sugars can also be replaced with sugar mimetic groups among others. In embodiments, one or more sugars of the nucleosides of the AC is replaced with a morpholine ring as shown as 19 in FIG. 2.

In embodiments, the AC includes one or more nucleosides that include a bicyclic modified sugar (BNA; sometimes called bridged nucleic acids). Examples of BNA's suitable for use in the ACs of the present disclosure include but are not limited to, LNA (4′-(CH₂)—O-2′ bridge), 2′-thio-LNA (4′-(CH₂)—S-2′ bridge), 2′-amino-LNA (4′-(CH₂)—NR-2′ bridge), ENA (4′-(CH₂)2-O-2′ bridge), 4′-(CH₂)₃-2′ bridged BNA, 4′-(CH₂CH(CH₃))-2′ bridged BNA, cEt (4′-(CH(CH₃)—O-2′ bridge), and cMOE BNAs (4′-(CH(CH₂OCH₃)—O-2′ bridge). BNA's have been prepared and disclosed in the patent literature as well as in scientific literature (See, e.g., Srivastava, et al. J. Am. Chem. Soc. (2007), ACS Advanced online publication, 10.1021/ja071106y; Albaek et al., J. Org. Chem., (2006), 71, 7731-7740; Fluiter, et al., Chembiochem (2005), 6, 1104-1109; Singh et al., Chem. Commun. (1998), 4, 455-456; Koshkin et al., Tetrahedron (1998), 54, 3607-3630; Wahlestedt et al., Proc. Natl. Acad. Sci. U.S.A. (2000), 97, 5633-5638; Kumar et al., Bioorg. Med. Chem. Lett. (1998), 8, 2219-2222; WO 94/14226; WO 2005/021570; Singh et al., J. Org. Chem. (1998), 63, 10035-10039; WO 2007/090071; U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807).

In embodiments, the AC includes one or more nucleosides that include a locked nucleic acid (LNA). In LNAs the 2′-hydroxyl group of the ribosyl sugar ring is linked to the 4′ carbon atom of the sugar ring thereby forming a 2′-C,4′-C-oxymethylene linkage to form the bicyclic sugar moiety (see for e.g., Elayadi et al., Curr. Opinion Invens. Drugs (2001), 2, 558-561; Braasch et al., Chem. Biol. (2001), 8 1-7; and Orum et al., Curr. Opinion Mol. Ther. (2001), 3, 239-243; see also U.S. Pat. Nos. 6,268,490 and 6,670,461). Some examples are show in in FIG. 2. The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun. (1998), 4, 455-456; ENA™; Morita et al., Bioorganic Medicinal Chemistry (2003), 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic ACs containing LNAs have been described (Wahlestedt et al., Proc. Natl. Acad. Sci. U.S.A. (2000), 97, 5633-5638).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research (2003), 21, 6365-6372).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNAs such as phosphorothioate-LNA and 2′-thio-LNAs, have also been prepared (Kumar et al., Bioorg. Med. Chem. Lett., 1998, 8, 2219-2222). Preparation of LNAs analogs containing oligodeoxyribonucleotide duplexes as substrates for nucleic acid polymerases has also been described (WO 99/14226). Furthermore, synthesis of 2′-amino-LNA, a conformationally restricted high-affinity oligonucleotide analog has been described (Singh et al., J. Org. Chem. (1998), 63, 10035-10039). In addition, 2′-amino- and 2′-methylamino-LNA's have been prepared and the thermal stability of their duplexes with complementary RNA and DNA strands has been previously reported.

In embodiments, the antisense compound is a “tricyclo-DNA (tc-DNA)”, which refers to a class of constrained DNA analogs in which each nucleotide is modified by the introduction of a cyclopropane ring to restrict conformational flexibility of the backbone and to enhance the backbone geometry of the torsion angle γ. Homobasic adenine- and thymine-containing tc-DNAs form extraordinarily stable A-T base pairs with complementary RNAs.

Methods for the preparations of other modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

### Internucleoside Linkages

Described herein are internucleoside linking groups that link the nucleosides or otherwise modified nucleoside monomer units together thereby forming an oligonucleotide and/or an oligonucleotide containing AC. The ACs may include naturally occurring internucleoside linkages, unnatural internucleoside linkages, or both.

In naturally occurring DNA and RNA, the internucleoside linking group is a phosphodiester that covalently links adjacent nucleosides to one another to form a linear polymeric compound. In naturally occurring DNA and RNA, phosphodiester is linked to the 2′, 3′ or 5′ hydroxyl moiety of the sugar. Within oligonucleotides, the phosphate groups are commonly referred to as forming the internucleoside backbone of the oligonucleotide. In naturally occurring DNA and RNA, the linkage or backbone of RNA and DNA, is a 3′ to 5′ phosphodiester linkage. In some embodiments, the internucleoside linking groups of the ACs are phosphodiesters. In some embodiments, the internucleoside linking groups of the ACs are 3′ to 5′ phosphodiester linkages.

The two main classes of unnatural internucleoside linking groups are defined by the presence or absence of a phosphorus atom. Representative phosphorus containing internucleoside linkages include, but are not limited to, phosphotriesters, methylphosphonates, phosphoramidate, and phosphorothioates. Representative non-phosphorus containing internucleoside linking groups include, but are not limited to, methylenemethylimino (—CH₂—N(CH)—O—CH₂—), thiodiester (—O—C(O)—S—), thionocarbamate (—O—C(O)(NH)—S—); siloxane (—O—Si(H₂—O—); and N,N′-dimethylhydrazine (-cH₂—N(CH₃)—N(CH₃)—). ACs having one or more non-phosphorus internucleoside linking groups are referred to as oligonucleosides. ACs having phosphorus internucleoside linking groups are referred to as oligonucleotides. Modified internucleoside linkages, compared to natural phosphodiester linkages, can be used to alter, typically increase, nuclease resistance of the antisense compound. Internucleoside linkages having a chiral atom can be prepared as racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, two or more nucleosides having modified sugars and/or modified nucleobases may be joined using a phosphoramidate. In embodiments, two or more nucleosides having a methylenemorpholine ring may be connected through a phosphoramidate internucleoside linkage as shown as 20 in FIG. 2 where B₁ and B₂ are modified or natural nucleobases. Antisense compounds that include nucleobases with a methylenemorpholine ring that are linked through phosphoramidate internucleoside linkage may be referred to as phosphoramidate morpholino oligomers (PMOs).

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge, and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, and dyes. In embodiments, the conjugate group is a polyethylene glycol (PEG), and the PEG is conjugated to either the AC or the CPP (CPP discussed elsewhere herein).

In embodiments, conjugate groups include lipid moieties such as a cholesterol moiety (Letsinger et al., Proc. Natl. Acad. Sci. USA (1989), 86, 6553); cholic acid (Manoharan et al., Bioorg. Med. Chem. Lett. (1994), 4, 1053); a thioether, e.g., hexyl-S-tritylthiol (Manoharan et al., Ann. N.Y. Acad. Sci. (1992), 660, 306; Manoharan et al., Bioorg. Med. Chem. Let. (1993), 3, 2765); a thiocholesterol (Oberhauser et al., Nucl. Acids Res. (1992), 20, 533); an aliphatic chain, e.g., dodecandiol or undecyl residues (Saison-Behmoaras et al., EMBO J. (1991), 10, 111; Kabanov et al., FEBS Lett. (1990), 259, 327; Svinarchuk et al., Biochimie (1993), 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett. (1995), 36, 3651; Shea et al., Nucl. Acids Res. (1990), 18, 3777); a polyamine or a polyethylene glycol chain (Manoharan et al., Nucleosides & Nucleotides (1995), 14, 969); adamantane acetic acid (Manoharan et al., Tetrahedron Lett. (1995), 36, 3651); a palmityl moiety (Mishra et al., Biochim. Biophys. Acta. (1995), 1264, 229); or an octadecylamine or hexylamino-carbonyl-oxycholesterol moiety (Crooke et al., J. Pharmacol. Exp. Ther. (1996), 277, 923).

### Types of Antisense Compounds

Various types of AC may be used, including, for example, an antisense oligonucleotide, siRNA, microRNA, antagomir, aptamer, ribozyme, supermir, miRNA mimic, miRNA inhibitor, or combinations thereof.

### Antisense Oligonucleotides

In embodiments, the AC is an antisense oligonucleotide (ASO). The term “antisense oligonucleotide” or simply “antisense” is meant to include oligonucleotides that are at least partially complementary to a target nucleotide sequence. ASOs include single strands of DNA, RNA, or DNA and RNA oligonucleotides that comprises a sequence complementary to a chosen sequence, e.g., a target nucleotide sequence. ASOs may include one or more modified DNA and/or RNA bases, modified sugars, and/or unnatural internucleoside linkages. In embodiments, the ASOs may include one or more phosphoramidate internucleoside linkages. In embodiments, the ASO is phosphoramidate morpholino oligomers (PMOs). An ASO may be of any length, any characteristic, function through any mechanism, and/or hybridize to any target nucleotide sequence as described relative to ACs.

Antisense oligonucleotides have been demonstrated to be effective as targeted inhibitors of protein synthesis, and, consequently, can be used to specifically inhibit protein synthesis by a targeted gene. The efficacy of ASO for inhibiting protein synthesis is well established. To date, these compounds have shown promise in several in vitro and in vivo models, including models of inflammatory disease, cancer, and HIV (Agrawal et al., Trends in Biotech. (1996), 14:376-387). Antisense can also affect cellular activity by hybridizing specifically with chromosomal DNA.

Methods of producing ASOs are known in the art and can be readily adapted to produce an ASO that binds to a target nucleotide sequence of the present disclosure. Selection of ASOs sequences specific for a given target nucleotide sequence is based upon analysis of the chosen target nucleotide sequence and determination of secondary structure, Tm, binding energy, and relative stability. Antisense oligonucleotides may be selected based upon their relative inability to form dimers, hairpins, or other secondary structures that would reduce or prohibit specific binding to the target nucleotide sequence in a host cell. These secondary structure analyses and target site selection considerations can be performed, for example, using v.4 of the OLIGO primer analysis software (Molecular Biology Insights) and/or the BLASTN 2.0.5 algorithm software (Altschul et ai, Nucleic Acids Res. 1997, 25(17):3389-402).

In embodiments, the AC comprises a gapmer. A gapmer is a short DNA ASO structure with RNA or RNA-mimic segments on either side of the DNA structure. The entire gapmer, or a portion thereof, may hybridize to the target nucleotide sequence. In embodiments, the RNA-mimic segments comprise LNAs. In embodiments, the LNA comprise 2′-OMe or 2′-F modified bases. Gapmers may mediate degradation of the target nucleic acid through the action of RNase H. The gapmer may be of any suitable length. In embodiments, the DNA structure of the gapmer is 5 to 15 nucleotides in length, such as 7 to 13 nucleotides in length, 9 to 11 nucleotides in length, or about 10 nucleotides in length. In embodiments, each RNA or RNA-mimic segment is 1 to 10 nucleotides in length, such as 2 to 8 nucleotides in length, 4 to 6 nucleotides in length, or about 5 nucleotides in length. In embodiments, the gapmer binds a target gene transcript at a location that includes at least a portion of a PSE or in sufficient proximity to the PSE to modulate polyadenylation of the target gene transcript. In embodiments, the gapmer binds a target gene transcript at location that does not modulate or substantially modulate polyadenylation. In embodiments, the gapmer mediates degradation of the target gene transcript. In embodiments, the gapmer mediates degradation of the target gene transcript through the action of RNase H. In embodiments, the gapmer binds a target IRF-5, DMPK, or DUX4 gene transcript.

### RNA Interference

In embodiments, the AC includes a molecule that mediates RNA interference (RNAi). As used herein, the phrase “mediates RNAi” refers to the ability to silence, in a sequence specific manner, a target transcript. While not wishing to be bound by theory, it is believed that silencing uses the RNAi machinery or process and a guide RNA, e.g., an siRNA compound of from about 21 to about 23 nucleotides. In embodiments, the AC targets the target transcript for degradation. As such, in embodiments, RNAi molecule may be used to disrupt the expression of a gene or polynucleotide of interest. In embodiments, RNAi molecule is used to induce degradation of the target transcript, such as a pre-mRNA or a mature mRNA.

In embodiments, the AC includes a small interfering RNA (siRNA) that elicits an RNAi response. siRNAs are nucleic acid duplexes normally from about 16 to about 30 nucleotides long that can associate with a cytoplasmic multi-protein complex known as RNAi-induced silencing complex (RISC). RISC loaded with siRNA mediates the degradation of homologous transcripts, therefore siRNA can be designed to knock down protein expression with high specificity. Unlike other antisense technologies, siRNA function through a natural mechanism evolved to control gene expression through non-coding RNA. A variety of RNAi reagents, including siRNAs targeting clinically relevant targets, are currently under pharmaceutical development, as described, e.g., in de Fougerolles, A. et al., Nature Reviews (2007) 6:443-453.

While the first described RNAi molecules were RNA:RNA hybrids that include both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., Molecular Biotechnology (2003), 24:111-119). In embodiments, RNAi molecules are used that include any of these different types of double-stranded molecules. In addition, it is understood that RNAi molecules may be used and introduced to cells in a variety of forms. Accordingly, as used herein, RNAi molecules encompasses any and all molecules capable of mediating RNAi in cells, including, but not limited to, double-stranded oligonucleotides that include two separate strands, i.e. a sense strand and an antisense strand, e.g., small interfering RNA (siRNA); double-stranded oligonucleotide that includes two separate strands that are linked together by non-nucleotidyl linker; oligonucleotides that include a hairpin loop of complementary sequences, which forms a double-stranded region, e.g., shRNAi molecules; and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least about 14, at least about 15, at least about 20, at least about 25, at least about 30, at least about 35, at least about 40, or up to about 50 nucleotides in length. In certain embodiments, the single strand siRNA is less than about 200, about 100, or about 60 nucleotides in length.

Hairpin siRNA compounds may have a duplex region equal to or at least about 17, about 18, about 19, about 20, about 21, about 22, about 23, about 24, or about 25 nucleotide pairs. The duplex region may be equal to or less than about 200, about 100, or about 50 nucleotide pairs in length. In certain embodiments, ranges for the duplex region are from about 15 to about 30, from about 17 to about 23, from about 19 to about 23, and from about 19 to about 21 nucleotides pairs in length. The hairpin may have a single strand overhang or terminal unpaired region. In certain embodiments, the overhangs are from about 2 to about 3 nucleotides in length. In embodiments, the overhang is at the same side of the hairpin and in some embodiments on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least about 14, about 15, about 16 about 17, about 18, about 19, about 20, about 25, about 30, about 40, or about 60 nucleotides in length. It may be equal to or less than about 200, about 100, or about 50 nucleotides in length. Ranges may be from about 17 to about 25, from about 19 to about 23, and from about 19 to about 21 nucleotides in length. As used herein, term “antisense strand” means the strand of an siRNA compound that is sufficiently complementary to a target molecule, e.g. the target nucleotide sequence of a target transcript.

The sense strand of a double stranded siRNA compound may be equal to or at least about 14, about 15, about 16, about 17, about 18, about 19, about 20, about 25, about 30, about 40, or about 60 nucleotides in length. It may be equal to or less than about 200, about 100, or about 50, nucleotides in length. Ranges may be from about 17 to about 25, from about 19 to about 23, and from about 19 to about 21 nucleotides in length.

The double strand portion of a double stranded siRNA compound may be equal to or at least about 14, about 15, about 16, about 17, about 18, about 19, about 20, about 21, about 22, about 23, about 24, about 25, about 30, about 40, or about 60 nucleotide pairs in length. It may be equal to or less than about 200, about 100, or about 50, nucleotides pairs in length. Ranges may be from about 15 to about 30, from about 17 to about 23, from about 19 to about 23, and from about 19 to about 21 nucleotides pairs in length.

In embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1 to 3 nucleotides. The overhangs can be the result of one strand being longer than the other, or the result of two strands of the same length being staggered. Some embodiments will have at least one 3′ overhang. In embodiments, both ends of an siRNA molecule will have a 3′ overhang. In embodiments, the overhang is 2 nucleotides.

In embodiments, the length for the duplexed region is from about 15 to about 30, or about 18, about 19, about 20, about 21, about 22, or about 23 nucleotides in length, e.g., in the ssiRNA (siRNA with sticky overhangs) compound range discussed above. ssiRNA compounds can resemble in length and structure the natural Dicer processed products from long dsiRNAs. Embodiments in which the two strands of the ssiRNA compound are linked, e.g., covalently linked are also included. In embodiments, hairpin, or other single strand structures which provide a double stranded region, and a 3′ over hangs are included.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene.

In embodiments, an siRNA compound is “sufficiently complementary” to a target transcript, such that the siRNA compound silences production of protein encoded by the target mRNA. In embodiments, the siRNA compound is “sufficiently complementary” to at least a portion of a polyadenylation sequence element of a target transcript, such that the siRNA compound silences production of the gene product encoded by the target transcript. In another embodiment, the siRNA compound is “exactly complementary” to a target nucleotide sequence (e.g., a portion of a target transcript) such that the target nucleotide sequence and the siRNA compound anneal, for example to form a hybrid made exclusively of Watson-Crick base pairs in the region of exact complementarity. A “sufficiently complementary” to a target nucleotide sequence can include an internal region (e.g., of at least about 10 nucleotides) that is exactly complementary to a target nucleotide sequence. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

The therapeutic applications of RNAi are extremely broad, since siRNA and miRNA constructs can be synthesized with any nucleotide sequence directed against a target gene transcript. To date, siRNA constructs have shown the ability to specifically down-regulate target proteins in both in vitro and in vivo models, as well as in clinical studies.

### MicroRNAs

In embodiments, the AC includes a microRNA molecule. MicroRNAs (miRNAs) are a highly conserved class of small RNA molecules that are transcribed from DNA in the genomes of plants and animals but are not translated into protein. Processed miRNAs are single stranded 17-25 nucleotide RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

### Antagomirs

In embodiments, the AC is an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes that include the antagomir and endogenous miRNA, thereby preventing miRNA-induced gene silencing. An example of antagomir-mediated miRNA silencing is the silencing of miR-122, described in Krutzfeldt et al., Nature (2005), 438: 685-689, which is expressly incorporated by reference herein in its entirety. Antagomir RNAs may be synthesized using standard solid phase oligonucleotide synthesis protocols (U.S. patent application Ser. Nos. 11/502,158 and 11/657,341; the disclosure of each of which are incorporated herein by reference).

An antagomir can include ligand-conjugated monomer subunits and monomers for oligonucleotide synthesis. Monomers are described in U.S. application Ser. No. 10/916,185. An antagomir can have a ZXY structure, such as is described in PCT Application No. PCT/US2004/07070. An antagomir can be complexed with an amphipathic moiety. Amphipathic moieties for use with oligonucleotide agents are described in PCT Application No. PCT/US2004/07070.

### Aptamers

In embodiments, the AC includes an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules (Eaton, Curr. Opin. Chem. Biol. 1: 10-16 (1997); Famulok, Curr. Opin. Struct. Biol. (1999), 9:324-9; and Hermann and Patel, Science (2000), 287:820-5). Aptamers may be RNA or DNA based and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues, and organisms. The aptamer may be prepared by any known method, including synthetic, recombinant, and purification methods, and may be used alone or in combination with other aptamers specific for the same target. Further, the term “aptamer” also includes “secondary aptamers” containing a consensus sequence derived from comparing two or more known aptamers to a given target. In embodiments, the aptamer is an “intracellular aptamer”, or “intramer”, which specifically recognize intracellular targets (Famulok et al., Chem Biol. (2001), 10:931-939; Yoon and Rossi, Adv Drug Deliv Rev. (2018), 134:22-35, each incorporated by reference herein).

### Ribozymes

In embodiments, the AC is a ribozyme. Ribozymes are RNA molecules complexes having specific catalytic domains that possess endonuclease activity (Kim and Cech, Proc. Natl. Acad. Sci. USA (1987), 84(24):8788-92; Forster and Symons, Cell (1987), 24, 49(2):211-20). For example, a large number of ribozymes accelerate phosphoester transfer reactions with a high degree of specificity, often cleaving only one of several phosphoesters in an oligonucleotide substrate (Cech et al., Cell (1981), 27(3 Pt 2):487-96; Michel and Westhof, J. Mol. Biol. (1990), 5,216(3):585-610; Reinhold-Hurek and Shub, Nature (1992) 14, 357(6374): 173-6). This specificity has been attributed to the requirement that the substrate bind via specific base-pairing interactions to the internal guide sequence (IGS) of the ribozyme prior to chemical reaction.

At least six basic varieties of naturally occurring enzymatic RNAs are known presently. Each can catalyze the hydrolysis of RNA phosphodiester bonds in trans (and thus can cleave other RNA molecules) under physiological conditions, In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al., Nucleic Acids Res. (1992), 20(17):4559-65. Examples of hairpin motifs are described by Eur. Pat. Appl. Publ. No. EP 0360257, Hampel and Tritz, Biochemistry (1989), 28(12):4929-33; Hampel et al., Nucleic Acids Res. (1990), 18(2):299-304; and U.S. Pat. No. 5,631,359. An example of the hepatitis virus motif is described by Perrotta and Been, Biochemistry (1992), 31(47): 11843-52; an example of the RNaseP motif is described by Guerrier-Takada et al., Cell (1983), 35(3 Pt 2):849-57; Neurospora VS RNA ribozyme motif is described by Collins (Saville and Collins, Cell (1990), 61(4):685-96; Saville and Collins, Proc. Natl. Acad. Sci. USA (1991), 88(19):8826-30; Collins and Olive, Biochemistry (1993), 32(11):2795-9); and an example of the Group I intron is described in U.S. Pat. No. 4,987,071. In embodiments, enzymatic nucleic acid molecules have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Methods of producing a ribozyme targeted to a polynucleotide sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein. In embodiments, the ribozyme is targeted to a target nucleotide sequence that includes one or more PSEs in a target transcript. In embodiments, the ribozyme is targeted to a polyadenylation sequence element (PSE) in a target transcript.

Ribozyme activity can be increased by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem H bases to shorten RNA synthesis times and reduce chemical requirements.

### Supermir

In embodiments, the AC is a supermir. A supermir refers to a single stranded, double stranded, or partially double stranded oligomer or polymer of RNA, polymer of DNA, or both, or modifications thereof which has a nucleotide sequence that is substantially identical to an miRNA and that is antisense with respect to its target, This term includes oligonucleotides composed of naturally-occurring nucleobases, sugars and covalent internucleoside (backbone) linkages and which contain at least one non-naturally-occurring portion which functions similarly. Such modified or substituted oligonucleotides have desirable properties such as, for example, enhanced cellular uptake, enhanced affinity for nucleic acid target and increased stability in the presence of nucleases. In embodiments, the supermir does not include a sense strand, and in another embodiment, the supermir does not self-hybridize to a significant extent. A supermir can have secondary structure, but it is substantially single-stranded under physiological conditions. A supermir that is substantially single-stranded is single-stranded to the extent that less than about 50% (e.g., less than about 40%, about 30%, about 20%, about 10%, or about 5%) of the supermir is duplexed with itself. The supermir can include a hairpin segment, e.g., sequence, for example, at the 3′ end can self-hybridize and form a duplex region, e.g., a duplex region of at least about 1, about 2, about 3, or about 4 or less than about 8, about 7, about 6, or about 5 nucleotides, or about 5 nucleotides. The duplexed region can be connected by a linker, e.g., a nucleotide linker, e.g., about 3, about 4, about 5, or about 6 dTs, e.g., modified dTs. In another embodiment the supermir is duplexed with a shorter oligo, e.g., of about 5, about 6, about 7, about 8, about 9, or about 10 nucleotides in length, e.g., at one or both of the 3′ and 5′ end or at one end and in the non-terminal or middle of the supermir.

miRNA Mimics

In embodiments, the AC is a miRNA mimic. miRNA mimics represent a class of molecules that can be used to imitate the gene silencing ability of one or more miRNAs. Thus, the term “microRNA mimic” refers to synthetic non-coding RNAs (e.g., the miRNA is not obtained by purification from a source of the endogenous miRNA) that are capable of entering the RNAi pathway and regulating gene expression. miRNA mimics can be designed as mature molecules (e.g., single stranded) or mimic precursors (e.g., pri- or pre-miRNAs). miRNA mimics can include nucleic acid (modified or modified nucleic acids) including oligonucleotides that include, without limitation, RNA, modified RNA, DNA, modified DNA, locked nucleic acids, or 2′-0,4′-C-ethylene-bridged nucleic acids (ENA), or any combination of the above (including DNA-RNA hybrids). In addition, miRNA mimics can include conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can include 2′ modifications (including 2′-0 methyl modifications and 2′ F modifications) on one or both strands of the molecule and internucleoside modifications (e.g., phosphorothioate modifications) that enhance nucleic acid stability and/or specificity. In addition, miRNA mimics can include overhangs. The overhangs can include from about 1 to about 6 nucleotides on either the 3′ or 5′ end of either strand and can be modified to enhance stability or functionality. In embodiments, a miRNA mimic includes a duplex region of from about 16 to about 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can include 2′ F modification of all of the Cs and Us, phosphorylation of the 5′ end of the oligonucleotide, and stabilized internucleoside linkages associated with a 2 nucleotide 3′ overhang.

miRNA Inhibitor

In embodiments, the AC is a miRNA inhibitor. The terms “antimir” “microRNA inhibitor”, “miR inhibitor”, or “miRNA inhibitor” are synonymous and refer to oligonucleotides or modified oligonucleotides that interfere with the ability of specific miRNAs. In general, the inhibitors are nucleic acid or modified nucleic acids in nature including oligonucleotides that include RNA, modified RNA, DNA, modified DNA, locked nucleic acids (LNAs), or any combination of the above.

Modifications include 2′ modifications (including 2′-0 alkyl modifications and 2′ F modifications) and internucleoside modifications (e.g., phosphorothioate modifications) that can affect delivery, stability, specificity, intracellular compartmentalization, or potency. In addition, miRNA inhibitors can include conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors include contain one or more sequences or portions of sequences that are complementary or partially complementary with the mature strand (or strands) of the miRNA to be targeted, in addition, the miRNA inhibitor may also include additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

### U1 Adaptor

In embodiments, the AC is a U1 adaptor. U1 adaptors inhibit poly(A) sites and are bifunctional oligonucleotides with a target domain complementarity to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of the 5′ end of U1 snRNA base pair with the 5′ss of the pre mRNA. In one embodiment, oligonucleotides are U1 adaptors. In one embodiment, the U1 adaptor can be administered in combination with at least one other iRNA agent.

### CRISPR Gene-Editing Machinery

In embodiments, the therapeutic moiety includes one or more elements of CRISPR gene-editing machinery. As used herein, “CRISPR gene-editing machinery” refers to protein, nucleic acids, or combinations thereof, which may be used to edit a genome. Non-limiting examples of gene-editing machinery include guide RNAs (gRNAs), nucleases, nuclease inhibitors, and combinations and complexes thereof. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

Attempts to modify gene expression by targeting polyadenylation using CRISPR gene-editing machinery have been performed. See, for example, Joubert R, Mariot V, Charpentier M, Concordet J P, Dumonceaux J. Gene Editing Targeting the DUX4 Polyadenylation Signal: A Therapy for FSHD? J Pers Med. 2020 Dec. 23; 11(1):7.

gRNA

In embodiments, the TM includes a gRNA. A gRNA targets a genomic locus in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer sequence has between about 40% to about 80% GC content.

In embodiments, the spacer binds to a target nucleotide sequence that immediately precedes a 5′ protospacer adjacent motif (PAM). The PAM sequence may be selected based on the desired nuclease. For example, the PAM sequence may be any one of the PAM sequences shown in Table 16 below, wherein N refers to any nucleic acid, R refers to A or G, Y refers to C or T, W refers to A or T, and V refers to A or C or G.

TABLE 16
Nucleases and PAM sequences

| PAM sequence (5′ to 3′) | Nuclease | Isolated from |
|---|---|---|
| NGG | SpCas9 | Streptococcus pyogenes |
| NGRRT or NGRRN | SaCas9 | Staphylococcus aureus |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer binds to a target nucleotide sequence of a mammalian target transcript of a target gene, such as a human gene. In embodiments, the spacer may bind to a target nucleotide sequence of a target transcript of a mutant target gene. In embodiments, the spacer may bind to a target nucleotide sequence that includes at least a portion of a polyadenylation sequence element (PSE) t. In embodiments, the spacer may bind to a target nucleotide sequence that includes at least one element of a PS of a target transcript. In embodiments, the spacer may bind to a target nucleotide sequence that includes target a polyadenylation signal (PAS), an intervening sequence (IS), a cleavage site (CS), a downstream element (DES), or a portion or combination thereof.

The scaffold sequence is the sequence within the sgRNA that is responsible for nuclease (e.g., Cas9) binding. The scaffold sequence does not include the spacer/targeting sequence. In embodiments, the scaffold may be about 1 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. In embodiments, the scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In embodiments, the scaffold may be at least 10, at least 20, at least 30, at least 40, at least 50, at least 60, at least 70, at least 80, at least 90, at least 100, at least 110, at least 120, or at least 125 nucleotides in length.

In embodiments, the gRNA is a dual-molecule guide RNA, e.g, crRNA and tracrRNA. In embodiments, the gRNA may further include a poly(A) tail.

In some embodiments, multiple gRNAs may be used a TMs in a single compound. In embodiments, the TM includes about 1, about 2, about 3, about 4, about 5, about 6, about 7, about 8, about 9, about 10, about 11, about 12, about 13, about 14, about 15, about 16, about 17, about 18, about 19, or about 20 gRNAs. In embodiments, the gRNAs recognize the same target. In embodiments, the gRNAs recognize different targets. In embodiments, the nucleic acid that includes a gRNA includes a sequence encoding a promoter, wherein the promoter drives expression of the gRNA.

### Nuclease

In embodiments, the TM includes a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease. In embodiments, the nuclease is a Cas9, Cas12a (CF3), Cas12b, Cas12c, Tnp-B like, Cas13a (C2c2), Cas13b, or Cas14 nuclease. For example, in some embodiments, the nuclease is a Cas9 nuclease or a Cpf1 nuclease.

In embodiments, the nuclease is a modified form or variant of a Cas9, Cas12a (Cpf1), Cas12b, Cas12c, Tnp-B like, Cas13a (C2c2), Cas13b, or Cas14 nuclease. In embodiments, the nuclease is a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. In embodiments, the nuclease may have at least about 80%, at least about 85%, at least about 90%, at least about 95%, at least about 98%, at least about 99%, or about 100% sequence identity to a naturally occurring Cas9, Cas12a (Cpf1), Cas12b, Cas12c, Tnp-B like, Cas13a (C2c2), Cas13b, Cas14 nuclease, or a TALEN, meganuclease, or zinc-finger nuclease. In embodiments, the nuclease is a Cas9 nuclease derived from S. pyogenes (SpCas9). In embodiments, a nuclease has at least about 90%, at least about 95%, at least about 96%, at least about 97%, at least about 98%, or at least about 99% sequence identity to a Cas9 nuclease derived from S. pyogenes (SpCas9). In embodiments, the nuclease is a Cas9 derived from S. aureus (SaCas9). In embodiments, the nuclease has at least about 90%, at least about 95%, at least about 96%, at least about 97%, at least about 98%, or at least about 99% sequence identity to a Cas9 derived from S. aureus (SaCas9). In embodiments, the Cpf1 is a Cpf1 enzyme from Acidarinococcus (species BV3L6, UniProt Accession No. U2UMQ6). In embodiments, the nuclease has at least about 90%, at least about 95%, at least about 96%, at least about 97%, at least about 98%, or at least about 99% sequence identity to a Cpf1 enzyme from Acidaminococcus (species BV3L6, UniProt Accession No. U2UMQ6).

In embodiments, the Cpf1 is a Cpf1 enzyme from Lachnospiraceae (species ND2006, UniProt Accession No. A0A182DWE3). In embodiments, the nuclease has at least about 90%, at least about 95%, at least about 96%, at least about 97%, at least about 98%, or at least about 99% sequence identity to a Cpf1 enzyme from Lachnospiraceae. In embodiments, a sequence encoding the nuclease is codon optimized for expression in mammalian cells. In embodiments, the sequence encoding the nuclease is codon optimized for expression in human cells or mouse cells.

In embodiments, the nuclease is a soluble protein.

In embodiments, the TM includes a nucleotide sequence that encodes a nuclease. In embodiments, the nucleic acid encoding a nuclease includes a sequence encoding a promoter, wherein the promoter drives expression of the nuclease.

gRNA and Nuclease Combinations

In embodiments, the compounds include a gRNA and a nuclease or a nucleotide sequence encoding a nuclease as TMs. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

In embodiments, the compounds include ribonucleoprotein (RNP) that includes a gRNA and a nuclease as a TM.

In embodiments, a composition that includes: (a) a first compound that includes a gRNA™ and (b) a second compound that is or encodes a nuclease, are delivered to a cell. In embodiments, a composition that includes: (a) a first compound that includes a nuclease or that encodes the nuclease as a TM, CPP and (b) a second molecule that is or encodes a gRNA are delivered to a cell. In embodiments, a composition that includes: (a) a first compound that includes a gRNA as a TM and (b) a second compound that includes a nuclease or encodes a nuclease as a TM are delivered to a cell.

### Genetic Element of Interest

In embodiments, the compounds disclosed herein include a genetic element of interest as a TM. In embodiments, a genetic element of interest replaces a genomic DNA sequence cleaved by a nuclease. Non-limiting examples of genetic elements of interest include genes, a single nucleotide polymorphism, promoter, or terminators.

### Nuclease Inhibitors

In embodiments, the compounds disclosed herein include a nuclease inhibitor as a TM. A limitation of gene editing is potential off-target editing. The delivery of a nuclease inhibitor will limit off-target editing. In embodiments, the nuclease inhibitor is a polypeptide, polynucleotide, or small molecule. Nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Polypeptides

In embodiments, the TM includes a polypeptide. In embodiments, the TM includes a protein or a fragment thereof. In embodiments, the therapeutic moiety includes an RNA binding protein or an RNA binding fragment thereof. In embodiments, the therapeutic moiety includes an enzyme. In embodiments, the therapeutic moiety includes an RNA-cleaving enzyme or an active fragment thereof. In embodiments, the therapeutic moiety includes an antibody or an antigen-binding fragment. Antibodies and antigen-binding fragments can be derived from any suitable source, including human, mouse, camelid (e.g., camel, alpaca, llama), rat, ungulates, or non-human primates (e.g., monkey, rhesus macaque).

The term “antibody” includes intact polyclonal or monoclonal antibodies and antigen-binding fragments thereof. For example, a native immunoglobulin molecule includes two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein that includes two heterologous polypeptide chains). The two heterodimeric proteins then associate by virtue of additional interchain disulfide bonds between the heavy chain polypeptides to form an immunoglobulin protein or polypeptide.

In embodiments, the therapeutic moiety is an antigen-binding fragment that binds to a transcript of interest (Ye et al. (2008) PNAS 105(1):82-87; and Jung et al. (2014) RNA. 20(6):805-814). In embodiments, an antigen-binding fragment includes 1, 2, 3, 4, 5, or all 6 CDRs of a variable heavy chain (VH) and/or a variable light chain (VL) sequence from an antibody that specifically binds to IRF-5, DMPK1, and/or DUX4. In embodiments, the antigen-binding fragment is a portion of a full-length antibody, such as Fab, F(ab′)2, Fab′, Fv fragments, minibodies, diabodies, single domain antibody (dAb), single-chain variable fragments (scFv), multispecific antibodies formed from antibody fragments, or any other modified configuration of the immunoglobulin molecule that includes an antigen-binding site or fragment of the required specificity.

### Endosomal Escape Vehicles (EEVs)

An endosomal escape vehicle (EEV) can be used to transport a cargo across a cellular membrane, for example, to deliver the cargo to the cytosol or nucleus of a cell. Cargo can include a TM. The EEV can comprise a cell penetrating peptide (CPP), for example, a cyclic cell penetrating peptide (cCPP). In embodiments, the EEV comprises a cCPP, which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO:1), KHKK (SEQ ID NO:2), KKHK (SEQ ID NO:3), KKKH (SEQ ID NO:4), KHKH (SEQ ID NO:5), HKHK (SEQ ID NO:6), KKKK (SEQ ID NO:7), KKRK (SEQ ID NO:8), KRKK (SEQ ID NO:9), KRRK (SEQ ID NO:10), RKKR (SEQ ID NO: 11), RRRR (SEQ ID NO:12), KGKK (SEQ ID NO:13), KKGK (SEQ ID NO:14), HBHBH (SEQ ID NO:15), HBKBH (SEQ ID NO:16), RRRRR (SEQ ID NO:17), KKKKK (SEQ ID NO:18), KKKRK (SEQ ID NO:19), RKKKK (SEQ ID NO:20), KRKKK (SEQ ID NO:21), KKRKK (SEQ ID NO:22), KKKKR (SEQ ID NO:23), KBKBK (SEQ ID NO:24), RKKKKG (SEQ ID NO:25), KRKKKG (SEQ ID NO:26), KKRKKG (SEQ ID NO:27), KKKKRG (SEQ ID NO:28), RKKKKB (SEQ ID NO:29), KRKKKB (SEQ ID NO:30), KKRKKB (SEQ ID NO:31), KKKKRB (SEQ ID NO:32), KKKRKV (SEQ ID NO:33), RRRRRR (SEQ ID NO:34), HHHHHH (SEQ ID NO:35), RHRHRH (SEQ ID NO:36), HRHRHR (SEQ ID NO:37), KRKRKR (SEQ ID NO:38), RKRKRK (SEQ ID NO:39), RBRBRB (SEQ ID NO:40), KBKBKB (SEQ ID NO:41), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO:18), KKKRK (SEQ ID NO:19), KBKBK (SEQ ID NO:24), KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can comprise PKKKRKV (SEQ ID NO:42), RR, RRR, RHR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO: 18), KKKRK (SEQ ID NO:19), KBKBK (SEQ ID NO:24), KKKRKV (SEQ ID NO:33), PKKKRKV (SEQ ID NO:42), PGKKRKV (SEQ ID NO:Z43), PKGKRKV (SEQ ID NO:Z44), PKKGRKV (SEQ ID NO:Z45), PKKKGKV (SEQ ID NO:46), PKKKRGV (SEQ ID NO:47), or PKKKRKG (SEQ ID NO:48). The EP can consist of PKKKRKV (SEQ ID NO:42), RR, RRR, RHR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO:42). The EP can consist of an NLS comprising the amino acid sequence PKKKRKV (SEQ ID NO:42). The EP can comprise an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NOZ55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66). The EP can consist of an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO:55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66).

All exocyclic sequences can also contain an N-terminal acetyl group. Hence, for example, the EP can have the structure: Ac-PKKKRKV (SEQ ID NO:42).

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

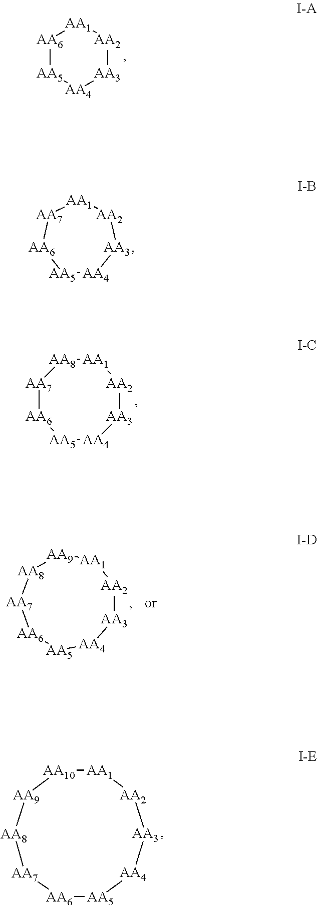

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| 2-[2-[2-aminoethoxy]ethoxy]acetic | AEBA, miniPEG | NA |
| acid | | |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (1) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (f) | phe (f) |
| phenylglycine | Phg (Ψ) | phg |
| 4-(phosphonodifluoromethyl)- | F₂Pmp (A) | f₂pmp |
| phenylalanine | | |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

As used herein, “polyethylene glycol” and “PEG” are used interchangeably. “PEGm,” and “PEGₘ,” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, n is 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—(OCH₂CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG4” is, or is derived from, HO(CO)—(CH₂)ₙ—(OCH₂CH₂)ₘ—NH₂ where n is 1 and m is 4.

The cCPP can comprise 4 to 20 amino acids, wherein: (i) at least one amino acid has a side chain comprising a guanidine group, or a protonated form thereof; (ii) at least one amino acid has no side chain or a side chain comprising

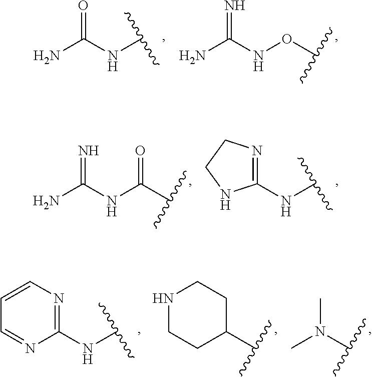

or a protonated form thereof, and (iii) at least two amino acids independently have a side chain comprising an aromatic or heteroaromatic group.

At least two amino acids can have no side chain or a side chain comprising

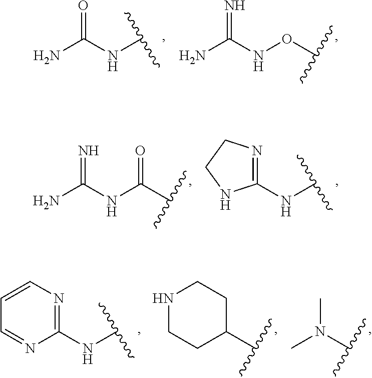

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

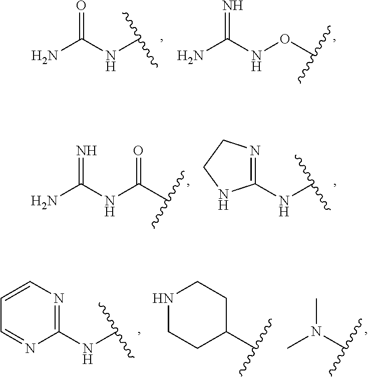

or a protonated form thereof.

The cCPP can comprise from 6 to 20 amino acid residues which form the cCPP, wherein: (i) at least two amino acid can independently be glycine, β-alanine, or 4-aminobutyric acid residues; (ii) at least one amino acid can have a side chain comprising an aryl or heteroaryl group; and (iii) at least one amino acid has a side chain comprising a guanidine group,

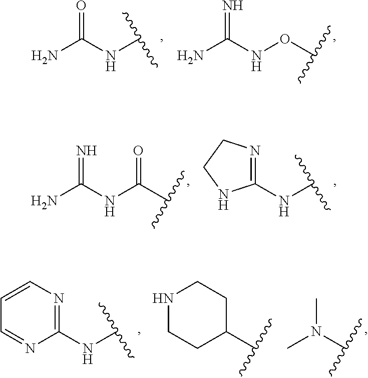

or a protonated form thereof.

The cCPP can comprise from 6 to 20 amino acid residues which form the cCPP, wherein: (i) at least three amino acids can independently be glycine, β-alanine, or 4-aminobutyric acid residues: (ii) at least one amino acid can have a side chain comprising an aromatic or heteroaromatic group; and (iii) at least one amino acid can have a side chain comprising a guanidine group,

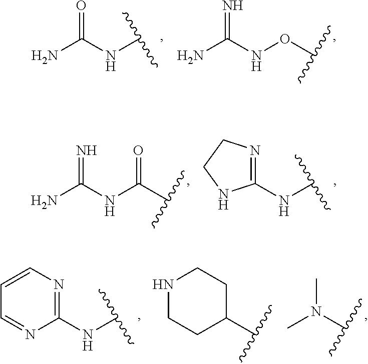

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

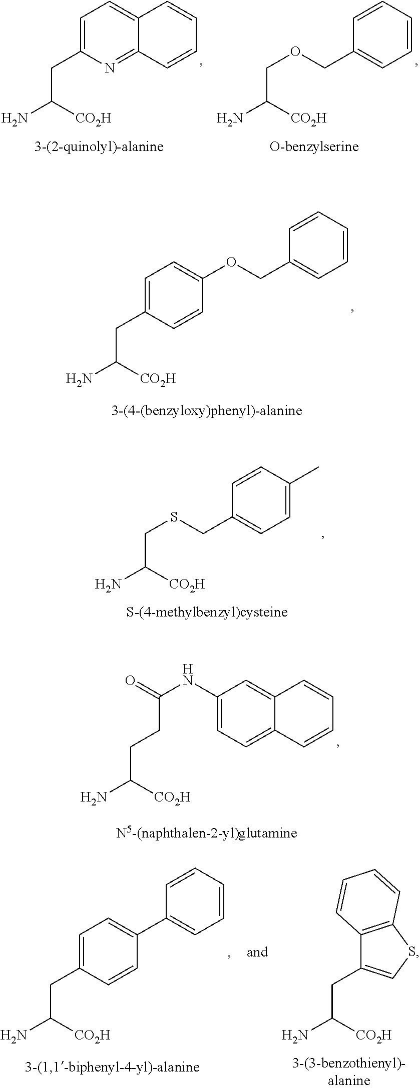

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenvlalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 2
Amino Acid Hydrophobicity

| Amino | | Eisenberg and | Engleman | Kyrie and | Hoop and | |
|---|---|---|---|---|---|---|
| Acid | Group | Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif; a cCPP having a Phe-Nal-Arg motif may exhibit improved cytosolic delivery efficiency compared to an otherwise identical cCPP having a Nal-Phe-Arg motif; and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Angstroms; A²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. The SASA values described herein are based on the theoretical values listed in Table 3 below, as reported by Tien, et al. (PLOS ONE 8(11): e80635, available at doi.org/10.1371/joumal.pone.0080635), which is herein incorporated by reference in its entirety for all purposes.

TABLE 3
Amino Acid SASA Values

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

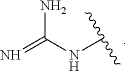 [SMILES: *NC(=N)N]

As used herein, a protonated form of guanidine refers to the structure:

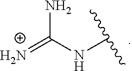 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

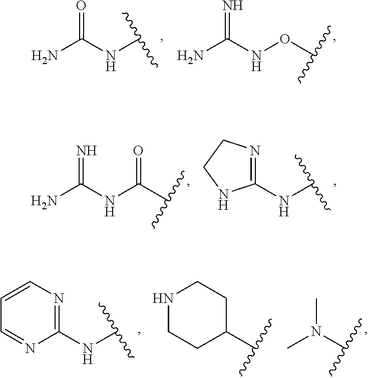

or a protonated form thereof.

The disclosure relates to a cCPP comprising from 4 to 20 amino acids residues, wherein: (i) at least one amino acid has a side chain comprising a guanidine group, or a protonated form thereof, (ii) at least one amino acid residue has no side chain or a side chain comprising

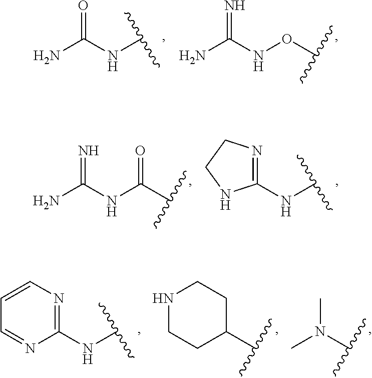

or a protonated form thereof: and (iii) at least two amino acids residues independently have a side chain comprising an aromatic or heteroaromatic group.

At least two amino acids residues can have no side chain or a side chain comprising

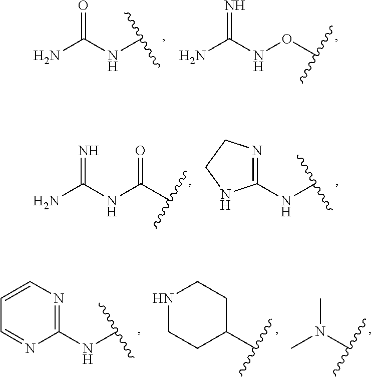

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

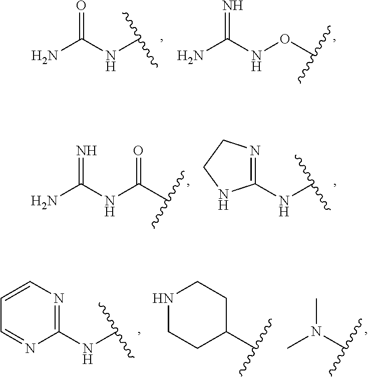

or a protonated form thereof.

The cCPP can comprise at least two amino acids each independently having one of the following moieties

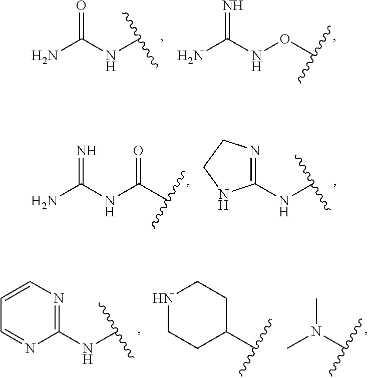

or a protonated form thereof. At least two amino acids can have a side chain comprising the same moiety selected from:

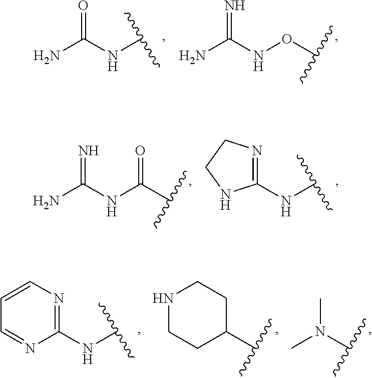

or a protonated form thereof. At least one amino acid can have a side chain comprising

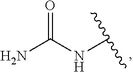 [SMILES: *NC(N)=O]

or a protonated form thereof. At least two amino acids can have a side chain comprising

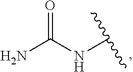 [SMILES: *NC(N)=O]

or a protonated form thereof. One, two, three, or four amino acids can have a side chain comprising

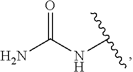 [SMILES: *NC(N)=O]

or a protonated form thereof. One amino acid can have a side chain comprising

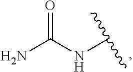 [SMILES: *NC(N)=O]

or a protonated form thereof. Two amino acids can have a side chain comprising

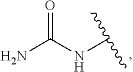 [SMILES: *NC(N)=O]

or a protonated form thereof.

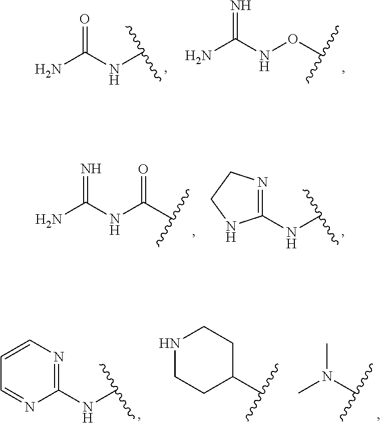

or a protonated form thereof, can be attached to the terminus of the amino acid side chain.

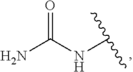 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

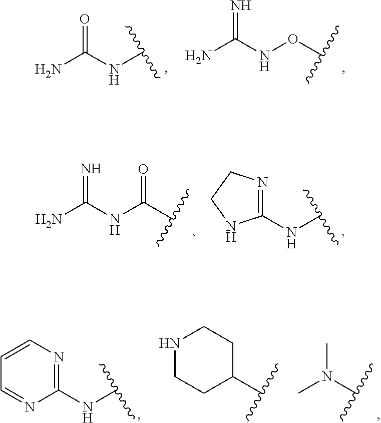

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamine acid, or homoglutamine. The cCPP can comprise a residue of asparagine. The cCPP can comprise a residue of glutamine.

The cCPP can comprise a residue of tyrosine, phenylalanine, I-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, s-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

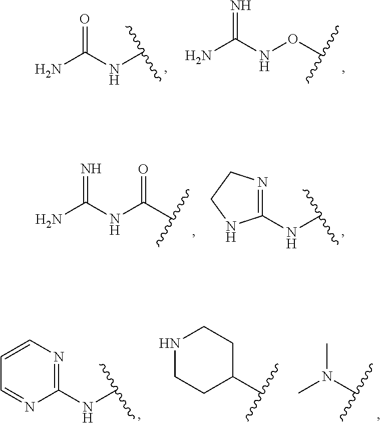

or a protonated form thereof, can be adjacent to an amino acid having a side chain comprising an aromatic or heteroaromatic group. An amino acid having a side chain comprising:

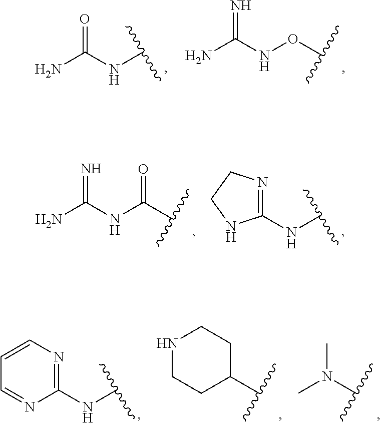

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

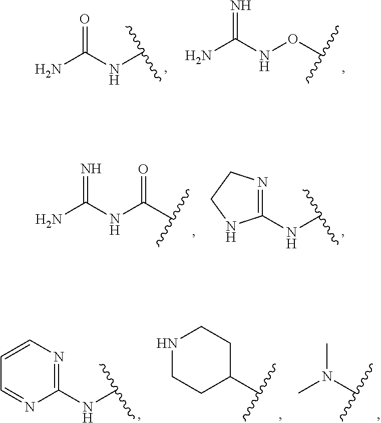

or protonated forms thereof, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

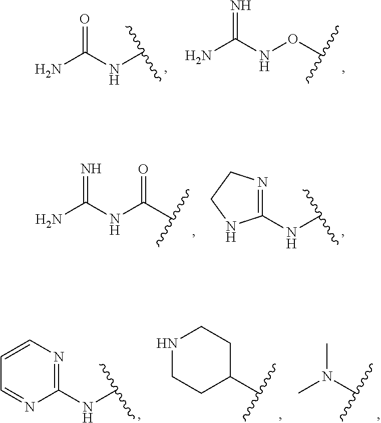

or a protonated form thereof. The cCPPs can comprise at least two contiguous amino acids having a side chain comprising an aromatic or heteroaromatic group and at least two non-adjacent amino acids having a side chain comprising

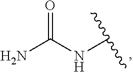 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

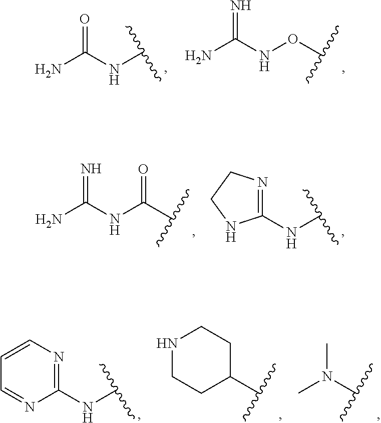

or a protonated form thereof, are alternating with at least two amino acids having a side chain comprising a guanidine group or protonated form thereof.

The cCPP can comprise the structure of Formula (A):

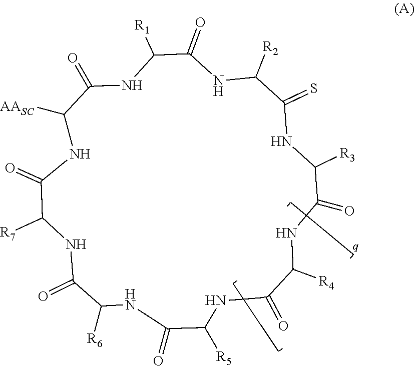

or a protonated form thereof,
wherein:
   R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cyclic peptide of Formula (A) is not FfΦRrRrQ (SEQ ID NO:67). In embodiments, the cyclic peptide of Formula (A) is FfΦRrRrQ (SEQ ID NO:67).

The cCPP can comprise the structure of Formula (I):

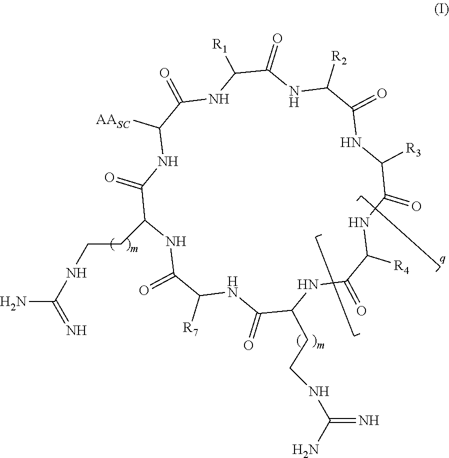

or a protonated form thereof,
wherein:
   R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer of 0, 1, 2, or 3.

R₁, R₂, and R; can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 1 or Table 3. R₄ can be H or an amino acid residue having a side chain comprising an aromatic group. R₄ can be H, —CH₂Ph, or -CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1 or Table 3. R₄ can be H or an amino acid residue having a side chain comprising an aromatic group. R₅ can be H, —CH₂Ph, or -CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃alkylene-heteroaryl. R₆ can be H or -alkylene-aryl. R₆ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1 or Table 3. R, can be H or an amino acid residue having a side chain comprising an aromatic group. R₆ can be H, —CH₂Ph, or -CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃ alkylene-heteroaryl. R₇ can be H or -alkylene-aryl. R₇ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 1 or Table 3. R₇ can be H or an amino acid residue having a side chain comprising an aromatic group. R₇ can be H, —CH₂Ph, or -CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₁, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to The glutamine Residue.

q can be 1, 2, Or 3. q can 1 or 2. q can be 1. q can be 2. q can be 3. Q can be 4.

m can be 1-3. m can be 1 or 2. m can be 0. m can be 1. m can be 2. m can be 3.

The cCPP of Formula (A) can comprise the structure of Formula (I)

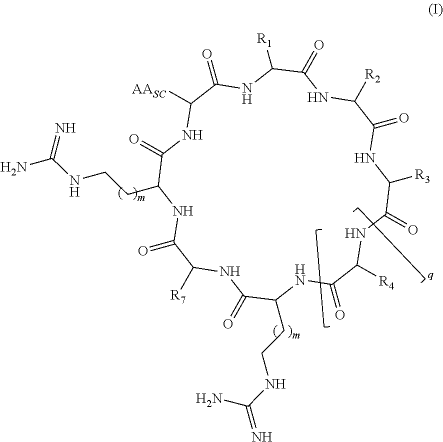

or protonated form thereof, wherein AA_{SC}, R₁, R₂, R₃, R₄, R₇, m, and q are as defined herein.

The cCPP of Formula (A) can comprise the structure of Formula (I-a) or Formula (I-b):

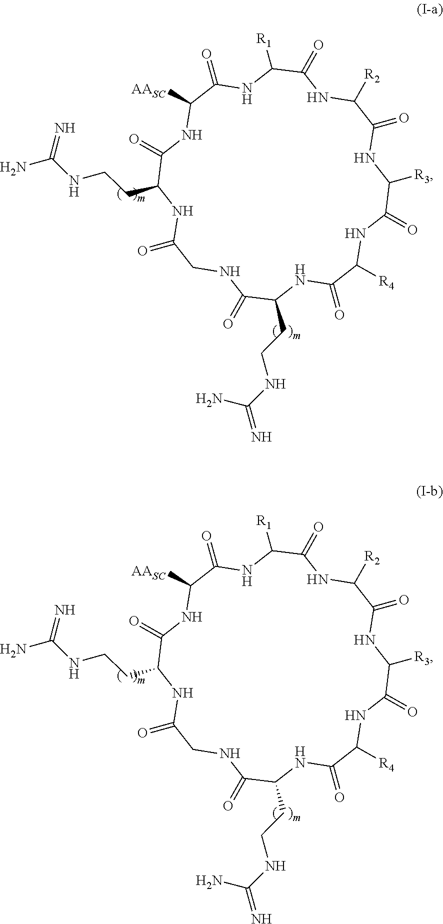

or protonated form thereof, wherein AA_{SC}, R₁, R₂, R₃, R₄, and m are as defined herein.

The cCPP of Formula (A) can comprise the structure of Formula (I-1), (I-2), (I-3), or (I-4):

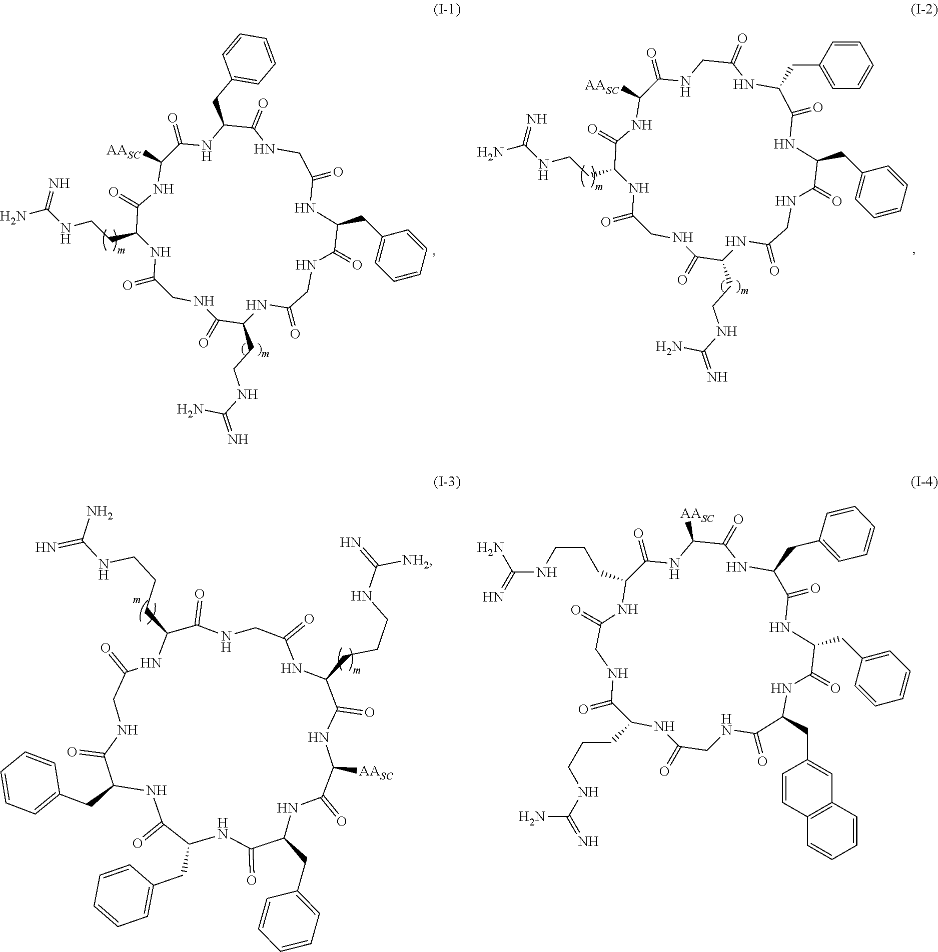

or protonated form thereof, wherein AA_{SC} and m are as defined herein.

The cCPP of Formula (A) can comprise the structure of Formula (I-5) or (1-6):

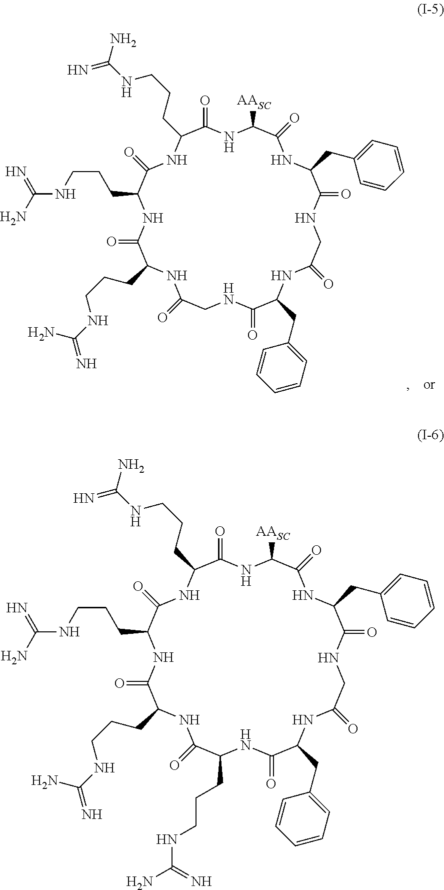

or protonated form thereof, wherein AA_{SC} is as defined herein.

The cCPP of Formula (A) can comprise the structure of Formula (I-1):

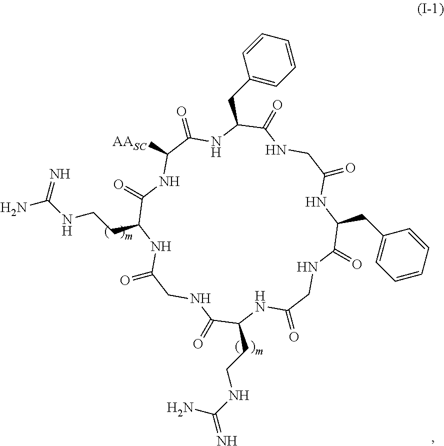

or a protonated form thereof, wherein AA_{SC} and m are as defined herein.

The cCPP of Formula (A) can comprise the structure of Formula (I-2):

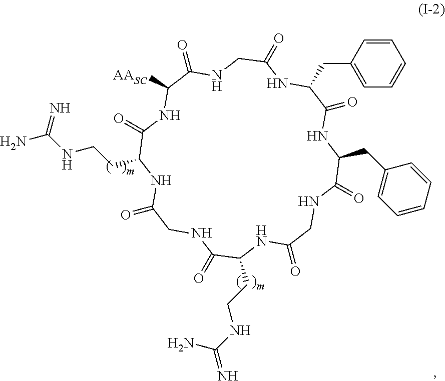

or a protonated form thereof, wherein AA_{SC} and m are as defined herein.

The cCPP of Formula (A) can comprise the structure of Formula (I-3):

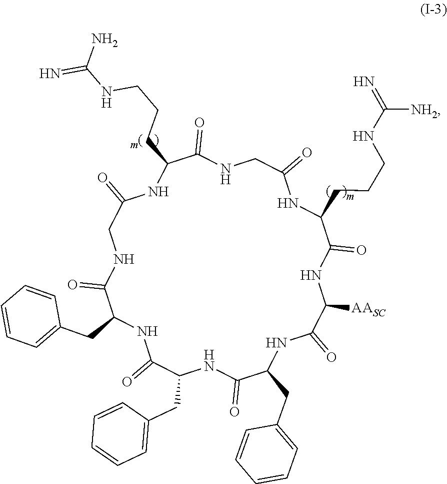

or a protonated form thereof, wherein AA_{SC} and m are as defined herein.

The cCPP of Formula (A) can comprise the structure of Formula (1-4):

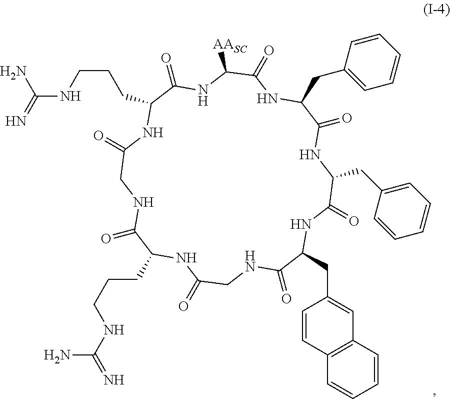

or a protonated form thereof, wherein AA_{SC} and m are as defined herein.

The cCPP of Formula (A) can comprise the structure of Formula (1-5):

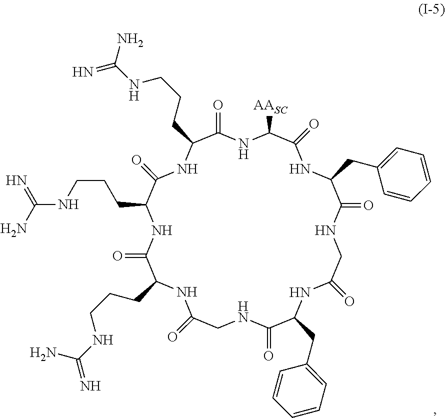

or a protonated form thereof, wherein AA_{SC} and m are as defined herein. The cCPP of Formula A can comprise the structure of Formula (I-6):

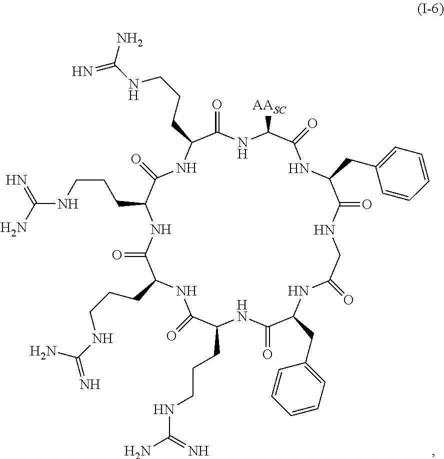

or a protonated form thereof, wherein AA_{SC} and m are as defined herein.

The cCPP can comprise one of the following sequences: FGFGRGR (SEQ ID NO:68); GfFGrGr (SEQ ID NO:69), FfΦGRGR (SEQ ID NO:70); FfFGRGR (SEQ ID NO:71); or FfΦGrGr (SEQ ID NO:72). The cCPP can have one of the following sequences: FGFΦ (SEQ ID NO:73); GfFGrGrQ (SEQ ID NO:74), FfΦGRGRQ (SEQ ID NO:75); FfFGRGRQ (SEQ ID NO:76); or FfΦGrGrQ (SEQ ID NO:77).

The disclosure also relates to a cCPP having the structure of Formula (II):

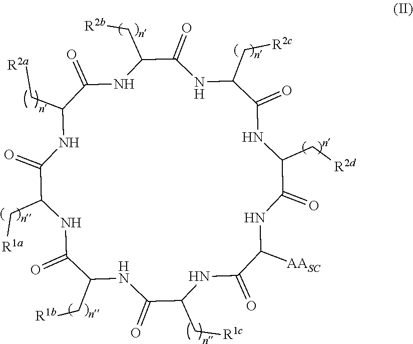

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

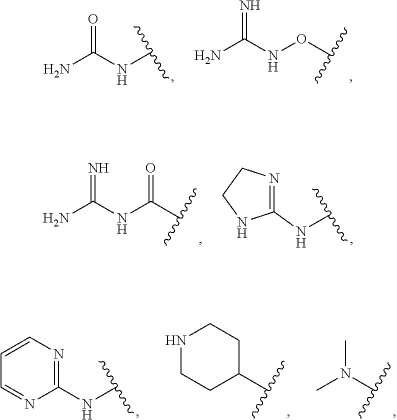

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

At least two of R^{2a}, R^{2b}, R^{2c} and R^{2d} can be

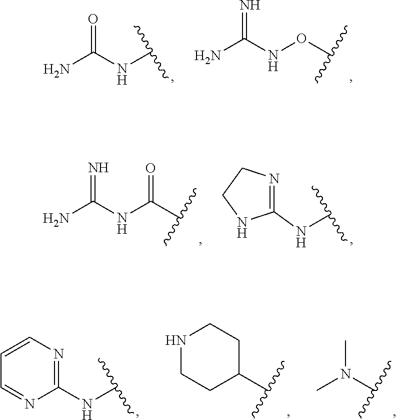

or a protonated form thereof. Two or three of R^{2a}, R^{2b}, R^{2c} and R^{2d} can be

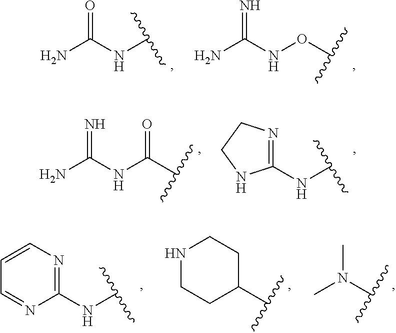

or a protonated form thereof one or R^{2a}, R^{2b}, R^{2c} and R^{2d} can be

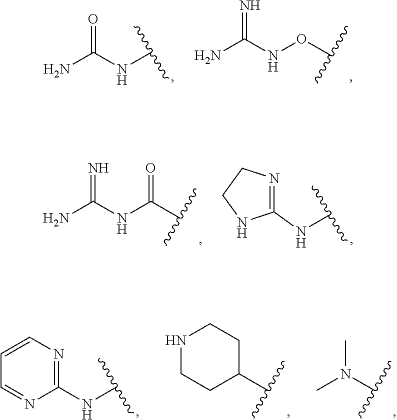

or a protonated form thereof. At least one of R^{2a}, R^{2b}, R^{2c} and R^{2d} can be

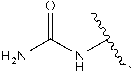 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

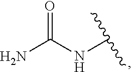 [SMILES: *NC(N)=O]

or a protonated form thereof, and the remaining of R^{2a}, R^{2b}, R^{2c} and R^{2d} can be guanidine, or a protonated form thereof.

All of R^{2a}, R_{2b}, R^{2c} and R^{2d} can be

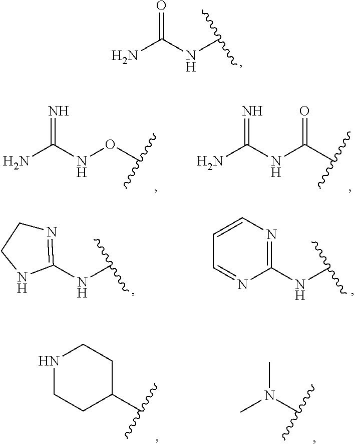

or a protonated form thereof. At least of R^{2a}, R^{2b}, R^{2c} and R^{2d} can be

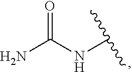 [SMILES: *NC(N)=O]

or a protonated form thereof, and the remaining of R^{2a}, R^{2b}, R^{2c} and R^{2d} can be guanidine or a protonated form thereof. At least two R^{2a}, R^{2b}, R^{2c} and R^{2d} groups can be

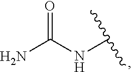 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

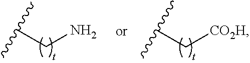

wherein t can be an integer from 0 to 5. AA_{SC} can be

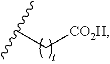

wherein t can Be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

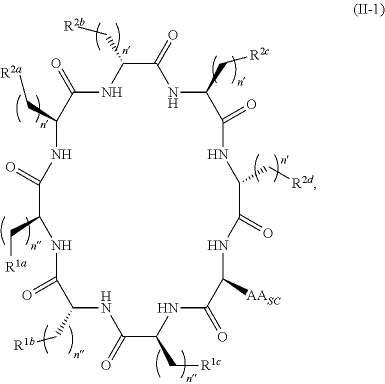

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

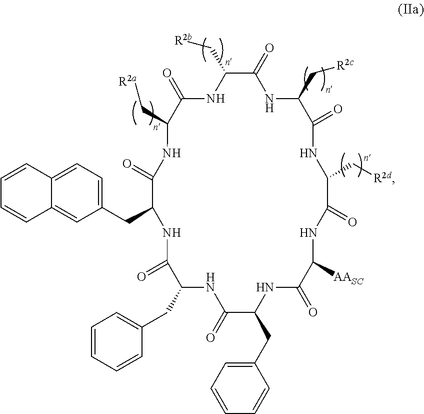

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (Ib):

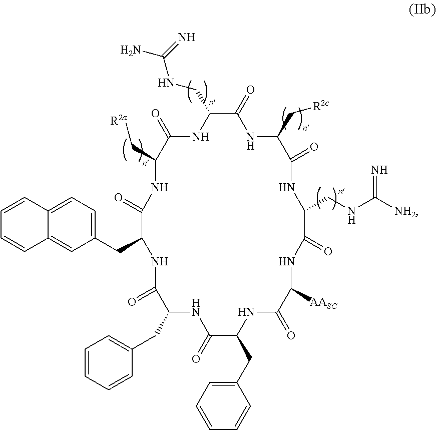

wherein R^{2a}, R^{2b}, AA_{SC}. and n′ are as defined herein.

The cCPP can have the structure of Formula (IIc):

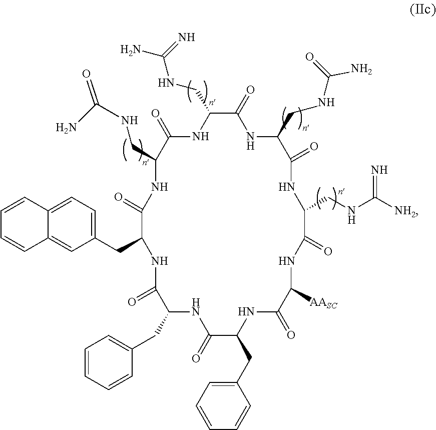

or a protonated form thereof, wherein:
AA_{SC} and n′ are as defined herein.

The cCPP of Formula (IIa) has one of the following structures:

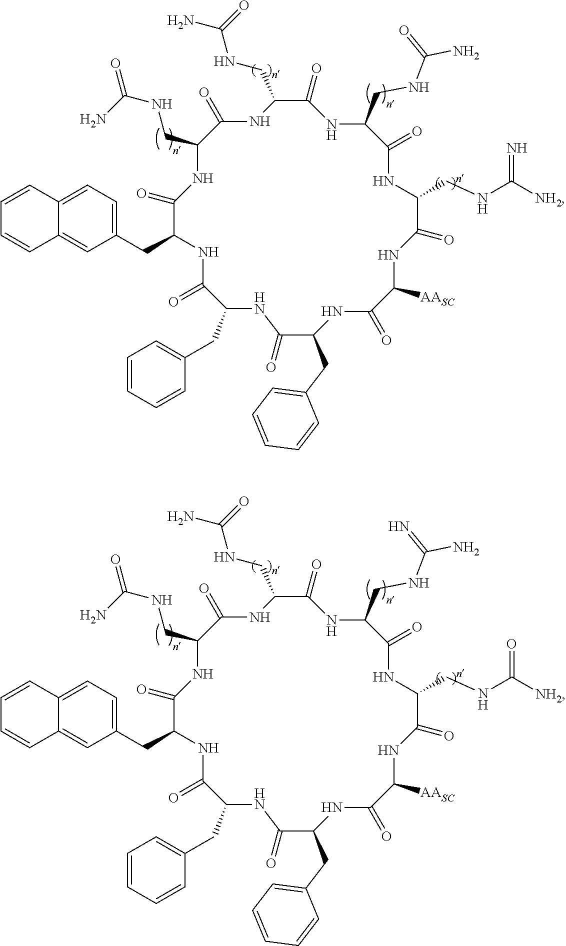

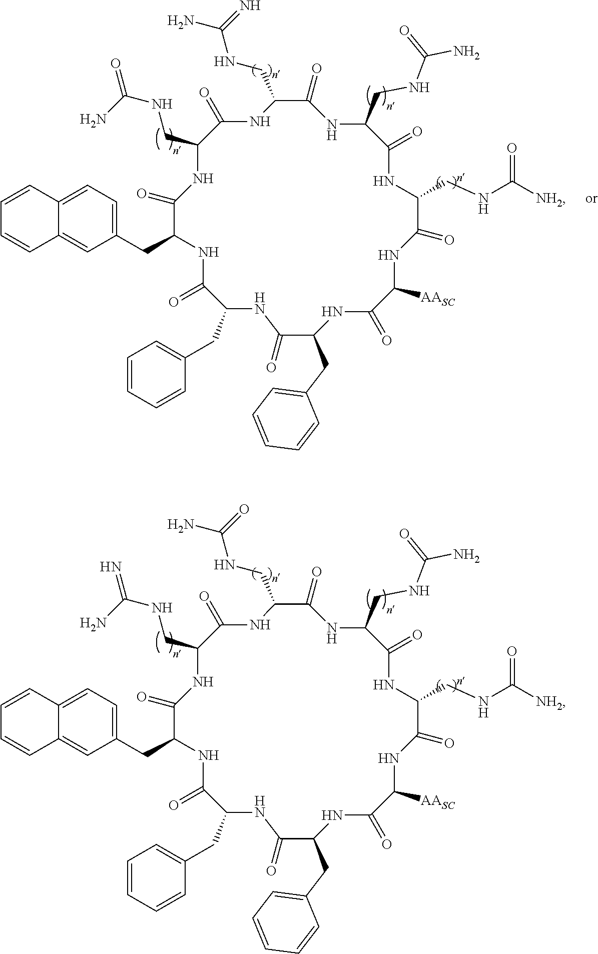

wherein AA_{SC} and n are as defined herein.

The cCPP of Formula (IIa) has one of the following structures:

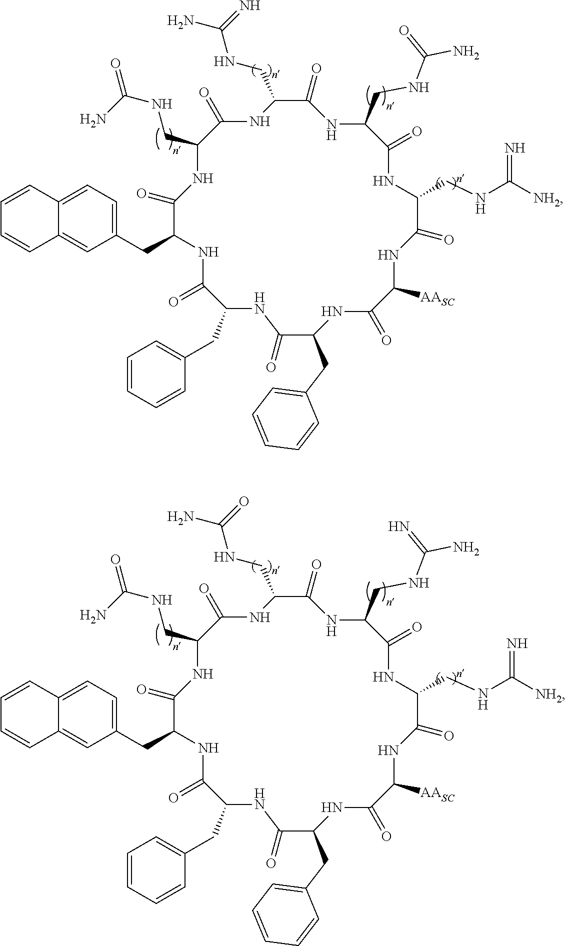

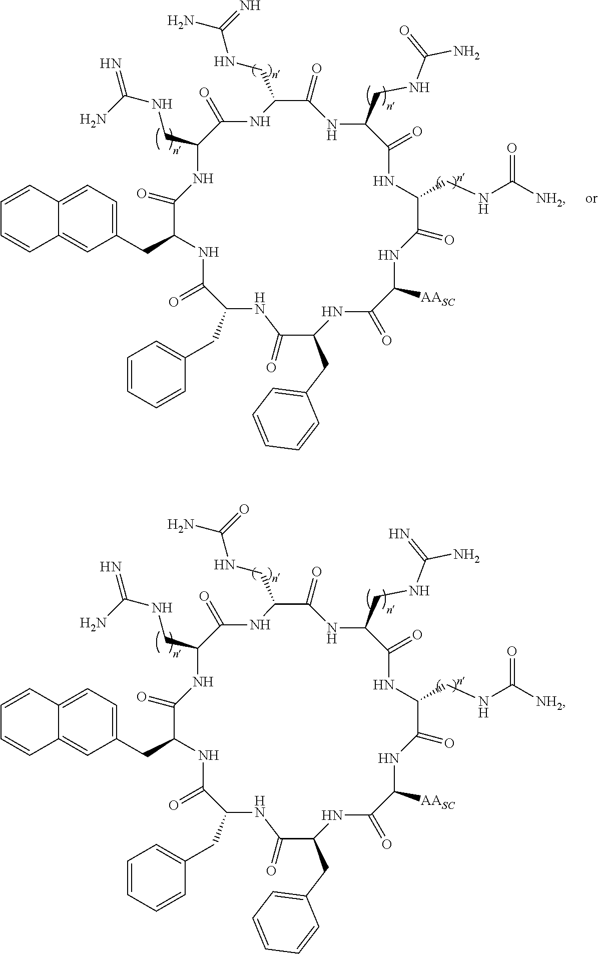

wherein AA_{SC} and n are as defined herein

The cCPP of Formula (IIa) has one of the following structures:

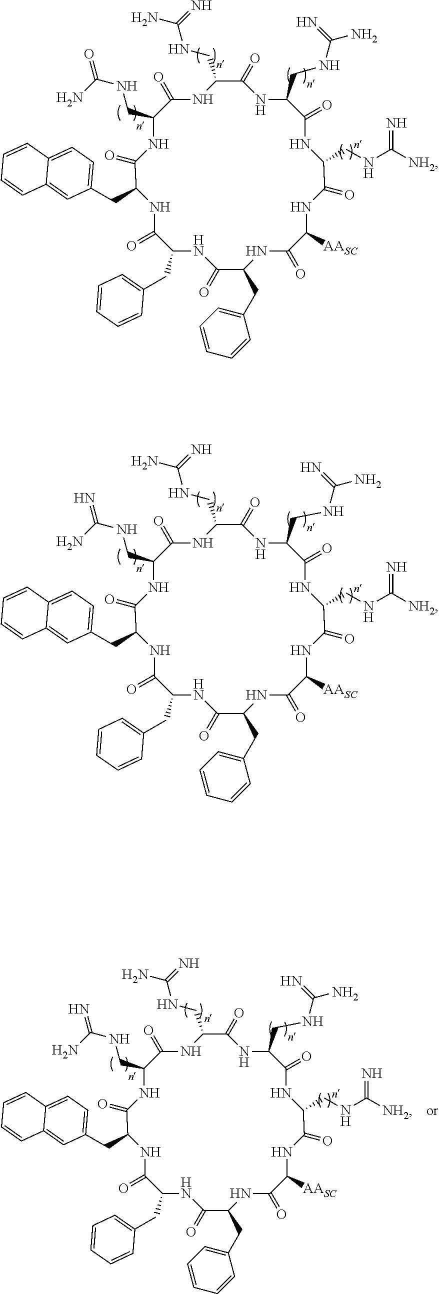

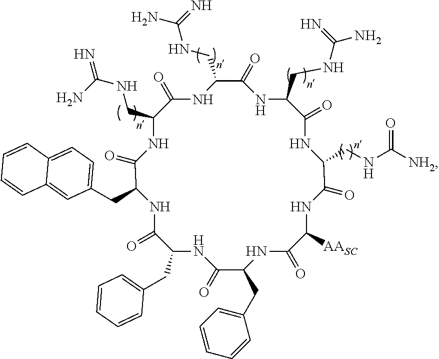

wherein AA_{SC} and n are as defined herein.

The cCPP of Formula (II) can have the structure:

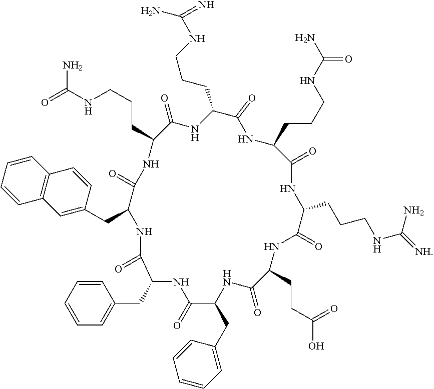 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

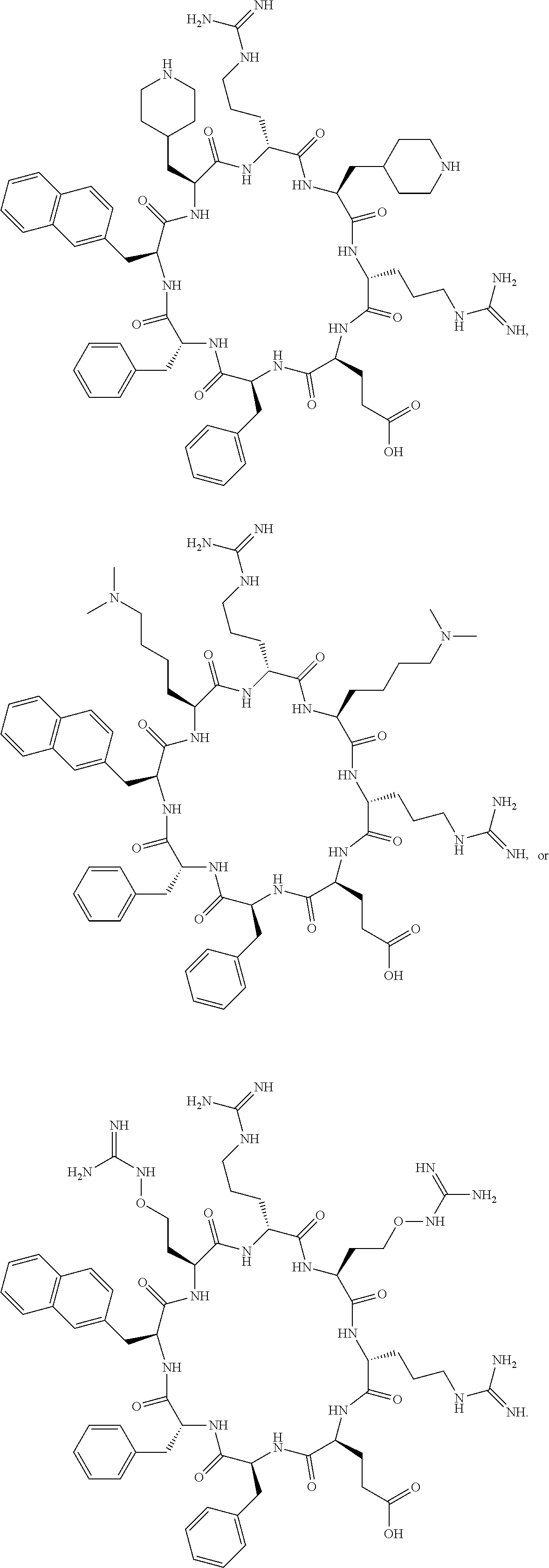

The cCPP can have the structure of Formula (III):

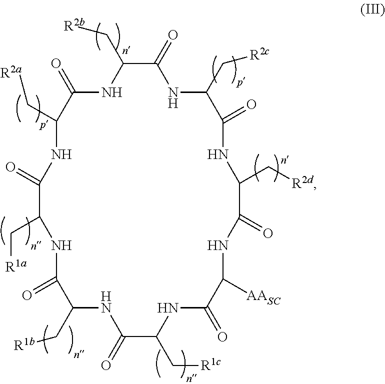

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

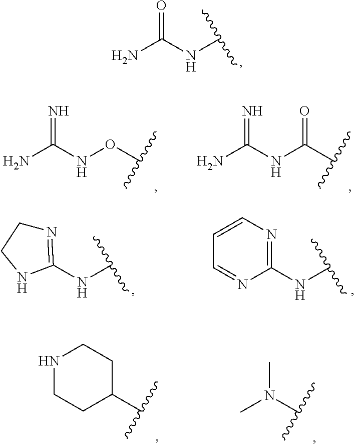

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

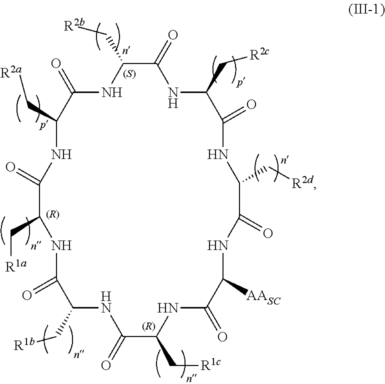

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

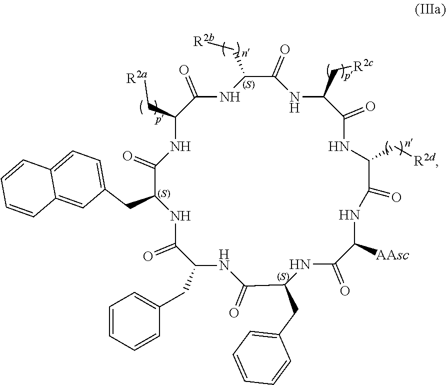

wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

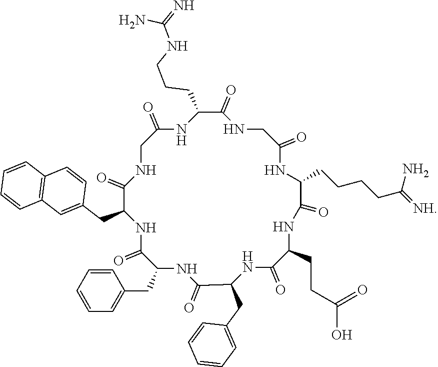 [SMILES: N=C(N)CCCC[C@H]1NC(=O)CNC(=O)[C@@H](CCCNC(=N)N)NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (A) can be selected from:

| CPP Sequence | SEQ ID NO: |
|---|---|
| (FfΦRrRrQ) | 78 |
| (FfΦCit-r-Cit-rQ) | 79 |
| (FfΦGrGrQ) | 80 |
| (FfFGRGRQ) | 81 |
| (FGFGRGRQ) | 82 |
| (GfFGrGrQ) | 83 |
| (FGFGRRRQ) | 84 |
| (FGFRRRRQ) | 85 |

The cCPP of Formula (A) can be selected from:

| CPP Sequence | SEQ ID NO: |
|---|---|
| FΦRRRRQ | 86 |
| fΦRrRrQ | 87 |
| FfΦRrRrQ | 78 |
| FfΦCit-r-Cit-rQ | 79 |
| FfΦGrGrQ | 80 |
| FfΦRGRGQ | 88 |
| FfFGRGRQ | 81 |
| FGFGRGRQ | 82 |
| GfFGrGrQ | 83 |
| FGFGRRRQ | 84 |
| FGFRRRRQ | 85 |

In embodiments, the cCPP is not selected from:

CPP sequences and SEQ ID NOs

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| FRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

In embodiments, the cCPP is not selected from:

CPP sequences and SEQ ID NOs

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| FRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦDRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹⁻J-R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹⁻-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹⁻J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹⁻J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

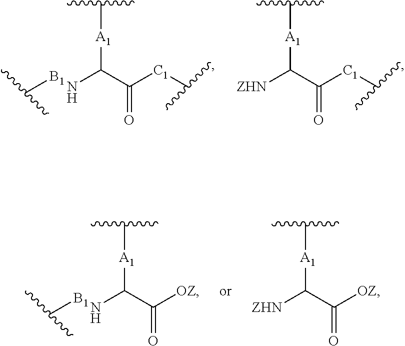

wherein At, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

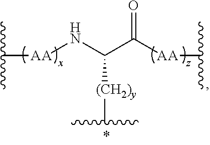

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

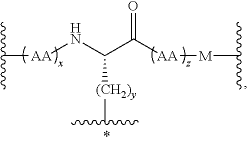

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

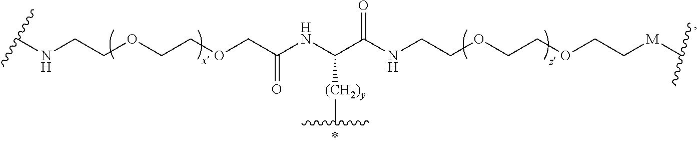

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

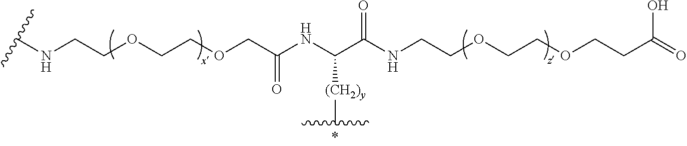

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges Therebetween. y can be an integer from 2-5. y can be an integer From 3-5. y Can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

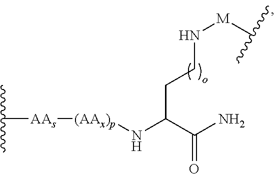

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

The linker can have a structure:

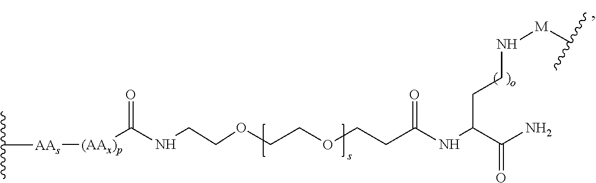

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

M can comprise an alkylene, alkenylene, alkynylene, carbocyclyl, or heterocyclyl, each of which is optionally substituted. M can be selected from:

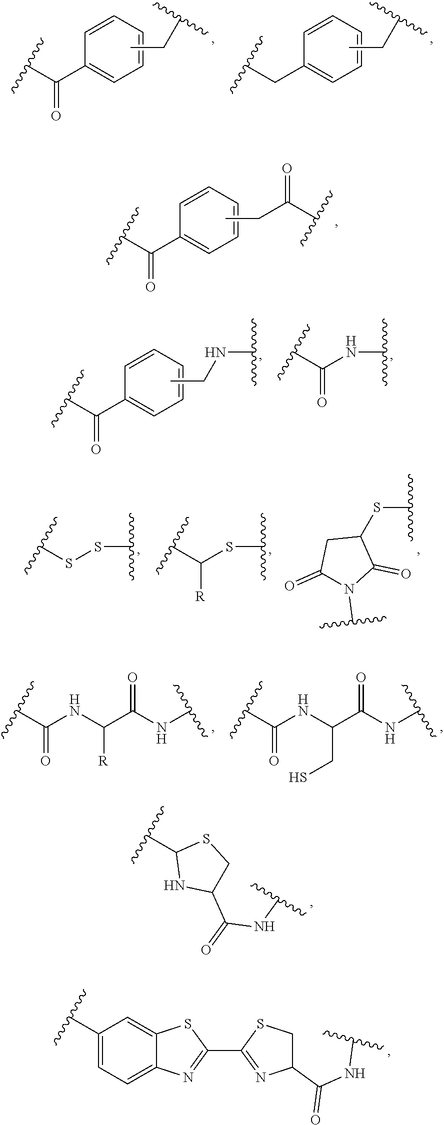

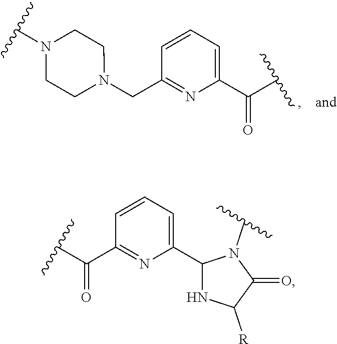

wherein R is alkyl, alkenyl, alkynyl, carbocyclyl, or heterocyclyl.

M can be selected from:

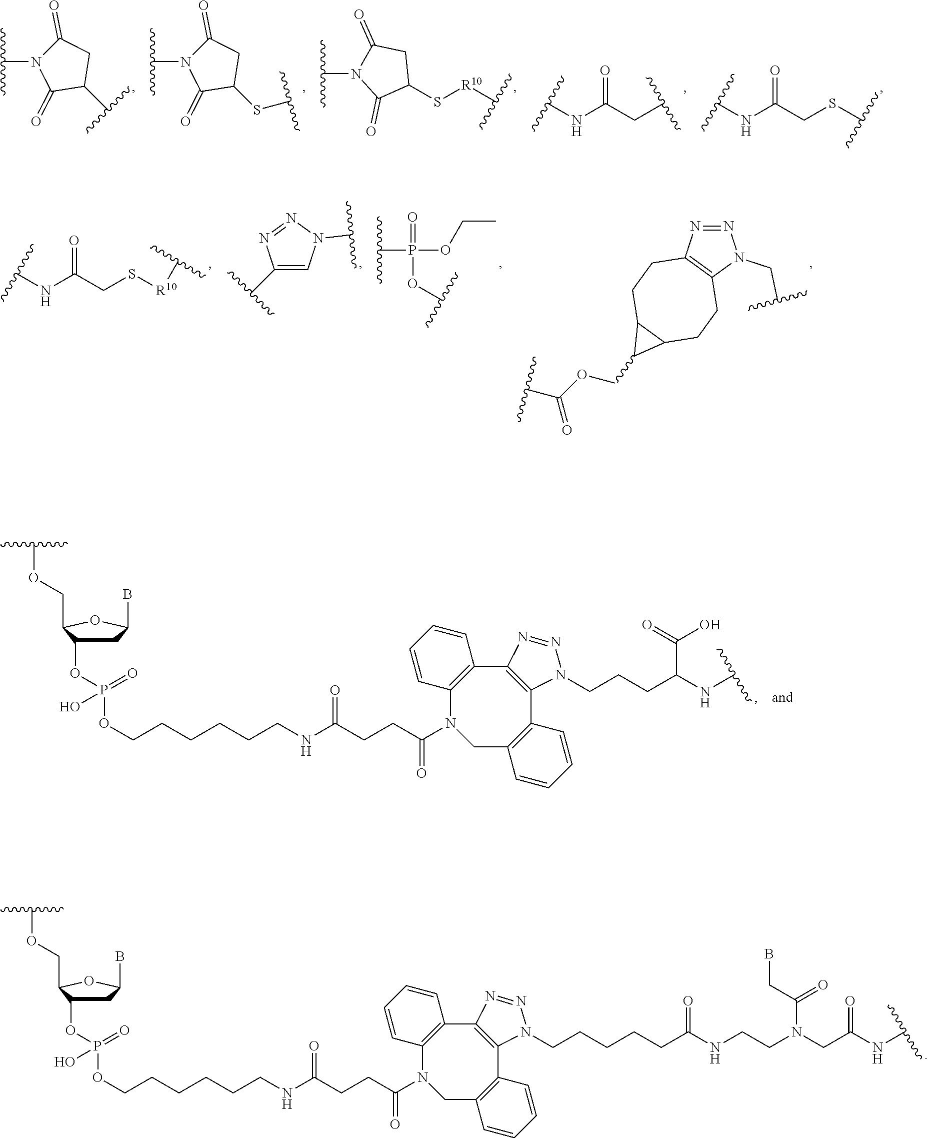

wherein: R¹⁰ is alkylene, cycloalkyl, or

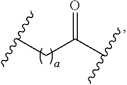

wherein a is 0 to 10.

M can be

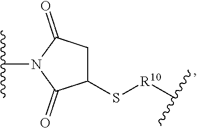

### R¹⁰ can be

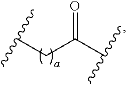

### and a is 0 to 10. M can be

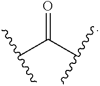 [SMILES: *C(*)=O]

M can be a heterobifunctional crosslinker, e.g.,

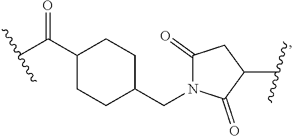 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The O-amino acid can be β-alanine.

o can be an integer from 0 to 10, E.g., 0, 1, 2, 3, 4, 5, 6, 7, 8, 9, And 10. o can be 0, 1, 2, or 3. o can be 0. o can be 1. o can be 2. o can be 3.

P can be 0 to 5, e.g., 0, 1, 2, 3, 4, or 5. p can be 0. p can be 1. p can be 2. p can be 3. p can be 4. p can be 5.

The linker can have the structure:

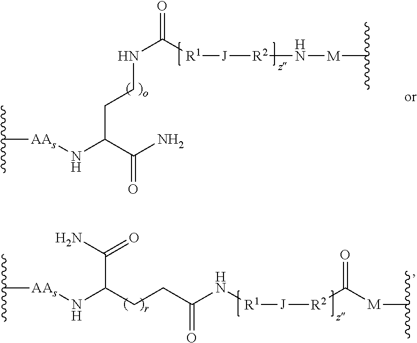

wherein M, AAₛ, each —(R¹⁻J-R²)z″-, o and z″ are defined herein; r can be 0 or 1.

r can be 0. r can be 1.

The linker can have the structure:

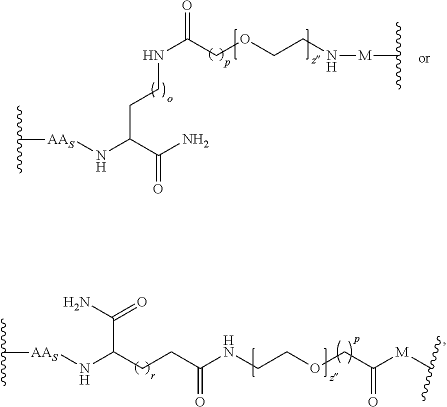

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

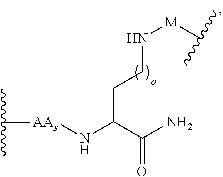

wherein:
M, AAₛ and o are as defined herein.

Other non-limiting examples of suitable linkers include:

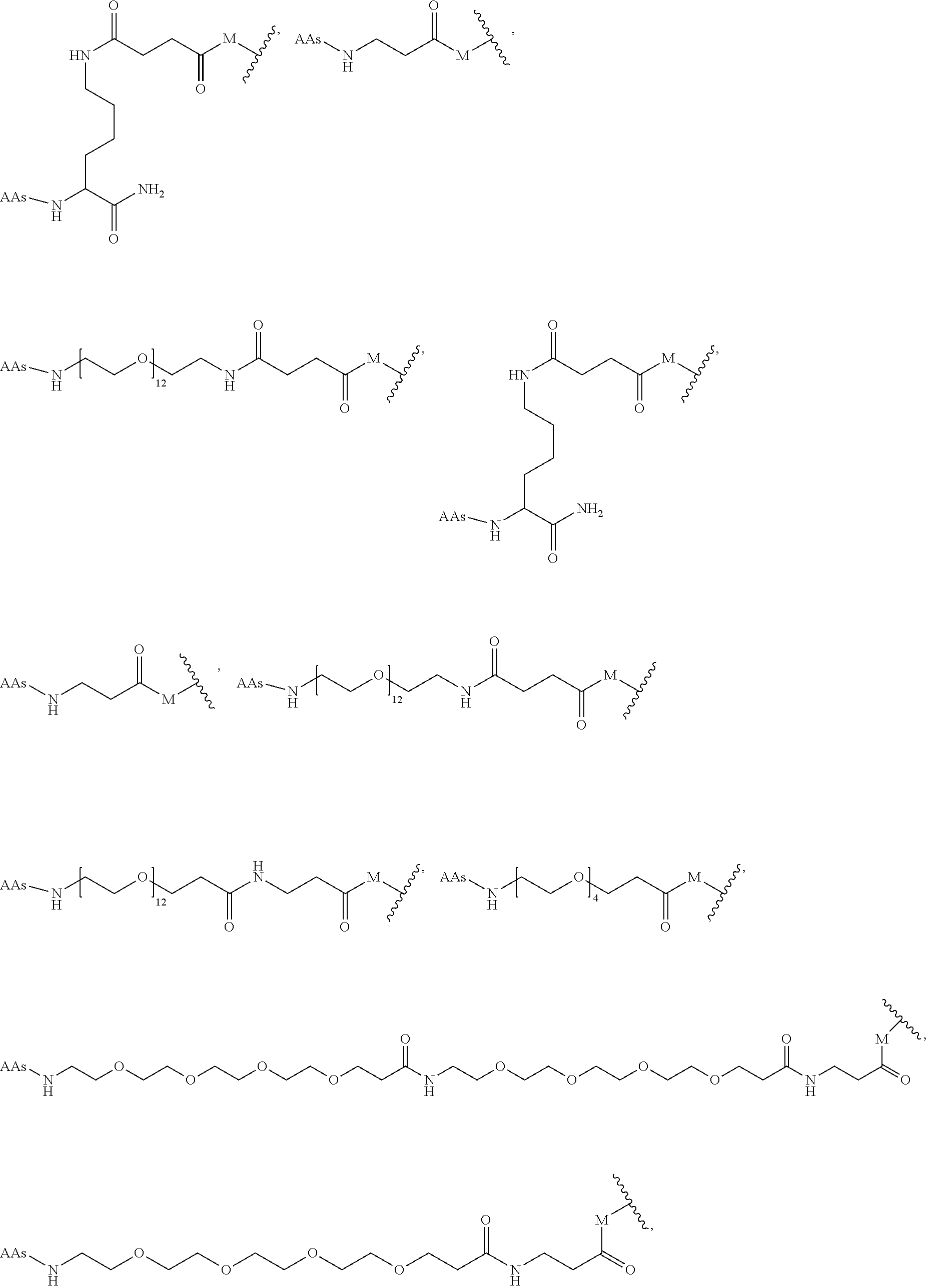

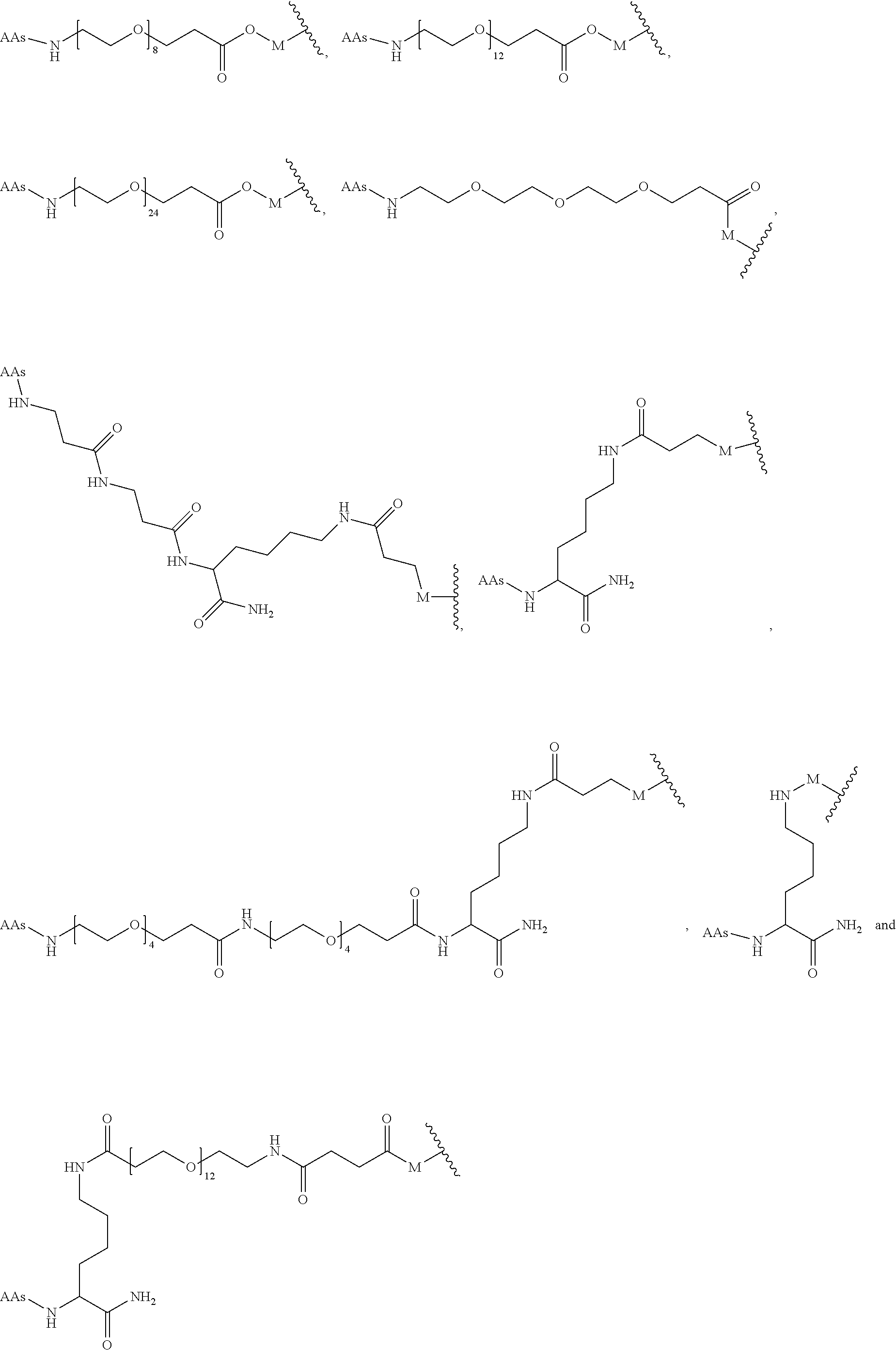

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

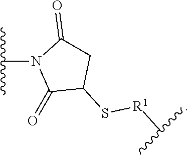

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

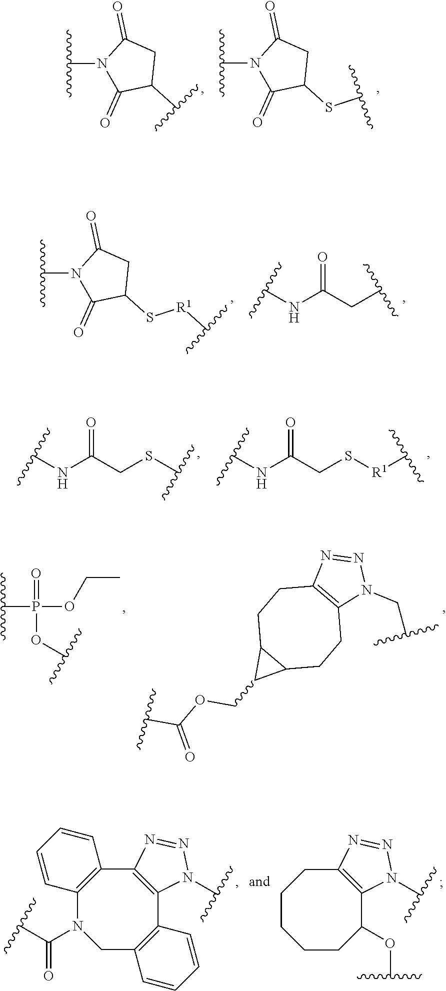

wherein: R¹ is alkylene, cycloalkyl, or

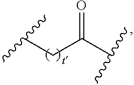

wherein t′ is 0 to 10 wherein each R is independently an alkyl, alkenyl, alkynyl, carbocyclyl, or heterocyclyl, wherein R¹ is

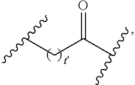 [SMILES: *CC(*)=O]

and t′ is 2.

The linker can have the structure:

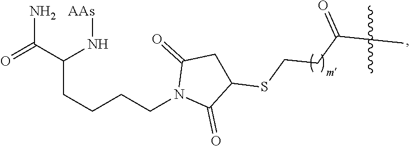

wherein AAₛ is as defined herein, and m′ is 0-10.

The linker can be of the formula:

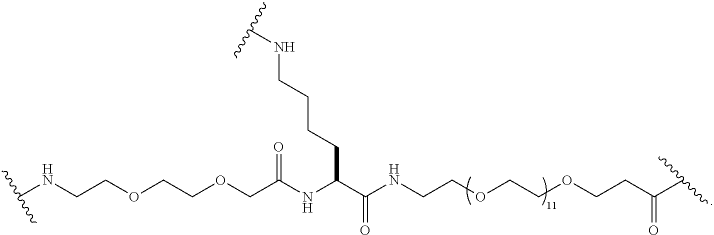

The linker can be of the formula:

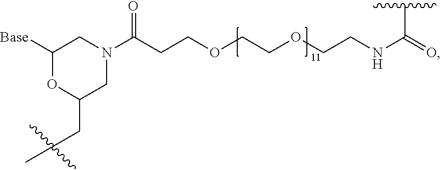

wherein “base” is a nucleobase at the 3′ end of a cargo phosphorodiamidate morpholino oligomer.

The linker can be of the formula:

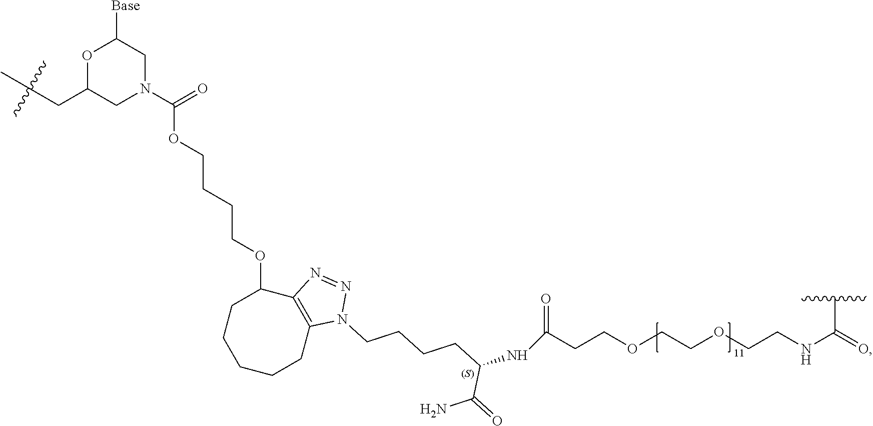

wherein “base” corresponds to a nucleobase at the 3′ end of a cargo phosphorodiamidate morpholino oligomer.

The linker can be of the formula:

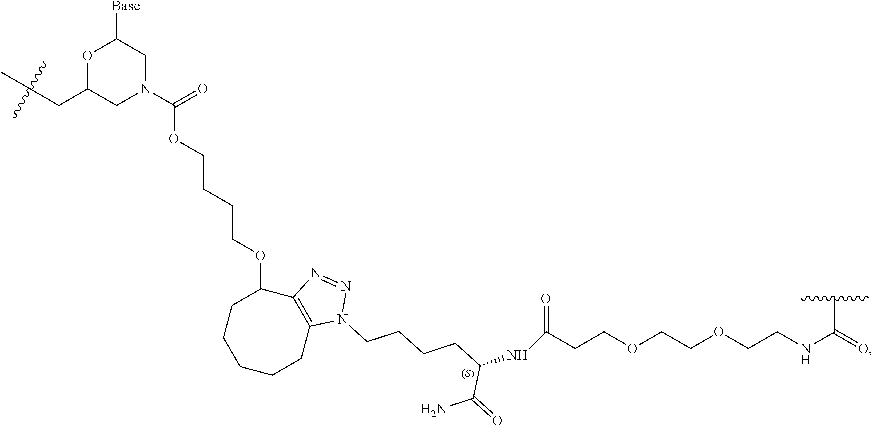

wherein “base” is a nucleobase at the 3′ end of a cargo phosphorodiamidate morpholino oligomer.

The linker can be of the formula:

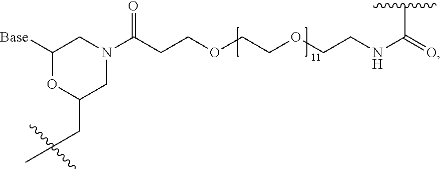

wherein “base” is a nucleobase at the 3′ end of a cargo phosphorodiamidate morpholino oligomer.

The linker can be of the formula:

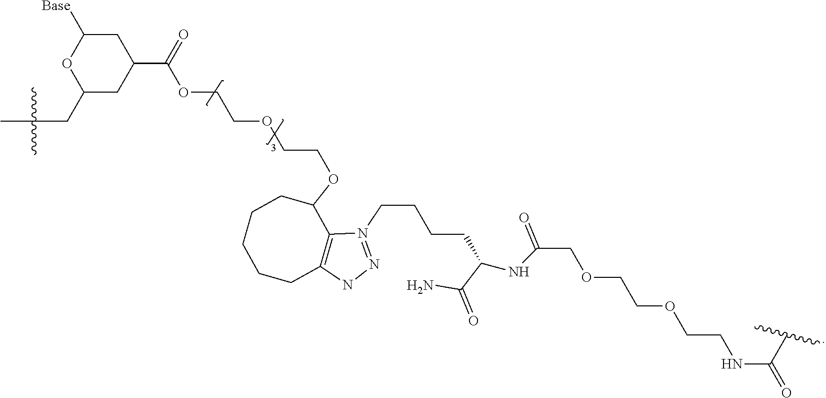

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 4:

TABLE 4
cCPP-linker conjugates and SEQ ID NOs

| | |
|---|---|
| cyclo(FfΦ-4gp-r-4gp-rQ)-PEG₄-K-NH₂ | cyclo(SEQ ID NO: 118)-PEG₄-K-NH₂ |
| cyclo(FfΦ-Cit-r-Cit-rQ)-PEG₄-K-NH₂ | cyclo(SEQ ID NO: 119)-PEG₄-K-NH₂ |
| cyclo(FfΦ-Pia-r-Pia-rQ)-PEG₄-K-NH₂ | cyclo(SEQ ID NO: 120)-PEG₄-K-NH₂ |
| cyclo(FfΦ-Dml-r-Dml-rQ)-PEG₄-K-NH₂ | cyclo(SEQ ID NO: 121)-PEG₄-K-NH₂ |
| cyclo(FfΦ-Cit-r-Cit-rQ)-PEG₁₂-OH | cyclo(SEQ ID NO: 122)-PEG₁₂-OH |
| cyclo(fΦR-Cit-R-Cit-Q)-PEG₁₂-OH | cyclo(SEQ ID NO: 123)-PEG₁₂-OH |
| cyclo(fΦ-Can-r-Can-r-Q)-PEG₁₂-OH | cyclo(SEQ ID NO: 124)-PEG₁₂-OH |
| cyclo(FfΦ-Can-r-Can-r-Q)-PEG₁₂-OH | cyclo(SEQ ID NO: 125)-PEG₁₂-OH |

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 5:

TABLE 5
cCPP-linker conjugate and SEQ ID NOs

| | |
|---|---|
| Ac-PKKK-Can-KV-PEG₂-K(cyclo(FfΦ-Can-r- | Ac-SEQ ID NO: 126-PEG₂-K(cyclo(SEQ ID |
| Can-r-Q)-PEG₁₂-K(N₃)-NH₂ | NO: 125)-PEG₁₂-K(N₃)-NH₂ |
| Ac-PKKKRKV-Lys(cyclo[FfΦ-R-r-Cit-rQ])- | Ac-SEQ ID NO: 42-Lys(cyclo[SEQ ID |
| PEG₁₂-K(N₃)-NH₂ | NO: 127])-PEG₁₂-K(N₃)-NH₂ |
| Ac-PKKKRKV-Lys(cyclo[FfΦ-Cit-r-R-rQ])- | Ac-SEQ ID NO: 42-Lys(cyclo[SEQ ID |
| PEG₁₂-K(N3)-NH₂ | NO: 128])-PEG₁₂-K(N₃)-NH₂ |
| Ac-PKKKRKV-K(cyclo(FfΦR-cit-R-cit-Q))- | Ac-SEQ ID NO: 42-K(cyclo(SEQ ID |
| PEG₁₂-K(N₃)-NH₂ | NO: 129)-PEG₁₂-K(N₃)-NH₂ |
| Ac-PKKKRKV-PEG2-Lys(cyclo[FfΦ-Cit-r-Cit- | Ac-SEQ ID NO: 42-PEG2-Lys(cyclo[SEQ |
| rQ])-B-k(N₃)-NH₂ | ID NO: 130])-B-k(N₃)-NH₂ |
| Ac-PKKKRKV-PEG2-Lys(cyclo[FfΦ-Cit-r-Cit- | Ac-SEQ ID NO: 42-PEG2-Lys(cyclo[SEQ |
| Q1)-PEG2-k(N₃)-NH₂ | ID NO: 130])-PEG2-k(N₃)-NH₂ |
| Ac-PKKKRKV-PEG2-Lys(cyclo[FfΦ-Cit-r-Cit- | Ac-SEQ ID NO: 42-PEG2-Lys(cyclo[SEQ |
| Q])-PEG4-k(N₃)-NH₂ | ID NO: 130])-PEG4-k(N₃)-NH₂ |
| Ac-PKKKRKV-Lys(cyclo[FfΦ-Cit-r-Cit-rQ])- | Ac-SEQ ID NO: 42-Lys(cyclo[SEQ ID |
| PEG12-k(N₃)-NH₂ | NO: 130])-PEG12-k(N₃)-NH₂ |
| Ac-pkkkrkv-PEG2-Lys(cyclo[FfΦ-Cit-r-Cit-rQ])- | Ac-SEQ ID NO: 131-PEG2-Lys(cyclo[SEQ |
| PEG12-k(N₃)-NH₂ | ID NO: 130])-PEG12-k(N₃)-NH₂ |
| Ac-rrv-PEG2-Lys(cyclo[FfΦ-Cit-r-Cit-rQ])- | Ac-rrv-PEG2-Lys(cyclo[SEQ ID NO: 130])- |
| PEG12-OH | PEG12-OH |
| Ac-PKKKRKV-PEG2-Lys(cyclo[FfΦ-Cit-r-Cit-f- | Ac-SEQ ID NO: 42-PEG2-Lys(cyclo[SEQ |
| Q]-PEG12-k(N₃)-NH₂ | ID NO: 130])-PEG12-k(N₃)-NH₂ |
| Ac-PKKK-Cit-KV-PEG2-Lys(cyclo[FfΦ-Cit-r- | Ac-SEQ ID NO: 126-PEG2-Lys(cyclo[SEQ |
| Cit-r-Q])-PEG12-k(N₃)-NH₂ | DD NO: 130])-PEG12-k(N₃)-NH₂ |
| Ac-PKKKRKV-PEG2-Lys(cyclo[FfΦ-Cit-r-Cit-r- | Ac-SEQ ID NO: 42-PEG2-Lys(cyclo[SEQ |
| Q]-PEG12-K(N₃)-NH₂ | ID NO: 130]-PEG12-K(N₃)-NH₂ |

EEVs comprising a cyclic cell penetrating peptide (cCPP), linker and exocyclic peptide (EP) are provided. An EEV can comprise the structure of Formula (B):

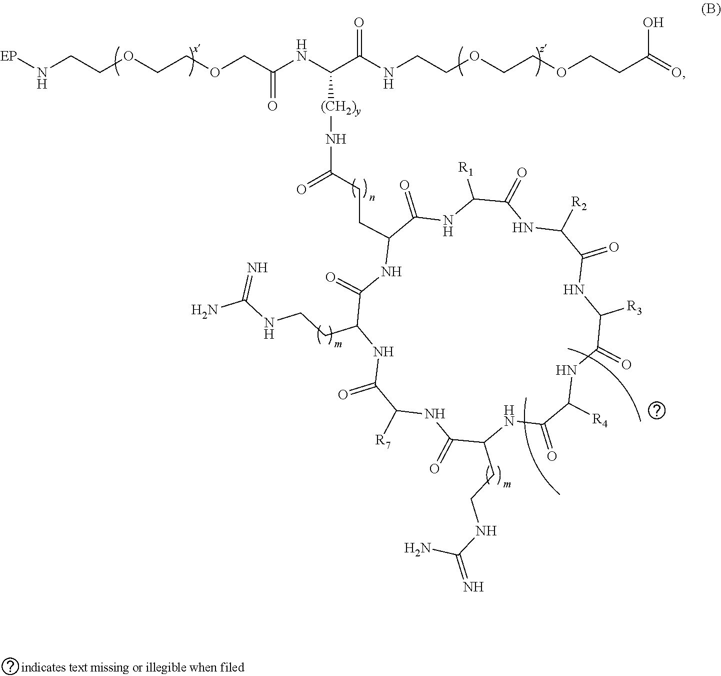

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
   z′ is an integer from 1-23.

R₁, R₂, R₃, R₄, R₇, EP, m, q, y, X′, z′ are as described herein.

n can be 0. n can be 1. n can be 2.

The EEV can comprise the structure of Formula (B-a) or (B-b):

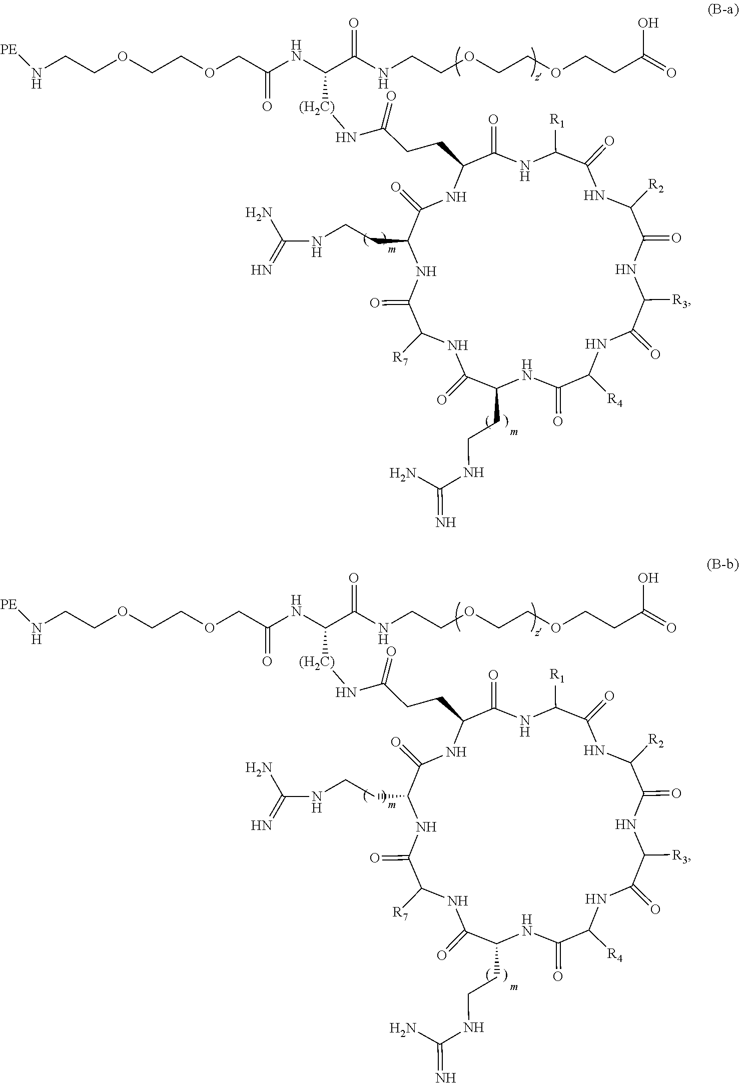

or a protonated form thereof, wherein EP (shown as “PE”), R¹, R², R³, R⁴, m and z′ are as defined above in Formula (B).

The EEV can comprises the structure of Formula (B-c):

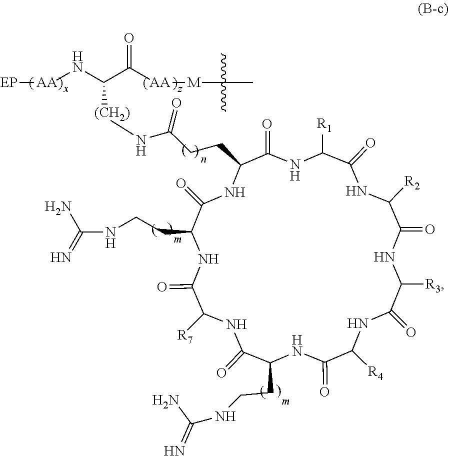

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

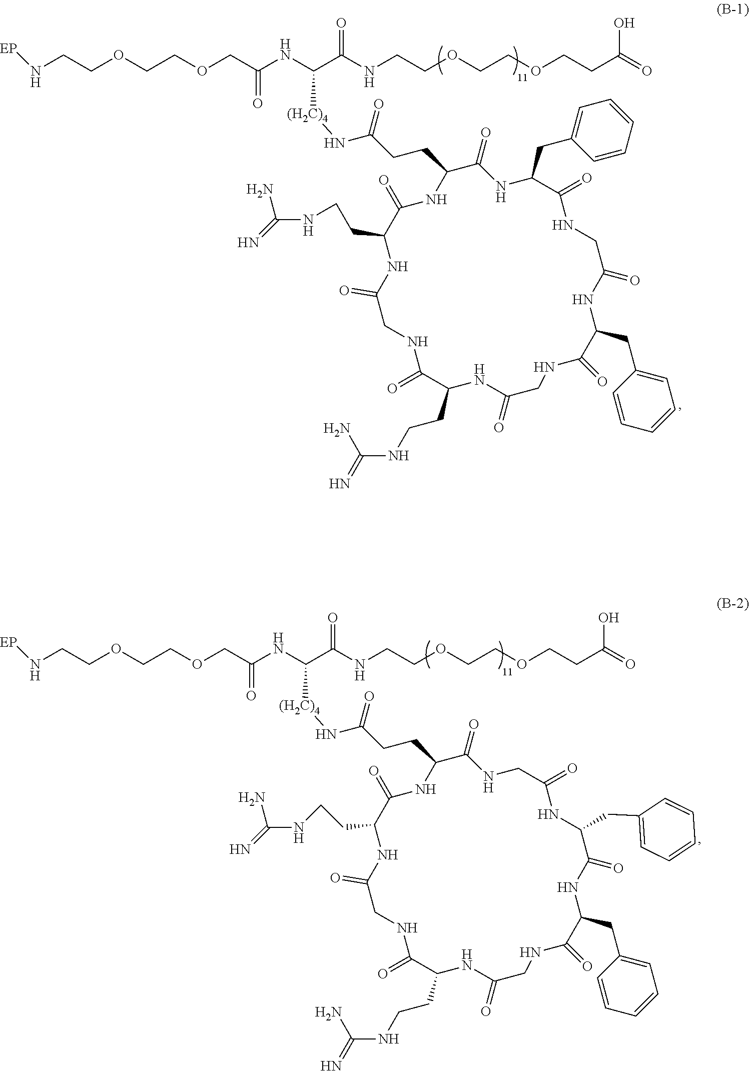

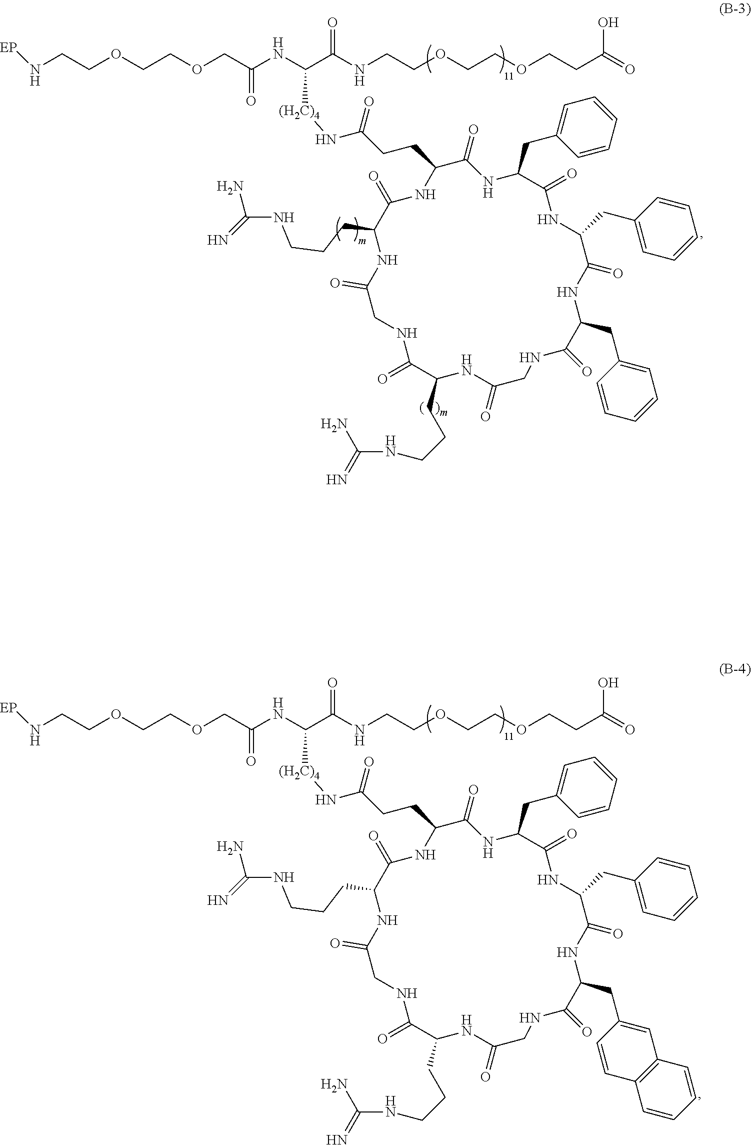

or a protonated form thereof, wherein EP is as defined above in Formula (B).

The EEV can comprise Formula (B) and can have the structure: Ac-PKKKRKVAEEA-K(cyclo[FGFGRGRQ])-PEG₁₂-OH (Ac-SEQ ID NO:132-K(cyclo[SEQ ID NO:82])-PEG₁₂-OH) or Ac-PK-KKR-KV-AEEA-K(cyclo[GfFGrGrQ])-PEG₁₂-OH (Ac-SEQ ID NO:133-K(cyclo[SEQ ID NO:83])-PEG₁₂-OH).

The EEV can comprise a cCPP of formula:

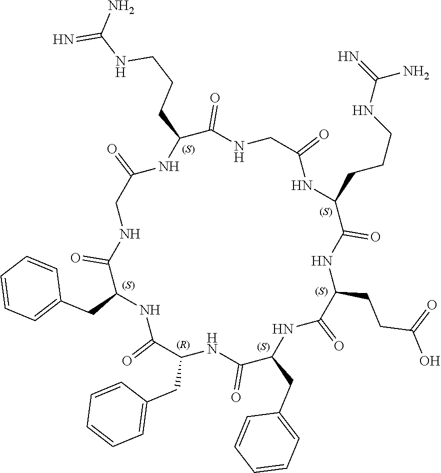

The EEV can comprise formula: Ac-PKKKRKV-miniPEG₂-Lys(cyclo(FfFGRGRQ)-PEG₂-K(N₃) (Ac-SEQ ID NO:42-miniPEG2-Lys(cyclo(SEQ ID NO:81)-PEG₂-K(N₃)).

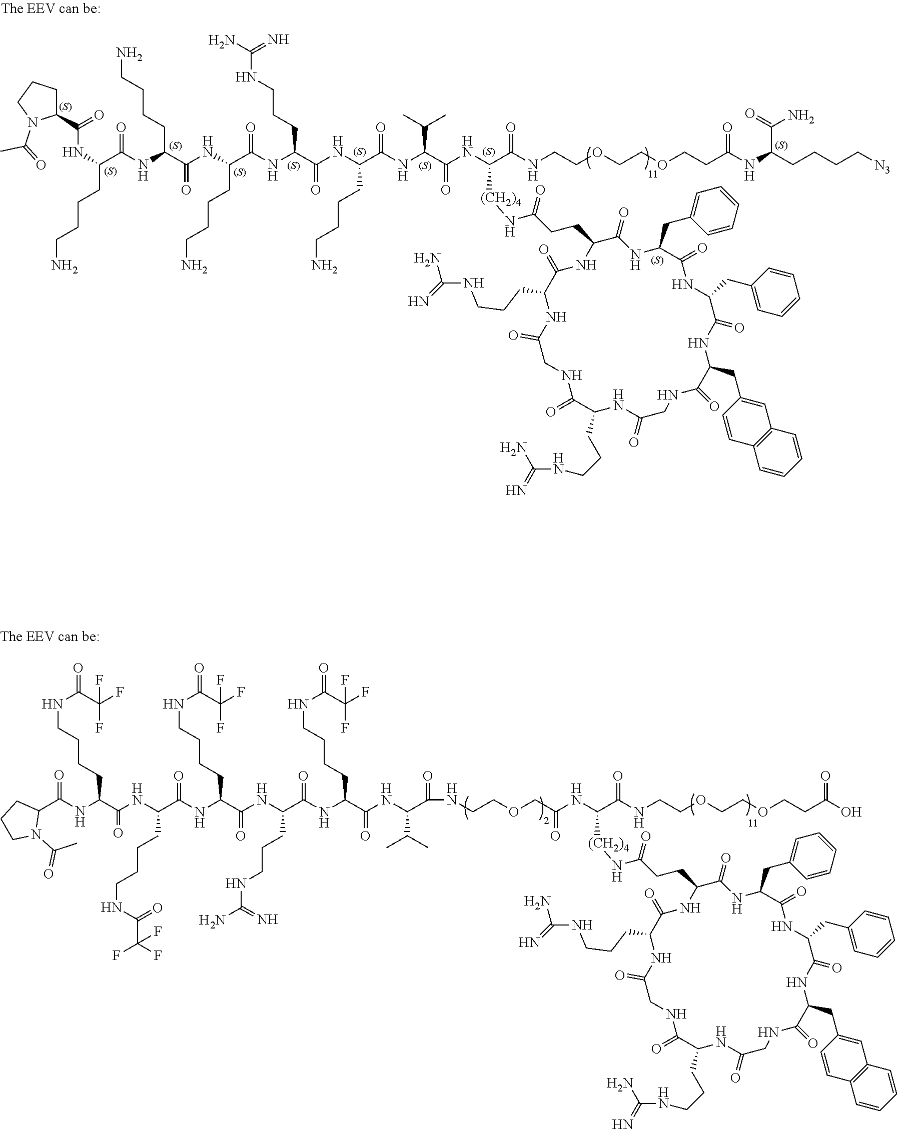

The EEV can be Ac—P-K(Tfa)-K(Tfa)-K(Tfa)-R-K(Tfa)-V-miniPEG₂-K(cyclo(Ff-Nal-GrGrQ)-PEG₁₂-OH (Ac-SEQ ID NO:134-miniPEG2-K(cyclo(SEQ ID NO:135)-PEG₁₂-OH).

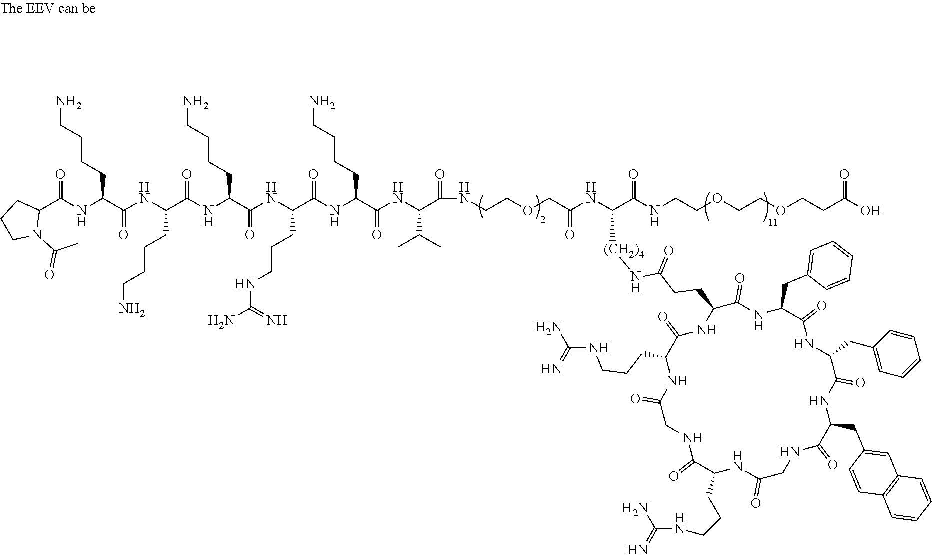

The EEV can be Ac—P—K—K—K—R—K—V-miniPEG2-K(cyclo(Ff-Nal-GrGrQ)-PEG₁₂-OH (Ac-SEQ ID NO:42-PEG₂-K(cyclo(SEQ ID NO:135)-PEG₁₂-OH).

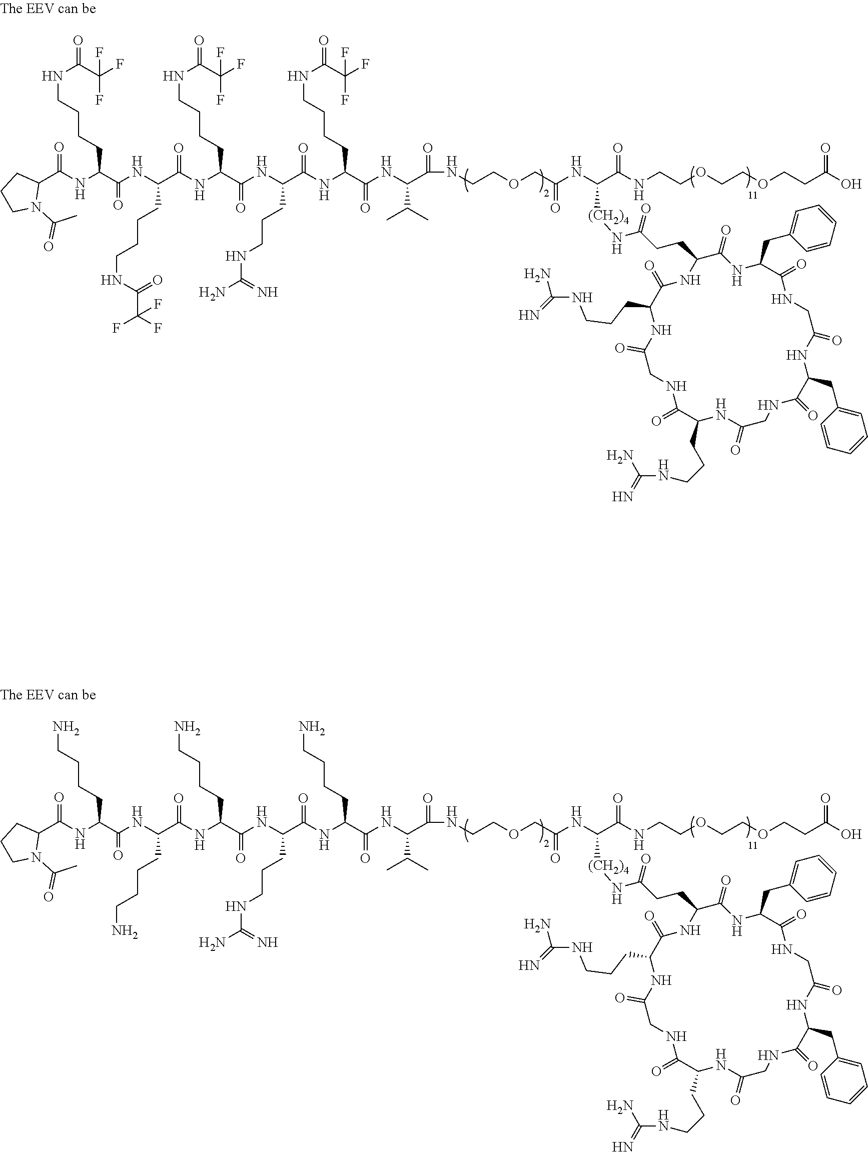

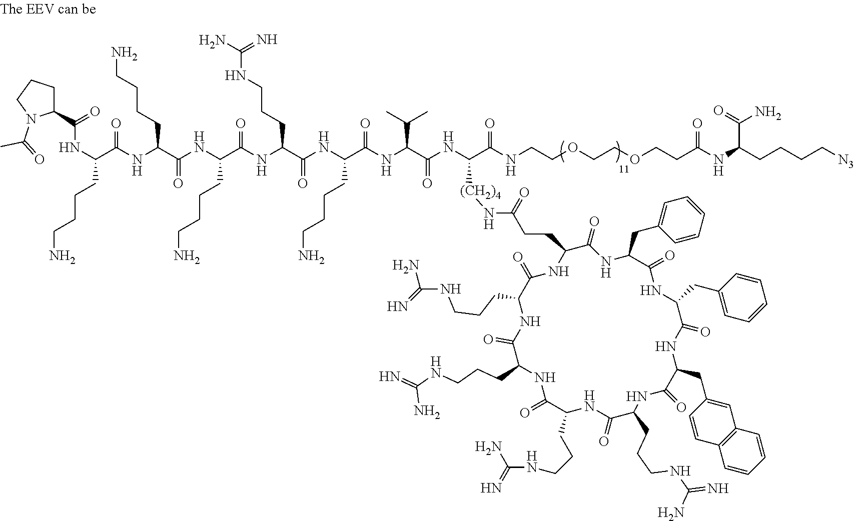

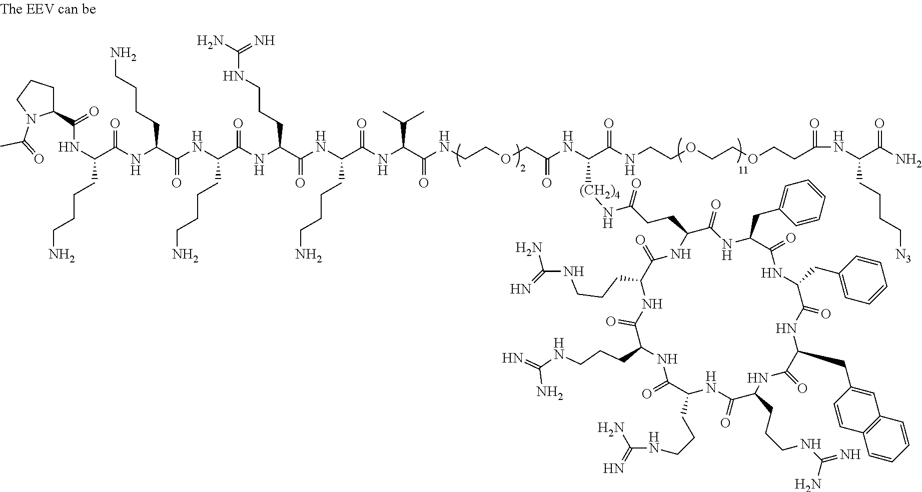

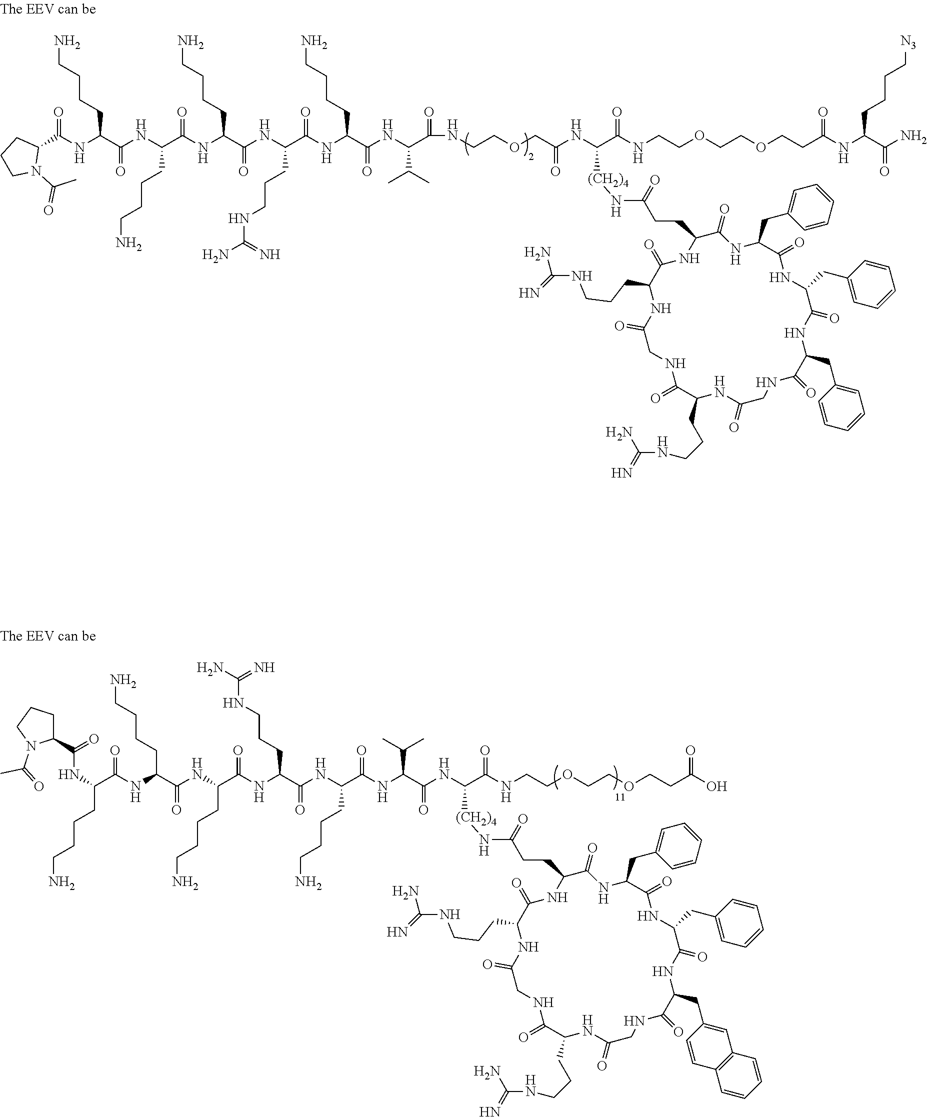

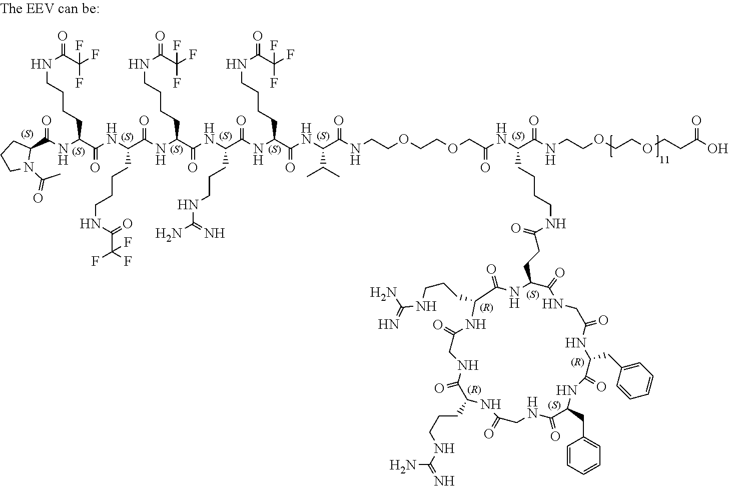

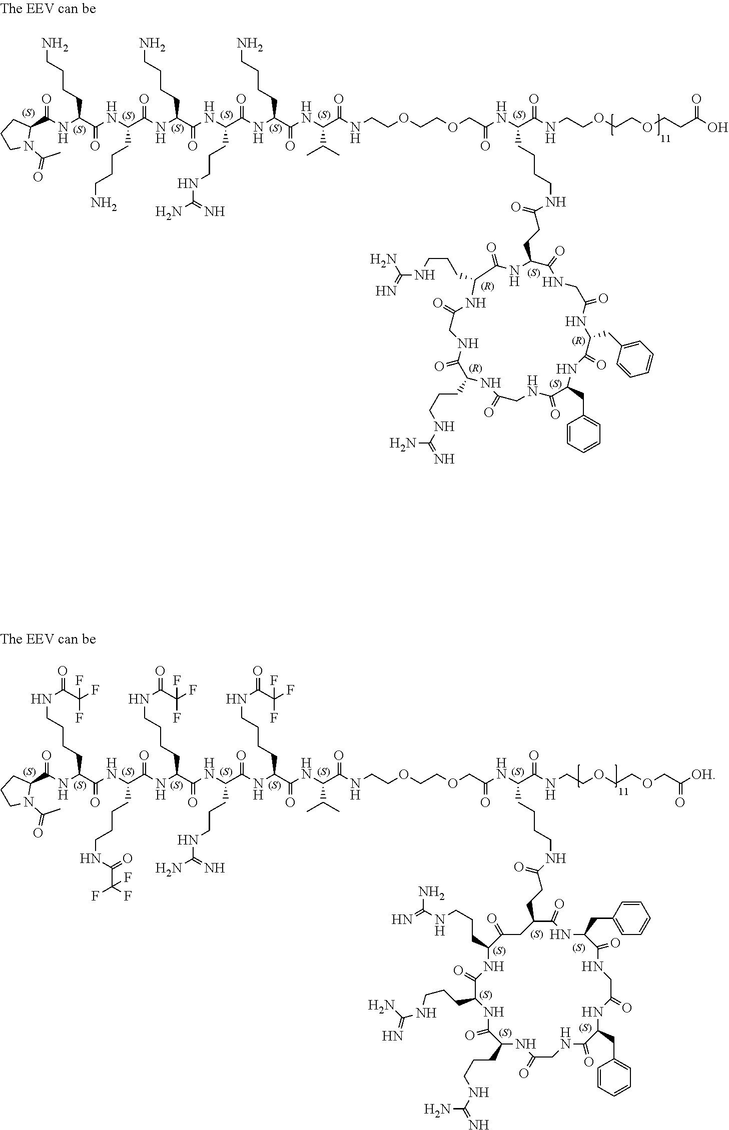

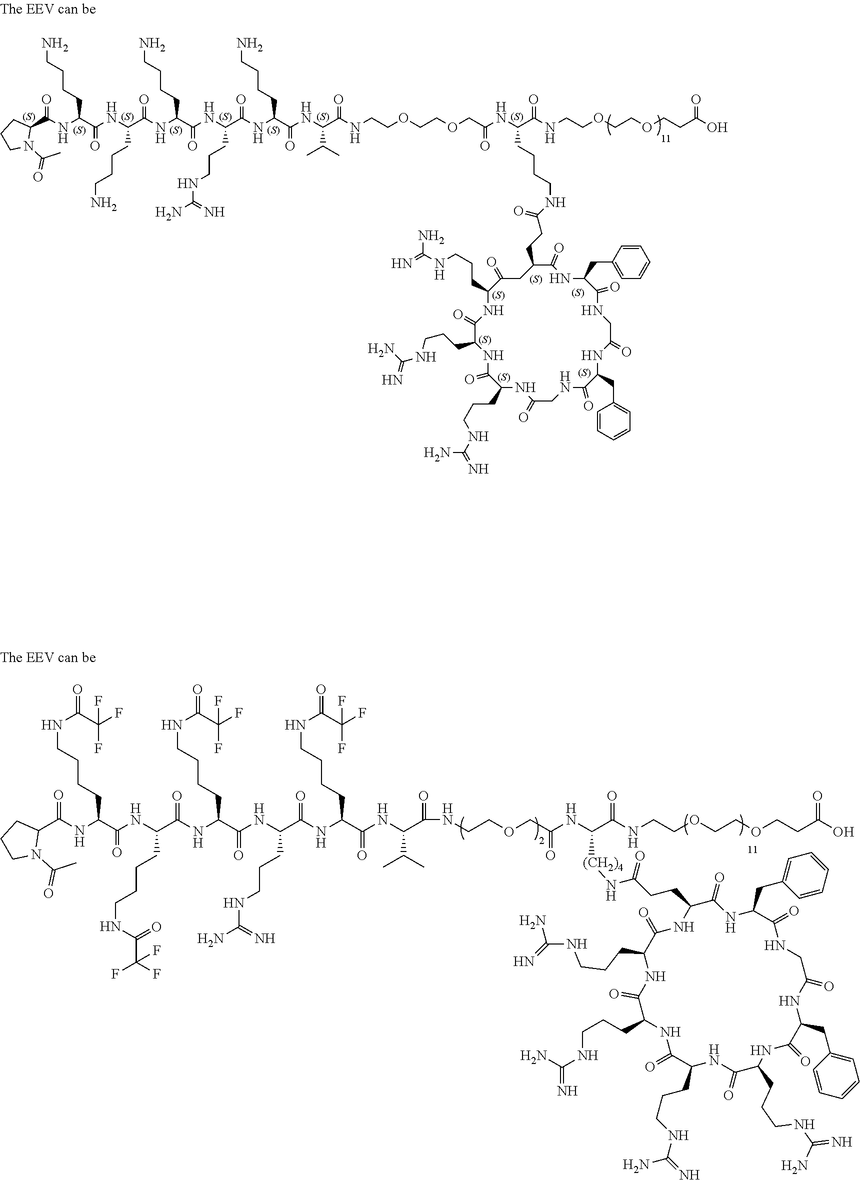

The EEV can be selected from

| | |
|---|---|
| Ac-rr-miniPEG₂-Dap(cyclo[FfΦ-Cit-r-Cit-rQ])-PEG₁₂-OH | Ac-rr-miniPEG2-Dap(cyclo[SEQ |
| | ID NO: 136])-PEG₁₂-OH |
| Ac-frr-PEG₂-Dap(cyclo[FfΦ-Cit-r-Cit-rQ])-PEG₁₂-OH | Ac-frr-PEG2-Dap(cyclo[SEQ ID |
| | NO: 136])-PEG₁₂-OH |
| Ac-rfr-PEG₂-Dap(cyclo[FfΦ-Cit-r-Cit-rQ])-PEG₁₂-OH | Ac-rfr-PEG2-Dap(cyclo[SEQ ID |
| | NO: 136])-PEG₁₂-OH |
| Ac-rbfbr-PEG₂-Dap(cyclo[FfΦ-Cit-r-Cit-rQ])-PEG₁₂-OH | Ac-SEQ ID NO: 137-PEG₂- |
| | Dap(cyclo[SEQ ID NO: 136])- |
| | PEG₁₂-OH |
| Ac-rrr-PEG₂-Dap(cyclo[FfΦ-Cit-r-Cit-rQ])-PEG₁₂-OH | Ac-rrr-PEG₂-Dap(cyclo[SEQ ID |
| | NO: 136])-PEG₁₂-OH |
| Ac-rbr-PEG₂-Dap(cyclo[FfΦ-Cit-r-Cit-rQ])-PEG₁₂-OH | Ac-rbr-PEG₂-Dap(cyclo[SEQ ID |
| | NO: 136])-PEG₁₂-OH |
| Ac-rbrbr-PEG₂-Dap(cyclo[FfΦ-Cit-r-Cit-rQ])-PEG₁₂-OH | Ac-SEQ ID NO: 138-PEG₂- |
| | Dap(cyclo[SEQ ID NO: 136])- |
| | PEG₁₂-OH |
| Ac-hh-PEG₂-Dap(cyclo[FfΦ-Cit-r-Cit-rQ])-PEG₁₂-OH | Ac-hh-PEG₂-Dap(cyclo[SEQ ID |
| | NO: 136])-PEG₁₂-OH |
| Ac-hbh-PEG₂-Dap(cyclo[FfΦ-Cit-r-Cit-rQ])-PEG₁₂-OH | Ac-hbh-PEG₂-Dap(cyclo[SEQ ID |
| | NO: 136])-PEG₁₂-OH |
| Ac-hbhbh-PEG₂-Dap(cyclo[FfΦ-Cit-r-Cit-rQ])-PEG₁₂-OH | Ac-SEQ IN NO: 139-PEG₂- |
| | Dap(cyclo[SEQ ID NO: 136])- |
| | PEG₁₂-OH |
| Ac-rbhbh-PEG₂-Dap(cyclo[FfΦ-Cit-r-Cit-rQ])-PEG₁₂-OH | Ac-SEQ ID NO: 140-PEG₂- |
| | Dap(cyclo[SEQ ID NO: 136])- |
| | PEG₁₂-OH |
| Ac-hbrbh-PEG₂-Dap(cyclo[FfΦ-Cit-r-Cit-rQ])-PEG₁₂-OH | Ac-SEQ ID NO: 141-PEG₂- |
| | Dap(cyclo[SEQ ID NO: 136])- |
| | PEG₁₂-OH |
| Ac-rr-Dap(cyclo[FfΦ-Cit-r-Cit-rQ])-b-OH | Ac-rr-Dap(cyclo[SEQ ID |
| | NO: 136]-b-OH |
| Ac-frr-Dap(cyclo[FfΦ-Cit-r-Cit-rQ])-b-OH | Ac-frr-Dap(cyclo[SEQ ID |
| | NO: 136])-b-OH |
| Ac-rfr-Dap(cyclo[FfΦ-Cit-r-Cit-rQ])-b-OH | Ac-rfr-Dap(cyclo[SEQ ID |
| | NO: 136])-b-OH |
| Ac-rbfbr-Dap(cyclo[FfΦ-Cit-r-Cit-rQ])-b-OH | Ac-SEQ ID NO: 137- |
| | Dap(cyclo[SEQ ID NO: 136])-b- |
| | OH |
| Ac-rrr-Dap(cyclo[FfΦ-Cit-r-Cit-rQ])-b-OH | Ac-rrr-Dap(cyclo[SEQ ID |
| | NO: 136])-b-OH |
| Ac-rbr-Dap(cyclo[FfΦ-Cit-r-Cit-rQ])-b-OH | Ac-rbr-Dap(cyclo[SEQ ID |
| | NO: 136])-b-OH |
| Ac-rbrbr-Dap(cyclo[FfΦ-Cit-r-Cit-rQ])-b-OH | Ac-SEQ ID NO: 138- |
| | Dap(cyclo[SEQ ID NO: 136])-b- |
| | OH |
| Ac-hh-Dap(cyclo[FfΦ-Cit-r-Cit-rQ])-b-OH | Ac-hh-Dap(cyclo[SEQ ID |
| | NO: 136])-b-OH |
| Ac-hbh-Dap(cyclo[FfΦ-Cit-r-Cit-rQ])-b-OH | Ac-hbh-Dap(cyclo[SEQ ID |
| | NO: 136])-b-OH |
| Ac-hbhbh-Dap(cyclo[FfΦ-Cit-r-Cit-rQ])-b-OH | Ac-SEQ IN NO: 139- |
| | Dap(cyclo[SEQ ID NO: 136])-b- |
| | OH |
| Ac-rbhbh-Dap(cyclo[FfΦ-Cit-r-Cit-rQ])-b-OH | Ac-SEQ ID NO: 140- |
| | Dap(cyclo[SEQ ID NO: 136])-b- |
| | OH |
| Ac-hbrbh-Dap(cyclo[FfΦ-Cit-r-Cit-rQ])-b-OH | Ac-SEQ ID NO: 141- |
| | Dap(cyclo[SEQ ID NO: 136])-b- |
| | OH |
| Ac-KKKK-miniPEG₂-Lys(cyclo[FfΦGrGrQ])-miniPEG₂- | Ac-SEQ ID NO: 7-miniPEG₂- |
| K(N₃)-NH₂ | Lys(cyclo[SEQ ID NO: Z80])- |
| | miniPEG₂-K(N₃)-NH₂ |
| Ac-KGKK-miniPEG₂-Lys(cyclo[FfΦGrGrQ])-miniPEG₂- | Ac-SEQ ID NO: 13-miniPEG₂- |
| K(N₃)-NH₂ | Lys(cyclo[SEQ ID NO: Z80])- |
| | miniPEG₂-K(N₃)-NH₂ |
| Ac-KKGK-miniPEG₂-Lys(cyclo[FfΦGrGrQ])-miniPEG₂- | Ac-SEQ ID NO: 14-miniPEG₂- |
| K(N₃)-NH₂ | Lys(cyclo[SEQ ID NO: Z80]- |
| | miniPEG₂-K(N₃)-NH₂ |
| Ac-KKK-miniPEG₂-Lys(cyclo[FfΦGrGrQ])-miniPEG₂- | Ac-KKK-miniPEG₂- |
| K(N₃)-NH₂ | Lys(cyclo[SEQ ID NO: 80]- |
| | miniPEG₂-K(N₃)-NH₂ |
| Ac-KKmini-PEG₂-Lys(cyclo[FfΦGrGrQ])-miniPEG₂- | Ac-KK-miniPEG₂-Lys(cyclo[SEQ |
| K(N₃)-NH₂ | ID NO: 80])-miniPEG₂-K(N₃)-NH₂ |
| Ac-KGK-miniPEG₂-Lys(cyclo[FfΦGrGrQ])-miniPEG₂- | Ac-KGK-miniPEG₂- |
| K(N₃)-NH₂ | Lys(cyclo[SEQ ID NO: 80])- |
| | miniPEG₂-K(N₃)-NH₂ |
| Ac-KBK-miniPEG₂-Lys(cyclo[FfΦGrGrQ])-miniPEG₂- | Ac-KBK-miniPEG₂- |
| K(N₃)-NH₂ | Lys(cyclo[SEQ ID NO: 80])- |
| | miniPEG₂-K(N₃)-NH₂ |
| Ac-KBKBK-miniPEG₂-Lys(cyclo[FfΦGrGrQ])-miniPEG₂- | Ac-SEQ ID NO: 24-miniPEG₂- |
| K(N₃)-NH₂ | Lys(cyclo[SEQ ID NO: 80])- |
| | miniPEG₂-K(N₃)-NH₂ |
| Ac-KR-miniPEG₂-Lys(cyclo[FfΦGrGrQ])-miniPEG₂- | Ac-KR-miniPEG₂-Lys(cyclo[SEQ |
| K(N₃)-NH₂ | ID NO: 80])-miniPEG₂-K(N₃)-NH₂ |
| Ac-KBR-miniPEG₂-Lys(cyclo[FfΦGrGrQ])-miniPEG₂- | Ac-KBR-miniPEG₂- |
| K(N₃)-NH₂ | Lys(cyclo[SEQ ID NO: 80]- |
| | miniPEG₂-K(N₃)-NH₂ |
| Ac-PKKKRKV-miniPEG₂-Lys(cyclo[FfΦGrGrQ])- | Ac-SEQ ID NO: 42-miniPEG₂- |
| miniPEG₂-K(N₃)-NH₂ | Lys(cyclo[SEQ ID NO: 80])- |
| | miniPEG₂-K(_{N3})-NH₂ |
| Ac-PKKKRKV-miniPEG₂-Lys(cyclo[FfΦGrGrQ])- | Ac SEQ ID NO: 42-miniPEG₂- |
| miniPEG₂-K(N₃)-NH₂ | Lys(cyclo[SEQ ID NO: 80])- |
| | miniPEG₂-K(N₃)-NH₂ |
| Ac-PGKKRKV-miniPEG₂-Lys(cyclo[FfΦGrGrQ])- | Ac-SEQ ID NO: 43-miniPEG₂- |
| miniPEG₂-K(N₃)-NH₂ | Lys(cyclo[SEQ ID NO: 80])- |
| | miniPEG₂-K(N₃)-NH₂ |
| Ac-PKGKRKV-miniPE_{G2}-Lys(cyclo[FfΦGrGrQ])- | Ac-SEQ ID NO: 44-miniPEG₂- |
| miniPEG₂-K(N₃)-NH₂ | Lys(cyclo[SEQ ID NO: 80]- |
| | miniPEG₂-K(N₃)-NH₂ |
| Ac-PKKGRKV-miniPEG₂-Lys(cyclo[FfΦGrGrQ])- | Ac-SEQ ID NO: 45-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKGKV-miniPEG₂-Lys(cyclo[FfΦGrGrQ])- | Ac-SEQ ID NO: 46-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKRGV-miniPEG₂-Lys(cyclo[FfΦGrGrQ])- | Ac-SEQ ID NO: 47-miniPEG₂- |
| miniPEG₂-K(N₃)-NH₂ | Lys(cyclo[SEQ ID NO: 80])- |
| | miniPEG₂-K(N₃)-NH₂ |
| Ac-PKKKRKG-miniPEG₂-Lys(cyclo[FfΦGrGrQ])- | Ac-SEQ ID NO: 48-miniPEG₂- |
| miniPEG₂-K(N₃)-NH₂ | Lys(cyclo[SEQ ID NO: 80])- |
| | miniPEG₂-K(N₃)-NH₂ |
| Ac-KKKRK-miniPEG₂-Lys(cyclo[FfΦGrGrQ])- | Ac-SEQ ID NO: 19-miniPEG₂- |
| miniPEG₂-K(N₃)-NH₂ | Lys(cyclo[SEQ ID NO: 80])- |
| | miniPEG₂-K(N₃)-NH₂ |
| Ac-KKRK-miniPEG₂-Lys(cyclo[FfΦGrGrQ])-miniPEG₂- | Ac-SEQ ID NO: 8-miniPEG₂- |
| K(N₃)-NH₂ and | Lys(cyclo[SEQ ID NO: 80])- |
| | miniPEG₂-K(N₃)-NH₂ and |
| Ac-KRK-miniPEG₂-Lys(cyclo[FfΦGrGrQ])-miniPEG₂- | Ac-KRK-miniPEG₂- |
| K(N₃)-NH₂. | Lys(cyclo[SEQ ID NO: 80])- |
| | miniPEG₂-K(N₃)-NH₂. |

The EEV can be selected from:

Ac-PKKKRKV-Lys(cyclo[FfΦGrGrQ])-PEG1₂-K(N₃)-NH₂
(Ac-SEQ ID NO: 42-Lys(cyclo[SEQ ID NO: 80])-PEG₁₂-K(N₃)-NH₂)
Ac-PKKKRKV-miniPEG₂-Lys(cyclo[FfΦGrGrQ])-miniPEG₂-K(N3)-NH₂
(Ac-SEQ ID NO: 42-miniPEG₂-Lys(cyclo[SEQ ID NO: 80])-miniPEG₂-K(N₃)-NH₂)
Ac-PKKKRKV-miniPEG₂-Lys(cyclo[FGFGRGRQ])-miniPEG₂-K(N₃)-NH₂
(Ac-SEQ ID NO: 42-miniPEG₂-Lys(cyclo[SEQ ID NO: 82])-miniPEG₂-K(N₃)-NH₂)
Ac-KR-PEG₂-K(cyclo[FGFGRGRQ])-PEG₂-K(N₃)-NH₂
(Ac-KR-PEG₂-K(cyclo[SEQ ID NO: 82])-PEG₂-K(N₃)-NH₂)
Ac-PKKKGKV-PEG₂-K(cyclo[FGFGRGRQ])-PEG₂-K(N₃)-NH₂
(Ac-SEQ ID NO: 46-PEG₂-K(cyclo[SEQ ID NO: 82])-PEG₂-K(N₃)-NH₂)
Ac-PKKKRKG-PEG₂-K(cyclo[FGFGRGRQ])-PEG₂-K(N₃)-NH₂
(Ac-SEQ ID NO: 48-PEG₂-K(cyclo[SEQ ID NO: 82])-PEG₂-K(N₃)-NH₂)
Ac-KKKRK-PEG₂-K(cyclo[FGFGRGRQ])-PEG₂-K(N₃)-NH₂
(Ac-SEQ ID NO: 19-PEG₂-K(cyclo[SEQ ID NO: 82])-PEG₂-K(N₃)-NH₂)
Ac-PKKKRKV-miniPEG₂-Lys(cyclo[FFΦGRGRQ])-miniPEG₂-K(N₃)-NH₂
(Ac-SEQ ID NO: 42-miniPEG₂-Lys(cyclo[SEQ ID NO: 80])-miniPEG₂-K(N₃)-NH₂)
Ac-PKKKRKV-miniPEG₂-Lys(cyclo[BhFfΦGrGrQ])-miniPEG₂-K(N₃)-NH₂
(Ac-SEQ ID NO: 42-miniPEG₂-Lys(cyclo[SEQ ID NO: 142])-miniPEG₂-K(N₃)-NH₂)
Ac-PKKKRKV-miniPEG₂-Lys(cyclo[FfΦSrSrQ])-miniPEG₂-K(N₃)-NH₂
(Ac-SEQ ID NO: 42-miniPEG₂-Lys(cyclo[SEQ ID NO: 143])-miniPEG₂-K(N₃)-NH₂).
The EEV can be selected from:
Ac-PKKKRKV-miniPEG₂-Lys(cyclo(GfFGrGrQ])-PEG₁₂-OH
(Ac-SEQ ID NO: 42-miniPEG₂-Lys(cyclo(SEQ ID NO: 133])-PEG₁₂-OH)
Ac-PKKKRKV-miniPEG₂-Lys(cyclo[FGFKRKRQ])-PEG₁₂-OH
(Ac-SEQ ID NO: 42-miniPEG₂-Lys(cyclo[SEQ ID NO: 144])-PEG₁₂-OH)
Ac-PKKKRKV-miniPEG₂-Lys(cyclo[FGFRGRGQ])-PEG₁2-OH
(Ac-SEQ ID NO: 42-miniPEG₂-Lys(cyclo[SEQ ID NO: 145])-PEG₁₂-OH)
Ac-PKKKRKV-miniPEG₂-Lys(cyclo[FGFGRGRGRQ])-PEG₁₂-OH
(Ac-SEQ ID NO: 42-miniPE_{G2}-Lys(cyclo[SEQ ID NO: 146])-PEG₁₂-OH)
Ac-PKKKRKV-miniPEG₂-Lys(cyclo[FGFGRrRQ])-PEG₁₂-OH
(Ac-SEQ ID NO: 42-miniPEG₂-Lys(cyclo[SEQ ID NO: 147])-PEG₁₂-OH)
Ac-PKKKRKV-miniPEG₂-Lys(cyclo[FGFGRRRQ])-PEG₁₂-OH
(Ac-SEQ ID NO: 42-miniPEG₂-Lys(cyclo[SEQ ID NO: 84])-PEG₁₂-OH)and
Ac-PKKKRKV-miniPEG₂-Lys(cyclo[FGFRRRRQ])-PEG₁₂-OH
(Ac-SEQ ID NO: 42-miniPEG₂-Lys(cyclo[SEQ ID NO: 85])-PEG₁₂-OH).
The EEV can be selected from:
Ac-K-K-K-R-K-G-miniPEG₂-K(cyclo[FGFGRGRQ])-PEG₁₂-OH
(Ac-SEQ ID NO: 148-miniPEG₂-K(cyclo[SEQ ID NO: 82])-PEG₁₂-OH)
Ac-K-K-K-R-K-miniPEG₂-K(cyclo[FGFGRGRQ])-PEG₁₂-OH
(Ac-SEQ ID NO: 19-miniPEG₂-K(cyclo[SEQ ID NO: 82])-PEG₁₂-OH)
Ac-K-K-R-K-K-miniPEG₄-K(cyclo[FGFGRGRQ])-PEG₁₂-OH
(Ac-SEQ ID NO: 22-PEG₄-K(cyclo[SEQ ID NO: 82])-PEG₁₂-OH)
Ac-K-R-K-K-K-PEG₄-K(cyclo[FGFGRGRQ])-PEG₁₂-OH
(Ac-SEQ ID NO: 21-PEG₄-K(cyclo[SEQ ID NO: 82])-PEG₁₂-OH)
Ac-K-K-K-K-R-PEG₄-K(cyclo[FGFGRGRQ])-PEG₁₂-OH
(Ac-SEQ ID NO: 23-PEG₄-K(cyclo[SEQ ID NO: 82])-PEG₁₂-OH)
Ac-R-K-K-K-K-PEG₄-K(cyclo[FGFGRGRQ])-PEG₁₂-OH
(Ac-SEQ ID NO: 20-PEG₄-K(cyclo[SEQ ID NO: 82])-PEG₁₂-OH)
and
Ac-K-K-K-R-K-PEG₄-K(cyclo[FGFGRGRQ])-PEG₁₂-OH
(Ac-SEQ ID NO: 19-PEG4-K(cyclo[SEQ ID NO: 82])-PEG₁₂-OH).
The EEV can be selected from:
Ac-PKKKRKV-PEG₂-K(cyclo[FGFGRGRQ])-PEG₂-K(N₃)-NH₂
(Ac-SEQ ID NO: 42-PEG₂-K(cyclo[SEQ ID NO: 82])-PEG₂-K(N₃)-NH₂)
Ac-PKKKRKV-PEG₂-K(cyclo[FGFGRGRQ])-PEG₁₂-OH
(Ac-SEQ ID NO: 42-PEG₂-K(cyclo[SEQ ID NO: 82])-PEG₁₂-OH)
Ac-PKKKRKV-PEG₂-K(cyclo[GfFGrGrQ])-PEG₂-K(N₃)-NH₂
(Ac-SEQ ID NO: 42-PEG₂-K(cyclo[SEQ ID NO: 133])-PEG₂-K(N₃)-NH₂)
and
Ac-PKKKRKV-PEG₂-K(cyclo[GfFGrGrQ])-PEG₁₂-OH
(Ac-SEQ ID NO: 42-PEG₂-K(cyclo[SEQ ID NO: 133])-PEG₁₂-OH).
The cargo can be a protein and the EEV can be selected from:
Ac-PKKKRKV-PEG₂-K(cyclo[FfΦGrGrQ])-PEG₁₂-OH
(Ac-SEQ ID NO: 42-PEG₂-K(cyclo[SEQ ID NO: 80])-PEG₁₂-OH)
Ac-PKKKRKV-PEG₂-K(cyclo[FfΦCit-r-Cit-rQ])-PEG₁₂-OH
(Ac-SEQ ID NO: 42-PEG₂-K(cyclo[SEQ ID NO: 79])-PEG₁₂-OH)
Ac-PKKKRKV-PEG₂-K(cyclo[FfFGRGRQ])-PEG₁₂-OH
(Ac-SEQ ID NO: 42-PEG₂-K(cyclo[SEQ ID NO: 81])-PEG₁₂-OH)
Ac-PKKKRKV-PEG₂-K(cyclo[FGFGRGRQ])-PEG₁₂-OH
(Ac-SEQ ID NO: 42-PEG₂-K(cyclo[SEQ ID NO: 82])-PEG₁₂-OH)
Ac-PKKKRKV-PEG₂-K(cyclo[GfFGrGrQ])-PEG₁₂-OH
(Ac-SEQ ID NO: 42-PEG₂-K(cyclo[SEQ ID NO: 133])-PEG₁₂-OH)
Ac-PKKKRKV-PEG₂-K(cyclo[FGFGRRRQ])-PEG₁₂-OH
(Ac-SEQ ID NO: 42-PEG₂-K(cyclo[SEQ ID NO: 84])-PEG₁₂-OH)
Ac-PKKKRKV-PEG₂-K(cyclo[FGFRRRRQ])-PEG₁₂-OH
(Ac-SEQ ID NO: 42-PEG₂-K(cyclo[SEQ ID NO: 85])-PEG₁₂-OH)
Ac-rr-PEG₂-K(cyclo[FfΦGrGrQ])-PEG₁₂-OH
(Ac-rr-PEG₂-K(cyclo[SEQ ID NO: 80])-PEG₁₂-OH)
Ac-rr-PEG₂-K(cyclo[FfΦCit-r-Cit-rQ])-PEG₁₂-OH
(Ac-rr-PEG₂-K(cyclo[SEQ ID NO: 79])-PEG₁₂-OH)
Ac-rr-PEG₂-K(cyclo[FfF-GRGRQ])-PEG₁₂-OH
(Ac-rr-PEG₂-K(cyclo[SEQ ID NO: 81])-PEG₁₂-OH)
Ac-rr-PEG₂-K(cyclo[FGFGRGRQ])-PEG₁₂-OH
(Ac-rr-PEG₂-K(cyclo[SEQ ID NO: 82])-PEG₁₂-OH)
Ac-rr-PEG₂-K(cyclo[GfFGrGrQ])-PEG₁₂-OH
(Ac-rr-PEG₂-K(cyclo[SEQ ID NO: 133])-PEG₁₂-OH)
Ac-rr-PEG₂-K(cyclo[FGFGRRRQ])-PEG₁₂-OH
(Ac-rr-PEG₂-K(cyclo[SEQ ID NO: 84])-PEG₁₂-OH)
Ac-rr-PEG₂-K(cyclo[FGFRRRRQ])-PEG₁₂-OH
(Ac-rr-PEG₂-K(cyclo[SEQ ID NO: 85])-PEG₁₂-OH)
Ac-rrr-PEG₂-K(cyclo[FfΦGrGrQ])-PEG₁₂-OH
(Ac-rrr-PEG₂-K(cyclo[SEQ ID NO: 80])-PEG₁₂-OH)
Ac-rrr-PEG₂-K(cyclo[FfΦCit-r-Cit-rQ])-PEG₁₂-OH
(Ac-rrr-PEG₂-K(cyclo[SEQ ID NO: 79])-PEG₁₂-OH)
Ac-rrr-PEG₂-K(cyclo[FfFGRGRQ])-PEG₁₂-OH
(Ac-rrr-PEG₂-K(cyclo[SEQ ID NO: 81])-PEG₁₂-OH)
Ac-rrr-PEG₂-K(cyclo[FGFGRGRQ])-PEG₁₂-OH
(Ac-rrr-PEG₂-K(cyclo[SEQ ID NO: 82])-PEG₁₂-OH)
Ac-rrr-PEG₂-K(cyclo[GfFGrGrQ])-PEG₁₂-OH
(Ac-rrr-PEG₂-K(cyclo[SEQ ID NO: 133])-PEG₁₂-OH)
Ac-rrr-PEG₂-K(cyclo[FGFGRRRQ])-PEG₁₂-OH
(Ac-rrr-PEG₂-K(cyclo[SEQ ID NO: 84])-PEG₁₂-OH)
Ac-rrr-PEG₂-K(cyclo[FGFRRRRQ])-PEG₁₂-OH
(Ac-rrr-PEG₂-K(cyclo[SEQ ID NO: 85])-PEG₁₂-OH)
Ac-rhr-PEG₂-K(cyclo[FfΦGrGrQ])-PEG₁₂-OH
(Ac-rhr-PEG₂-K(cyclo[SEQ ID NO: 80])-PEG₁₂-OH)
Ac-rhr-PEG₂-K(cyclo[FfΦCit-r-Cit-rQ])-PEG₁₂-OH
(Ac-rhr-PEG₂-K(cyclo[SEQ ID NO: 79])-PEG₁₂-OH)
Ac-rhr-PEG₂-K(cyclo[FfFGRGRQ])-PEG₁₂-OH
(Ac-rhr-PEG₂-K(cyclo[SEQ ID NO: 81])-PEG₁₂-OH)
Ac-rhr-PEG₂-K(cyclo[FGFGRGRQ])-PEG₁₂-OH
(Ac-rhr-PEG₂-K(cyclo[SEQ ID NO: 82])-PEG₁₂-OH)
Ac-rhr-PEG₂-K(cyclo[GfFGrGrQ])-PEG₁₂-OH
(Ac-rhr-PEG₂-K(cyclo[SEQ ID NO: 133])-PEG₁₂-OH)
Ac-rhr-PEG₂-K(cyclo[FGFGRRRQ])-PEG₁₂-OH
(Ac-rhr-PEG₂-K(cyclo[SEQ ID NO: 84])-PEG₁₂-OH)
Ac-rhr-PEG₂-K(cyclo[FGFRRRRQ])-PEG₁₂-OH
(Ac-rhr-PEG₂-K(cyclo[SEQ ID NO: 85])-PEG₁₂-OH)
Ac-rbr-PEG₂-K(cyclo[FfΦGrGrQ])-PEG₁₂-OH
(Ac-rbr-PEG₂-K(cyclo[SEQ ID NO: 80])-PEG₁₂₋OH)
Ac-rbr-PEG₂-K(cyclo[FfΦCit-r-Cit-rQ])-PEG₁₂-OH
(Ac-rbr-PEG₂-K(cyclo[SEQ ID NO: 79])-PEG₁₂-OH)
Ac-rbr-PEG₂-K(cyclo[FfFGRGRQ])-PEG₁₂-OH
(Ac-rbr-PEG₂-K(cyclo[SEQ ID NO: 81])-PEG₁₂-OH)
Ac-rbr-PEG₂-K(cyclo[FGFGRGRQ])-PEG₁₂-OH
(Ac-rbr-PEG₂-K(cyclo[SEQ ID NO: 82])-PEG₁₂-OH)
Ac-rbr-PEG₂-K(cyclo[GfFGrGrQ])-PEG₁₂-OH
(Ac-rbr-PEG₂-K(cyclo[SEQ ID NO: 133])-PEG₁₂-OH)
Ac-rbr-PEG₂-K(cyclo[FGFGRRRQ])-PEG₁₂-OH
(Ac-rbr-PEG₂-K(cyclo[SEQ ID NO: 84])-PEG₁₂-OH)
Ac-rbr-PEG₂-K(cyclo[FGFRRRRQ])-PEG₁₂-OH
(Ac-rbr-PEG₂-K(cyclo[SEQ ID NO: 85])-PEG₁₂-OH)
Ac-rbrbr-PEG₂-K(cyclo[FfΦGrGrQ])-PEG₁₂-OH
(Ac-SEQ ID NO: 138-PEG₂-K(cyclo[SEQ ID NO: 80])-PEG₁₂-OH)
Ac-rbrbr-PEG₂-K(cyclo[FfΦCit-r-Cit-rQ])-PEG₁₂-OH
(Ac-SEQ ID NO: 138-PEG₂-K(cyclo[SEQ ID NO: 79])-PEG₁₂-OH)
Ac-rbrbr-PEG₂-K(cyclo[FfFGRGRQ])-PEG₁₂-OH
(Ac-SEQ ID NO: 138-PEG₂-K(cyclo[SEQ ID NO: 81])-PEG₁₂-OH)
Ac-rbrbr-PEG₂-K(cyclo[FGFGRGRQ])-PEG₁₂-OH
(Ac-SEQ ID NO: 138-PEG₂-K(cyclo[SEQ ID NO: 82])-PEG₁₂-OH)
Ac-rbrbr-PEG₂-K(cyclo[GfFGrGrQ])-PEG₁₂-OH
(Ac-SEQ ID NO: 138-PEG₂-K(cyclo[SEQ ID NO: 133])-PEG₁₂-OH)
Ac-rbrbr-PEG₂-K(cyclo[FGFGRRRQ])-PEG₁₂-OH
(Ac-SEQ ID NO: 138-PEG₂-K(cyclo[SEQ ID NO: 84])-PEG₁₂-OH)
Ac-rbrbr-PEG₂-K(cyclo[FGFRRRRQ])-PEG₁₂-OH
(Ac-SEQ ID NO: 138-PEG₂-K(cyclo[SEQ ID NO: 85])-PEG₁₂-OH)
Ac-rbhbr-PEG₂-K(cyclo[FfΦGrGrQ])-PEG₁₂-OH
(Ac-SEQ ID NO: 149-PEG₂-K(cyclo[SEQ ID NO: 80])-PEG₁₂-OH)
Ac-rbhbr-PEG₂-K(cyclo[FfΦCit-r-Cit-rQ])-PEG₁₂-OH
(Ac-SEQ ID NO: 149-PEG₂-K(cyclo[SEQ ID NO: 79])-PEG₁₂-OH)
Ac-rbhbr-PEG₂-K(cyclo[FfFGRGRQ])-PEG₁₂-OH
(Ac-SEQ ID NO: 149-PEG₂-K(cyclo[SEQ ID NO: 81])-PEG₁₂-OH)
Ac-rbhbr-PEG₂-K(cyclo[FGFGRGRQ])-PEG₁₂-OH
(Ac-SEQ ID NO: 149-PEG₂-K(cyclo[SEQ ID NO: 82])-PEG₁₂-OH)
Ac-rbhbr-PEG₂-K(cyclo[GfFGrGrQ])-PEG₁₂-OH
(Ac-SEQ ID NO: 149-PEG₂-K(cyclo[SEQ ID NO: 133])-PEG₁₂-OH)
Ac-rbhbr-PEG₂-K(cyclo[FGFGRRRQ])-PEG₁₂-OH
(Ac-SEQ ID NO: 149-PEG₂-K(cyclo[SEQ ID NO: 84])-PEG₁₂-OH)
Ac-rbhbr-PEG₂-K(cyclo[FGFRRRRQ])-PEG₁₂-OH
(Ac-SEQ ID NO: 149-PEG₂-K(cyclo[SEQ ID NO: 85])-PEG₁₂-OH)
Ac-hbrbh-PEG₂-K(cyclo[FfΦGrGrQ])-PEG₁₂-OH
(Ac-SEQ ID NO: 141-PEG₂-K(cyclo[SEQ ID NO: 80])-PEG₁₂-OH)
Ac-hbrbh-PEG₂-K(cyclo[FfΦCit-r-Cit-rQ])-PEG₁₂-OH
(Ac-SEQ ID NO: 141-PEG₂-K(cyclo[SEQ ID NO: 79])-PEG₁₂-OH)
Ac-hbrbh-PEG₂-K(cyclo[FfFGRGRQ])-PEG₁₂-OH
(Ac-SEQ ID NO: 141-PEG₂-K(cyclo[SEQ ID NO: 81])-PEG₁₂-OH)
Ac-hbrbh-PEG₂-K(cyclo[FGFGRGRQ])-PEG₁₂-OH
(Ac-SEQ ID NO: 141-PEG₂-K(cyclo[SEQ ID NO: 82])-PEG₁₂-OH)
Ac-hbrbh-PEG₂-K(cyclo[GfFGrGrQ])-PEG₁₂-OH
(Ac-SEQ ID NO: 141-PEG₂-K(cyclo[SEQ ID NO: 133])-PEG₁₂-OH)
Ac-hbrbh-PEG₂-K(cyclo[FGFGRRRQ])-PEG₁₂-OH
(Ac-SEQ ID NO: 141-PEG₂-K(cyclo[SEQ ID NO: 84])-PEG₁₂-OH)
Ac-hbrbh-PEG₂-K(cyclo[FGFRRRRQ])-PEG₁₂-OH
(Ac-SEQ ID NO: 141-PEG₂-K(cyclo[SEQ ID NO: 85])-PEG₁₂-OH),

wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. As used herein, “cargo” is a compound or moiety for which delivery into a cell is desired. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

In embodiments, the amino acid side chain comprises a chemically reactive group to which the linker or cargo is conjugated comprises. The chemically reactive group can comprise an amine group, a carboxylic acid, an amide, a hydroxyl group, a sulfhydryl group, a guanidinyl group, a phenolic group, a thioether group, an imidazolyl group, or an indolyl group. In embodiments, the amino acid of the cCPP to which the cargo is conjugated comprises lysine, arginine, aspartic acid, glutamic acid, asparagine, glutamine, homoglutamine, serine, threonine, tyrosine, cysteine, arginine, tyrosine, methionine, histidine or tryptophan.

The cargo can comprise one or more detectable moieties, one or more therapeutic moieties (TMs), one or more targeting moieties, or any combination thereof. In embodiments, the cargo comprises a TM. In embodiments, the TM comprises an antisense compound (AC). In embodiments, the AC binds to at least a portion of polyadenylation sequence element (PSE) of a target gene transcript or in sufficient proximity to the PSE of the target gene transcript to modulate polyadenylation of the target gene transcript. In embodiments, the AC binds to at least a portion of a PSE of a target IRF-5, DPMK, or DUX4 gene transcript. In embodiments, the AC binds in sufficient proximity to a PSE of a target IRF-5, DPMK, or DUX4 gene transcript to modulate polyadenylation of the target IRF-5, DPMK, or DUX4 gene transcript.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to the linker at the terminal carbonyl group to provide the following structure:

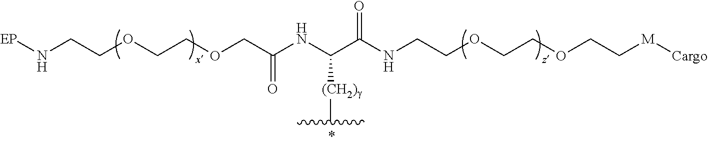

wherein:
EP is an exocyclic peptide and M, AA_{SC}, Cargo, x′, y, and z′ are as defined above, * is the point of attachment to the AA_{SC_}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

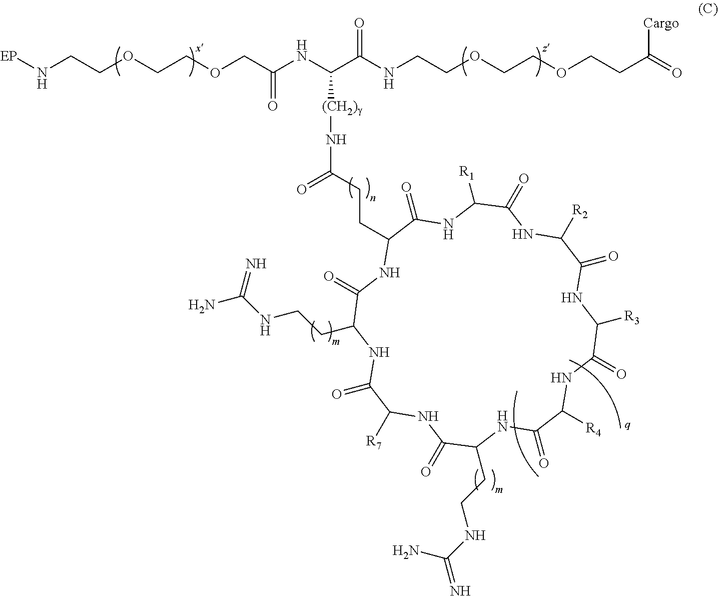

or a protonated form thereof,
wherein:
   R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

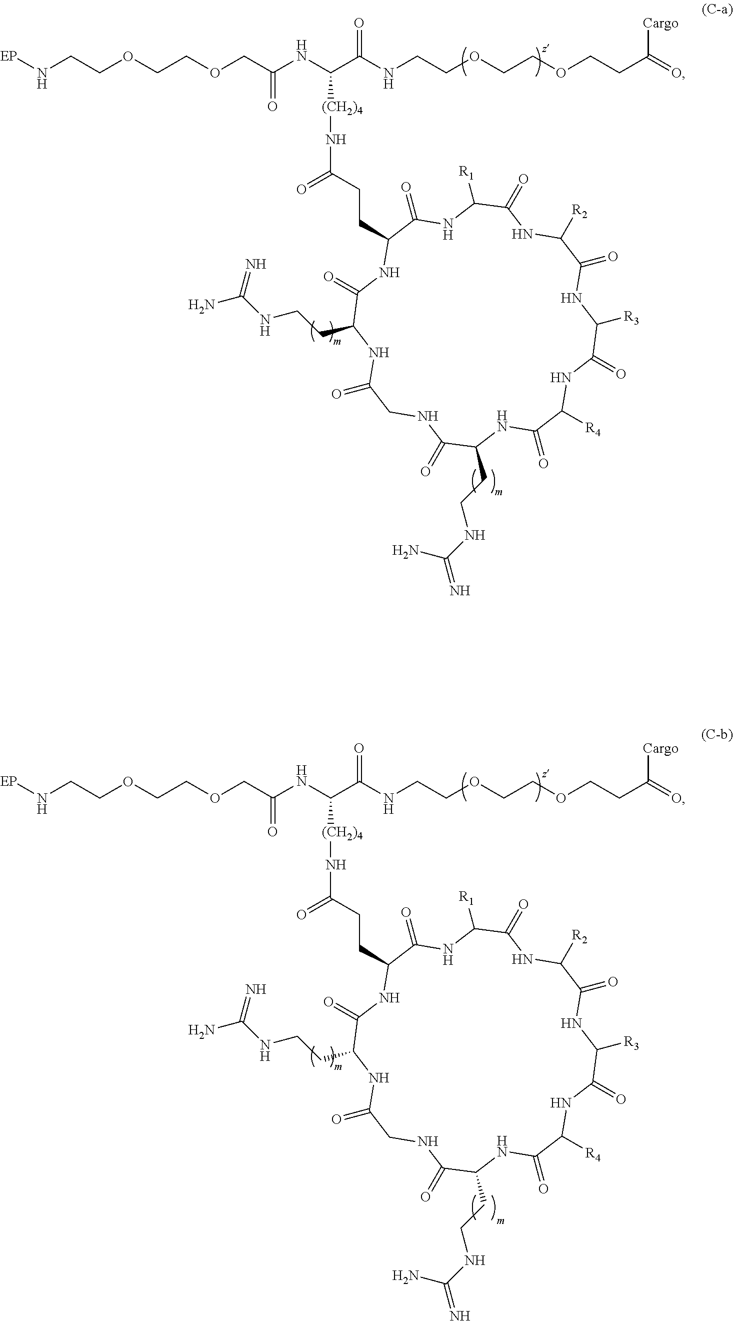

or a protonated form thereof, wherein EP, m and z are as defined above in Formula (C).

The EEV can be conjugated to a cargo and the EEV-conjugate can comprise the structure of Formula (C-c):

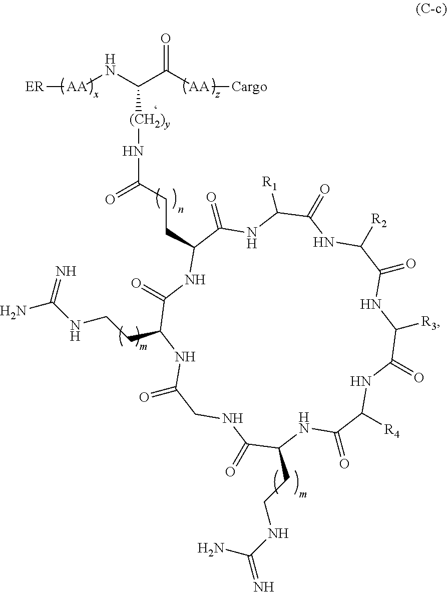

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

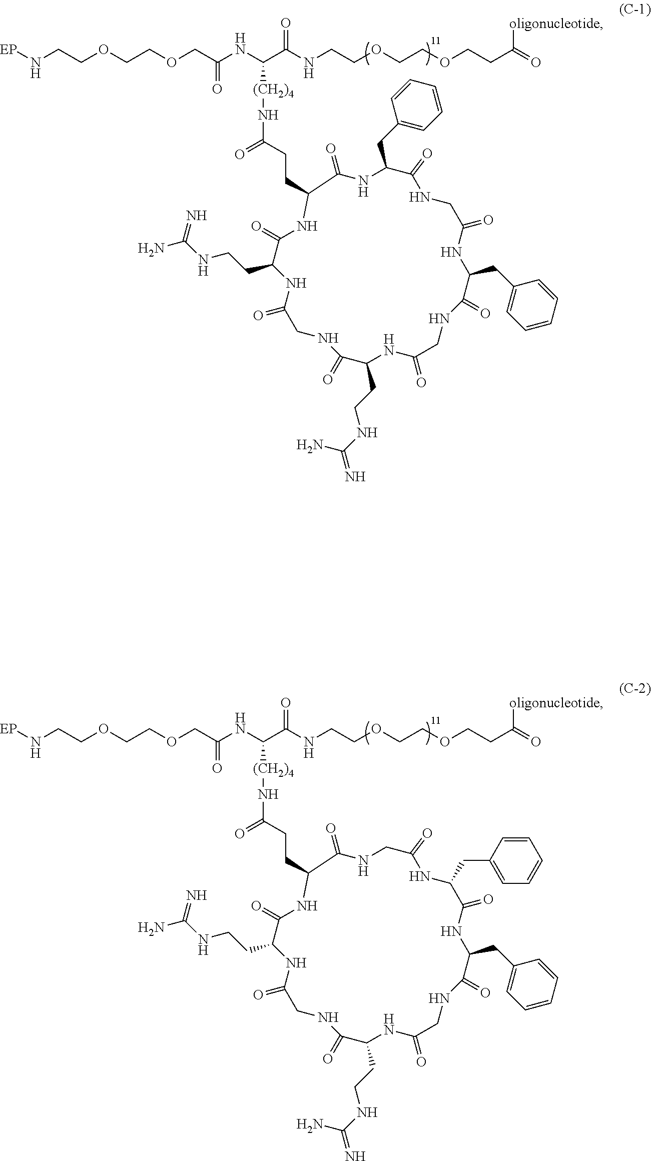

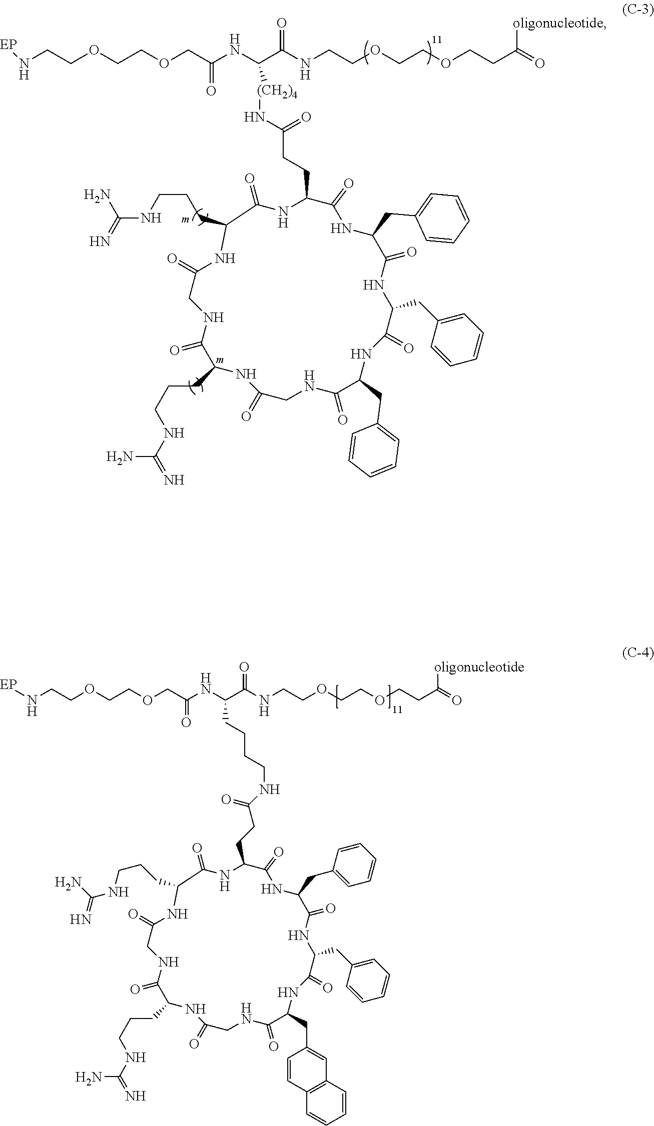

The EEV can be conjugated to an oligonucleotide cargo and the EEV-conjugate can comprise the structure:

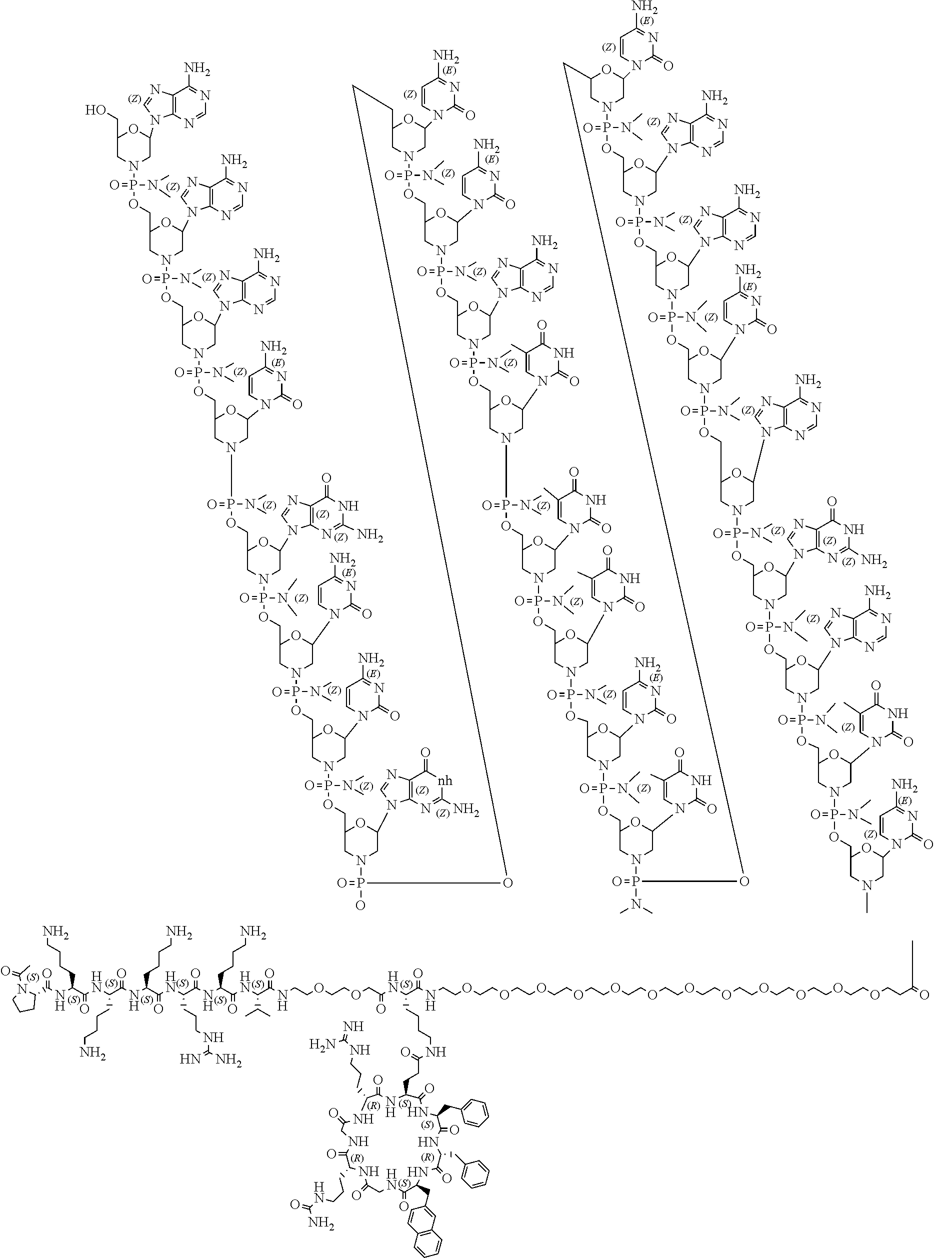

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ, SEQ ID NO:150), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. The relative cytosolic delivery efficiency of the cCPPs can be improved by greater than about 600% compared to a cyclic peptide comprising cyclo(FfΦRrRrQ, SEQ ID NO:150).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Detectable Moiety

In embodiments, the compound disclosed herein includes a detectable moiety. In embodiments, the detectible moiety is attached to the cell penetrating peptide at the amino group, the carboxylate group, or the side chain of any of the amino acids of the cell penetrating peptide moiety (e.g., at the amino group, the carboxylate group, or the side chain of any amino acid in the CPP). In embodiments, the therapeutic moiety includes a detectable moiety. The detectable moiety can include any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In embodiments, the label is detectable without the addition of further reagents.

In embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetraphenyl metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); 146 enzothiazole) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluor'sc'in; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (H) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can include Rhodamine B (Rho), fluorescein isothiocyanate (FITC), 7-amino-4-methylcourmarin (Amc), green fluorescent protein (GFP), or derivatives or combinations thereof.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, e.g., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCI), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to an oligonucleotide, such as an AC, under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the AC by reductive alkylation, the polymer selected should have a single reactive aldehyde so that the degree of polymerization is controlled. See, for example, Kinstler et al., Adv. Drug. Delivery Rev. (2002), 54: 477-485; Roberts et al., Adv. Drug Delivery Rev. (2002), 54: 459-476; and Zalipsky et al., Adv. Drug Delivery Rev. (1995), 16: 157-182.

In order to direct covalently link the AC or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making AC and conjugating AC to linear CPP are generally described in US Pub. No. 2018/0298383, which is herein incorporated by reference for all purposes. The methods may be applied to the cyclic CPPs disclosed herein.

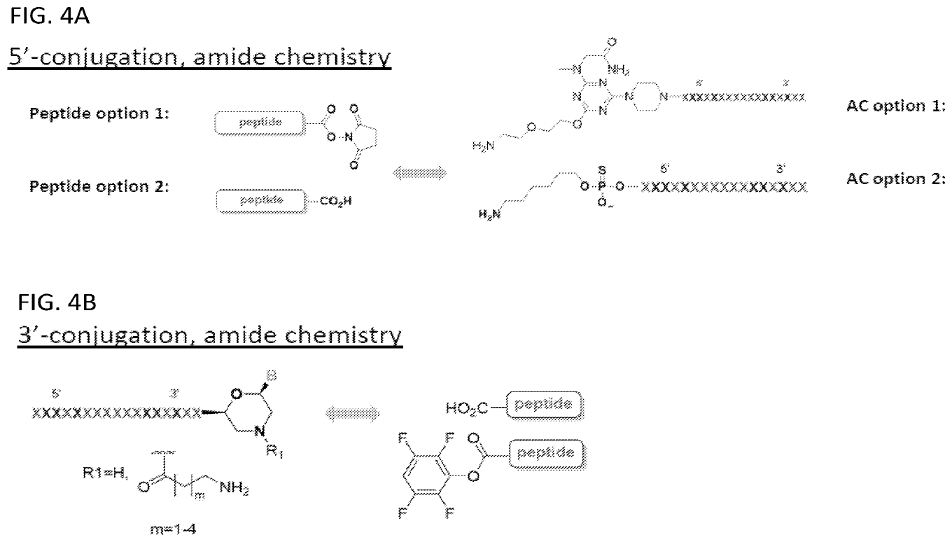

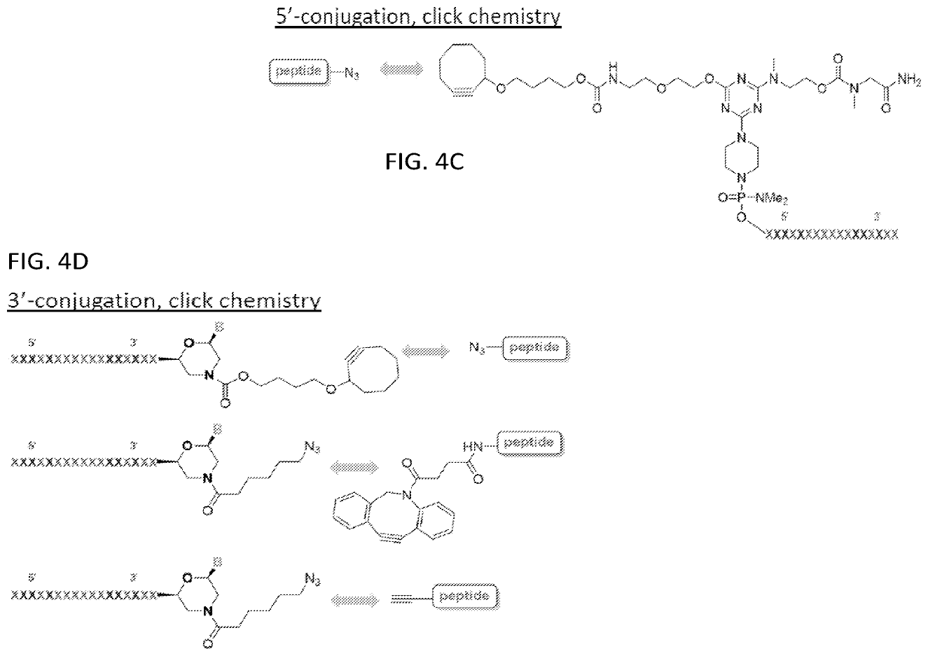

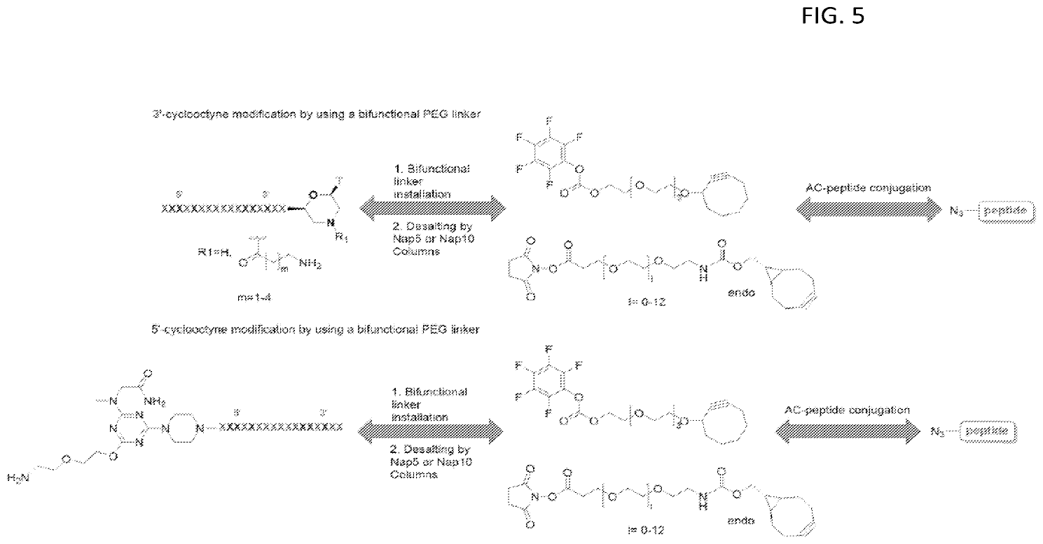

Synthetic schemes are provided in FIG. 4A-4D, FIG. 5.

Non-limiting examples of compounds that include a CPPs and a reactive group useful for conjugation to an AC are shown in Table 6. Example linker groups are also shown. Example reactive groups include tetrafluorophenyl ester (TFP), free carboxylic acid (COOH), and azide (N₃). In Table 6, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo [6.1.0]nonyne.

TABLE 6
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)- PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP (ENTRD 802)
CPP-C₆-TFP (ENTRD 696)
CPP-PEGₙ-K(CPP)-PEGₙ-TFP
CPP-PEGₙ-COOH
CPP-C₁₂-TFP
palmitoyl-PEGₙ-K(CPP)-PEGₙ-TFP
CPP-PEGₙ-K(N₃)
Ac-T9-PEGₙ-K(CPP)-K(N₃)
Ac-MSP-PEGₙ-K(CPP-PEGₙ)-K(N₃)
Ac-NLS-K(CPP)-PEGₙ-K(N₃)
K(N3)-PEGₙ-NLS-ss-PEGₙ-CPP
K(N3)-PEGₙ-NLS-K-βA-βA-CPP

In embodiments, the CPPs have free carboxylic acid groups that may be utilized for conjugation to an AC. In embodiments, the EEVs have free carboxylic acid groups that may be utilized for conjugation to an AC.

The structure below is a 3′ cyclooctyne modified PMO used for a click reaction with a compound that includes an azide:

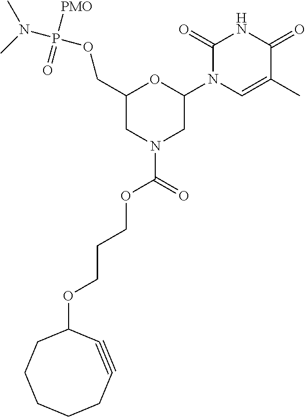

An example scheme of conjugation of a CPP and linker to the 3′ end of an AC via an amide bond is shown below.

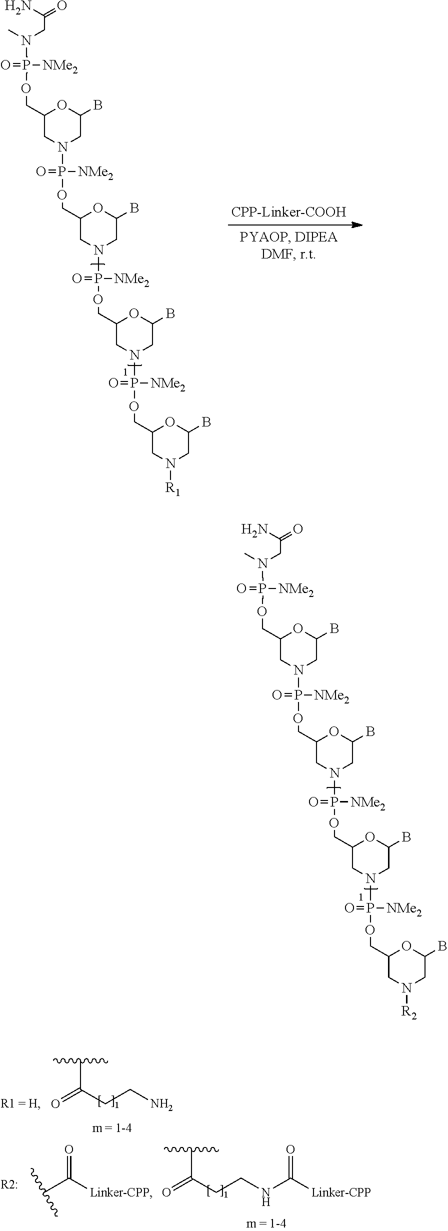

An example scheme of conjugation of a CPP and linker to a 3′-cyclooctyne modified PMO via strain-promoted azide-alkyne cycloaddition is shown below:

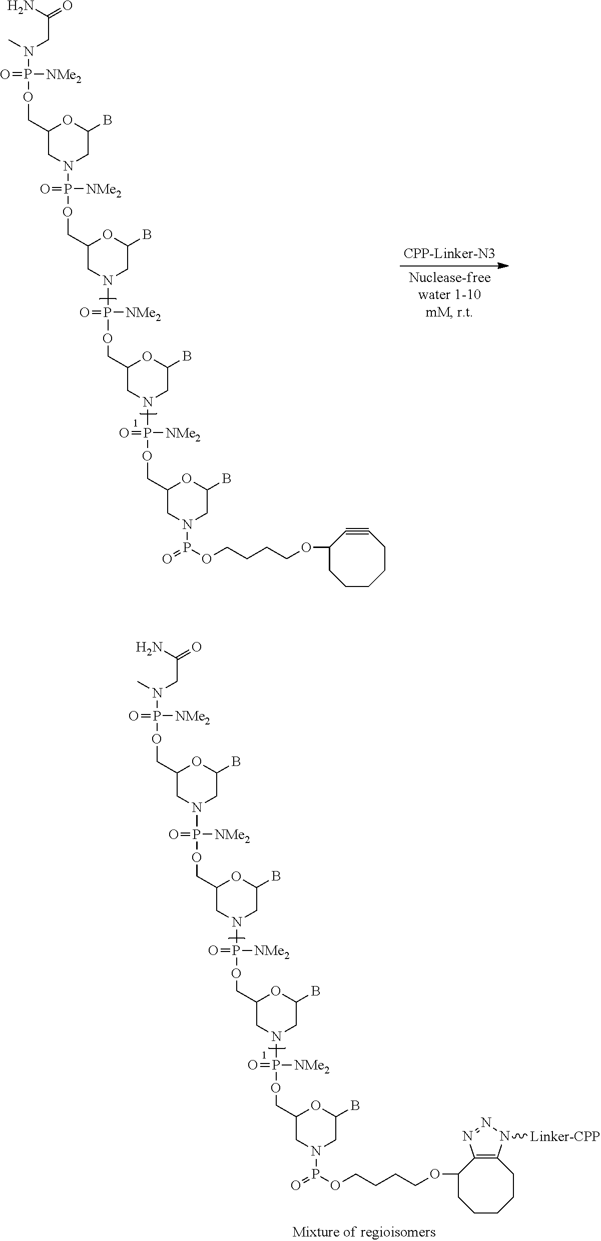

An example of the conjugation chemistry used to connect an AC and CPP with an additional linker containing a polyethylene glycol moiety is shown below:

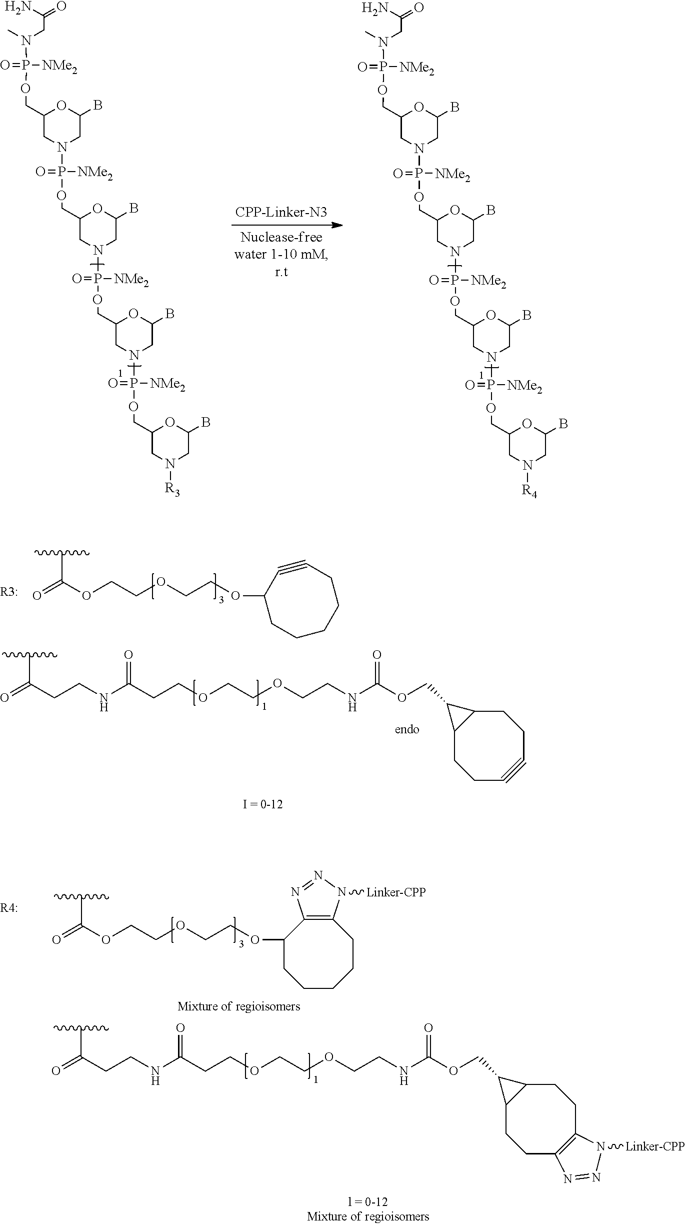

An example of conjugation of a CPP-linker to a 5′-cyclooctyne modified PMO via strain-promoted azide-alkyne cycloaddition (click chemistry) is shown below:

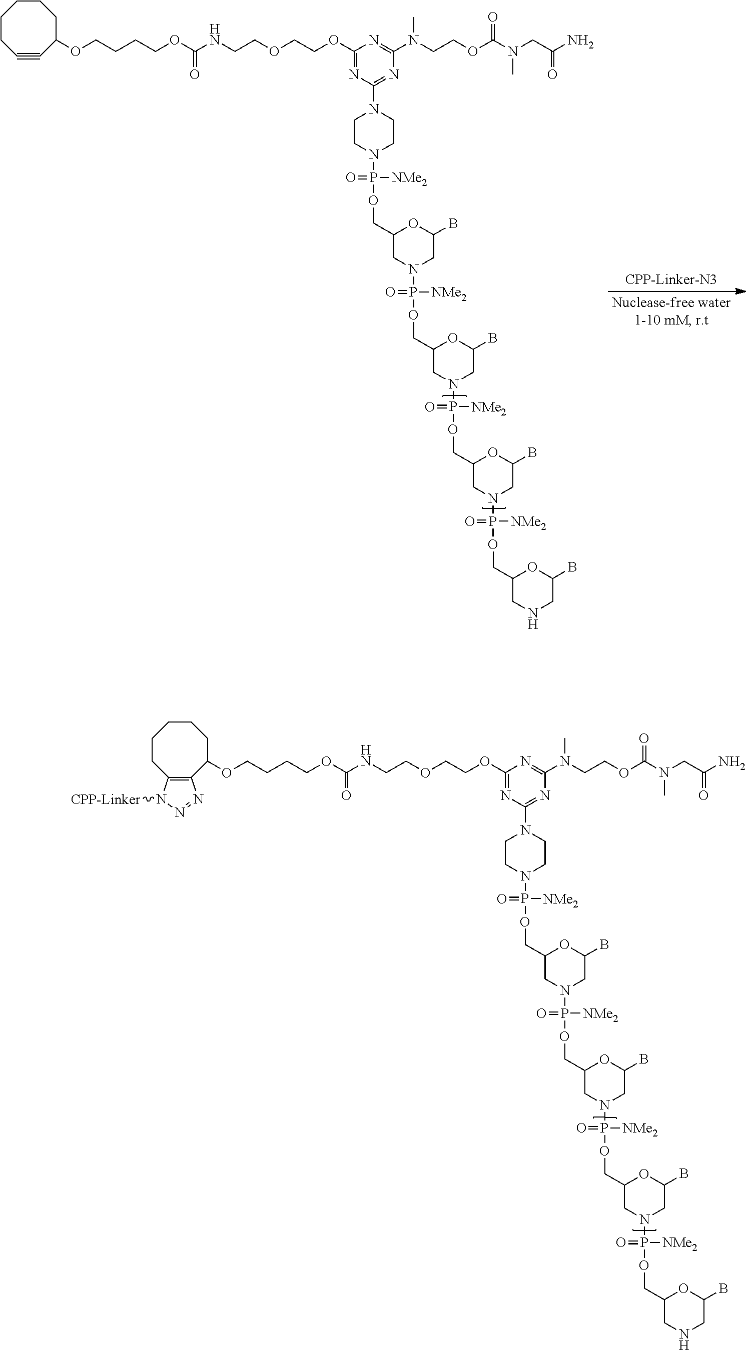

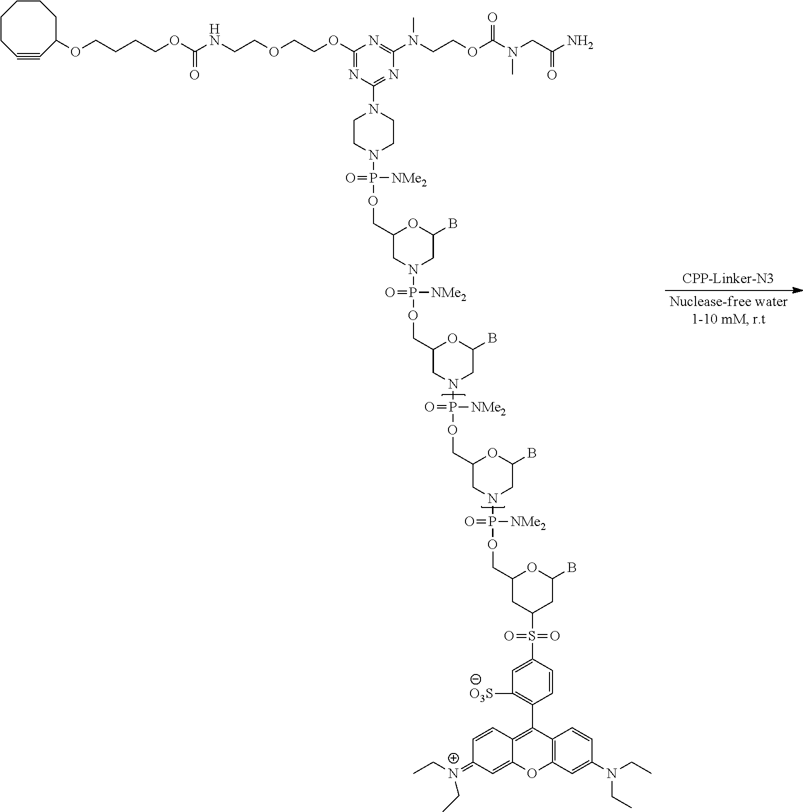

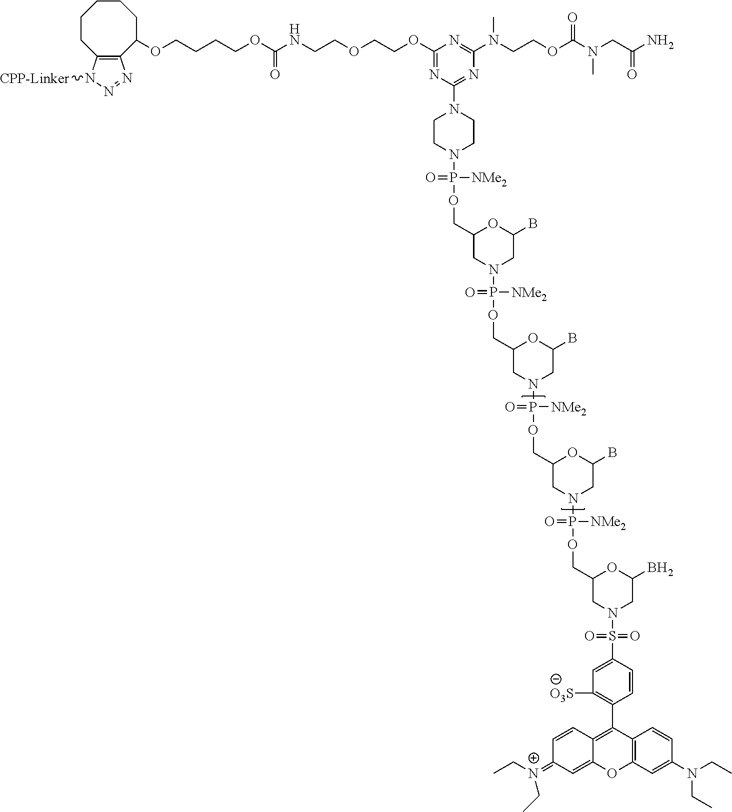

Methods of synthesizing oligomeric antisense compounds are known in the art. The present disclosure is not limited by the method of synthesizing the AC. In embodiments, provided herein are compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

### Diseases

In some embodiments, various diseases or conditions can be treated, prevented, or ameliorated with a composition that includes one or more of the compounds described herein. In embodiments, the disease to be treated, prevented, or ameliorated with a composition of the present disclosure is a disease for which downregulation of expression of a target gene may be beneficial. In embodiments, the compounds may be used to modulate polyadenylation of a target gene transcript. In embodiments, modulation of polyadenylation of a target gene transcript results in reduced expression of a gene product associated with the gene transcript.

In embodiments, the compounds disclosed herein are used for treating, preventing, or ameliorating a disease or condition. Illustrative diseases or conditions that can be treated, prevented, or modulated using compounds of the present disclosure can include, but are not limited to cancers, including for example acute myeloid leukemia, B-cell leukemia/lymphoma, bladder cancer, breast cancer, chronic lymphocytic leukemia, colon cancer, colorectal cancer, esophageal squamous cell carcinoma, fanconi anemia, gastric cancer, glioblastoma, hepatocellular carcinoma, lung cancer, lynch syndrome, mantle cell lymphoma, melanoma, nasopharyngeal carcinoma, neuroblastoma, ovarian cancer, pancreatic ductal adenocarcinoma, proliferative conditions, prostate cancer, and small intestinal neuroendocrine cancer; cardiovascular conditions including for example atherosclerosis, cardiac hypertrophy, dilated cardiomyopathy, hypertension, ischemia/reperfusion injury, thrombosis (deep vein), and thrombosis (venous); congenital abnormalities including microphthalmia, mullerian aplasia, bone fragility (osteogenesis imperfecta), and rickets; endocrine disorders including neonatal diabetes and type 2 diabetes; hematological disorders including glanzmann thrombasthenia, α-thalassemia, and β-thalassemia; immunological disorders including IPEX syndrome, nasal polyps, severe combined immunodeficiency, systemic lupus erythematosus, and Wiskott-Aldrich syndrome; lung conditions including pulmonary fibrosis; musculoskeletal conditions including muscle fibrosis, facioscapulohumeral muscular dystrophy, oculopharyngeal muscular dystrophy, myotonic dystrophy, and oculopharyngeal muscular dystrophy; neurological conditions including Alzheimer disease, amyotrophic lateral sclerosis, anxiety disorders, fabry disease, fragile X syndrome, friedrich's ataxia, huntington's disease, metachromatic leukodystrophy, pseudodeficiency, neuropsychiatric disease, Parkinson disease, and suicidal behavior; stress; and Zellweger syndrome (See Nourse et al. (2020) Biomolecules 10(915) doi.10.3390/biom10060915.

In embodiments, disease to be treated, prevented, or ameliorated with a composition of the present disclosure is associated with alternative polyadenylation.

In embodiments, the compounds disclosed herein are used for treating, preventing, or ameliorating a disease associated with aberrant gene transcription, splicing and/or translation. In embodiments, the compounds disclosed herein are used for treating, preventing or ameliorating a disease associated with aberrant IRF-5 transcription, splicing and/or translation. In embodiments, the compounds disclosed herein are used for treating, preventing or ameliorating a disease associated with IRF-5 upregulation, IRF-5 polymorphisms, accumulation of mutant IRF-5 RNA, or combinations thereof (See Kristjansdottir et al. (2008) J. Med. Genet. 45:362-369; Thompson et al. (2018) Front. Immunol. doi.org/10.3389/fimmu.2018.02622; Almuttaqi and Udalova, (2019) FEBS J. 286:1624-1637; Sicot and Gomes-Pereira, Biochimica et Biophysica Acta 1832 (2013) 1390-1409).

In embodiments, the compounds disclosed herein are used for treating, preventing, or ameliorating a disease associated with abnormal expansion of trinucleotide repeat sequences, including, but not limited to, fragile-X syndrome, spinobulbar muscular atrophy (SBMA) or Kennedy disease. In embodiments, the compounds disclosed herein are used for treating, preventing or ameliorating a disease associated with expansion of trinucleotide repeats at specific chromosomal loci, including, but not limited to, as myotonic dystrophy type 1 (DM1), Huntington disease (HD), Friedreich ataxia (FRDA), dentatorubral-pallidoluysian atrophy (DRPLA) and many spinocerebellarataxias (SCAs; e.g., SCA1, SCA2, SCA3/MJD, SCA7, and SCA17)(Oberle et al., Science 252 (1991) 1097-1102; Kremer et al., Science 252 (1991) 1711-1714; Yu et al., Science 252 (1991) 1179-1181; Verkerk et al., Cell 65 (1991) 905-914; La Spada et al., Nature 352 (1991) 77-79; Pearson et al., Nat. Rev. Genet. 6 (2005) 729-742; Gomes-Pereira and Monckton, Mutat. Res. 598 (2006) 15-34; Gatchel and Zoghbi, Nat. Rev. Genet. 6 (2005) 743-755). In embodiments, the compounds disclosed herein are used for treating, preventing, and/or ameliorating a disease associated with either gain of function or loss of function mechanisms cause by an expanded CAG repeat that encodes a polyglutamine tract within the protein-coding sequence (Takahashi et al., J. Mol. Cell Biol. 2 (2010), 180-191).

In embodiments, the compounds disclosed herein are used for treating, preventing, or ameliorating a disease associated with non-coding triplet repeats, including, for example, diseases associated with repeat expansions mapping to the 5′ or 3′untranslated regions (UTRs), promoters, introns, or combinations thereof of the affected gene (Sakamoto et al., Mol. Cell 3 (1999) 465-475, Pieretti et al., Cell 66 (1991), 817-822).

In embodiments, the compounds disclosed herein are used for treating, preventing or ameliorating a disease associated with expansion of non-coding repeat sequences, including, but not limited to, DM type 2 (DM2), fragile X tremor ataxia syndrome (FXTAS), SCA8 SCA8, SCA10, SCA12, SCA31, SCA36, Huntington disease-like 2 (HDL2) and amyotrophic lateral sclerosis (ALS) (Liquori et al., Science 293 (2001) 864-867, Hagerman and Hagerman, Am. j. Hum. Genet. 74 (2004) 805-816; Daughters et al., PLoS Genet. 5 (2009) e1000600; White et al., PLoS Genet. 6 (2010) e1000984; Holmes et al., Brain Res. Bull. 56 (2001) 397-403; Sato et al., Am. J. Hum. Genet. 85 (2009) 544-557; Kobayashi et al., Am. J. Hum. Genet. 89 (2011) 121-130; Rudnicki et al., Ann. Neurol. 61 (2007) 272-282; DeJesus-Hernandez et al., Neuron 72 (2011) 245-256. Toxic RNAs have also been implicated in polyglutamine expansion disorders primarily mediated by proteotoxicity, suggesting that the contribution of RNA toxicity to disease might have wider implications than previously thought, and participate in multiple human conditions (Wojciechowska and Krzyzosiak, RNA Biol. 8 (2011) 565-571.

### Interferon Regulatory Factor-5 (IRF-5)

In embodiments, a compound is provided for modulating the activity of Interferon Regulatory Factor-5 (IRF-5). IRF-5 is a member of the IRF family of transcription factors that is highly expressed in monocytes, macrophages, B cells, and dendritic cells. IRF-5 is involved in innate and adaptive immunity, macrophage polarization, cell growth regulation and differentiation and apoptosis.

Aberrant IRF-5 expression is associated with a variety of diseases. For example, upregulation of IRF-5 can lead to increased production of IFNs, which is linked to the development of numerous inflammatory diseases, including autoimmune disease, infectious disease, cancer, obesity, neuropathic pain, cardiovascular disease (e.g., artherosclerosis) and metabolic dysfunction (Banga et al. (2020) Sci. Adv. 6:eaay1057). Additionally, IRF-5 gene polymorphisms related to higher IRF-5 expression are associated with susceptibility to inflammatory and autoimmune diseases including rheumatoid arthritis (RA), inflammatory bowel disease (IBD), multiple sclerosis (MS) inflammatory bowel disease (IBD), systemic lupus erythematosus (SLE) and Sjögrens syndrome (Almuttaqi and Udalova, FEBS J. (2018), 286:1624-1637; Thompson et al., Front. Immunol. (2018), 9:2622).

IRF-5 exists in multiple isoforms that are generated by three alternative non-coding 5′ exons and at least nine alternatively spliced mRNAs. The sequences for the IRF-5 isoforms are publicly available, for example, through the online UniProt database. The isoforms show cell-type specific expression, subcellular localization and function. Some isoforms are associated with risk of autoimmune disease. For example, Isoform 2 is linked to overexpression of IRF-5 and susceptibility to autoimmune disease such as systemic lupus erythematosus. Additionally, polymorphisms, including single nucleotide polymorphisms, in the gene encoding IRF-5 that led to higher mRNA expression are associated with many autoimmune diseases (Krausgruber et al., Nat. Immunol. (2010), 12(3):231-238); Kozyrev et al., Arthritis and Rheumatology (2007), 56(4):1234-1241).

IRF-5 activation, mechanisms of action, signaling pathway, and regulatory elements have been reviewed (Song et al. (2020) “Inhibition of IRF-5 hyperactivation protects from lupus onset and severity,” J. Clin. Invest. 130(12):6700-6717; Almutaqqi and Udalova (2018) FEBS J. 286:1624-1637; Banga et al. (2020), Sci. Adv. 6:eaay1057; Thompson et al. Front. Immunol., 2018, 9:2622)

In embodiments, a compound is provided that is capable of reducing or suppressing IRF-5 expression, activity, and/or function. In embodiments, the compound includes an antisense compound (AC), such as an antisense oligonucleotide (ASO). In embodiments, the compound includes an AC that binds to an IRF-5 transcript and increases transcript degradation, thereby decreasing the amount of, and thus, activity of IRF-5 in a cell, such as an immune cell, a myeloid cell, and/or a macrophage. In embodiments, the compound includes a selective inhibitor of IRF-5 activity. A “selective inhibitor of IRF-5 activity” is a compound that preferentially inhibits IRF-5 activity over the activity of other members of the IRF family including, but not limited to IRF-1, IRF-2, IRF-3, IRF4, etc.

In embodiments, IRF-5 is encoded by a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6, the sequences of which are provided below:

HUMAN Interferon regulatory factor - 5(IRF-5)(Isoform 1)
(SEQ ID NO: 151)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTEDVKWPPTLQPPTLRPPTLQPPTLQPPVVLGP
PAPDPSPLAPPPGNPAGFRELLSEVLEPGPLPASLPPAGEQLLPDLLISPHMLPLTDLEIKFQY
RGRPPRALTISNPHGCRLFYSQLEATQEQVELFGPISLEQVREPSPEDIPSDKQRFYTNQLLDV
LDRGLILQLQGQDLYAIRLCQCKVFWSGPCASAHDSCPNPIQREVKTKLESLEHFLNELILFQK
GQTNTPPPFEIFFCFGEEWPDRKPREKKLITVQVVPVAARLLLEMESGELSWSADSIRLQISNP
DLKDRMVEQFKELHHIWQSQQRLQPVAQAPPGAGLGVGQGPWPMHPAGM,
HUMAN Interferon regulatory factor - 5(IRF-5)(Isoform 2)
(SEQ ID NO: 152)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTDAVQSGPHMTPYSLLKEDVKWPPTLQPPTLRP
PTLQPPTLQPPVVLGPPAPDPSPLAPPPGNPAGFRELLSEVLEPGPLPASLPPAGEQLLPDLLI
SPHMLPLTDLEIKFQYRGRPPRALTISNPHGCRLFYSQLEATQEQVELFGPISLEQVRFPSPED
IPSDKQRFYTNQLLDVLDRGLILQLQGQDLYAIRLCQCKVFWSGPCASAHDSCPNPIQREVKTK
LFSLEHFLNELILFQKGQTNTPPPFEIFFCFGEEWPDRKPREKKLITVQVVPVAARLLLEMFSG
ELSWSADSIRLQISNPDLKDRMVEQFKELHHIWQSQQRLQPVAQAPPGAGLGVGQGPWPMHPAG
MQ,
HUMAN Interferon regulatory factor - 5(IRF-5)(Isoform 3)
(SEQ ID NO: 153)
MNQSIPVAPTPPRRVRLKPWLVAQVNSCQYPGLQWVNGEKKLFCIPWRHATRHGPSQDGDNTIE
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTDAVQSGPHMTPYSLLKEDVKWPPTLQPPTLQP
PVVLGPPAPDPSPLAPPPGNPAGFRELLSEVLEPGPLPASLPPAGEQLLPDLLISPHMLPLTDL
EIKFQYRGRPPRALTISNPHGCRLFYSQLEATQEQVELFGPISLEQVREPSPEDIPSDKQRFYT
NQLLDVLDRGLILQLQGQDLYAIRLCQCKVFWSGPCASAHDSCPNPIQREVKTKLFSLEHFLNE
LILFQKGQTNTPPPFEIFFCFGEEWPDRKPREKKLITVQVVPVAARLLLEMFSGELSWSADSIR
LQISNPDLKDRMVEQFKELHHIWQSQQRLQPVAQAPPGAGLGVGQGPWPMHPAGMQ,
HUMAN Interferon regulatory factor - 5(IRF-5)(Isoform 4)
(SEQ ID NQ: 154)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTEDVKWPPTLQPPTLQPPVVLGPPAPDPSPLAP
PPGNPAGFRELLSEVLEPGPLPASLPPAGEQLLPDLLISPHMLPLTDLEIKFQYRGRPPRALTI
SNPHGCRLFYSQLEATQEQVELFGPISLEQVRFPSPEDIPSDKQRFYTNQLLDVLDRGLILQLQ
GQDLYAIRLCQCKVFWSGPCASAHDSCPNPIQREVKIKLFSLEHFLNELILFQKGQTNTPPPFE
IFFCFGEEWPDRKPREKKLITVQVVPVAARLLLEMFSGELSWSADSIRLQISNPDLKDRMVEQF
KELHHIWQSQQRLQPVAQAPPGAGLGVGQGPWPMHPAGMQ,
HUMAN Interferon regulatory factor - 5(IRF-5)(Isoform 5)
(SEQ ID NO: 155)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYEVCSNGPAPT
DSQPPEDYSFGAGEEEEEEEELQRMLPSLSLTVIDLEIKFQYRGRPPRALTISNPHGCRLFYSQ
LEATQEQVELEGPISLEQVREPSPEDIPSDKQRFYTNQLLDVLDRGLILQLQGQDLYAIRLCQC
KVFWSGPCASAHDSCPNPIQREVKIKLFSLEHFLNELILFQKGQTNTPPPFEIFFCFGEEWPDR
KPREKKLITVQVVPVAARLLLEMESGELSWSADSIRLQISNPDLKDRMVEQFKELHHIWQSQQR
LQPVAQAPPGAGLGVGQGPWPMHPAGMQ,
and
HUMAN Interferon regulatory factor - 5(IRF-5)(Isoform 6)
(SEQ ID NO: 156)
MNQSIPVAPTPPRRVRLKPWLVAQVNSCQYPGLQWVNGEKKLFCIPWRHATRHGPSQDGDNTIF
KAWAKETGKYTEGVDEADPAKWKANLRCALNKSRDFRLIYDGPRDMPPQPYKIYETPSPLRITL
LVQERRRKKRKSCRGCCQA.

In embodiments, a nucleotide sequence encoding IRF-5 differs by one or more nucleic acids from a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6. In embodiments the nucleotide sequence encoding IRF-5 differs by one or more polymorphisms (e.g., Single Nucleotide Polymorphisms or SNPs). In embodiments, the nucleotide sequence encoding IRF-5 shares less than 100% sequence identity with a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6. In embodiments, IRF-5 is encoded by nucleotide sequence that is at least about 80%, about 85%, about 90%, about 91%, about 92%, about 93%, about 94%, about 95%, about 96%, about 97%, about 98%, or about 99% identical to a nucleic acid sequence encoding IRF-5 Isoform 1, IRF-5 Isoform 2, IRF-5 Isoform 3, IRF-5 Isoform 4, IRF-5 Isoform 5, or IRF-5 Isoform 6. In embodiments, IRF-5 is encoded by nucleotide sequence that is 80% to 100%, 90% to 100%, 95% to 100%, or 99% to 100% identical to a nucleic acid sequence encoding IRF-5 Isoform 1, IRF-5 Isoform 2, IRF-5 Isoform 3, IRF-5 Isoform 4, IRF-5 Isoform 5, or IRF-5 Isoform 6

IRF-5 has been shown to influence inflammatory macrophage phenotype (Almuttaqi and Udalova, FEBS J., 2018, 286:1624-1637). Macrophages can be classified as M1 (classically activated macrophages) or M2 (alternatively activated macrophages) and can be converted to each other depending on the tissue microenvironment. There are three classes of alternately activated macrophages (M2a, M2b and M2c). In normal tissue, the ratio of M1 to M2 macrophages is highly regulated. An imbalance between M1 and M2 macrophages can result in pathologies such as asthma, chronic pulmonary disease, artherosclerosis, or osteoclastogenesis in rheumatoid arthritis. IRF-5 is a major regulator of proinflammatory M1 macrophage polarization (Weiss et al., Mediators of Inflammation., 2013, Dx.doi.org/10.1155/2013/245804).

IRF-5 expression in macrophages is reversibly induced by inflammatory stimulate and contributes to macrophage polarization. IRF-5 upregulates expression of M1 macrophages and downregulates expression of M2 macrophages (Krausgruber et al., Nat. Immunol., 2010 12(3):231-238). In embodiments, the compounds disclosed herein modulate IRF-5 activity in an immune cell (e.g., a macrophage).

Provided herein are compositions and methods for downregulation IRF-5 expression. In one embodiment, a compound is provided for treating a disease associated with aberrant IRF-5 expression. In embodiments, the compound includes an AC. The AC may be any AC and have any AC characteristics as described elsewhere herein. In embodiments, the AC is an ASO. In embodiments, the ASO is a PMO. The AC may bind to any sequence element of an IRF-5 target transcript as described elsewhere herein.

In embodiments, an AC binds to a target nucleotide sequence that includes 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases in the RNA transcript of the following IRF-5 DNA sequence:

(SEQ IN NO: 157)
TCATATCAGATGCTCAAGGCTGGCAGCTACCCCCTTCTTGAGAGTCCAAG
AACCTGGAGCAGAAATAATTTTTATGTATTTTTGGATTAATGAATGTTAA
AAACAGACTCAGCTGTTTCTTTCCTTTTACTACTACCAGTTGCTCCCATG
CTGCTCCACCAGGCCCTGTTTCGGATGCCAACTGGCCCACTCCCCAAGCA
CTTGCCCCCAGCTTGCGACCATTGGCACTGGGAGGGCCTGGCTTCTGGGC
TGATGGGTCAGTTGGGCCTTCATAAACACTCACCTGGCTGGCTTTGCCTT
CCAGGAGGAAGCTGGCTGAAGCAAGGGTGTGGAATTTTAAATGTGTGCAC
AGTCTGGAAAACTGTCAGAATCAGTTTTCCCATAAAAGGGGGGCTAGCAT
TGCAGCTGCATTTGGGACCATTCAAATCTGTCACTCTCTTGTGTATATTC
CTGTGCTATTAAATATATCAGGGCAGTGCATGTAAATCATCCTGATATAT
TTAATATATTTATTATATTGTCCCCCGAGGTGGGGACAGTGAGTGAGTTC
TCTTAGTCCCCCCAGAGCTGGTTGTTAAAGAGCCTGGCACCTACCCGCTC
TCACTTCATCTGTGTCATCTCTGCACACTCCAGCCCACTTTCTGCCTTCA
GCCATTGAGTGGAAGCTGCCCCAGGCCCTTACCAGGTGCAGATGCCCAAT
CTTGATGCCCAGCCATCAGAACTGTGAGCCAAATAAACCTTTTTCTGTAT
AAATTA,

where a single underline in bold and italics indicates hexamer polyadenylation signal (PAS) and double underline in bold and italics indicates a cleavage site (CS).

In embodiments, an AC binds to a target nucleotide sequence that includes 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of SEQ ID NO:157 or of variants thereof having 80% or more, 90% or more, 95% or more, 96% or more, 97% or more, 98% or more, 99% or more, 99% or less, 98% or less, 97% or less, 96% or less, 95% or less, 90% or less, 80% to 99%, 90% to 99%, 95% to 99%, 96% to 99%, 97% to 99%, or 98% to 99% sequence identity to at least a portion of SEQ ID NO:157.

In embodiments, an AC binds to a target nucleotide sequence that includes 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of

(nucleotides 712-757 of SEQ ID NO: 157)
5′...GCCATCAGAACTGTGAGCCAAATAAACCTTTTTCTGTATAAAT
TA...3′.

In embodiments, the AC is any one of antisense sequences in Table 7, a portion thereof, or a variant thereof that has 80% to 99%, 90 to 99%, 95%-99% sequence identity of a given antisense compound. The ACs in Table 7 target the IRF-5 polyadenylation signal (PS) and/or the CS in which the hexamer PS sequence and CS are in bold. The antisense sequence is the reverse complement of the target nucleotide sequence.

TABLE 7
ASOs of 10, 15, 20, and 30 bases in
length for targeting IRF-5

| | SEQ | | SEQ |
|---|---|---|---|
| Antisense Sequence | ID | Target Sequence | ID |
| (5′-3′) | NO: | (5′-3′) | NO: |
| ACACTCGGTT | 158 | TGTGAGCCAA | 261 |
| CACTCGGTTT | 159 | GTGAGCCAAA | 262 |
| ACTCGGTTTA | 160 | TGAGCCAAAT | 263 |
| CTCGGTTTAT | 161 | GAGCCAAATA | 264 |
| TCGGTTTATT | 162 | AGCCAAATAA | 265 |
| CGGTTTATTT | 163 | GCCAAATAAA | 266 |
| GGTTTATTTG | 164 | CCAAATAAAC | 267 |
| GTTTATTTGG | 165 | CAAATAAACC | 268 |
| TTTATTTGGA | 166 | AAATAAACCT | 269 |
| TTATTTGGAA | 167 | AATAAACCTT | 270 |
| TATTTGGAAA | 168 | ATAAACCTTT | 271 |
| ATTTGGAAAA | 169 | TAAACCTTTT | 272 |
| TTTGGAAAAA | 170 | AAACCTTTTT | 273 |
| TTGGAAAAAG | 171 | AACCTTTTTC | 274 |
| TCTTGACACTCGGTT | 172 | AGAACTGTGAGCCAA | 275 |
| CTTGACACTCGGTTT | 173 | GAACTGTGAGCCAAA | 276 |
| TTGACACTCGGTTTA | 174 | AACTGTGAGCCAAAT | 277 |
| TGACACTCGGTTTAT | 175 | ACTGTGAGCCAAATA | 278 |
| GACACTCGGTTTATT | 176 | CTGTGAGCCAAATAA | 279 |
| ACACTCGGTTTATTT | 177 | TGTGAGCCAAATAAA | 280 |
| CACTCGGTTTATTTG | 178 | GTGAGCCAAATAAAC | 281 |
| ACTCGGTTTATTTGG | 179 | TGAGCCAAATAAACC | 282 |
| CTCGGTTTATTTGGA | 180 | GAGCCAAATAAACCT | 283 |
| TCGGTTTATTTGGAA | 181 | AGCCAAATAAACCTT | 284 |
| CGGTTTATTTGGAAA | 182 | GCCAAATAAACCTTT | 285 |
| GGTTTATTTGGAAAA | 183 | CCAAATAAACCTTTT | 286 |
| GTTTATTTGGAAAAA | 184 | CAAATAAACCTTTTT | 287 |
| TTTATTTGGAAAAAG | 185 | AAATAAACCTTTTTC | 288 |
| TTATTTGGAAAAAGA | 186 | AATAAACCTTTTTCT | 289 |
| TATTTGGAAAAAGAC | 187 | ATAAACCTTTTTCTG | 290 |
| ATTTGGAAAAAGACA | 188 | TAAACCTTTTTCTGT | 291 |
| TTTGGAAAAAGACAT | 189 | AAACCTTTTTCTGTA | 292 |
| TTGGAAAAAGACATA | 190 | AACCTTTTTCTGTAT | 293 |
| TGGAAAAAGACATAT | 191 | ACCTTTTTCTGTATA | 294 |
| GGTAGTCTTGACACTCGGTT | 192 | CCATCAGAACTGTGAGCCAA | 295 |
| GTAGTCTTGACACTCGGTTT | 193 | CATCAGAACTGTGAGCCAAA | 296 |
| TAGTCTTGACACTCGGTTTA | 194 | ATCAGAACTGTGAGCCAAAT | 297 |
| AGTCTTGACACTCGGTTTAT | 195 | TCAGAACTGTGAGCCAAATA | 298 |
| GTCTTGACACTCGGTTTATT | 196 | CAGAACTGTGAGCCAAATAA | 299 |
| TCTTGACACTCGGTTTATTT | 197 | AGAACTGTGAGCCAAATAAA | 300 |
| CTTGACACTCGGTTTATTTG | 198 | GAACTGTGAGCCAAATAAAC | 301 |
| TTGACACTCGGTTTATTTGG | 199 | AACTGTGAGCCAAATAAACC | 302 |
| TGACACTCGGTTTATTTGGA | 200 | ACTGTGAGCCAAATAAACCT | 303 |
| GACACTCGGTTTATTTGGAA | 201 | CTGTGAGCCAAATAAACCTT | 304 |
| ACACTCGGTTTATTTGGAAA | 202 | TGTGAGCCAAATAAACCTTT | 305 |
| CACTCGGTTTATTTGGAAAA | 203 | GTGAGCCAAATAAACCTTTT | 306 |
| ACTCGGTTTATTTGGAAAAA | 204 | TGAGCCAAATAAACCTTTTT | 307 |
| CTCGGTTTATTTGGAAAAAG | 205 | GAGCCAAATAAACCTTTTTC | 308 |
| TCGGTTTATTTGGAAAAAGA | 206 | AGCCAAATAAACCTTTTTCT | 309 |
| CGGTTTATTTGGAAAAAGAC | 207 | GCCAAATAAACCTTTTTCTG | 310 |
| GGTTTATTTGGAAAAAGACA | 208 | CCAAATAAACCTTTTTCTGT | 311 |
| GTTTATTTGGAAAAAGACAT | 209 | CAAATAAACCTTTTTCTGTA | 312 |
| TTTATTTGGAAAAAGACATA | 210 | AAATAAACCTTTTTCTGTAT | 313 |
| TTATTTGGAAAAAGACATAT | 211 | AATAAACCTTTTTCTGTATA | 314 |
| TATTTGGAAAAAGACATATT | 212 | ATAAACCTTTTTCTGTATAA | 315 |
| ATTTGGAAAAAGACATATTT | 213 | TAAACCTTTTTCTGTATAAA | 316 |
| TTTGGAAAAAGACATATTTA | 214 | AAACCTTTTTCTGTATAAAT | 317 |
| TTGGAAAAAGACATATTTAA | 215 | AACCTTTTTCTGTATAAATT | 318 |
| CGGTAGTCTTGACACTCGGT | 216 | GCCATCAGAACTGTGAGCCA | 319 |
| TTATT | | AATAA | |
| GGTAGTCTTGACACTCGGTT | 217 | CCATCAGAACTGTGAGCCAA | 320 |
| TATTT | | AATAA | |
| GTAGTCTTGACACTCGGTTT | 218 | CATCAGAACTGTGAGCCAAA | 321 |
| ATTTG | | TAAAC | |
| TAGTCTTGACACTCGGTTTA | 219 | ATCAGAACTGTGAGCCAAAT | 322 |
| TTTGG | | AAACC | |
| AGTCTTGACACTCGGTTTAT | 220 | TCAGAACTGTGAGCCAAATA | 323 |
| TTGGA | | CAACT | |
| GTCTTGACACTCGGTTTATT | 221 | CAGAACTGTGAGCCAAATAA | 324 |
| TGGAA | | ACCTT | |
| TCTTGACACTCGGTTTATTT | 222 | AGAACTGTGAGCCAAATAAA | 325 |
| GGAAA | | CCTTT | |
| CTTGACACTCGGTTTATTTG | 223 | GAACTGTGAGCCAAATAAAC | 326 |
| GAAAA | | TCTTT | |
| TTGACACTCGGTTTATTTGG | 224 | AACTGTGAGCCAAATAAACC | 327 |
| AAAAA | | TTTTT | |
| TGACACTCGGTTTATTTGGA | 225 | ACTGTGAGCCAAATAAACCT | 328 |
| AAAAG | | TTTTC | |
| GACACTCGGTTTATTTGGAA | 226 | CTGTGAGCCAAATAAACCTT | 329 |
| GAAAA | | TTTCT | |
| ACACTCGGTTTATTTGGAAA | 227 | TGTGAGCCAAATAAACCTTT | 330 |
| AAGAC | | GTTCT | |
| CACTCGGTTTATTTGGAAAA | 228 | GTGAGCCAAATAAACCTTTT | 331 |
| AGACA | | TCTGT | |
| ACTCGGTTTATTTGGAAAAA | 229 | TGAGCCAAATAAACCTTTTT | 332 |
| GACAT | | CTGTA | |
| CTCGGTTTATTTGGAAAAAG | 230 | GAGCCAAATAAACCTTTTTC | 333 |
| ACATA | | TGTAT | |
| TCGGTTTATTTGGAAAAAGA | 231 | AGCCAAATAAACCTTTTTCT | 334 |
| CATAT | | GTATA | |
| CGGTTTATTTGGAAAAAGAC | 232 | GCCAAATAAACCTTTTTCTG | 335 |
| ATATT | | TATAA | |
| GGTTTATTTGGAAAAAGACA | 233 | CCAAATAAACCTTTTTCTGT | 336 |
| TTATT | | ATAAA | |
| GTTTATTTGGAAAAAGACAT | 234 | CAAATAAACCTTTTTCTGTA | 337 |
| ATTTA | | TAAAT | |
| TTTATTTGGAAAAAGACATA | 235 | AAATAAACCTTTTTCTGTAT | 338 |
| TTTAA | | TAAAT | |
| TTATTTGGAAAAAGACATAT | 236 | AATAAACCTTTTTCTGTATA | 339 |
| TTAAT | | AATTA | |
| CGGTAGTCTTGACACTCGGT | 237 | GCCATCAGAACTGTGAGCCA | 340 |
| TTATTTGGAA | | AATAAACCTT | |
| GGTAGTCTTGACACTCGGTT | 238 | CCATCAGAACTGTGAGCCAA | 341 |
| TATTTGGAAA | | ATAAACCTTT | |
| GTAGTCTTGACACTCGGTTT | 239 | CATCAGAACTGTGAGCCAAA | 342 |
| ATTTGGAAAA | | TAAACCTTTT | |
| TAGTCTTGACACTCGGTTTA | 240 | ATCAGAACTGTGAGCCAAAT | 343 |
| TTTGGAAAAA | | AAACCTTTTT | |
| AGTCTTGACACTCGGTTTAT | 241 | TCAGAACTGTGAGCCAAATA | 344 |
| TTGGAAAAAG | | AACCTTTTTC | |
| GTCTTGACACTCGGTTTATT | 242 | CAGAACTGTGAGCCAAATAA | 345 |
| TGGAAAAAGA | | ACCTTTTTCT | |
| TCTTGACACTCGGTTTATTT | 243 | AGAACTGTGAGCCAAATAAA | 346 |
| GGAAAAAGAC | | CCTTTTTCTG | |
| CTTGACACTCGGTTTATTTG | 244 | GAACTGTGAGCCAAATAAAC | 347 |
| GAAAAAGACA | | CTTTTTCTGT | |
| TTGACACTCGGTTTATTTGG | 245 | AACTGTGAGCCAAATAAACC | 348 |
| AAAAAGACAT | | TTTTTCTGTA | |
| TGACACTCGGTTTATTTGGA | 246 | ACTGTGAGCCAAATAAACCT | 349 |
| AAAAGACATA | | TTTTCTGTAT | |
| GACACTCGGTTTATTTGGAA | 247 | CTGTGAGCCAAATAAACCTT | 350 |
| AAAGACATAT | | TTTCTGTATA | |
| ACACTCGGTTTATTTGGAAA | 248 | TGTGAGCCAAATAAACCTTT | 351 |
| AAGACATATT | | TTCTGTATAA | |
| CACTCGGTTTATTTGGAAAA | 249 | GTGAGCCAAATAAACCTTTT | 352 |
| AGACATATTT | | TCTGTATAAA | |
| ACTCGGTTTATTTGGAAAAA | 250 | TGAGCCAAATAAACCTTTTT | 353 |
| GACATATTTA | | CTGTATAAAT | |
| CTCGGTTTATTTGGAAAAAG | 251 | GAGCCAAATAAACCTTTTTC | 354 |
| ACATATTTAA | | TGTATAAATT | |
| TCGGTTTATTTGGAAAAAGA | 252 | AGCCAAATAAACCTTTTTCT | 355 |
| CATATTTAAT | | GTATAAATTA | |
| GCTGAGTCTGTTTTTAACAT | 253 | AATGAATGTTAAAAACAGAC | 356 |
| TCATT | | TCAGC | |
| TGAGTCTGTTTTTAACATTC | 254 | TTAATGAATGTTAAAAACAG | 357 |
| ATTAA | | ACTCA | |
| AGTCTGTTTTTAACATTCAT | 255 | GATTAATGAATGTTAAAAAC | 358 |
| TAATC | | AGACT | |
| TCTGTTTTTAACATTCATTA | 256 | TGGATTAATGAATGTTAAAA | 359 |
| ATCCA | | ACAGA | |
| AATTTATACAGAAAAAGGTT | 257 | AATAAACCTTTTTCTGTATA | 360 |
| TATT | | AATTA | |
| ATTTATACAGAAAAAGGTTT | 258 | CAAATAAACCTTTTTCTGTA | 361 |
| ATTTG | | TTAAA | |
| TTATACAGAAAAAGGTTTAT | 259 | GCCAAATAAACCTTTTTCTG | 362 |
| TTGGC | | TATAA | |
| TACAGAAAAAGGTTTATTTG | 260 | GAGCCAAATAAACCTTTTTC | 363 |
| GCTC | | TGTA | |

Diseases Associated with Interferon Regulatory Factor-5

In embodiments, a method is provided for treating, preventing, or ameliorating a disease or disorder associated with IRF-5. In embodiments, the disease or disorder is associated with IRF-5 genetic variation. In embodiments, the disease or disorder is associated with a genetic mutation in the IRF-5 gene. In embodiments, the genetic mutation in IRF-5 results IRF-5 overexpression. In embodiments, the genetic mutation results in alternate isoform expression. In embodiments, the disease or disorder is associated with IRF-5 overexpression. In embodiments, the disease or disorder is associated with IRF-5 isoform expression. In embodiments, a method is provided for treating, preventing, or ameliorating inflammation, autoantibody production, inflammatory cell infiltration, collagen deposits, or inflammatory cytokine production in a patient.

IRF-5 involvement in various diseases has been document (see for example, Graham et al., Nat Genet. (2006), 38(0.5):550-5; Rueda et al., Arthritis Rheum. (2006), 54(12):3815-9; Henrique da Mota, Clin Rheumatol. (2015), 34(9):1495-501; Sigurdsson et al., Hum Mol Genet. (2008), 17(6):872-81; Peng, et al., Nephrology (Carlton) (2010), 15(7):710-3; Ishimura et al., J Clin Immunol. (2011), 31(6): 946-51; Summers et al., J. Rheumatol. (2008), 35(11):2106-18; Ni et al., Inflammation (2019), 2(5):1821-1829; Dideberg et al., Hum Mol Genet. (2007), 16(24):3008-16; Lim et al., J. Dig. Dis. (2015), 16(4):205-16; Nordal et al., Ann. Rheum. Dis. (2012), 71(7):1197-202; Rebora, Int. J. Dermatol. (2016), 55(4):408-16; Zhao et al., Rheumatol. Int. (2017), 37(8):1303-131; Carmona et al., PLoS One (2013), 8(1):e54419; Flesch et al., Tissue Antigens (2011), 78(1):65-8; Heijde et al., Arthritis Rheum. (2007), 56(12):3989-94; Hafler et al., Genes Immun. (2009), 10(1):68-76; Balasa et al., Eur. Cytokine Netw. (2012), 23(4):166-72; Byre et al., Mucosal Immunol. (2017), 10(3):716-726; Wang et al., Gene (2012), 10, 504(2):220-5; Pimenta et al., Mol. Cancer (2015), 14(1):32; Rambod et al., Clin Rheumatol. (2018), 37(10):2661-2665; Davi et al., J Rheumatol. (2011), 38(4):769-74; Zimmerman et al., Kidney 360 (2020), 1(3): 179-190; Pandey et al., Mucosal. Immunol. (2019), 12(4):874-887; Masuda et al., Nat. Commun. (2014), 5: 3771; Alzaid et al., JCI Insight (2016), 1(20): e88689; Senevirante et al., Circulation (2017), 136(12): 1140-1154; Cevik et al., J. Biol. Chem. (2017), 292(52):21676-21689; Sharif et al., Ann. Rheum. Dis. (2012), 71(7):1197-1202; and Yang et al., J Pediatr. Surg. (2017), 52(12):1984-1988).

In embodiments, a method of downregulating IRF-5 expression in a patient is provided using one or more of the compounds disclosed herein. In embodiments, IRF-5 expression in a macrophage, in a Kupffer cell, gastrointestinal tract, liver, lung, kidney, joints, central nervous system, or combinations thereof is reduced.

In embodiments, the compounds disclosed herein are used for treating, preventing, or ameliorating a disease associated with IRF-5. Examples of diseases associated with IRF-5 include, but are not limited to, inflammatory bowel disease (IBD), ulcerative colitis, Crohn's disease, systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), Nonalcoholic steatohepatitis (NASH), liver fibrosis, asthma, severe asthma, and combinations thereof. In embodiments, the compounds disclosed herein are used to reduce inflammation, cirrhosis, fibrosis, proteinuria, joint inflammation, autoantibody production, inflammatory cell infiltration, collagen deposits, inflammatory cytokine production, or combinations thereof in a patient. In embodiments, the compounds disclosed herein are used to reduce inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, unintended weight loss, or combinations thereof.

In embodiments, the compounds disclosed herein are used for treating, preventing, or ameliorating an inflammatory disease. “Inflammatory disease” refers to diseases in which activation of the innate or adaptive immune response is a prominent contributor to the clinical condition. Inflammatory diseases include, but are not limited to, acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, ischemic heart disease, type I diabetes, diabetic nephropathy, or combinations thereof.

In embodiments, the compounds disclosed herein are used for treating, preventing, or ameliorating an autoimmune disease. “Autoimmune disease” refers to a disease or disorder in which a patient's immune system attacks the patient's own tissues. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); systemic sclerosis (scleroderma); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, dermatomyositis; granulomatosis and vasculitis; primary biliary cirrhosis; pernicious anemia (Addison's disease); autoimmune gastritis; autoimmune hepatitis; diseases involving leukocyte diapedesis; central nervous system (CNS) inflammatory disorder; vitiligo; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP); autoimmune thrombocytopenia; autoimmune encephalomyelitis; nonalcoholic steatohepatitis (NASH); ankylosing spondylitis; pulmonary fibrosis; or combinations thereof.

In embodiments, the compounds disclosed herein are used for treating, preventing or ameliorating cardiovascular disease. In embodiments, the cardiovascular disease is associated with inflammation. In embodiments, the cardiovascular disease includes systemic scleroderma, aneurysm; angina; atherosclerosis; cerebrovascular accident (Stroke); cerebrovascular disease; congestive heart failure; coronary artery disease; myocardial infarction (heart attack); peripheral vascular disease; or combinations thereof.

In embodiments, the compounds disclosed herein are used for treating, preventing, or ameliorating a gastrointestinal disease. In embodiments, the gastrointestinal disease includes Crohn's disease, primary biliary cirrhosis, sclerosing cholangitis, ulcerative colitis, inflammatory bowel disease, Sjögren's syndrome, or combinations thereof.

In embodiments, the compounds disclosed herein are used for treating, preventing, or ameliorating a urinary system disease. In embodiments, the urinary system disease includes systemic lupus erythematosus, systemic scleroderma, or both.

In embodiments, the compounds disclosed herein are used for treating, preventing, or ameliorating a genetic, familial, or congenital disease. In embodiments, the genetic, familial, or congenital disease includes Crohn's disease, primary biliary cirrhosis, systemic scleroderma, systemic lupus erythematosus, ulcerative colitis, psoriasis, inflammatory bowel disease, or combinations thereof.

In embodiments, the compounds disclosed herein are used for treating, preventing, or ameliorating an endocrine system disease. In embodiments, the endocrine system disease includes thyroid gland adenocarcinoma, primary biliary cirrhosis, sclerosing cholangitis, hypothyroidism, or combinations thereof.

In embodiments, the compounds disclosed herein are used for treating, preventing, or ameliorating a cell proliferation disorder. In embodiments, the cell proliferation disorder includes primary biliary cirrhosis, thyroid gland adenocarcinoma, neoplasm, or combinations thereof.

In embodiments, the compounds disclosed herein are used for treating, preventing, or ameliorating an immune system disease. In embodiments, the immune system disease includes Sjögren's syndrome, inflammatory bowel disease, psoriasis, myositis, systemic scleroderma, autoimmune disease, systemic lupus erythematosus, rheumatoid arthritis, Crohn's disease, ulcerative colitis, ankylosing spondylitis, or combinations thereof.

In embodiments, the compounds disclosed herein are used for treating, preventing, or ameliorating a hematologic disease. In embodiments, the hematologic disease includes systemic lupus erythematosus.

In embodiments, the compounds disclosed herein are used for treating, preventing, or ameliorating a musculoskeletal or connective tissue disease. In embodiments, the musculoskeletal or connective tissue disease includes myositis, systemic scleroderma, systemic lupus erythematosus, rheumatoid arthritis, ankylosing spondylitis, adolescent idiopathic scoliosis, or combinations thereof.

In embodiments, the compounds disclosed herein are used for treating, preventing, or ameliorating neuroinflammatory disease. In embodiments, the neuroinflammatory disease or disorder includes inflammation due to traumatic brain injury, acute disseminated encephalomyelitis (ADEM), autoimmune encephalitis, acute optic neuritis (AON), chronic meningitis, anti-myelin oligodendrocyte glycoprotein (MOG) disease, transverse myelitis, neuromyelitis optica (NMO), Alzheimer's disease, Parkinson's disease, multiple sclerosis (MS), or combinations thereof.

In embodiments, the compounds disclosed herein are used for treating, preventing, or ameliorating inflammation due to infection by microorganisms such as viruses, bacteria, fungi, parasites, or combinations thereof

In embodiments, the compounds disclosed herein are used for treating, preventing, or ameliorating a disease associated with fibrosis, which is referred to herein as a fibrotic disease. “Fibrosis” refers to a pathological formation of fibrous connective tissue, for example, due to injury, irritation, or chronic inflammation and includes fibroblast accumulation and collagen deposition in excess of normal amounts in a tissue. “Fibrotic disease” refers to a disease associated with pathological fibrosis. Examples of fibrotic disease include, but are not limited to, idiopathic pulmonary fibrosis; scleroderma; scleroderma of the skin; scleroderma of the lungs; a collagen vascular disease (e.g., lupus; rheumatoid arthritis; scleroderma); genetic pulmonary fibrosis (e.g., Hermansky-Pudlak Syndrome); radiation pneumonitis; asthma; asthma with airway remodeling; chemotherapy-induced pulmonary fibrosis (e.g., bleomycin, methotrexate, or cyclophosphamide-induced); radiation fibrosis; Gaucher's disease; interstitial lung disease; retroperitoneal fibrosis; myelofibrosis; interstitial or pulmonary vascular disease; fibrosis or interstitial lung disease associated with drug exposure; interstitial lung disease associated with exposures such as asbestosis, silicosis, and grain exposure; chronic hypersensitivity pneumonitis; an adhesion; an intestinal or abdominal adhesion; cardiac fibrosis; kidney fibrosis; cirrhosis; nonalcoholic steatohepatitis (NASH)-induced fibrosis; and combinations thereof. In embodiments, the fibrotic disease includes non-alcoholic steatohepatitis NASH.

In embodiments, the compounds disclosed herein are used for treating, preventing, or ameliorating a respiratory or thoracic disease such as systemic scleroderma. In embodiments, the compounds disclosed herein are used for treating, preventing, or ameliorating an integumentary system disease such as psoriasis or systemic scleroderma. In embodiments, the compounds disclosed herein are used for treating, preventing, or ameliorating a disease of the visual system such as Sjögren's syndrome or systemic scleroderma. In embodiments, the compounds disclosed herein are used for treating, preventing, or ameliorating a disease associated with eosinophil count, glomerular filtration rate, systolic blood pressure, eosinophil percentage of leukocytes, or combinations thereof. In embodiments, the compounds disclosed herein are used for treating, preventing, or ameliorating an ulcer disease or an oral ulcer.

### Double Homeobox 4 (DUX4) Gene

Facioscapulohumeral muscular dystrophy (FSHD) is the third most common form of inherited muscular dystrophy. It is caused by incomplete repression of the transcription factor double homeobox (DUX4) in skeletal muscle. DUX4 overexpression in myogenic cells induces different toxic cascades including an increase in oxidative stress, nonsense-mediated decay inhibition, and inhibition of myogenesis (Bouwman et al., Curr. Opin. Neurol. (2020) 33(5):635-640).

The DUX4 gene is located near the end of chromosome 4 in a region known as D4Z4. The noted region contains from 11 to more than 100 repeated segments, each of which is about 3,300 DNA bases (3.3 kb) long. Each of the repeated segments in the D4Z4 region contains a copy of the DUX4 gene. The copy closest to the end of the chromosome is called DUX4, while the other copies are referred to as “DUX4-like” or DUX4L.

FSHD is characterized by the contraction of the D4Z4 array located in the sub-telomeric region of chromosome 4, leading to aberrant expression of the DUX4 transcription factor and the mis-regulation of hundreds of genes (Marsollier et al., Int. J. Mol. Sci. (2018), 19, 1347, doi:10.3390/ijms19051347).

There are four (4) variants of the Human DUX4 gene: NM_001306068.3 (referred to herein as variant 1), NM_001293798.3 (referred to herein as variant 2), NM_001363820.2 (referred to herein as variant 4), and NR_137167.1 (referred to herein as variant 3). Both DUX4 variant 1 and 2 encode the longer isoform (DUX4-fl) (DUX4-fl). DUX4 variant 2 lacks an alternate segment in the 3′ UTR compared to variant 1. DUX4 variant 4 lacks a large portion of the coding region compared to variants 1 and 2. The resulting isoform (DUX4-s) has a shorter and distinct C-terminus compared to isoform DUX4-fl. DUX4 variant 3 has multiple differences in the 3′ end compared to variant 1, including a distinct 3′ terminus. This variant is represented as non-coding because the use of the 5′-most expected translational start codon renders the transcript a candidate for nonsense-mediated mRNA decay (NMD). Variants 1, 2 and 4 share the last exon, containing a PAS. DUX4 has a non-canonical cleavage site (GAUCCU) located 16 to 22 bases downstream from the polyadenylation signal (Marsollier et al., Human molecular genetics (2016), 25(8), 1468-1478).

The DNA sequences of variants 1, 2, and 4 are shown in Table 8 where bases that are bolded are a part of the PAS. In embodiments, the AC compounds bind at least a portion of the RNA transcript corresponding to SEQ ID NOS:364-366, or combinations thereof.

TABLE 8
Variants of DUX4

| SEQ | | |
|---|---|---|
| ID | Var- | |
| NO: | iant | Sequence |
| 364 | 1 | ATGGCCCTCCCGACACCCTCGGACAGCACCCTCCCCGCGG |
| | | AAGCCCGGGGACGAGGACGGCGACGGAGACTCGTTTGGAC |
| | | CCCGAGCCAAAGCGAGGCCCTGCGAGCCTGCTTTGAGCGG |
| | | AACCCGTACCCGGGCATCGCCACCAGAGAACGGCTGGCCC |
| | | AGGCCATCGGCATTCCGGAGCCCAGGGTCCAGATTTGGTT |
| | | TCAGAATGAGAGGTCACGCCAGCTGAGGCAGCACCGGCGG |
| | | GAATCTCGGCCCTGGCCCGGGAGACGCGGCCCGCCAGAAG |
| | | GCCGGCGAAAGCGGACCGCCGTCACCGGATCCCAGACCGC |
| | | CCTGCTCCTCCGAGCCTTTGAGAAGGATCGCTTTCCAGGC |
| | | ATCGCCGCCCGGGAGGAGCTGGCCAGAGAGACGGGCCTCC |
| | | CGGAGTCCAGGATTCAGATCTGGTTTCAGAATCGAAGGGC |
| | | CAGGCACCCGGGACAGGGTGGCAGGGCGCCCGCGCAGGCA |
| | | GGCGGCCTGTGCAGCGCGGCCCCCGGCGGGGGTCACCCTG |
| | | CTCCCTCGTGGGTCGCCTTCGCCCACACCGGCGCGTGGGG |
| | | AACGGGGCTTCCCGCACCCCACGTGCCCTGCGCGCCTGGG |
| | | GCTCTCCCACAGGGGGCTTTCGTGAGCCAGGCAGCGAGGG |
| | | CCGCCCCCGCGCTGCAGCCCAGCCAGGCCGCGCCGGCAGA |
| | | GGGGATCTCCCAACCTGCCCCGGCGCGCGGGGATTTCGCC |
| | | TACGCCGCCCCGGCTCCTCCGGACGGGGCGCTCTCCCACC |
| | | CTCAGGCTCCTCGGTGGCCTCCGCACCCGGGCAAAAGCCG |
| | | GGAGGACCGGGACCCGCAGCGCGACGGCCTGCCGGGCCCC |
| | | TGCGCGGTGGCACAGCCTGGGCCCGCTCAAGCGGGGCCGC |
| | | AGGGCCAAGGGGTGCTTGCGCCACCCACGTCCCAGGGGAG |
| | | TCCGTGGTGGGGCTGGGGCCGGGGTCCCCAGGTCGCCGGG |
| | | GCGGCGTGGGAACCCCAAGCCGGGGCAGCTCCACCTCCCC |
| | | AGCCCGCGCCCCCGGACGCCTCCGCCTCCGCGCGGCAGGG |
| | | GCAGATGCAAGGCATCCCGGCGCCCTCCCAGGCGCTCCAG |
| | | GAGCCGGCGCCCTGGTCTGCACTCCCCTGCGGCCTGCTGC |
| | | TGGATGAGCTCCTGGCGAGCCCGGAGTTTCTGCAGCAGGC |
| | | GCAACCTCTCCTAGAAACGGAGGCCCCGGGGGAGCTGGAG |
| | | GCCTCGGAAGAGGCCGCCTCGCTGGAAGCACCCCTCAGCG |
| | | AGGAAGAATACCGGGCTCTGCTGGAGGAGCTTTAGGACGC |
| | | GGGGTTGGGACGGGGTCGGGTGGTTCGGGGCAGGGCGGTG |
| | | GCCTCTCTTTCGCGGGGAACACCTGGCTGGCTACGGAGGG |
| | | GCGTGTCTCCGCCCCGCCCCCTCCACCGGGCTGACCGGCC |
| | | TGGGATTCCTGCCTTCTAGGTCTAGGCCCGGTGAGAGACT |
| | | CCACTCCGCGGAGAACTGCCTTTCTTTCCTGGGCATCCCG |
| | | GGGATCCCAGAGCCGGCCCAGGTACCAGCAGACCTGCGCG |
| | | CAGTGCGCACCCCGGCTGACGTGCAAGGGAGCTCGCTGGC |
| | | CTCTCTGTGCCCTTGTTCTTCCGTGAAATTCTGGCTGAAT |
| | | GTCTCCCCCCACCTTCCGACGCTGTCTAGGCAAACCTGGA |
| | | TTAGAGTTACATCTCCTGGATGATTAGTTCAGAGATATAT |
| | | TAAAATGCCCCCTCCCTGTGGATCCTATAG |
| 365 | 2 | ATGGCCCTCCCGACACCCTCGGACAGCACCCTCCCCGCGG |
| | | AAGCCCGGGGACGAGGACGGCGACGGAGACTCGTTTGGAC |
| | | CCCGAGCCAAAGCGAGGCCCTGCGAGCCTGCTTTGAGCGG |
| | | AACCCGTACCCGGGCATCGCCACCAGAGAACGGCTGGCCC |
| | | AGGCCATCGGCATTCCGGAGCCCAGGGTCCAGATTTGGTT |
| | | TCAGAATGAGAGGTCACGCCAGCTGAGGCAGCACCGGCGG |
| | | GAATCTCGGCCCTGGCCCGGGAGACGCGGCCCGCCAGAAG |
| | | GCCGGCGAAAGCGGACCGCCGTCACCGGATCCCAGACCGC |
| | | CCTGCTCCTCCGAGCCTTTGAGAAGGATCGCTTTCCAGGC |
| | | ATCGCCGCCCGGGAGGAGCTGGCCAGAGAGACGGGCCTCC |
| | | CGGAGTCCAGGATTCAGATCTGGTTTCAGAATCGAAGGGC |
| | | CAGGCACCCGGGACAGGGTGGCAGGGCGCCCGCGCAGGCA |
| | | GGCGGCCTGTGCAGCGCGGCCCCCGGCGGGGGTCACCCTG |
| | | CTCCCTCGTGGGTCGCCTTCGCCCACACCGGCGCGTGGGG |
| | | AACGGGGCTTCCCGCACCCCACGTGCCCTGCGCGCCTGGG |
| | | GCTCTCCCACAGGGGGCTTTCGTGAGCCAGGCAGCGAGGG |
| | | CCGCCCCCGCGCTGCAGCCCAGCCAGGCCGCGCCGGCAGA |
| | | GGGGATCTCCCAACCTGCCCCGGCGCGCGGGGATTTCGCC |
| | | TACGCCGCCCCGGCTCCTCCGGACGGGGCGCTCTCCCACC |
| | | CTCAGGCTCCTCGGTGGCCTCCGCACCCGGGCAAAAGCCG |
| | | GGAGGACCGGGACCCGCAGCGCGACGGCCTGCCGGGCCCC |
| | | TGCGCGGTGGCACAGCCTGGGCCCGCTCAAGCGGGGCCGC |
| | | AGGGCCAAGGGGTGCTTGCGCCACCCACGTCCCAGGGGAG |
| | | TCCGTGGTGGGGCTGGGGCCGGGGTCCCCAGGTCGCCGGG |
| | | GCGGCGTGGGAACCCCAAGCCGGGGCAGCTCCACCTCCCC |
| | | AGCCCGCGCCCCCGGACGCCTCCGCCTCCGCGCGGCAGGG |
| | | GCAGATGCAAGGCATCCCGGCGCCCTCCCAGGCGCTCCAG |
| | | GAGCCGGCGCCCTGGTCTGCACTCCCCTGCGGCCTGCTGC |
| | | TGGATGAGCTCCTGGCGAGCCCGGAGTTTCTGCAGCAGGC |
| | | GCAACCTCTCCTAGAAACGGAGGCCCCGGGGGAGCTGGAG |
| | | GCCTCGGAAGAGGCCGCCTCGCTGGAAGCACCCCTCAGCG |
| | | AGGAAGAATACCGGGCTCTGCTGGAGGAGCTTTAGGACGC |
| | | GGGGTCTAGGCCCGGTGAGAGACTCCACTCCGCGGAGAAC |
| | | TGCCTTTCTTTCCTGGGCATCCCGGGGATCCCAGAGCCGG |
| | | CCCAGGTACCAGCAGACCTGCGCGCAGTGCGCACCCCGGC |
| | | TGACGTGCAAGGGAGCTCGCTGGCCTCTCTGTGCCCTTGT |
| | | TCTTCCGTGAAATTCTGGCTGAATGTCTCCCCCCACCTTC |
| | | CGACGCTGTCTAGGCAAACCTGGATTAGAGTTACATCTCC |
| | | TGGATGATTAGTTCAGAGATATATTAAAATGCCCCCTCCC |
| | | TGTGGATCCTATAG |
| 366 | 4 | ATGGCCCTCCCGACACCCTCGGACAGCACCCTCCCCGCGG |
| | | AAGCCCGGGGACGAGGACGGCGACGGAGACTCGTTTGGAC |
| | | CCCGAGCCAAAGCGAGGCCCTGCGAGCCTGCTTTGAGCGG |
| | | AACCCGTACCCGGGCATCGCCACCAGAGAACGGCTGGCCC |
| | | AGGCCATCGGCATTCCGGAGCCCAGGGTCCAGATTTGGTT |
| | | TCAGAATGAGAGGTCACGCCAGCTGAGGCAGCACCGGCGG |
| | | GAATCTCGGCCCTGGCCCGGGAGACGCGGCCCGCCAGAAG |
| | | GCCGGCGAAAGCGGACCGCCGTCACCGGATCCCAGACCGC |
| | | CCTGCTCCTCCGAGCCTTTGAGAAGGATCGCTTTCCAGGC |
| | | ATCGCCGCCCGGGAGGAGCTGGCCAGAGAGACGGGCCTCC |
| | | CGGAGTCCAGGATTCAGATCTGGTTTCAGAATCGAAGGGC |
| | | CAGGCACCCGGGACAGGGTGGCAGGGCGCCCGCGCAGGTC |
| | | TAGGCCCGGTGAGAGACTCCACTCCGCGGAGAACTGCCTT |
| | | TCTTTCCTGGGCATCCCGGGGATCCCAGAGCCGGCCCAGG |
| | | TACCAGCAGACCTGCGCGCAGTGCGCACCCCGGCTGACGT |
| | | GCAAGGGAGCTCGCTGGCCTCTCTGTGCCCTTGTTCTTCC |
| | | GTGAAATTCTGGCTGAATGTCTCCCCCCACCTTCCGACGC |
| | | TGTCTAGGCAAACCTGGATTAGAGTTACATCTCCTGGATG |
| | | ATTAGTTCAGAGATATATTAAAATGCCCCCTCCCTGTGGA |
| | | TCCTATAG |

In embodiments, modulation of DUX4 such as decreased target transcript or protein levels, can be monitored by analyzing the activity and/or transcript and/or protein levels of downstream genes regulated by DUX4. For example, the expression of DUX4-FL in FSHD lymphoblastoid cell lines correlates with increased expression of DUCX4-FL downstream target genes such as MBD3L2, TRIM43, and ZSCAN4 (Jonas et al., Neuromuscular Disorders (2017), 27(3):221-38). In embodiments, the downstream genes regulated by DUX4 that may be analyzed to assess DUX4 activity and/or mRNA and/or protein levels include, but are not limited to, MBD3L2, TRIM43, ZSCAN4, FRG1, WFDC3, CASP3, MYH3, and/or PAX7.

Because FSHD is caused by a gain of function mutation, DUX4 suppression is a promising treatment strategy. However, numerous highly homologous copies of DUX4 can be found in the human genome, and the D4Z4 repeat is extremely GC-rich, making DUX4 a difficult target. At this time, there is no therapy that prevents or delays disease progression in patients with FSHD (Bouwman et al., Curr. Opin. Neurol. (2020), 33(5):635-640).

U.S. Pat. No. 10,907,157 and Canadian Patent No. 2999192 describe the use of antisense agents and RNA interference agents to decrease expression of DUX4 or DUX4c. Phosphorodiamidate morpholino oligomers targeting various PSEs of DUX4 have demonstrated the ability to alter the expression of DUX4 downstream genes (Marsollier et al., Human molecular genetics, (2016), 25(8), 1468-1478; and Lu-Nguyen et al., Hum Mol Genet. (2021), 30(15): 1398-1412).

DUX4c has also been identified to be upregulated in FSHD (Ansseau et al., PLoS One. (2009), 4(10):e7482, doi:10.1371/journal.pone.0007482). DUX4c has been mapped to a 42 kb centromeric of the D4Z4 region. DUX4c encodes a 47 kb protein that is identical to DUX4 except in the carboxy-terminal region.

Provided herein are compounds, compositions, and methods for downregulation of DUX4 or DUX4c expression. In embodiments, a compound is provided for treating a disease associated with aberrant DUX4 or DUX4c expression. In embodiments, the compound includes an AC. The AC may be any AC and/or have any AC characteristic as described elsewhere herein. The AC may bind to any PSE, or proximate to any PSE, of a DUX4 and/or DUX4c target transcript as described elsewhere herein. For example, the AC may bind at least a portion of a PSE of a DUX4 and/or DUX4c target transcript or may bind a DUX4 and/or DUX4c target transcript in sufficient proximity to a PSE to modulate polyadenylation of the DUX4 and/or DUX4c target transcript. In embodiments, the AC is an ASO. In embodiments, the ASO is a PMO.

Provided herein are compounds, compositions, and methods for treating FSHD. In embodiments, the methods comprise administering a compound or composition described herein to a patient having, or at risk of having, FSHD. In embodiments, the compound or composition downregulates DUX4 or DUX4c expression. In embodiments, the compound includes an AC. The AC may be any AC and/or have any AC characteristic as described elsewhere herein. The AC may bind to any sequence element of a DUX4 and/or DUX4c target transcript as described elsewhere herein. For example, the AC may bind at least a portion of a PSE of a DUX4 and/or DUX4c target transcript or may bind a DUX4 and/or DUX4c target transcript in sufficient proximity to a PSE to modulate polyadenylation of the DUX4 and/or DUX4c target transcript. In embodiments, the AC is an ASO. In embodiments, the ASO is a PMO.

In embodiments, an AC binds to a target nucleotide sequence that includes 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of the RNA transcripts of the DNA sequences of SEQ ID NO: S208, SEQ ID NO: S209, SEQ ID NO: S210 or of variants thereof having 80% or more, 90% or more, 95% or more, 96% or more, 97% or more, 98% or more, 99%/o or more, 99% or less, 98% or less, 97% or less, 96% or less, 95% or less, 90% or less, 80% to 99%, 90% to 99%, 95% to 99%, 96% to 99%, 97% to 99%, or 98% to 99% sequence identity to at least a portion of the RNA transcripts of the DNA sequences of SEQ ID NO: 364, SEQ ID NO: 365, SEQ ID NO: 366.

In embodiments, an AC binds to a target nucleotide sequence that includes at 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of the RNA transcript of the DNA sequence

(nucleotides 1512-1573 of SEQ ID NO: 365)
ACATCTCCTGGATGATTAGTTCAGAGATATATTAAAATGCCCECTCCETG
TGGATCCTATAG;
(nucleotides 1522-1568 of SEQ ID NO: 365)
GATGATTAGTTCAGAGATATATTAAAATGCCCCCTCCCTGTGGATCC;
(nucleotides 1648-1710 of SEQ ID NO: 364)
ACATCTCCTGGATGATTAGTTCAGAGATATATTAAAATGCCCCCTCCCTG
TGGATCCTATAG;
(nucleotides 1658-1703 of SEQ ID NO: 364)
GATGATTAGTTCAGAGATATATTAAAATGCCCCCTCCCTGTGGATCC;
(nucleotides 1708-1768 of SEQ ID NO: 366)
ATCTCCTGGATGATTAGTTCAGAGATATATTAAAATGCCCCCTCCCTGTG
GATCCTATAG;
(nucleotides 1718-1762 of SEQ ID NO: 366)
TGATTAGTTCAGAGATATATTAAAATGCCCCCTCCCTGTGGATCC;
or
combinations thereof (PAS sequence is bolded).

In embodiments, the AC is any one of the antisense sequences in Table 9, portion thereof, or a variant thereof that has 80% to 99%, 90 to 99%, 95%-99% sequence identity of a given antisense compound. SEQ ID NOs: S211-S221 are from Marsollier et al., Human molecular genetics (2016), 25(8), 1468-1478. In most cases the antisense sequence is the exact reverse compliment of the target sequence. In some cases, a mutation, or multiple mutations are introduced in the antisense sequence (bolded in Table 9).

TABLE 9
Antisense Sequences targeting DUX4

| | SEQ | Target Nucleotide | SEQ |
|---|---|---|---|
| Antisense Sequence | ID | Sequence | ID |
| (5′-3′) | NO: | (5′-3′) | NO: |
| GGGCATTTTAATATATCTC | 367 | AGTTCAGAGATATATTAAA | 381 |
| TGAACT | | ATGCCC | |
| CTATAGGATCCACAGGGCA | 368 | GATATTAAAATGCCCTGTG | 382 |
| TTTTAATATC | | GATCCTATAG | |
| GGATCCACAGGGAGGAGGC | 369 | TATTAAAATGCCTCCTCCC | 383 |
| ATTTTAATA | | TGTGGATCC | |
| TATAGGATCCACAGGGAGG | 370 | TTAAAATGCCTCCTCCCTG | 384 |
| AGGCATTTTAA | | TGGATCCTATA | |
| CATCACACAAAAGATGCAA | 371 | GAAGATTTGCATCTTTTGT | 385 |
| ATCTTC | | GTGATG | |
| TATAGGATCCACAGGGAGG | 372 | TTAAAATGCCCCCTCCCTG | 386 |
| AGGCATTTTAA | | TGGATCCTATA | |
| AGGATCCACAGGGAGGGGG | 373 | ATATTAAAATGCCCCCTCC | 387 |
| CATTTTAATAT | | CTGTGGATCCT | |
| TAGGATCCACAGGGAGGGG | 374 | TATTAAAATGCCCCCTCCC | 388 |
| GCATTTTAATA | | TGTGGATCCTA | |
| AGGATCCACAGGGAGGAGG | 375 | ATATTAAAATGCCCCCTCC | 389 |
| CATTTTAATAT | | CTGTGGATCCT | |
| TAGGATCCACAGGGAGGAG | 376 | TATTAAAATGCCCCCTCCC | 390 |
| GCATTTTAATA | | TGTGGATCCTA | |
| AGGATCCACAGGGAGGAGG | 377 | ATTAAAATGCCCCCTCCCT | 391 |
| CATTTTAAT | | GTGGATCCT | |
| GGG CAT TTT AAT ATA | 378 | A GTT CAG AGA TAT | 392 |
| TCT CTG AACT | | ATT AAA ATG CCC | |
| TAT AGG ATC CAC AGG | 379 | TA AAA TGC CTC CTC | 393 |
| GAG GAG GCA TTT TAA | | CCT GTG GAT CCT ATA | |
| TAG GAT CCA CAG GGA | 380 | TAT TAA AAT GCC TCC | 394 |
| GGA GGC ATT TTA ATA | | TCC CTG TGG ATC CTA | |

In embodiments, the AC comprises a gapmer targeting a DUX4 gene transcript. In embodiments, the gapmer comprises a short DNA ASO structure with RNA-mimic segments on either side of the DNA structure. In embodiments, the RNA-mimic segments are LNA segments. In embodiments, the DNA structure of the gapmer is 5 to 15 nucleotides in length. In embodiments, each RNA or RNA-mimic segment is 1 to 10 nucleotides in length, such as 2 to 6 nucleotides in length, 2 to 4 nucleotides in length, or about 3 nucleotides in length. In embodiments, the gapmer binds a target gene transcript at a location that includes at least a portion of a PSE or in sufficient proximity to the PSE to modulate polyadenylation of the target gene transcript. In embodiments, the gapmer binds a target gene transcript at location that does not modulate or substantially modulate polyadenylation. In embodiments, the gapmer mediates degradation of the target gene transcript.

Examples of gapmers that target a DUX4 gene transcript but do not modulate polyadenylation are described in Lim et al., Proc. Ntl. Acad. Sci (2020); 117(28).-16509-15 and are shown in Table 10 below.

TABLE 10

Gapmers targeting DUX4 gene transcript

| SEQ ID NO: | Sequence (5′ to 3′) |
|---|---|
| 395 | AGCGTCGGAAGGTGG |
| 396 | ATAGGATCCACAGGGA |
| 397 | AGATCCCCTCTGCC |
| 398 | CAGCGTCGGAAGGTG |
| 399 | ACAGCGTCGGAAGGTC |
| 400 | GACAGCGTCGGAAGGT |
| 401 | AGACAGCGTCGGAAGG |
| 402 | CGTGCAAGGTG |
| 403 | AGCTCGGAAGGTG |
| 404 | ACACGTCGGAAGGTG |

All sequence are fully phosphorothiorated; bold indicates LNA, nonbold indicates DNA

Additional gapmers that may target a DUX4 gene transcript are shown below Table 11 below.

TABLE 11
Additional gapmers targeting
DUX4 gene transcript

| SEQ ID NO: | Sequence (5′ to 3′) |
|---|---|
| 405 | TAGACAGCGTCGGAAGGTGG |
| 406 | CTAGACAGCGTCGGAAGGTG |
| 407 | CCTAGACAGCGTCGGAAGGT |

The gapmers in Table 11 may have 1 to 5 2′-MOE nucleotides on the 5′ end and may have 1 to 5 2′-MOE nucleotides on the 3′ end. The remainder of the nucleotides may be DNA nucleotides. In embodiments, the gapmers are as described in Lim et al., (2021), Molecular Therapy, 29(2), 848-858.

### DM1 Protein Kinase (DMPK) Gene

Myotonic dystrophy protein kinase (DMPK) is a member of the AGC super family of serine/threonine protein kinases. The DMPK gene encodes several alternative spliced protein products that are mainly expressed in skeletal, heart and smooth muscle and brain. Myotonic dystrophy 1 (DM1), the most common form of muscular dystrophy, is caused by the expansion of an unstable (CTG)˜ repeat in the 3′ untranslated region (3′-UTR) of the DMPK gene. In healthy individuals, the CTG tract is polymorphic with alleles ranging from 5 to 37 in repeats length. Individuals carrying a tract containing between 38 and 49 CTG repeats, are generally asymptomatic but are at risk of transmitting a pathological expanded mutation. A CTG expansion between 50 and 4000 CTG repeats results in DM1 disease (MagaiSa et al., Advances in Protein Kinases (2012), doi: 10.5772/37238).

The DMPK gene includes 15 exons that encode a full-length protein of 692 amino acids. The (CTG)ₙ repeat lies within the 3′UTR of the gene, in exon 15, downstream of the translation stop signal and approximately 500 bp upstream of the poly(A) tail. Additional information regarding the DMPK gene may be found on the National Center for Biotechnology Information (NCBI) website. Human DMPK=NCBI Gene ID 1760. Nuclear accumulation of CUG repeat-containing RNA transcripts interfere with alternative splicing and gene expression (Magafita et al., Advances in Protein Kinases (2012), doi: 10.5772/37238).

Prior methods for treating DM1 include targeting the (CTG)ₙ repeats, for example, with a site-specific RNA endonuclease (Zhang et al. Mol. Ther. (2014) 22(2):312-320) or a small molecule that cleaves the (CUG)ₙ repeats (Angelbello et al., PNAS (2019), 116(16):7799-7804; U.S. Pat. Nos. 10,106,796; 10,111,962: U.S. Patent Publication No. 2015/00803111).

The excessive number of CUG repeats impart toxic activity, referred to as a toxic gain-of-function. Multiple key proteins are misprocessed, and this contributes to the multisystemic nature of the disease, which includes generalized limb weakness, respiratory muscle impairment, cardiac abnormalities, fatigue, gastrointestinal complications, cataracts, incontinence and excessive daytime sleepiness.

Provided herein are compounds, compositions, and methods for downregulation of DMPK expression. In embodiments, a compound is provided for treating a disease associated with aberrant DMPK expression, such as CUG repeat expansion. In embodiments, the compound includes an AC. The AC may be any AC and/or have any AC characteristic as described elsewhere herein. The AC may bind to any sequence element of a DMPK target transcript as described elsewhere herein. For example, the AC may bind at least a portion of a PSE of a DMPK target transcript or may bind a DMPK target transcript in sufficient proximity to a PSE to modulate polyadenylation of the DMPK target transcript. In embodiments, the AC is an ASO. In embodiments, the ASO is a PMO.

Provided herein are compounds, compositions, and methods for treating DM1. In embodiments, the methods comprise administering a compound or composition described herein to a patient having, or at risk of having, DM1. In embodiments, the compound or composition downregulates DMPK expression. In embodiments, the compound includes an AC. The AC may be any AC and/or have any AC characteristic as described elsewhere herein. The AC may bind to any sequence element of a DMPK target transcript as described elsewhere herein. For example, the AC may bind at least a portion of a PSE of a DMPK target transcript or may bind a DMPK target transcript in sufficient proximity to a PSE to modulate polyadenylation of the DMPK target transcript. In embodiments, the AC is an ASO. In embodiments, the ASO is a PMO.

In embodiments, the AC is any one of the antisense sequences in Table 12, portion thereof, or a variant thereof that has 80% to 99%, 90 to 99%, 95% to 99% sequence identity of a given antisense compound.

TABLE 12
AC compounds and target sequences for DMPK1

| | SEQ | | SEQ |
|---|---|---|---|
| Antisense Compound | ID | | ID |
| (5′-3′) | NO: | Target Sequence | NO: |
| GTCCTAGGTGGGGACAGAC | 408 | TTTATTGTCTGTCCCCACC | 449 |
| AATAAA | | TAGGAC | |
| GGGTCCTAGGTGGGGACAG | 409 | TATTGTCTGTCCCCACCTA | 450 |
| ACAATA | | GGACCC | |
| GGGGGTCCTAGGTGGGGAC | 410 | TTGTCTGTCCCCACCTAGG | 451 |
| AGACAA | | ACCCCC | |
| GTGGGGGTCCTAGGTGGGG | 411 | GTCTGTCCCCACCTAGGAC | 452 |
| ACAGAC | | CCCCAC | |
| GGGTGGGGGTCCTAGGTGG | 412 | CTGTCCCCACCTAGGACCC | 453 |
| GGACAG | | CCACCC | |
| GGGGGTGGGGGTCCTAGGT | 413 | GTCCCCACCTAGGACCCCC | 454 |
| GGGGAC | | ACCCCC | |
| TCGGGGGTGGGGGTCCTAG | 414 | CCCCACCTAGGACCCCCAC | 455 |
| GTGGGG | | CCCCGA | |
| GGTCGGGGGTGGGGGTCCT | 415 | CCACCTAGGACCCCCACCC | 456 |
| AGGTGG | | CCGACC | |
| AGGGTCGGGGGTGGGGGTC | 416 | ACCTAGGACCCCCACCCCC | 457 |
| CTAGGT | | GACCCT | |
| CGAGGGTCGGGGGTGGGGG | 417 | CTAGGACCCCCACCCCCGA | 458 |
| TCCTAG | | CCCTCG | |
| CGCGAGGGTCGGGGGTGGG | 418 | AGGACCCCCACCCCCGACC | 459 |
| GGTCCT | | CTCGCG | |
| TTCGCGAGGGTCGGGGGTG | 419 | GACCCCCACCCCCGACCCT | 460 |
| GGGGTC | | CGCGAA | |
| TATTCGCGAGGGTCGGGGG | 420 | CCCCCACCCCCGACCCTCG | 461 |
| TGGGGG | | CGAATA | |
| TTTATTCGCGAGGGTCGGG | 421 | CCCACCCCCGACCCTCGCG | 462 |
| GGTGGG | | AATAAA | |
| CTTTTATTCGCGAGGGTCG | 422 | CACCCCCGACCCTCGCGAA | 463 |
| GGGGTG | | TAAAAG | |
| GCCTTTTATTCGCGAGGGT | 423 | CCCCCGACCCTCGCGAATA | 464 |
| CGGGGG | | AAAGGC | |
| GGGCCTTTTATTCGCGAGG | 424 | CCCGACCCTCGCGAATAAA | 465 |
| GTCGGG | | AGGCCC | |
| GAGGGCCTTTTATTCGCGA | 425 | CGACCCTCGCGAATAAAAG | 466 |
| GGGTCG | | GCCCTC | |
| TGGAGGGCCTTTTATTCGC | 426 | ACCCTCGCGAATAAAAGGC | 467 |
| GAGGGT | | CCTCCA | |
| GATGGAGGGCCTTTTATTC | 427 | CCTCGCGAATAAAAGGCCC | 468 |
| GCGAGG | | TCCATC | |
| CAGATGGAGGGCCTTTTAT | 428 | TCGCGAATAAAAGGCCCTC | 469 |
| TCGCGA | | CATCTG | |
| GGCAGATGGAGGGCCTTTT | 429 | GCGAATAAAAGGCCCTCCA | 470 |
| ATTCGC | | TCTGCC | |
| TGGGCAGATGGAGGGCCTT | 430 | GAATAAAAGGCCCTCCATC | 471 |
| TTATTC | | TGCCCA | |
| TTTGGGCAGATGGAGGGCC | 431 | ATAAAAGGCCCTCCATCTG | 472 |
| TTTTAT | | CCCAAA | |
| GCTTTGGGCAGATGGAGGG | 432 | AAAAGGCCCTCCATCTGCC | 473 |
| CCTTTT | | CAAAGC | |
| GAGCTTTGGGCAGATGGAG | 433 | AAGGCCCTCCATCTGCCCA | 474 |
| GGCCTT | | AAGCTC | |
| CAGAGCTTTGGGCAGATGG | 434 | GGCCCTCCATCTGCCCAAA | 475 |
| AGGGCC | | GCTCTG | |
| TCCAGAGCTTTGGGCAGAT | 435 | CCCTCCATCTGCCCAAAGC | 476 |
| GGAGGG | | TCTGGA | |
| AGTCCAGAGCTTTGGGCAG | 436 | CTCCATCTGCCCAAAGCTC | 477 |
| ATGGAG | | TGGACT | |
| GGAGTCCAGAGCTTTGGGC | 437 | CCATCTGCCCAAAGCTCTG | 478 |
| AGATGG | | GACTCC | |
| GTGGAGTCCAGAGCTTTGG | 438 | ATCTGCCCAAAGCTCTGGA | 479 |
| GCAGAT | | CTCCAC | |
| CTGTGGAGTCCAGAGCTTT | 439 | CTGCCCAAAGCTCTGGACT | 480 |
| GGGCAG | | CCACAG | |
| CACTGTGGAGTCCAGAGCT | 440 | GCCCAAAGCTCTGGACTCC | 481 |
| TTGGGC | | ACAGTG | |
| GACACTGTGGAGTCCAGAG | 441 | CCAAAGCTCTGGACTCCAC | 482 |
| CTTTGG | | AGTGTC | |
| CGGACACTGTGGAGTCCAG | 442 | AAAGCTCTGGACTCCACAG | 483 |
| AGCTTT | | TGTCCG | |
| CGCGGACACTGTGGAGTCC | 443 | AGCTCTGGACTCCACAGTG | 484 |
| AGAGCT | | TCCGCG | |
| ACCGCGGACACTGTGGAGT | 444 | CTCTGGACTCCACAGTGTC | 485 |
| CCAGAG | | CGCGGT | |
| AAACCGCGGACACTGTGGA | 445 | CTGGACTCCACAGTGTCCG | 486 |
| GTCCAG | | CGGTTT | |
| GCAAACCGCGGACACTGTG | 446 | GGACTCCACAGTGTCCGCG | 487 |
| GAGTCC | | GTTTGC | |
| ACGCAAACCGCGGACACTG | 447 | ACTCCACAGTGTCCGCGGT | 488 |
| TGGAGT | | TTGCGT | |
| CAACGCAAACCGCGGACAC | 448 | TCCACAGTGTCCGCGGTTT | 489 |
| TGTGGA | | GCGTTG | |

### Methods of Treatment

The present disclosure provides a method of treating disease in a patient in need thereof, that includes administering a compound disclosed herein. In embodiments, the disease is any of the diseases provided in the present disclosure. In embodiments, a method of treating a disease associated with IRF-5, DUX4, DMPK, or combinations thereof includes administering to the patient a compound disclosed herein, thereby treating the disease. In embodiments, the patient is identified as having, or at risk of having, a disease associated with IRF-5, DUX4, DMPK, or a combination thereof.

In embodiments, the disease or disorder is associated with IRF-5, DUX4, DMPK, or combinations thereof is a disease associated with a genetic variation. In embodiments, the disease or disorder is associated with a genetic mutation in the IRF-5 gene, DUX4 gene, DMPK gene, or combinations thereof. In embodiments, the genetic mutation results in overexpression of IRF-5, DUX4, DMPK, or combinations thereof. In embodiments, the genetic mutation results in the expression of an alternate isoform of IRF-5, DUX4, DMPK, or combinations thereof. In embodiments, the disease or disorder is associated with over expression of IRF-5, DUX4, DMPK, or combinations thereof.

In various embodiments, treatment refers to partial or complete alleviation, amelioration, relief, inhibition, delaying onset, reducing severity and/or incidence of one or more symptoms in a patient.

In embodiments, a method is provided for altering the expression of a disease in a patient in need thereof, that includes administering a compound disclosed herein. In embodiments, treatment results in modulation of IRF-5 activity in a patient. In embodiments, treatment results in modulation of IRF-5 activity in an immune cell of a patient. In embodiments, treatment results in modulation of IRF-5 expression in a patient. In embodiments, treatment results in modulation of IRF-5 expression in an immune cell of a patient. In embodiments, treatment result in a decrease in IRF-5 activity. In embodiments, treatment result in a decrease in IRF-5 expression.

In embodiments, treatment modulates activity of IRF-5 in a patient in need thereof. In embodiments, treatment modulates activity of IRF-5 in a cell of a patient. In embodiments, treatment modulates activity of IRF-5 in an immune cell of a patient. In embodiments, immune cell is a monocyte, a lymphocyte or a dendritic cell. In embodiments, the lymphocyte is a B-lymphocyte. In embodiments, the monocyte is a macrophage. In embodiments, the macrophage is a resident tissue macrophage. In embodiments, the macrophage is a monocyte-derived macrophage. In embodiments, the macrophage is a Kupffer cell, an intraglomerular mesangial cell, an alveolar macrophage, a sinus histiocyte, a hofbauer cell, microglia or langerhan cell. In embodiments, the immune cell is a Kupffer cell.

In embodiments, treatment modulates activity of DUX4 in a patient in need thereof. In embodiments, treatment modulates activity of DUX4 in a cell of a patient. In embodiments, treatment modulates activity of DUX4 in a muscle cell of a patient. In embodiments, muscle cell is a skeletal muscle cell.

In embodiments, treatment modulates activity of DMPK in a patient in need thereof. In embodiments, treatment modulates activity of DMPK in a cell of a patient. In embodiments, treatment modulates activity of a muscle cell of a patient. In embodiments, the muscle cell is a skeletal, heart or smooth muscle cell. In embodiments, treatment modulates activity of DMPK in a cell of the central nervous system of a patient. In embodiments, treatment modulates activity of DMPK in a neuron of a patient. In embodiments, treatment modulates activity of DMPK in a glial cell of a patient.

In embodiments, the method of treatment includes targeted inhibition of mutation-driven IRF-5 overexpression. In embodiments, the method of treatment includes targeted inhibition of mutation-driven DUX4 overexpression. In embodiments, the method of treatment includes targeted inhibition of mutation-driven DMPK overexpression.

In embodiments, treatment according to the present disclosure results in decreased IRF-5, DUX4, or DMPK activity and/or expression in a patient by 5% or more, 10% or more, 20%, or more, 30% or more, 40% or more, 50% or more, 60% or more, 70% or more, 80% or more, 90% or more as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient by 99% or less, 100% or less, 90% or less, 80% or less, 70% or less, 60% or less, 50% or less, 40% or less, 20% or less, or 10% or less as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient by 5% to 10%, 5% to 20%, 5% to 30%, 5% to 40%, 5% to 50%, 5% to 60%, 5% to 70%, 5% to 80%, 5% to 90%, or 5% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient by 20% to 30%, 20% to 40%, 20% to 50%, 20% to 60%, 20% to 70%, 20% to 80%, 20% to 90%, or 20% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4, or DMPK activity and/or expression in a patient by 30% to 40%, 30% to 50%, 30% to 60%, 30% to 70%, 30% to 80%, 30% to 90%, or 30% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4, or DMPK activity and/or expression in a patient by 40% to 50%, 40% to 60%, 40% to 70%, 40% to 80%, 40% to 90%, or 40% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient 50% to 60%, 50% to 70%, 50% to 80%, 50% to 90%, or 50% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient 60% to 70%, 60% to 80%, 60% to 90%, or 60% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, DUX4, or DMPK activity and/or expression in a patient 70% to 80%, 70% to 90%, or 70% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4, or DMPK activity and/or expression in a patient 80% to 90% or 80% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5, DUX4, or DMPK activity and/or expression in a patient 90% to 100% as compared to the average level and/or activity of the target protein in the patient before the treatment, of one or more control individuals with similar disease without treatment, or compared to treatment with a therapeutic moiety not conjugated to a CPP disclosed herein.

In embodiments, treatment according to the present disclosure results in decreased IRF-5 expression in an immune cell of a patient by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of IRF-5 expression in the immune cell of the patient before the treatment, compared to one or more control individuals with similar disease without treatment, or compared to treatment with an therapeutic moiety not conjugated to a CPP disclosed herein.

In embodiments, treatment according to the present disclosure results in decreased IRF-5 expression and/or activity in an immune cell in a patient by 5% or more, 10% or more, 20%, or more, 30% or more, 40% or more, 50% or more, 60% or more, 70% or more, 80% or more, 90% or more as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient by 99% or less, 100% or less, 90% or less, 80% or less, 70% or less, 60% or less, 50% or less, 40% or less, 20% or less, or 10% or less as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient by 5% to 10%, 5% to 20%, 5% to 30%, 5% to 40%, 5% to 50%, 5% to 60%, 5% to 70%, 5% to 80%, 5% to 90%, or 5% to 100% as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient by 20% to 30%, 20% to 40%, 20% to 50%, 20% to 60%, 20% to 70%, 20% to 80%, 20% to 90%, or 20% to 100% as compared to the average level and/or activity of IRF-5 in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 expression and/or activity in an immune cell in a patient by 30% to 40%, 30% to 50%, 30% to 60%, 30% to 70%, 30% to 80%, 30% to 90%, or 30% to 100% as compared to the average level and/or activity of IRF-5 in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 expression and/or activity in an immune cell in a patient by 40% to 50%, 40% to 60%, 40% to 70%, 40% to 80%, 40% to 90%, or 40% to 100% as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient 50% to 60%, 50% to 70%, 50% to 80%, 50% to 90%, or 50% to 100% as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient 60% to 70%, 60% to 80%, 60% to 90%, or 60% to 100% as compared to the average level and/or activity of IRF-5 in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 expression and/or activity in an immune cell in a patient 70% to 80%, 70% to 90%, or 70% to 100% as compared to the average level and/or activity of IRF-5 in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 expression and/or activity in an immune cell in a patient 80% to 90% or 80% to 100% as compared to the average level and/or activity of the IRF-5 in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 expression and/or activity in an immune cell in a patient 90% to 100% as compared to the average level and/or activity of IRF-5 in the patient before the treatment, of one or more control individuals with similar disease without treatment, or compared to treatment with a therapeutic moiety not conjugated to a CPP disclosed herein.

In embodiments, treatment according to the present disclosure results in decreased DUX4 and/or DMPK activity and/or expression in a muscle cell in a patient by 5% or more, 10% or more, 20%, or more, 30% or more, 40% or more, 50% or more, 60% or more, 70% or more, 80% or more, 90% or more as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient by 99% or less, 100% or less, 90% or less, 80% or less, 70% or less, 60% or less, 50% or less, 40% or less, 20% or less, or 10% or less as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient by 5% to 10%, 5% to 20%, 5% to 30%, 5% to 40%, 5% to 50%, 5% to 60%, 5% to 70%, 5% to 80%, 5% to 90%, or 5% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient by 20% to 30%, 20% to 40%, 20% to 50%, 20% to 60%, 20% to 70%, 20% to 80%, 20% to 90%, or 20% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased DUX4 and/or DMPK activity and/or expression in a muscle cell in a patient by 30% to 40%, 30% to 50%, 30% to 60%, 30% to 70%, 30% to 80%, 30% to 90%, or 30% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased DUX4 and/or DMPK activity and/or expression in a muscle cell in a patient by 40% to 50%, 40% to 60%, 40% to 70%, 40% to 80%, 40% to 90%, or 40% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient 50% to 60%, 50% to 70%, 50% to 80%, 50% to 90%, or 50% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient 60% to 70%, 60% to 80%, 60% to 90%, or 60% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient 70% to 80%, 70% to 90%, or 70% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased DUX4 and/or DMPK activity and/or expression in a muscle cell in a patient 80% to 90% or 80% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased DUX4 and/or DMPK activity and/or expression in a muscle cell in a patient 90% to 100% as compared to the average level and/or activity of the target protein in the patient before the treatment, of one or more control individuals with similar disease without treatment, or compared to treatment with a therapeutic moiety not conjugated to a CPP disclosed herein.

In embodiments, treatment according to the present disclosure results in decreased DMPK expression and/or activity in a brain cell in a patient by 5% or more, 10% or more, 20%, or more, 30% or more, 40% or more, 50% or more, 60% or more, 70% or more, 80% or more, 90% or more as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in decreased DMPK expression and/or activity in a brain cell in a patient by 99% or less, 100% or less, 90% or less, 80% or less, 70% or less, 60% or less, 50% or less, 40% or less, 20% or less, or 10% or less as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in decreased DMPK expression and/or activity in a brain cell in a patient by 5% to 10%, 5% to 20%, 5% to 30%, 5% to 40%, 5% to 50%, 5% to 60%, 5% to 70%, 5% to 80%, 5% to 90%, or 5% to 100% as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in decreased DMPK expression and/or activity in a brain cell in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in DMPK expression and/or activity in a brain cell in a patient by 20% to 30%, 20% to 40%, 20% to 50%, 20% to 60%, 20% to 70%, 20% to 80%, 20% to 90%, or 20% to 100% as compared to the average level and/or activity of IRF-5 in the patient before the treatment, of one or more control individuals with similar disease without treatment, or compared to treatment with a therapeutic moiety not conjugated to a CPP disclosed herein. In embodiments, treatment according to the present disclosure results in decreased DMPK expression and/or activity in a brain cell in a patient by 30% to 40%, 30% to 50%, 30% to 60%, 30% to 70%, 30% to 80%, 30% to 90%, or 30% to 100% as compared to the average level and/or activity of DMPK in the patient before the treatment, of one or more control individuals with similar disease without treatment, or compared to treatment with a therapeutic moiety not conjugated to a CPP disclosed herein. In embodiments, treatment according to the present disclosure results in DMPK expression and/or activity in a brain cell in a patient by 40% to 50%, 40% to 60%, 40% to 70%, 40% to 80%, 40% to 90%, or 40% to 100% as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in decreased DMPK expression and/or activity in a brain cell in a patient 50% to 60%, 50% to 70%, 50% to 80%, 50% to 90%, or 50% to 100% as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in decreased DMPK expression and/or activity in a brain cell in a patient 60% to 70%, 60% to 80%, 60% to 90%, or 60% to 100% as compared to the average level and/or activity of DMPK in the patient before the treatment, of one or more control individuals with similar disease without treatment, or compared to treatment with an therapeutic moiety not conjugated to a CPP disclosed herein. In embodiments, treatment according to the present disclosure results in DMPK expression and/or activity in a brain cell in a patient 70% to 80%, 70% to 90%, or 70% to 100% as compared to the average level and/or activity of DMPK in the patient before the treatment, of one or more control individuals with similar disease without treatment, or compared to treatment with a therapeutic moiety not conjugated to a CPP disclosed herein. In embodiments, treatment according to the present disclosure results in decreased DMPK expression and/or activity in a brain cell in a patient 80% to 90% or 80% to 100% as compared to the average level and/or activity of the DMPK in the patient before the treatment, of one or more control individuals with similar disease without treatment, or compared to treatment with a therapeutic moiety not conjugated to a CPP disclosed herein. In embodiments, treatment according to the present disclosure results in decreased DMPK expression and/or activity in a brain cell in a patient 90% to 100% as compared to the average level and/or activity of DMPK in the patient before the treatment, of one or more control individuals with similar disease without treatment, or compared to treatment with a therapeutic moiety not conjugated to a CPP disclosed herein.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are less than about 1-99% of normal protein expression or activity levels in an individual not afflicted with the disease. In embodiments, the range includes, but is not limited to less than about 80-99%, less than about 65-80%, less than about 50-65%, less than about 30-50%, less than about 25-30%, less than about 20-25%, less than about 15-20%, less than about 10-15%, less than about 5-10%, less than about 1-5% of normal thymidine phosphorylase expression or activity levels. In embodiments, the individual may have target protein expression or activity levels that are 1-500% higher than normal wild type target protein expression or activity levels. In embodiments, the range includes, but is not limited to, greater than about 1-10%, about 10-50%, about 50-100%, about 100-200%, about 200-300%, about 300-400%, about 400-500%, or about 500-1000%.

In embodiments, the individual is a patient who has been recently diagnosed with the disease. Typically, early treatment (treatment commencing as soon as possible after diagnosis) reduces the effects of the disease and to increase the benefits of treatment.

### Compositions and Methods of Administration

In embodiments, compositions are provided that include one or more of the compounds described herein.

In embodiments, pharmaceutically acceptable salts and/or prodrugs of the disclosed compounds are provided. Pharmaceutically acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions that include them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously include between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions that include them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell includes attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders that include the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium that includes, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a patient's skin to reduce the size (and can include complete removal) of malignant or benign growths, or to treat an infection site. Compounds and agents disclosed herein can be applied directly to the growth or infection site. In embodiments, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to improve the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

Also disclosed are pharmaceutical compositions that include a compound disclosed herein in combination with a pharmaceutically acceptable carrier. In embodiments, the pharmaceutical composition is adapted for oral, topical or parenteral administration. The dose administered to a patient, particularly a human, should be sufficient to achieve a therapeutic response in the patient over a reasonable time frame, without lethal toxicity, and without causing more than an acceptable level of side effects or morbidity. One skilled in the art will recognize that dosage will depend upon a variety of factors including the condition (health) of the patient, the body weight of the patient, kind of concurrent treatment, if any, frequency of treatment, therapeutic ratio, as well as the severity and stage of the pathological condition.

Also disclosed are kits that include a compound disclosed herein in one or more containers. The disclosed kits can optionally include pharmaceutically acceptable carriers and/or diluents. In embodiments, a kit includes one or more other components, adjuncts, or adjuvants as described herein. In another embodiment, a kit includes one or more anti-cancer agents, such as those agents described herein. In embodiments, a kit includes instructions or packaging materials that describe how to administer a compound or composition of the kit. Containers of the kit can be of any suitable material, e.g., glass, plastic, metal, etc., and of any suitable size, shape, or configuration. In embodiments, a compound and/or agent disclosed herein is provided in the kit as a solid, such as a tablet, pill, or powder form. In another embodiment, a compound and/or agent disclosed herein is provided in the kit as a liquid or solution. In embodiments, the kit includes an ampoule or syringe containing a compound and/or agent disclosed herein in liquid or solution form.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “cell penetrating peptide” or “CPP” refers to a peptide that facilitates delivery of a cargo, e.g., a therapeutic moiety (TM) into a cell. In embodiments, the CPP is cyclic, and is represented as “cCPP”. In embodiments, the cCPP is capable of directing a therapeutic moiety to penetrate the membrane of a cell. In embodiments, the cCPP delivers the therapeutic moiety to the cytosol of the cell. In embodiments, the cCPP delivers an antisense compound (AC) to a cellular location where a pre-mRNA is located.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide, or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO:42), the nucleoplasmin bipartite NLS with the sequence NLSKRPAAIKKAGQAKKKK (SEQ ID NO:52), the c-myc nuclear localization sequence having the amino acid sequence PAAKRVKLD (SEQ ID NO:53) or RQRRNELKRSF (SEQ ID NO:54), the sequence RMRKFKNKGKDTAELRRRRVEVSVELRKAKKDEQILKRRNV (SEQ ID NO:50) of the IBB domain from importin-alpha, the sequences VSRKRPRP (SEQ ID NO:57) and PPKKARED (SEQ ID NO:58) of the myoma T protein, the sequence PQPKKKPL (SEQ ID NO:59) of human p53, the sequence SALIKKKKKMAP (SEQ ID NO:60) of mouse c-abl IV, the sequences DRLRR (SEQ ID NO:61) and PKQKKRK (SEQ ID NO:62) of the influenza virus NS1, the sequence RKLKKKIKKL (SEQ ID NO:63) of the Hepatitis virus delta antigen and the sequence REKKKFLKRR (SEQ ID NO:64) of the mouse Mxl protein, the sequence KRKGDEVDGVDEVAKKKSKK (SEQ ID NO:65) of the human poly(ADP-ribose) polymerase and the sequence RKCLQAGMNLEARKTKK (SEQ ID NO:66) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

As used herein, the term “contiguous” refers to two amino acids, which are connected by a covalent bond. For example, in the context of a representative cyclic cell penetrating peptide (cCPP) such as

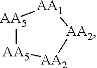

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

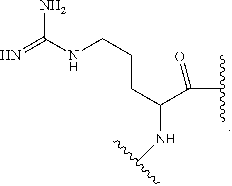 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

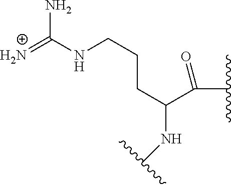 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein below.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes Cu and C₁₂ alkyls. Non-limiting examples of C₁-C₁₂ alkyl include methyl, ethyl, n-propyl, i-propyl, sec-propyl, n-butyl, i-butyl, sec-butyl, t-butyl, in-pentyl, t-amyl, in-hexyl, n-heptyl, n-octyl, i-nonyl, n-decyl, n-undecyl, and n-dodecyl. Unless stated otherwise specifically in the specification, an alkyl group can be optionally substituted.

“Alkylene”, “alkylene chain″ or “alkylene group” refers to a fully saturated, straight or branched divalent hydrocarbon chain radical, having from one to forty carbon atoms. Non-limiting examples of C₂-C₄₀ alkylene include ethylene, propylene, in-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain″ or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRaR₄, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “ [symbol] ” infers that when R³ is “XY”, the point of attachment bond is the same bond as the bond by which R³ is depicted as being bonded to CH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutically acceptable carrier can be a sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized antisense oligonucleotide. In embodiments, a dosage unit is a vial that includes reconstituted antisense oligonucleotide.

The term “therapeutic moiety” (TM) refers to a compound that can be used for treating, at least one symptom of a disease or disorder and can include, but is not limited to, therapeutic polypeptides, oligonucleotides, small molecules and other agents that can be used to treat, prevent, or ameliorate at least one symptom of a disease or disorder. In embodiments, the therapeutic moiety modulates expression or activity of a target protein. In embodiments, the therapeutic moiety targets a polyadenylation sequence element of a target gene or an RNA transcript of a target gene. In embodiments, the therapeutic moiety targets a polyadenylation sequence element of an mRNA transcript of a target gene. In embodiments, the therapeutic moiety downregulates expression or activity of a target protein. In embodiments, the therapeutic moiety upregulates expression or activity of a target protein.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In embodiments, the compound disclosed herein includes a therapeutic moiety (TM) that downregulates expression, function and/or activity of a target protein. In embodiments, the compound disclosed herein includes a therapeutic moiety that upregulates expression, function and/or activity of a target protein.

“Amino acid” refers to an organic compound that includes an amino group and a carboxylic acid group and has the general formula

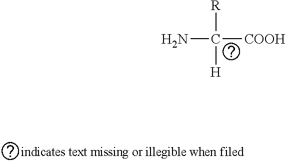

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain″ or “side chain″ refers to the characterizing substituent (“R”) bound to the α-carbon of a natural or non-natural α-amino acid. An amino acid may be incorporated into a polypeptide via a peptide bond.

As used herein, the term “sequence identity” refers to the percentage of nucleic acids or amino acids between two oligonucleotide or polypeptide sequences, respectively, that are the same and in the same relative position. As such one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, R^{1c}e et al., Trends Genet. (2000), 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residues×100)/(Length of Alignment-Total Number of Gaps in Alignment)

In other embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Such programs am described in Altschul, et al., J. Mol. Biol., (1990), 215(3): 403-410: Altschul, et al., Nucleic Acids Res. (1997), 25:3389-3402; Baxevanis et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, “cell targeting moiety” refers to a molecule or macromolecule that specifically binds to a molecule, such as a receptor, on the surface of a target cell. In embodiments, the cell surface molecule is expressed only on the surface of a target cell. In embodiments, the cell surface molecule is also present on the surface of one or more non-target cells, but the amount of cell surface molecule expression is higher on the surface of the target cells. Examples of a cell targeting moiety include, but are not limited to, an antibody, a peptide, a protein, an aptamer or a small molecule.

As used herein, the terms “antisense compound” and “AC” are used interchangeably to refer to a polymeric nucleic acid structure which is at least partially complementary to a target nucleic acid molecule to which it (the AC) hybridizes. The AC may be a short (in embodiments, less than 50 bases) polynucleotide or polynucleotide homologue that includes a sequence complimentary to a target sequence. In embodiments, the AC is a polynucleotide or polynucleotide homologue that includes a sequence complimentary to a target sequence in a target pre-mRNA strand. The AC may be formed of natural nucleic acids, synthetic nucleic acids, nucleic acid homologues, or any combination thereof. In embodiments, the AC includes oligonucleosides. In embodiments, AC includes antisense oligonucleotides. In embodiments, the AC includes conjugate groups. Nonlimiting examples of ACs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In embodiments, an AC modulates (increases, decreases, or changes) expression of a target nucleic acid.

As used herein, the terms “targeting” or “targeted to” refer to the association of a therapeutic moiety, for example, an antisense compound with a target nucleic acid molecule or a region of a target nucleic acid molecule. In embodiments, the therapeutic moiety includes an antisense compound that is capable of hybridizing to a target nucleic acid under physiological conditions. In embodiments, the antisense compound targets a specific portion or site within the target nucleic acid, for example, a portion of the target nucleic acid having at least one identifiable structure, function, or characteristic, for example, selected nucleobases or motifs within one or more polyadenylation sequence element (PSE).

As used herein, the terms “target nucleic acid” refers to the nucleic acid sequence to which the antisense compound binds or hybridizes. Target nucleic acids include, but are not limited to, RNA (including, but not limited to pre-mRNA and mRNA or portions thereof), cDNA derived from such RNA, as well as non-translated RNA, such as miRNA. For example, in embodiments, a target nucleic acid can be a cellular gene (or mRNA transcribed from such gene) whose expression is associated with a particular disorder or disease state. The term “portion″ refers to a defined number of contiguous (i.e., linked) nucleobases of a nucleic acid.

As used herein, “proximate” means that the AC binds to a nucleic acid sequence that is within about 25, about 20, about 15, about 10, about 5, about 4, about 3, about 2 or about 1 nucleotides of a polyadenylation sequence element (PSE).

The term “target RNA” refers to an RNA molecule to which a therapeutic moiety binds. In one embodiment, the target RNA is mRNA. In one embodiment, the target RNA is pre-mRNA. In one embodiment, the target RNA includes a PSE or a portion thereof.

As used herein, the term “mRNA” refers to an RNA molecule that encodes a protein and includes pre-mRNA and mature mRNA. “Pre-mRNA” refers to a newly synthesized eukaryotic mRNA molecule directly after DNA transcription. In embodiments, a pre-mRNA is capped with a 5′ cap, modified with a 3′ poly-A tail, and/or spliced to produce a mature mRNA sequence. In embodiments, pre-mRNA includes one or more introns. In one embodiment, the pre-mRNA undergoes a process known as splicing to remove introns and join exons. In embodiments, pre-mRNA includes a PSE.

The term “target protein” refers to the amino acid sequence encoded by the target mRNA.

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (tRNA) and ribosomal RNA. Gene expression of a polypeptide includes transcription of the gene to form a pre-mRNA, processing of the pre-mRNA to form a mature mRNA, translocating the mature mRNA from the nucleus to the cytoplasm, translation of the mature mRNA into the polypeptide, and assembly of the encoded polypeptide.

As used herein, “modulation of gene expression” or the like refers to modulation of one or more of the processes associated with gene expression. For example, modification of gene expression may include modification of one or more of gene transcription, RNA processing, RNA translocation from the nucleus to the cytoplasm, and translation of mRNA into a protein.

As used herein, the term “gene” refers to a nucleic acid sequence that encompasses a 5′ promoter region associated with the expression of the gene product, and any intron and exon regions and 3′ untranslated regions (“UTR”) associated with the expression of the gene product.

The term “immune cell” refers to a cell of hematopoietic origin and that plays a role in the immune response. Immune cells include, but are not limited to, lymphocytes (e.g., B cells and T cells), natural killer (NK) cells, and myeloid cells. The term “myeloid cells” includes monocytes, macrophages and granulocytes (e.g., basophils, neutrophils, eosinophils and mast cells). Monocytes are lymphocytes that circulate through the blood for 1-3 days, after which time, they either migrate into tissues and differentiate into macrophages or inflammatory dendritic cells or die. The term “macrophage” as used herein includes fetal-derived macrophages (which also can be referred to as resident tissue macrophages) and macrophages derived from monocytes that have migrated from the bloodstream into a tissue in the body (which can be referred to as monocyte-derived macrophages). Depending on which tissue the macrophage is located, it be referred to as a Kupffer cell (liver), an intraglomular mesangial cell (kidney), an alveolar macrophage (lungs), a sinus histiocyte (lymph nodes), a hofbauer cell (placenta), microglia (brain and spinal cord), or langerhans (skin), among others.

As used herein, the term “transcript” or “gene transcript” refers an RNA molecule transcribed from DNA and includes, but is not limited to mRNA, pre-mRNA, and partially processed RNA.

As used herein, “splicing” refers to the modification of a pre-mRNA following transcription in which introns are removed and exons are joined. Splicing occurs in a series of reactions that are catalyzed by a large RNA-protein complex that includes five small nuclear ribonucleoproteins (snRNPs), referred to as a spliceosome. Splice regulatory elements include a 3′ splice site, a 5′ splice site, and a branch site. Ward and Cooper (2011) “The pathobiology of splicing,” J. Pathol. 220(2):152-163.

As used herein “polyadenylation” refers to the cellular process in which a chain of adenosine bases (referred to as a poly(A) tail) is added to an RNA transcript, for example, a pre-mRNA. Polyadenylation is a two-step reaction that includes specific endonucleolytic cleavage of the 3′end of an RNA transcript at and addition of the poly(A) tail. The processing of most human poly(A) sites involves the recognition of a canonical hexamer sequence by a cleavage and polyadenylation specific factor (CPSF), coupled with the binding of cleavage stimulatory factor (CstF) to a GU-rich downstream element (DSE). See, Venkataraman et al., Genes and Dev. (2005), 19:1315-1327.

As used herein a “poly(A) tail” or “polyadenosine tail” refers to a chain of adenosine bases on the 3′ end of a mRNA sequence. The length of poly(A) tails is generally specific to a species. For example, poly(A) tails can range from 150 to 250 adenosines in mammals and from 55 to 90 adenosines in yeasts (See, Tian et al. (2005) “A large-scale analysis of mRNA polyadenylation of human and mouse genes,” Nuc. Acid. Res. 33(1):201-212 and Neve et al. (2017) “Cleavage and polyadenylation: ending the message expands gene regulation,” RNA Biology, 14(7).865-890 and Brown, C. E. and Sachs, A. B. (1998) Poly(A) tail length control in Saccharomyces cerevisiae occurs by message-specific deadenylatoin. Mol. Cell. Biol., 18, 6548-6559).

As used herein, the term “polyadenylation sequence element” refers to recurring nucleotide sequence motif that is associated with polyadenylation. Polyadenylation sequence elements include a polyadenylation signal (PAS), an intervening sequence (IS), a cleavage site (CS), and a downstream element (DSE). See, FIG. 1. As used herein, “cleavage site(s)” refers to sequence comprising a nucleotide pair between which cleavage takes place. Following cleavage, a poly(A) tail is added to the 3′ end resulting from the cleavage. The CS may include a canonical sequence or a variant thereof. The “polyadenylation signal” (PAS) is an adenosine-rich hexamer sequence. The polyadenylation signal PAS may include a canonical hexamer sequence or a variant thereof. The polyadenylation signal (PAS) is typically found from about 10 to about 35, or about 10 to about 20, or at least about 10, about 11, about 12, about 13, about 14, about 15, about 16, about 17, about 18, about 19, about 20, about 21, about 22, about 23, about 24 or about 25 and up to about 30 or about 35 nucleotides upstream of the cleavage site (CS), separated by the intervening sequence (IS). The hexamer sequence serves as the binding site for the cleavage and polyadenylation specificity factor (CPSF). The downstream sequence element (DSE) includes a U-rich or U/G-rich element. The DSE is typically found from about 10 to about 40, about 10 to about 30, about 20 to about 40, or about 20 to about 30, or at least about 20, about 21, about 22, about 23, about 24, or about 25 and up to about 26, about 27, about 28, about 29, about 30, about 35 or about 40 nucleotides downstream of the cleavage site. The U-rich or U/G-rich element serves as a binding site for the cleavage stimulation factor (CstF). The DSE may be followed by a stretch of 3 or more uracil residues (U) downstream of the cleavage site, often within 40 nucleotides of the cleavage site. In mammals, CA and UA are the most frequent dinucleotides that precede the cleavage site, although the actual cleavage site is known to be heterogeneous. The polyadenylation sequence elements can also include other auxiliary elements, such as upstream U-rich elements (USE). See, Tian et al. (2005) “A large-scale analysis of mRNA polyadenylation of human and mouse genes,” Nuc. Acid. Res. 33(1):201-212 and Neve et al. (2017) “Cleavage and polyadenylation: ending the message expands gene regulation,” RNA Biology, 14(7):865-890.

As used herein, “cleavage and polyadenylation” or “CPA” refers to a two-step process involving generation of a 3′ end through an initial endonucleolytic cleavage of RNA followed by addition of a chain of adenosine bases (a poly(A) tail) to an RNA sequence.

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

As used herein, the term “nucleoside” refers to a glycosylamine that includes a nucleobase and a sugar. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein “oligonucleoside” refers to an oligonucleotide in which the internucleoside linkages do not contain a phosphorus atom.

As used herein, the term “oligonucleotide” refers to an oligomeric compound that includes a plurality of linked nucleotides or nucleosides. In certain embodiment, one or more nucleotides of an oligonucleotide is modified. In embodiments, an oligonucleotide includes ribonucleic acid (RNA) or deoxyribonucleic acid (DNA). In embodiments, oligonucleotides are composed of natural and/or modified nucleobases, sugars and covalent internucleoside linkages, and may further include non-nucleic acid conjugates.

As used herein “internucleoside linkage” refers to a covalent linkage between adjacent nucleosides.

As used herein “natural internucleoside linkage” refers to a 3′ to 5′ phosphodiester linkage.

As used herein, the term “modified internucleoside linkage” refers to any linkage between nucleosides or nucleotides other than a naturally occurring internucleoside linkage.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general, a chimeric oligomeric compound will have modified nucleosides that can be in isolated positions or grouped together in regions that will define a particular motif. Any combination of modifications and or mimetic groups can include a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleoside linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may include up to about 20% nucleotides that are mismatched (i.e., are not nucleobase complementary to the corresponding nucleotides of the target). In embodiments, the antisense compounds contain no more than about 15%, for example, not more than about 10%, for example, not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99%, or 100% nucleobase complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “2′-modified” or “2′-substituted” means a sugar that includes substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O-C1-C10 alkyl, —OCF3, O—(CH2)2-O—CH3, 2′-O(CH2)2SCH3, O—(CH2)2-O—N(Rm)(Rn), or O—CH₂—C(═O)—N(Rm)(Rn), where each Rm and Rn is, independently, H or substituted or unsubstituted C1-C10 alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound that includes the modified nucleotide to a target nucleic acid. High-affinity modifications include, but are not limited to, BNAs, LNAs and 2′-MOE.

As used herein the term “mimetic” refers to groups that are substituted for a sugar, a nucleobase, and/or internucleoside linkage in an AC. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH3)-CH2-4′) and Aminooxy BNA (2′-N(CH3)-O-CH2-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of an AC.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1. Construction of a Cell-Penetrating Peptide—Antisense Compound Conjugate

An antisense compound (AC) of any one of SEQ ID NOs:158 to 260; 367 to 380; or 408 to 448 designed to bind to and block mRNA expression of IRF-5, DMPK, or DUX is constructed as a phosphorodiamidate morpholino oligomer (PMO) with a C6-thiol 5′ modification. In embodiments, the AC includes a 3′ or a 5′ functional group that allows for conjugation to an EEV, for example, using click chemistry.

An EEV is formulated that includes a CCP. A cell-penetrating peptide is formulated using Fmoc chemistry and conjugated to the AC, for example, as described in International Application No. PCT/US20/66459, filed by Entrada Therapeutics, Inc., on Dec. 21, 2021, entitled “COMPOSITIONS FOR DELIVERY OF ANTISENSE COMPOUNDS,” the disclosure of which is hereby incorporated in its entirety herein. In embodiments, the cCPP includes the amino acid sequence FfΦRrRrQ (SEQ ID NO:78). In embodiments EEV includes an exocyclic peptide having the sequence KKKRKV (SEQ ID NO:33). In embodiments, the EEV includes KKKRKV-PEG₂-K-(cyclo(FfΦRrRr)) (SEQ ID NO:33-PEG₂-K-(cyclo(SEQ ID NO:78)) or KKKRKV-PEG₂-K-(cyclo(FfΦRrRrQ))-PEG₁₂-K(N3) (SEQ ID NO:33-PEG₂-K-(cyclo(SEQ ID NO:78))-PEG₁₂-K(N₃)). In embodiments, the AC compound is conjugated to the EEV using click chemistry. In embodiments, the compound includes KKKRKV-PEG₂-K-(cyclo(FfΦRrRrQ))-PEG₁₂-K-linker-3′-AC-5′ (SEQ ID NO:33-PEG₂-K-(cyclo(SEQ ID NO:78))-PEG₁₂-K-linker-3′-AC-5′) where the linker includes the product of a strain promoted click reaction between an azide and a cyclooctyne. The linker may also include other groups such as a carbon chain, PEG chain, carbamate, urea, and the like.

### Example 2. Sequence Evaluation of PMOs Targeting the IRF-5 Polyadenylation Signal

Various PMOs were evaluated for targeting the IRF-5 polyadenylation signal. The PMOs tested included SEQ ID NO:253 (PAS-1), SEQ ID NO:254 (PAS-2), SEQ ID NO:255 (PAS-3), SEQ ID NO:256 (PAS-4), SEQ ID NO:257 (PAS-1b), SEQ ID NO:258 (PAS-2b), SEQ ID NO:259 (PAS-3b), and SEQ ID NO:260 (PAS-4b). The PMOs targeted SEQ ID NOs:356 to 363 as shown in Table 7.

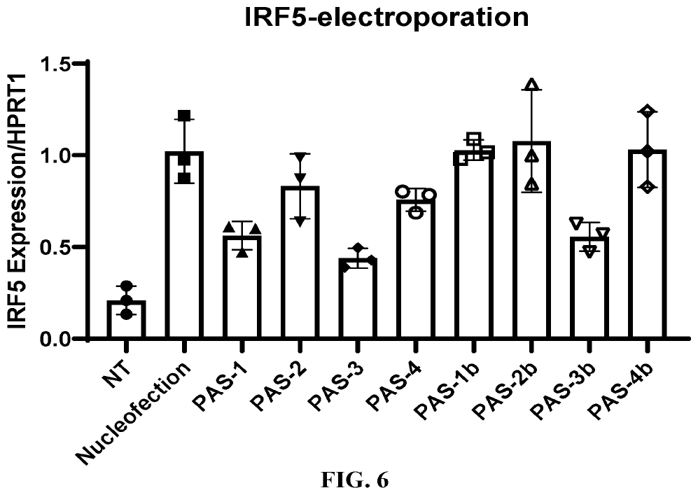

THP1 cells are monocytes isolated from a human acute monocytic leukemia patient and are available from ATCC as TIB-202™ cells. THP1 cells are often used to study monocyte and macrophage functions and mechanisms. THP1 cells were transfected with 5 μM PMO by nucleofection. Various PMOs demonstrated reduced IRF-5 expression (FIG. 6) indicating that targeting one or more IRF-5 PSEs is a viable strategy for downregulating gene expression.

### Example 3: Sequence Evaluation of PMOs Targeting the DMPK Polyadenylation Signal

Various AC compounds evaluated for targeting the DMPK polyadenylation signal. The AC compounds were 2′-OMe ASOs with a phosphorothioate backbone. The ACs tested are shown in Tables 13 and 14. In Tables 13 and 14 “AC” is the antisense compound that binds to the target nucleotide sequence.

TABLE 13
ACs targeting the polyadenylation signal of DMPK

| | Target nucleotide | Target | | Tm |
|---|---|---|---|---|
| AC SEQ ID NO: | SEQ ID NO: | site | GC % | (° C.) |
| 408 | 449 | −45 − 21 | 48 | 58 |
| 409 | 450 | −43 − 19 | 56 | 61.4 |
| 410 | 451 | −41 − 17 | 64 | 65.6 |
| 411 | 452 | −39 − 15 | 68 | 66.1 |
| 412 | 453 | −37 − 13 | 72 | 68.2 |
| 413 | 454 | −35 − 11 | 76 | 70.3 |
| 414 | 455 | −33 − 9 | 76 | 71.6 |
| 415 | 456 | −31 − 7 | 76 | 70.2 |
| 416 | 457 | −29 − 5 | 72 | 70.9 |
| 417 | 458 | −27 − 3 | 76 | 69.8 |
| 418 | 459 | −25 − 1 | 80 | 74 |
| 419 | 460 | −23 + 2 | 76 | 72.1 |
| 420 | 461 | −21 + 4 | 72 | 70 |
| 421 | 462 | −19 + 6 | 64 | 66.5 |
| 422 | 463 | −17 + 8 | 60 | 63.5 |
| 423 | 464 | −15 + 10 | 64 | 65.6 |

TABLE 14
ACs targeting the polyadenylation signal of DMPK

| | AC | Target | | | | ΔG for | ΔG for |
|---|---|---|---|---|---|---|---|
| | SEQ | SEQ | Target | GC | Tm | binding | homo dimers |
| ID | ID NO: | ID NO: | site | % | (° C.) | (kcal/mol) | (kcal/mol) |
| DM17; (PAS17) | 424 | 465 | −1 | 64 | 65.6 | −58.07 | −13.51 |
| DM18; (PAS18) | 425 | 466 | 3 | 60 | 63.4 | −55.11 | −13.51 |
| DM19; (PAS19) | 426 | 467 | 7 | 56 | 64.2 | −54.95 | −13.51 |
| DM20; (PAS20) | 427 | 468 | 11 | 56 | 61.6 | −53.58 | −13.51 |
| DM21; (PAS21) | 428 | 469 | 15 | 52 | 61 | −52.47 | −13.51 |
| DM22; (PAS22) | 429 | 470 | 19 | 56 | 62.1 | −53.49 | −9.28 |
| DM23; (PAS23) | 430 | 471 | 23 | 52 | 61.1 | −51.76 | −9.28 |
| DM24; (PAS24) | 431 | 472 | 28 | 48 | 60.8 | −52.13 | −9.28 |
| DM25; (PAS25) | 432 | 473 | 32 | 56 | 63 | −54.44 | −9.28 |
| DM26; (PAS26) | 433 | 474 | 36 | 60 | 65.2 | −53.72 | −9.28 |
| DM27; (PAS27) | 434 | 475 | 40 | 64 | 65.7 | −53.73 | −9.28 |
| DM28; (PAS28) | 435 | 476 | 44 | 60 | 64.5 | −52.16 | −6.6 |
| DM29; (PAS29) | 436 | 477 | 48 | 56 | 62.5 | −48.97 | −6.6 |
| DM30: (PAS30) | 437 | 478 | 52 | 60 | 63.3 | −50.44 | −6.34 |
| DM31: (PAS31) | 438 | 479 | 56 | 56 | 62.7 | −48.71 | −6.6 |
| DM32; (PAS32) | 439 | 480 | 60 | 60 | 63.4 | −49.21 | −6.6 |
| DM33; (PAS33) | 440 | 481 | +20 + 44 | 60 | 63.5 | −49.96 | −6.6 |
| DM34; (PAS34) | 441 | 482 | +22 + 46 | 56 | 61.2 | −45.67 | −6.6 |
| DM35; (PAS35) | 442 | 483 | +24 + 48 | 56 | 62.4 | −47.32 | −6.6 |
| DM36; (PAS36) | 443 | 484 | +26 + 50 | 64 | 66.1 | −50.19 | 10.36 |
| DM37; (PAS37) | 444 | 485 | +28 + 52 | 64 | 66 | −49.86 | −16.5 |
| DM38; (PAS38) | 445 | 486 | +30 + 54 | 60 | 64.8 | −50.57 | −16.5 |
| DM39; (PAS39) | 446 | 487 | +32 + 56 | 64 | 65.8 | −52.11 | −16.5 |
| DM40; (PAS40) | 447 | 488 | +34 + 58 | 60 | 66.7 | −52.42 | −16.5 |
| DM41; (PAS41) | 448 | 489 | +36 + 60 | 60 | 65.7 | −53.38 | −16.5 |

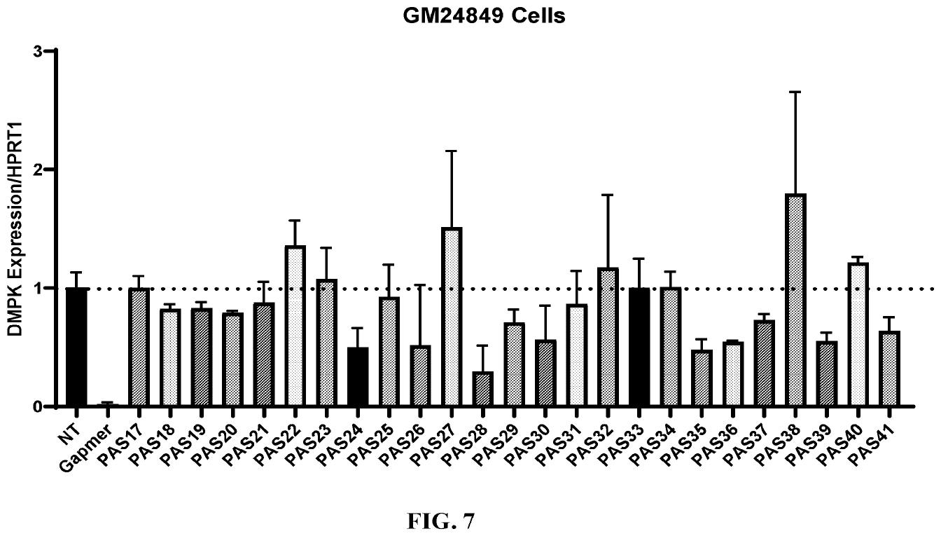

DM1 patient-derived fibroblasts were transfected with 100 nM of various ACs using RNAiMAX transfection. A gapmer positive of sequence 5′-ACA GAC AAT AAA TAC CGA GG-3′ (SEQ ID NO: 490), where underlined bases are 2′-MOE, other bases are DNA, and the entire backbone it PS was used a positive control. PAS24, PAS26, PAS28, PAS30, PAS35, PAS36 and PAS39 can effectively knockdown human DMPK mRNA levels (FIG. 7).

### Example 4. Evaluation of PMOs and PMO-EEV Conjugates Targeting DUX4

Various PMOs and PMO-EEV conjugates were evaluated for targeting DUX4. Table 15 shows the sequences of PMOs targeting DUX4 polyadenylation sequence elements (PSEs). The PMOs in Table 15 included at least a portion of a PSE of the DUX4 sequence. The sequence for the EEV for conjugate PAS-EEV (127-777) is AcPKKKRKVK-(cyclo(FfΦRrRrQ) (Ac-SEQ ID No:42-K-(cyclo(SEQ ID NO:78)) (EEV 777). The general structure for the PAS-EEV conjugate is shown in FIG. 11.

TABLE 15
PMOs and PMO-EEV construct for targeting DUX4 PSEs

| | PMO SEQ | | Target nucleotide | Target |
|---|---|---|---|---|
| ID | ID NO: | EEV | SEQ ID NO: | Region |
| PAS; 127 | 378 | no | 391 | −13 + 12 |
| PAS-EEV; | 378 | yes (777) | 391 | −13 + 12 |
| 127-777 | | | | |
| CS3 | 379 | no | 392 | +2 + 31 |
| PACS4 | 379 | no | 393 | −1 + 29 |

FSHD patient line GM16283 (F, 30 YR), and WT lines GM16275 and GM16281 (F, 20 YR); were treated with 1 μM, 2 μM, 5 μM, or 10 μM of a PMO or PMO-EEV construct of table T7 via Lonza nucleofection, free uptake, or nucleofection. One day post treatment, the RNA was extracted and the mRNA level of DUX4-FL, DUX4-3′UTR and DUX4 downstream genes MBD3L2, ZSCAN4, and TRIM43 relative to RPL19 was quantified. RPL19 was previously determined to be a good reference gene.

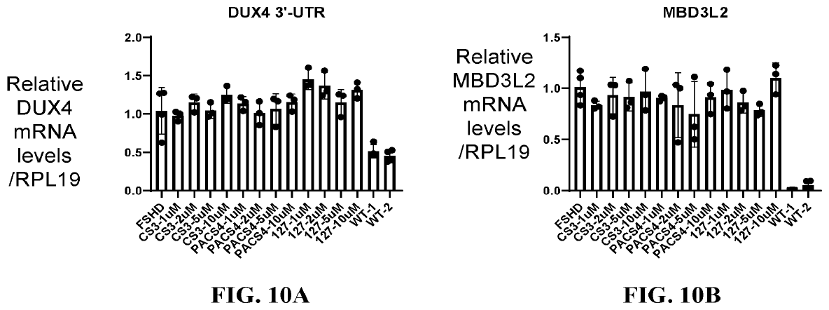

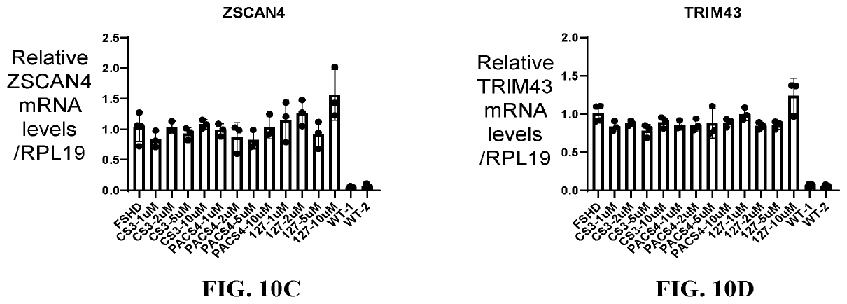

Cells treated with various concentration of PAS (127), CS3, and PACS4 via endoporter transfection showed no decrease in mRNA levels of DUX4 3′UTR (FIG. 10A), MBD3L2 (FIG. 10B), ZSCAN4 (FIG. 10C), and TRIM43 (FIG. 10D).

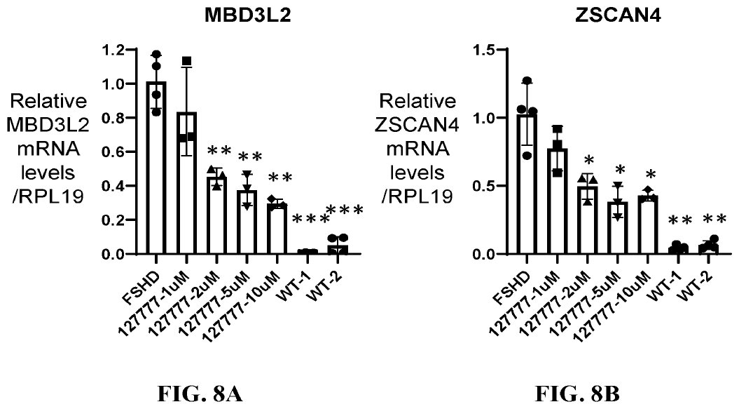

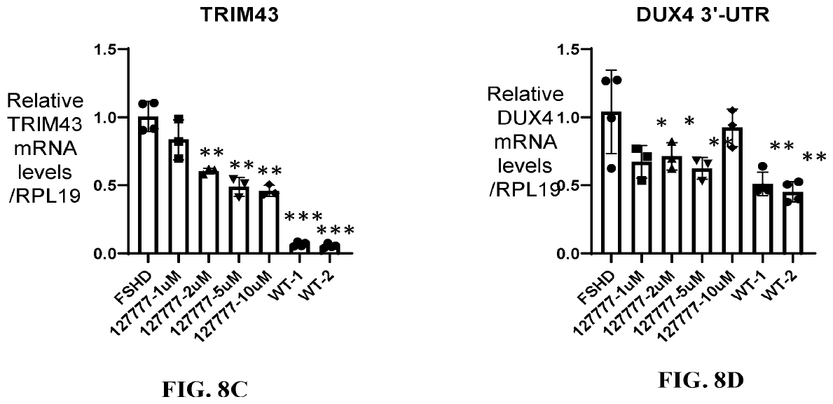

Treatment via fee uptake with PAS-EEV (127-777) result in a dose dependent decrease of MBD3L2 (FIG. 8A), ZSCAN4 (FIG. 8B), and TRIM4 (FIG. 8C), downstream genes regulated by DUX4. The levels of DUX4 3′-UTR (FIG. 8D) did not show a dose dependent decrease. DUX4 is challenging to measure which may have resulted in the variable DUX 4 levels.

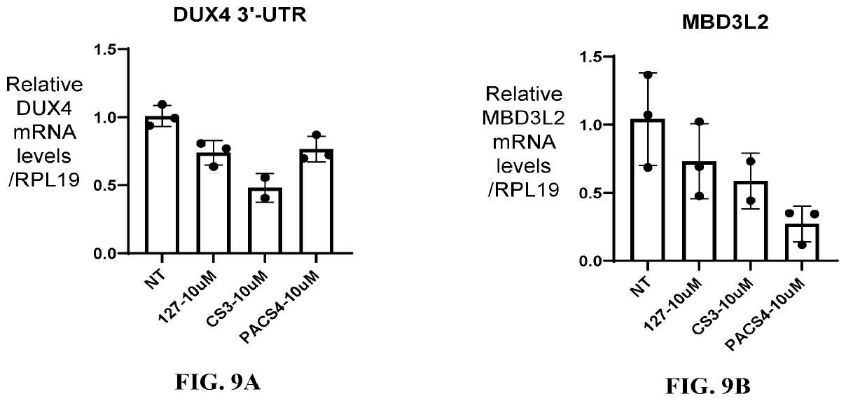

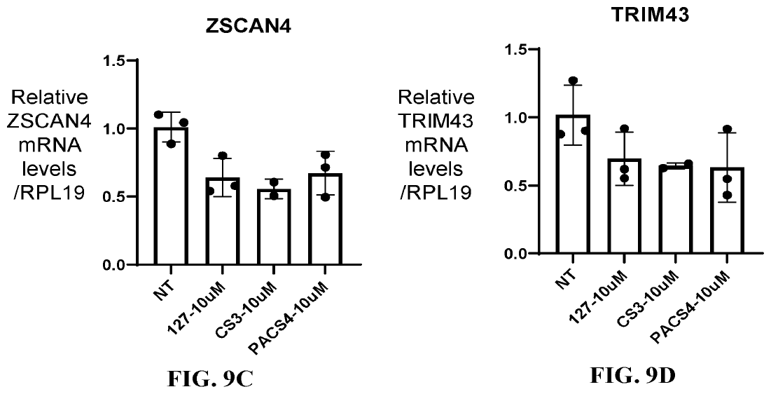

PMOs CS3, PAS (127), and PACS4 showed varying abilities to decrease the mRNA levels of DUX4 3′-UTR (FIG. 9A), MBD3L2 (FIG. 9B), ZSCAN4 (FIG. 8C), and TRIM4 (FIG. 8D). The PAS-EEV construct showed a greater decrease in the mRNA levels of MBD3L2, ZSCAN4, and TRIM4 compared to the PAS PMO alone.

In a similar study, immortalized myoblasts from FSHD (AB1080), and unaffected individuals (KM1421; AB1190) were treated with varying concentrations of PAS-EEV (127-777).

The FSHD patient myoblasts contain a 6.3 D4Z4 contraction, indicating the cells are from FSHD patients. In contrast, healthy people have 11-100 D4Z4 repeats (3.3 kilobases/repeat) which are modified by CpG methylation and silence DUX4 expression. FSHD 1 patients have fewer D4Z4 repeats which causes hypomethylation and stable expression of toxic DUX4 protein.

Myoblasts were cultured in a growth medium consisting of Skeletal Muscle Cell Growth Medium (PomoCell), 2% horse serum (Gibco), 1% chick embryo extract (USB), and 0.5 mg/mL penicillin/streptomycin (Gibco). For myogenic differentiation, confluent cultures were switched to differentiation medium consisting of DMEM supplemented with 2% horse serum and cultured for 5 (FSHD) days. For treatment of FSHD cultures, EEV-PMO was serially diluted in differentiation medium and added to confluent myoblasts at the onset of myogenic differentiation. Myoblasts were exposed to EEV-PMO throughout differentiation and harvested at five days. RNA isolation and PCR. Total RNA was isolated with the Qiagen RNEASY Mini Kit according to the manufacturer's instructions. For exon inclusion, 100 ng of RNA was reverse transcribed and used for PCR (ONESTEP RT-PCR Kit, Qiagen). Samples were analyzed by LABCHIP (PerkinElmer) with the HT DNA High Sensitivity Assay Kit (PerkinElmer). For relative quantification of DUX4 target genes, 500 ng RNA was reverse transcribed with the SuperScript IV first-strand synthesis system (Invitrogen) in a 20 μL reaction. Two microliters of cDNA were used for qPCR (SYBR Green Master Mix, Applied Biosystems).

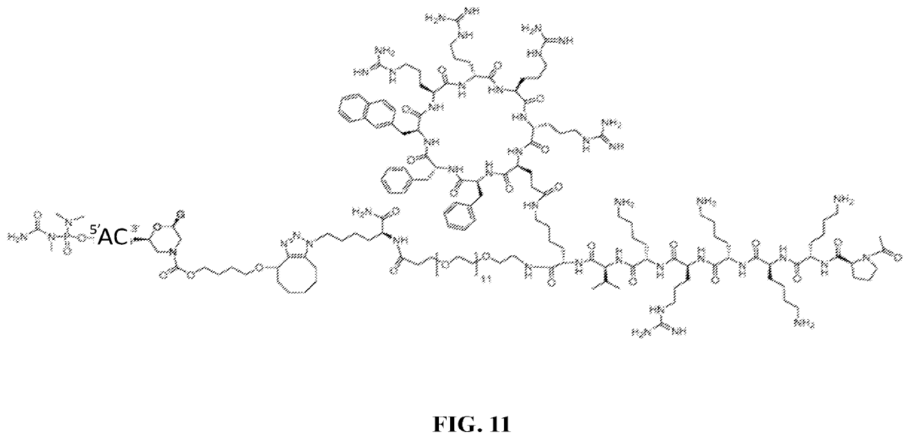

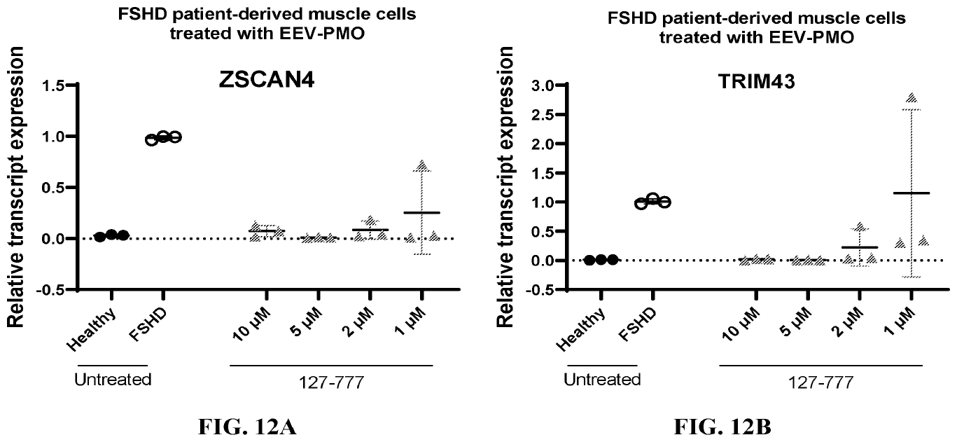

Treatment with PAS-EEV (127-777) showed a decrease in ZSCAN4 (FIG. 12A) and TRIM34 (FIG. 12B) in patient derived muscle cells. Microscopy studies indicated that treatment with PAS-EEV (127-777) induced cell death.

### Example 5: Evaluation of Gapmers Targeting DUX4

Patient derived muscle cells were treated with gapmers targeting DUX4. The following gapmers were used: “L1” CGTCGCAAGGTG (SEQ ID NO:402); “L2” AGCGTCGGAAGGTG (SEQ ID NO:403); and “L3” ACAGCGTCGGAAGGTG (SEQ ID NO:404), where bolded indicates a LNA and where nonbolded indicated DNA. The gapmers did not target a DUX4 polyadenylation sequence element (PSE).

Myoblasts were nucleofected with the gapmers at 10 micromolar, 1 micromolar, and 0.1 micromolar concentrations. The compounds were removed 24 hours post-differentiation. The myoblasts were differentiated for 5 days. Downstream biomarkers, ZSCAN4 and TRIM43 were assessed as discussed above in Example 4.

Gapmer L2 induced overt cell death at the 1 micromolar and 10 micromolar concentrations. Gapmer L3 produced variable results at the 0.1 micromolar concentration. Otherwise, the gapmers resulted in reduced ZSCAN4 and TRIM43 expression, particularly at higher concentrations (data not shown).

A number of embodiments have been described herein. Nevertheless, it will be understood that various modifications may be made without departing from the spirit and scope of the invention. Accordingly, other embodiments are within the scope of the following claims.

1-121. (canceled)

122. A compound comprising:
(a) a cyclic cell penetrating peptide of the formula: 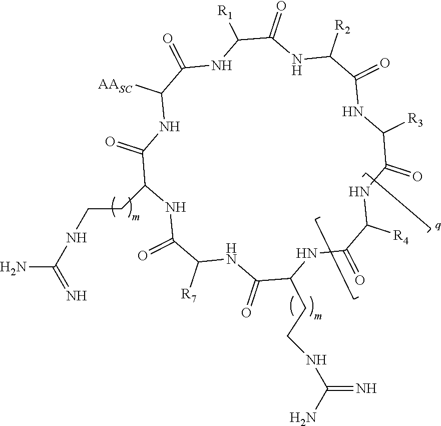
or a protonated form or salt thereof, wherein:
R₁, R₂, and R₃ are each independently H or an aromatic or heteroaromatic side chain of an amino acid;
at least two of R₁, R₂, and R₃ are the side chain of phenylalanine;
R₄ and R₇ are independently H or an amino acid side chain;
each m is independently an integer from 0 to 3;
AA_{SC} is an amino acid side chain; and
q is 1, 2, 3, or 4;
(b) an exocyclic peptide comprising from 2 to 10 amino acid residues, wherein 2, 3, or 4 of the residues are lysine residues;
(c) a linker of formula: 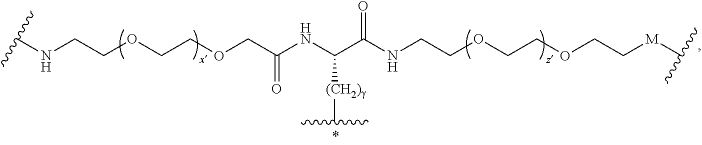
wherein:
x′ is an integer from 1-23;
y is an integer from 1-5;
z′ is an integer from 1-23;
* is the point of attachment to the AA_{SC} of the cyclic peptide;
and M is a bonding group;
(d) an oligonucleotide comprising an antisense compound that is complementary to a target nucleotide sequence comprising at least a portion of a polyadenylation sequence element of a target transcript of a target gene.

123. The compound of claim 122, wherein the exocyclic peptide comprises at least 2 amino acid residues with a hydrophobic side chain.

124. The compound of claim 122, wherein the exocyclic peptide comprises PKKKRKV.

125. The compound of claim 122, wherein z′ is 11.

126. The compound of claim 122, wherein x′ is 1.

127. The compound of claim 122, wherein M comprises

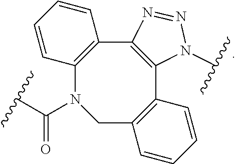 [SMILES: *C(=O)N1Cc2ccccc2-c2c(nnn2*)-c2ccccc21]

128. The compound of claim 122, wherein M comprises

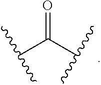 [SMILES: *C(*)=O]

129. The compound of claim 122, wherein the cyclic cell penetrating peptide is 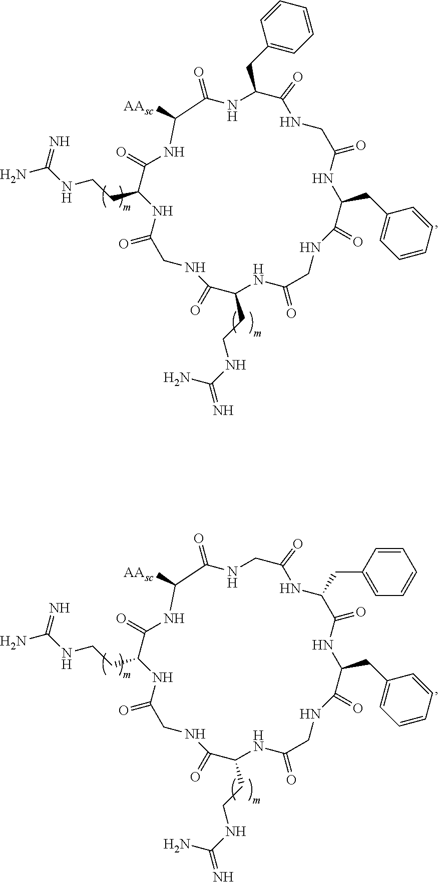 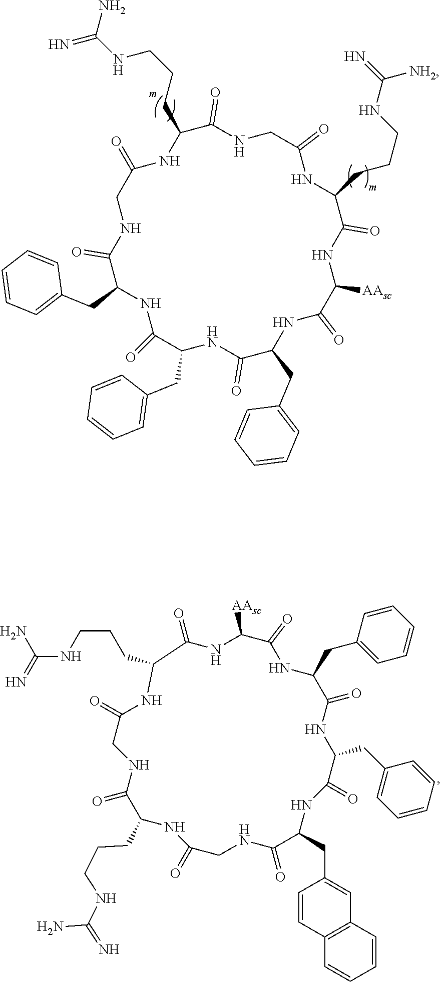 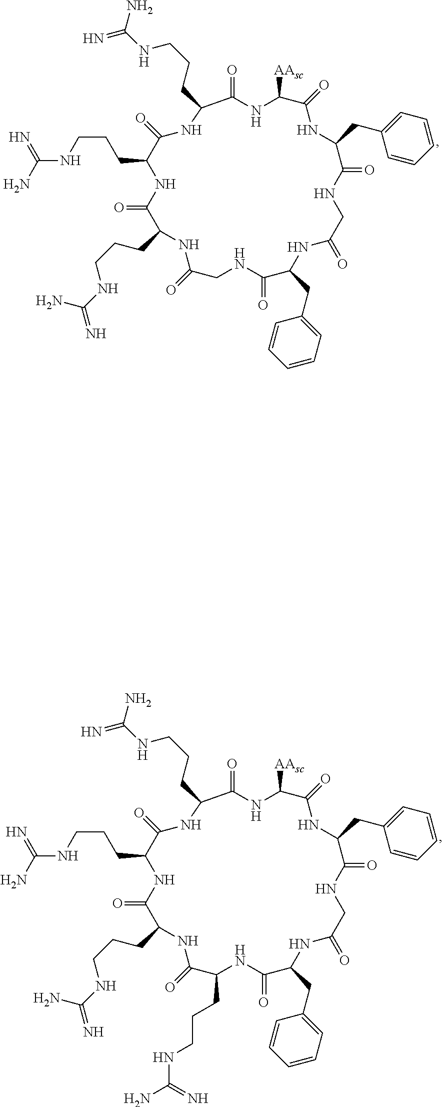 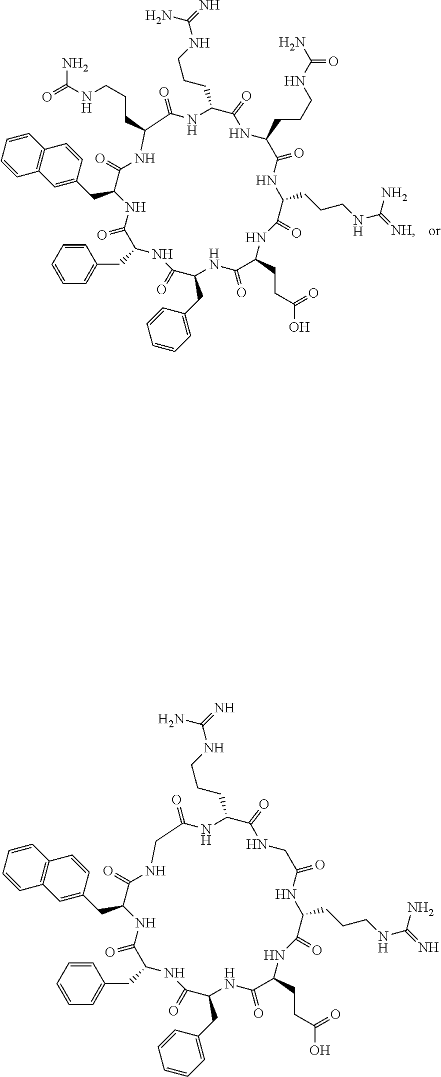 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]
or a protonated form or salt thereof, wherein if the structure does not include AA_{sc} then at least one atom of an amino acid side chain is replaced by the linker or at least one lone pair forms a bond to the linker.

130. The compound of claim 122, comprising an oligonucleotide conjugated to an endosomal escape vehicle with a sequence selected from: 2
Ac-PKKKRKV-PEG₂-K(cyclo[Ff-Nal-Cit-r-Cit-r-Q])-
PEG₁₂-lys(N₃)-NH₂;
Ac-PKKKRKV-K(cyclo[Ff-Nal-GrGrQ])-PEG₁₂-Lys(N₃)-
NH₂;
Ac-PKKKRKV-miniPEG₂-Lys(cyclo(FfFGRGRQ)-PEG₂-
K(N₃)-NH₂;
Ac-PKKKRKV-PEG₂-K(cyclo[FGFGRGRQ])-PEG₂-Lys(N₃)-
NH₂;
Ac-PKKKRKV-PEG₂-K(cyclo[GfFGrGrQ])-PEG₂-Lys(N₃)-
NH₂;
Ac-PKKKRKV-PEG₂-Lys(cyclo[FfFGRGRQ)-miniPEG2-
K(N₃)-NH₂;
Ac-PKKKRKV-PEG₂-K(cyclo(Ff-Nal-GrGrQ)-PEG₁₂-OH;
Ac-PKKKRKV-PEG₂-K(cyclo[FGFGRGRQ])-PEG₁₂-OH;
Ac-PKKKRKV-PEG₂-K(cyclo[GfFGrGrQ])-PEG₁₂-OH;
Ac-PKKKRKV-PEG₂-K(cyclo[FGFGRRRQ])-PEG₁₂-OH;
and
Ac-PKKKRKV-PEG₂-K(cyclo[FGFRRRRQ])-PEG₁₂-OH.

131. The compound of claim 122, wherein the oligonucleotide comprises a phosphorodiamidate morpholino oligomer.

132. The compound of claim 122, wherein the oligonucleotide comprises 5 to 50 nucleotides in length.

133. The compound of claim 122, wherein the oligonucleotide decreases expression of the target gene.

134. The compound of claim 122, wherein the polyadenylation sequence element comprises a polyadenylation signal, an intervening sequence, a cleavage site, a downstream element, or a portion thereof, or a combination thereof.

135. The compound of claim 122, wherein target transcript comprises a double homeobox 4 transcript (DUX4).

136. The compound of claim 135, wherein the antisense compound comprises any one of SEQ ID NO: 367 to SEQ ID NO: 380.

137. A pharmaceutical composition comprising the compound of claim 122 and a pharmaceutically acceptable carrier.

138. A method for treating a genetic disease in a subject in need thereof, comprising administering to the subject a compound of any one of claim 122 to the subject.

139. The method of claim 138, wherein the genetic disease is associated with aberrant expression of DUX4, wherein the target gene is DUX4.

140. The method of claim 139, wherein the disease is Facioscapulohumeral muscular dystrophy (FSHD).


SEQUENCE LISTING (501 sequences)

SEQ ID NO: 1 - protein, 4 aa, Artificial Sequence
  Description of Artificial Sequence: Synthetic peptide
HHHH

SEQ ID NO: 2 - protein, 4 aa, Artificial Sequence
  Description of Artificial Sequence: Synthetic peptide
KHKK

SEQ ID NO: 3 - protein, 4 aa, Artificial Sequence
  Description of Artificial Sequence: Synthetic peptide
KKHK

SEQ ID NO: 4 - protein, 4 aa, Artificial Sequence
  Description of Artificial Sequence: Synthetic peptide
KKKH

SEQ ID NO: 5 - protein, 4 aa, Artificial Sequence
  Description of Artificial Sequence: Synthetic peptide
KHKH

SEQ ID NO: 6 - protein, 4 aa, Artificial Sequence
  Description of Artificial Sequence: Synthetic peptide
HKHK

SEQ ID NO: 7 - protein, 4 aa, Artificial Sequence
  Description of Artificial Sequence: Synthetic peptide
KKKK

SEQ ID NO: 8 - protein, 4 aa, Artificial Sequence
  Description of Artificial Sequence: Synthetic peptide
KKRK

SEQ ID NO: 9 - protein, 4 aa, Artificial Sequence
  Description of Artificial Sequence: Synthetic peptide
KRKK

SEQ ID NO: 10 - protein, 4 aa, Artificial Sequence
  Description of Artificial Sequence: Synthetic peptide
KRRK

SEQ ID NO: 11 - protein, 4 aa, Artificial Sequence
  Description of Artificial Sequence: Synthetic peptide
RKKR

SEQ ID NO: 12 - protein, 4 aa, Artificial Sequence
  Description of Artificial Sequence: Synthetic peptide
RRRR

SEQ ID NO: 13 - protein, 4 aa, Artificial Sequence
  Description of Artificial Sequence: Synthetic peptide
KGKK

SEQ ID NO: 14 - protein, 4 aa, Artificial Sequence
  Description of Artificial Sequence: Synthetic peptide
KKGK

SEQ ID NO: 15 - protein, 5 aa, Artificial Sequence
  Description of Artificial Sequence: Synthetic peptide
  MOD_RES (2)..(2): Beta-alanine
  MOD_RES (4)..(4): Beta-alanine
HAHAH

SEQ ID NO: 16 - protein, 5 aa, Artificial Sequence
  Description of Artificial Sequence: Synthetic peptide
  MOD_RES (2)..(2): Beta-alanine
  MOD_RES (4)..(4): Beta-alanine
HAKAH

SEQ ID NO: 17 - protein, 5 aa, Artificial Sequence
  Description of Artificial Sequence: Synthetic peptide
RRRRR

SEQ ID NO: 18 - protein, 5 aa, Artificial Sequence
  Description of Artificial Sequence: Synthetic peptide
KKKKK

SEQ ID NO: 19 - protein, 5 aa, Artificial Sequence
  Description of Artificial Sequence: Synthetic peptide
KKKRK

SEQ ID NO: 20 - protein, 5 aa, Artificial Sequence
  Description of Artificial Sequence: Synthetic peptide
RKKKK

SEQ ID NO: 21 - protein, 5 aa, Artificial Sequence
  Description of Artificial Sequence: Synthetic peptide
KRKKK

SEQ ID NO: 22 - protein, 5 aa, Artificial Sequence
  Description of Artificial Sequence: Synthetic peptide
KKRKK

SEQ ID NO: 23 - protein, 5 aa, Artificial Sequence
  Description of Artificial Sequence: Synthetic peptide
KKKKR

SEQ ID NO: 24 - protein, 5 aa, Artificial Sequence
  Description of Artificial Sequence: Synthetic peptide
  MOD_RES (2)..(2): Beta-alanine
  MOD_RES (4)..(4): Beta-alanine
KAKAK

SEQ ID NO: 25 - protein, 6 aa, Artificial Sequence
  Description of Artificial Sequence: Synthetic peptide
RKKKKG

SEQ ID NO: 26 - protein, 6 aa, Artificial Sequence
  Description of Artificial Sequence: Synthetic peptide
KRKKKG

SEQ ID NO: 27 - protein, 6 aa, Artificial Sequence
  Description of Artificial Sequence: Synthetic peptide
KKRKKG

SEQ ID NO: 28 - protein, 6 aa, Artificial Sequence
  Description of Artificial Sequence: Synthetic peptide
KKKKRG

SEQ ID NO: 29 - protein, 6 aa, Artificial Sequence
  Description of Artificial Sequence: Synthetic peptide
  MOD_RES (6)..(6): Beta-alanine
RKKKKA

SEQ ID NO: 30 - protein, 6 aa, Artificial Sequence
  Description of Artificial Sequence: Synthetic peptide
  MOD_RES (6)..(6): Beta-alanine
KRKKKA

SEQ ID NO: 31 - protein, 6 aa, Artificial Sequence
  Description of Artificial Sequence: Synthetic peptide
  MOD_RES (6)..(6): Beta-alanine
KKRKKA

SEQ ID NO: 32 - protein, 6 aa, Artificial Sequence
  Description of Artificial Sequence: Synthetic peptide
  MOD_RES (6)..(6): Beta-alanine
KKKKRA

SEQ ID NO: 33 - protein, 6 aa, Artificial Sequence
  Description of Artificial Sequence: Synthetic peptide
KKKRKV

SEQ ID NO: 34 - protein, 6 aa, Artificial Sequence
  Description of Artificial Sequence: Synthetic peptide
RRRRRR

SEQ ID NO: 35 - protein, 6 aa, Artificial Sequence
  Description of Artificial Sequence: Synthetic peptide
HHHHHH

SEQ ID NO: 36 - protein, 6 aa, Artificial Sequence
  Description of Artificial Sequence: Synthetic peptide
RHRHRH

SEQ ID NO: 37 - protein, 6 aa, Artificial Sequence
  Description of Artificial Sequence: Synthetic peptide
HRHRHR

SEQ ID NO: 38 - protein, 6 aa, Artificial Sequence
  Description of Artificial Sequence: Synthetic peptide
KRKRKR

SEQ ID NO: 39 - protein, 6 aa, Artificial Sequence
  Description of Artificial Sequence: Synthetic peptide
RKRKRK

SEQ ID NO: 40 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
RARARA

SEQ ID NO: 41 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
KAKAKA

SEQ ID NO: 42 - protein, 7 aa, Artificial Sequence
  Description of Artificial Sequence: Synthetic peptide
PKKKRKV

SEQ ID NO: 43 - protein, 7 aa, Artificial Sequence
  Description of Artificial Sequence: Synthetic peptide
PGKKRKV

SEQ ID NO: 44 - protein, 7 aa, Artificial Sequence
  Description of Artificial Sequence: Synthetic peptide
PKGKRKV

SEQ ID NO: 45 - protein, 7 aa, Artificial Sequence
  Description of Artificial Sequence: Synthetic peptide
PKKGRKV

SEQ ID NO: 46 - protein, 7 aa, Artificial Sequence
  Description of Artificial Sequence: Synthetic peptide
PKKKGKV

SEQ ID NO: 47 - protein, 7 aa, Artificial Sequence
  Description of Artificial Sequence: Synthetic peptide
PKKKRGV

SEQ ID NO: 48 - protein, 7 aa, Artificial Sequence
  Description of Artificial Sequence: Synthetic peptide
PKKKRKG

SEQ ID NO: 49 - protein, 5 aa, Artificial Sequence
  Description of Artificial Sequence: Synthetic peptide
  MOD_RES (2)..(2): Beta-alanine
  MOD_RES (4)..(4): Beta-alanine
RARAR

SEQ ID NO: 50 - protein, 5 aa, Artificial Sequence
  Description of Artificial Sequence: Synthetic peptide
  MOD_RES (2)..(2): Beta-alanine
  MOD_RES (4)..(4): Beta-alanine
RAHAR

SEQ ID NO: 51 - protein, 5 aa, Artificial Sequence
  Description of Artificial Sequence: Synthetic peptide
  MOD_RES (2)..(2): Beta-alanine
  MOD_RES (4)..(4): Beta-alanine
HARAH

SEQ ID NO: 52 - protein, 19 aa, Artificial Sequence
  Description of Artificial Sequence: Synthetic peptide
NLSKRPAAIKKAGQAKKKK

SEQ ID NO: 53 - protein, 9 aa, Artificial Sequence
  Description of Artificial Sequence: Synthetic peptide
PAAKRVKLD

SEQ ID NO: 54 - protein, 11 aa, Artificial Sequence
  Description of Artificial Sequence: Synthetic peptide
RQRRNELKRSF

SEQ ID NO: 55 - protein, 27 aa, Artificial Sequence
  Description of Artificial Sequence: Synthetic peptide
RMRKFKNKGKDTAELRRRRVEVSVELR

SEQ ID NO: 56 - protein, 14 aa, Artificial Sequence
  Description of Artificial Sequence: Synthetic peptide
KAKKDEQILKRRNV

SEQ ID NO: 57 - protein, 8 aa, Artificial Sequence
  Description of Artificial Sequence: Synthetic peptide
VSRKRPRP

SEQ ID NO: 58 - protein, 8 aa, Artificial Sequence
  Description of Artificial Sequence: Synthetic peptide
PPKKARED

SEQ ID NO: 59 - protein, 8 aa, Artificial Sequence
  Description of Artificial Sequence: Synthetic peptide
PQPKKKPL

SEQ ID NO: 60 - protein, 12 aa, Artificial Sequence
  Description of Artificial Sequence: Synthetic peptide
SALIKKKKKMAP

SEQ ID NO: 61 - protein, 5 aa, Artificial Sequence
  Description of Artificial Sequence: Synthetic peptide
DRLRR

SEQ ID NO: 62 - protein, 7 aa, Artificial Sequence
  Description of Artificial Sequence: Synthetic peptide
PKQKKRK

SEQ ID NO: 63 - protein, 10 aa, Artificial Sequence
  Description of Artificial Sequence: Synthetic peptide
RKLKKKIKKL

SEQ ID NO: 64 - protein, 10 aa, Artificial Sequence
  Description of Artificial Sequence: Synthetic peptide
REKKKFLKRR

SEQ ID NO: 65 - protein, 20 aa, Artificial Sequence
  Description of Artificial Sequence: Synthetic peptide
KRKGDEVDGVDEVAKKKSKK

SEQ ID NO: 66 - protein, 17 aa, Artificial Sequence
  Description of Artificial Sequence: Synthetic peptide
RKCLQAGMNLEARKTKK

SEQ ID NO: 67 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 68 - protein, 7 aa, Artificial Sequence
  Description of Artificial Sequence: Synthetic peptide
FGFGRGR

SEQ ID NO: 69 - protein, 7 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGR

SEQ ID NO: 70 - protein, 7 aa, Artificial Sequence
  Description of Artificial Sequence: Synthetic peptide
  MOD_RES (2)..(2): D-phenylalanine
  MOD_RES (3)..(3): L-naphthylalanine
FFAGRGR

SEQ ID NO: 71 - protein, 7 aa, Artificial Sequence
  Description of Artificial Sequence: Synthetic peptide
  MOD_RES (2)..(2): D-phenylalanine
FFFGRGR

SEQ ID NO: 72 - protein, 7 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGR

SEQ ID NO: 73 - protein, 4 aa, Artificial Sequence
  Description of Artificial Sequence: Synthetic peptide
  MOD_RES (4)..(4): L-naphthylalanine
FGFA

SEQ ID NO: 74 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 75 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFAGRGRQ

SEQ ID NO: 76 - protein, 8 aa, Artificial Sequence
  Description of Artificial Sequence: Synthetic peptide
  MOD_RES (2)..(2): D-phenylalanine
FFFGRGRQ

SEQ ID NO: 77 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 78 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 79 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (4)..(4): Citrulline
  MOD_RES (5)..(5): D-arginine
  MOD_RES (6)..(6): Citrulline
  MOD_RES (7)..(7): D-arginine
FFAXRXRQ

SEQ ID NO: 80 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 81 - protein, 8 aa, Artificial Sequence
  Description of Artificial Sequence: Synthetic peptide
  MOD_RES (2)..(2): D-phenylalanine
FFFGRGRQ

SEQ ID NO: 82 - protein, 8 aa, Artificial Sequence
  Description of Artificial Sequence: Synthetic peptide
FGFGRGRQ

SEQ ID NO: 83 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 84 - protein, 8 aa, Artificial Sequence
  Description of Artificial Sequence: Synthetic peptide
FGFGRRRQ

SEQ ID NO: 85 - protein, 8 aa, Artificial Sequence
  Description of Artificial Sequence: Synthetic peptide
FGFRRRRQ

SEQ ID NO: 86 - protein, 7 aa, Artificial Sequence
  Description of Artificial Sequence: Synthetic peptide
  MOD_RES (2)..(2): L-naphthylalanine
FARRRRQ

SEQ ID NO: 87 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 88 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFARGRGQ

SEQ ID NO: 89 - protein, 6 aa, Artificial Sequence
  Description of Artificial Sequence: Synthetic peptide
  MOD_RES (2)..(2): L-naphthylalanine
FARRRQ

SEQ ID NO: 90 - protein, 6 aa, Artificial Sequence
  Description of Artificial Sequence: Synthetic peptide
  MOD_RES (2)..(2): L-naphthylalanine
FARRRC

SEQ ID NO: 91 - protein, 6 aa, Artificial Sequence
  Description of Artificial Sequence: Synthetic peptide
  MOD_RES (2)..(2): L-naphthylalanine
  MOD_RES (6)..(6): Selenocysteine
FARRRX

SEQ ID NO: 92 - protein, 6 aa, Artificial Sequence
  Description of Artificial Sequence: Synthetic peptide
  MOD_RES (4)..(4): L-naphthylalanine
RRRAFQ

SEQ ID NO: 93 - protein, 6 aa, Artificial Sequence
  Description of Artificial Sequence: Synthetic peptide
  MOD_RES (5)..(5): L-naphthylalanine
RRRRAF

SEQ ID NO: 94 - protein, 6 aa, Artificial Sequence
  Description of Artificial Sequence: Synthetic peptide
  MOD_RES (2)..(2): L-naphthylalanine
FARRRR

SEQ ID NO: 95 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
  MOD_RES (7)..(7): D-glutamine
FARRRRQ

SEQ ID NO: 96 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
FARRRRQ

SEQ ID NO: 97 - protein, 7 aa, Artificial Sequence
  Description of Artificial Sequence: Synthetic peptide
  MOD_RES (2)..(2): L-naphthylalanine
FARRRRQ

SEQ ID NO: 98 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 99 - protein, 7 aa, Artificial Sequence
  Description of Artificial Sequence: Synthetic peptide
  MOD_RES (5)..(5): L-naphthylalanine
RRFRARQ

SEQ ID NO: 100 - protein, 7 aa, Artificial Sequence
  Description of Artificial Sequence: Synthetic peptide
  MOD_RES (6)..(6): L-naphthylalanine
FRRRRAQ

SEQ ID NO: 101 - protein, 7 aa, Artificial Sequence
  Description of Artificial Sequence: Synthetic peptide
  MOD_RES (1)..(1): D-arginine
  MOD_RES (5)..(5): L-naphthylalanine
RRFRARQ

SEQ ID NO: 102 - protein, 7 aa, Artificial Sequence
  Description of Artificial Sequence: Synthetic peptide
  MOD_RES (3)..(3): L-naphthylalanine
RRAFRRQ

SEQ ID NO: 103 - protein, 8 aa, Artificial Sequence
  Description of Artificial Sequence: Synthetic peptide
CRRRRFWQ

SEQ ID NO: 104 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 105 - protein, 8 aa, Artificial Sequence
  Description of Artificial Sequence: Synthetic peptide
  MOD_RES (3)..(3): L-naphthylalanine
FFARRRRQ

SEQ ID NO: 106 - protein, 8 aa, Artificial Sequence
  Description of Artificial Sequence: Synthetic peptide
  MOD_RES (6)..(6): L-naphthylalanine
RFRFRARQ

SEQ ID NO: 107 - protein, 8 aa, Artificial Sequence
  Description of Artificial Sequence: Synthetic peptide
  MOD_RES (1)..(1): Selenocysteine
XRRRRFWQ

SEQ ID NO: 108 - protein, 8 aa, Artificial Sequence
  Description of Artificial Sequence: Synthetic peptide
CRRRRFWQ

SEQ ID NO: 109 - protein, 8 aa, Artificial Sequence
  Description of Artificial Sequence: Synthetic peptide
  MOD_RES (2)..(2): L-naphthylalanine
FARRRRQK

SEQ ID NO: 110 - protein, 8 aa, Artificial Sequence
  Description of Artificial Sequence: Synthetic peptide
  MOD_RES (2)..(2): L-naphthylalanine
FARRRRQC

SEQ ID NO: 111 - protein, 8 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRRQ

SEQ ID NO: 112 - protein, 8 aa, Artificial Sequence
  Description of Artificial Sequence: Synthetic peptide
  MOD_RES (2)..(2): L-naphthylalanine
FARRRRRQ

SEQ ID NO: 113 - protein, 9 aa, Artificial Sequence
  Description of Artificial Sequence: Synthetic peptide
  MOD_RES (5)..(5): L-naphthylalanine
  MOD_RES (8)..(8): Norleucine
RRRRAFDXC

SEQ ID NO: 114 - protein, 5 aa, Artificial Sequence
  Description of Artificial Sequence: Synthetic peptide
  MOD_RES (2)..(2): L-naphthylalanine
FARRR

SEQ ID NO: 115 - protein, 5 aa, Artificial Sequence
  Description of Artificial Sequence: Synthetic peptide
FWRRR

SEQ ID NO: 116 - protein, 5 aa, Artificial Sequence
  Description of Artificial Sequence: Synthetic peptide
  MOD_RES (4)..(4): L-naphthylalanine
RRRAF

SEQ ID NO: 117 - protein, 5 aa, Artificial Sequence
  Description of Artificial Sequence: Synthetic peptide
RRRWF

SEQ ID NO: 118-149 - skipped (32 entries)

SEQ ID NO: 150 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 151 - protein, 497 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGM

SEQ ID NO: 152 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 153 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 154 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 155 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 156 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 157 - DNA, 757 bp, Homo sapiens
tcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaatta

SEQ ID NO: 158 - DNA, 10 bp, Artificial Sequence
  Description of Artificial Sequence: Synthetic oligonucleotide
acactcggtt

SEQ ID NO: 159 - DNA, 10 bp, Artificial Sequence
  Description of Artificial Sequence: Synthetic oligonucleotide
cactcggttt

SEQ ID NO: 160 - DNA, 10 bp, Artificial Sequence
  Description of Artificial Sequence: Synthetic oligonucleotide
actcggttta

SEQ ID NO: 161 - DNA, 10 bp, Artificial Sequence
  Description of Artificial Sequence: Synthetic oligonucleotide
ctcggtttat

SEQ ID NO: 162 - DNA, 10 bp, Artificial Sequence
  Description of Artificial Sequence: Synthetic oligonucleotide
tcggtttatt

SEQ ID NO: 163 - DNA, 10 bp, Artificial Sequence
  Description of Artificial Sequence: Synthetic oligonucleotide
cggtttattt

SEQ ID NO: 164 - DNA, 10 bp, Artificial Sequence
  Description of Artificial Sequence: Synthetic oligonucleotide
ggtttatttg

SEQ ID NO: 165 - DNA, 10 bp, Artificial Sequence
  Description of Artificial Sequence: Synthetic oligonucleotide
gtttatttgg

SEQ ID NO: 166 - DNA, 10 bp, Artificial Sequence
  Description of Artificial Sequence: Synthetic oligonucleotide
tttatttgga

SEQ ID NO: 167 - DNA, 10 bp, Artificial Sequence
  Description of Artificial Sequence: Synthetic oligonucleotide
ttatttggaa

SEQ ID NO: 168 - DNA, 10 bp, Artificial Sequence
  Description of Artificial Sequence: Synthetic oligonucleotide
tatttggaaa

SEQ ID NO: 169 - DNA, 10 bp, Artificial Sequence
  Description of Artificial Sequence: Synthetic oligonucleotide
atttggaaaa

SEQ ID NO: 170 - DNA, 10 bp, Artificial Sequence
  Description of Artificial Sequence: Synthetic oligonucleotide
tttggaaaaa

SEQ ID NO: 171 - DNA, 10 bp, Artificial Sequence
  Description of Artificial Sequence: Synthetic oligonucleotide
ttggaaaaag

SEQ ID NO: 172 - DNA, 15 bp, Artificial Sequence
  Description of Artificial Sequence: Synthetic oligonucleotide
tcttgacactcggtt

SEQ ID NO: 173 - DNA, 15 bp, Artificial Sequence
  Description of Artificial Sequence: Synthetic oligonucleotide
cttgacactcggttt

SEQ ID NO: 174 - DNA, 15 bp, Artificial Sequence
  Description of Artificial Sequence: Synthetic oligonucleotide
ttgacactcggttta

SEQ ID NO: 175 - DNA, 15 bp, Artificial Sequence
  Description of Artificial Sequence: Synthetic oligonucleotide
tgacactcggtttat

SEQ ID NO: 176 - DNA, 15 bp, Artificial Sequence
  Description of Artificial Sequence: Synthetic oligonucleotide
gacactcggtttatt

SEQ ID NO: 177 - DNA, 15 bp, Artificial Sequence
  Description of Artificial Sequence: Synthetic oligonucleotide
acactcggtttattt

SEQ ID NO: 178 - DNA, 15 bp, Artificial Sequence
  Description of Artificial Sequence: Synthetic oligonucleotide
cactcggtttatttg

SEQ ID NO: 179 - DNA, 15 bp, Artificial Sequence
  Description of Artificial Sequence: Synthetic oligonucleotide
actcggtttatttgg

SEQ ID NO: 180 - DNA, 15 bp, Artificial Sequence
  Description of Artificial Sequence: Synthetic oligonucleotide
ctcggtttatttgga

SEQ ID NO: 181 - DNA, 15 bp, Artificial Sequence
  Description of Artificial Sequence: Synthetic oligonucleotide
tcggtttatttggaa

SEQ ID NO: 182 - DNA, 15 bp, Artificial Sequence
  Description of Artificial Sequence: Synthetic oligonucleotide
cggtttatttggaaa

SEQ ID NO: 183 - DNA, 15 bp, Artificial Sequence
  Description of Artificial Sequence: Synthetic oligonucleotide
ggtttatttggaaaa

SEQ ID NO: 184 - DNA, 15 bp, Artificial Sequence
  Description of Artificial Sequence: Synthetic oligonucleotide
gtttatttggaaaaa

SEQ ID NO: 185 - DNA, 15 bp, Artificial Sequence
  Description of Artificial Sequence: Synthetic oligonucleotide
tttatttggaaaaag

SEQ ID NO: 186 - DNA, 15 bp, Artificial Sequence
  Description of Artificial Sequence: Synthetic oligonucleotide
ttatttggaaaaaga

SEQ ID NO: 187 - DNA, 15 bp, Artificial Sequence
  Description of Artificial Sequence: Synthetic oligonucleotide
tatttggaaaaagac

SEQ ID NO: 188 - DNA, 15 bp, Artificial Sequence
  Description of Artificial Sequence: Synthetic oligonucleotide
atttggaaaaagaca

SEQ ID NO: 189 - DNA, 15 bp, Artificial Sequence
  Description of Artificial Sequence: Synthetic oligonucleotide
tttggaaaaagacat

SEQ ID NO: 190 - DNA, 15 bp, Artificial Sequence
  Description of Artificial Sequence: Synthetic oligonucleotide
ttggaaaaagacata

SEQ ID NO: 191 - DNA, 15 bp, Artificial Sequence
  Description of Artificial Sequence: Synthetic oligonucleotide
tggaaaaagacatat

SEQ ID NO: 192 - DNA, 20 bp, Artificial Sequence
  Description of Artificial Sequence: Synthetic oligonucleotide
ggtagtcttgacactcggtt

SEQ ID NO: 193 - DNA, 20 bp, Artificial Sequence
  Description of Artificial Sequence: Synthetic oligonucleotide
gtagtcttgacactcggttt

SEQ ID NO: 194 - DNA, 20 bp, Artificial Sequence
  Description of Artificial Sequence: Synthetic oligonucleotide
tagtcttgacactcggttta

SEQ ID NO: 195 - DNA, 20 bp, Artificial Sequence
  Description of Artificial Sequence: Synthetic oligonucleotide
agtcttgacactcggtttat

SEQ ID NO: 196 - DNA, 20 bp, Artificial Sequence
  Description of Artificial Sequence: Synthetic oligonucleotide
gtcttgacactcggtttatt

SEQ ID NO: 197 - DNA, 20 bp, Artificial Sequence
  Description of Artificial Sequence: Synthetic oligonucleotide
tcttgacactcggtttattt

SEQ ID NO: 198 - DNA, 20 bp, Artificial Sequence
  Description of Artificial Sequence: Synthetic oligonucleotide
cttgacactcggtttatttg

SEQ ID NO: 199 - DNA, 20 bp, Artificial Sequence
  Description of Artificial Sequence: Synthetic oligonucleotide
ttgacactcggtttatttgg

SEQ ID NO: 200 - DNA, 20 bp, Artificial Sequence
  Description of Artificial Sequence: Synthetic oligonucleotide
tgacactcggtttatttgga

SEQ ID NO: 201 - DNA, 20 bp, Artificial Sequence
  Description of Artificial Sequence: Synthetic oligonucleotide
gacactcggtttatttggaa

SEQ ID NO: 202 - DNA, 20 bp, Artificial Sequence
  Description of Artificial Sequence: Synthetic oligonucleotide
acactcggtttatttggaaa

SEQ ID NO: 203 - DNA, 20 bp, Artificial Sequence
  Description of Artificial Sequence: Synthetic oligonucleotide
cactcggtttatttggaaaa

SEQ ID NO: 204 - DNA, 20 bp, Artificial Sequence
  Description of Artificial Sequence: Synthetic oligonucleotide
actcggtttatttggaaaaa

SEQ ID NO: 205 - DNA, 20 bp, Artificial Sequence
  Description of Artificial Sequence: Synthetic oligonucleotide
ctcggtttatttggaaaaag

SEQ ID NO: 206 - DNA, 20 bp, Artificial Sequence
  Description of Artificial Sequence: Synthetic oligonucleotide
tcggtttatttggaaaaaga

SEQ ID NO: 207 - DNA, 20 bp, Artificial Sequence
  Description of Artificial Sequence: Synthetic oligonucleotide
cggtttatttggaaaaagac

SEQ ID NO: 208 - DNA, 20 bp, Artificial Sequence
  Description of Artificial Sequence: Synthetic oligonucleotide
ggtttatttggaaaaagaca

SEQ ID NO: 209 - DNA, 20 bp, Artificial Sequence
  Description of Artificial Sequence: Synthetic oligonucleotide
gtttatttggaaaaagacat

SEQ ID NO: 210 - DNA, 20 bp, Artificial Sequence
  Description of Artificial Sequence: Synthetic oligonucleotide
tttatttggaaaaagacata

SEQ ID NO: 211 - DNA, 20 bp, Artificial Sequence
  Description of Artificial Sequence: Synthetic oligonucleotide
ttatttggaaaaagacatat

SEQ ID NO: 212 - DNA, 20 bp, Artificial Sequence
  Description of Artificial Sequence: Synthetic oligonucleotide
tatttggaaaaagacatatt

SEQ ID NO: 213 - DNA, 20 bp, Artificial Sequence
  Description of Artificial Sequence: Synthetic oligonucleotide
atttggaaaaagacatattt

SEQ ID NO: 214 - DNA, 20 bp, Artificial Sequence
  Description of Artificial Sequence: Synthetic oligonucleotide
tttggaaaaagacatattta

SEQ ID NO: 215 - DNA, 20 bp, Artificial Sequence
  Description of Artificial Sequence: Synthetic oligonucleotide
ttggaaaaagacatatttaa

SEQ ID NO: 216 - DNA, 25 bp, Artificial Sequence
  Description of Artificial Sequence: Synthetic oligonucleotide
cggtagtcttgacactcggtttatt

SEQ ID NO: 217 - DNA, 25 bp, Artificial Sequence
  Description of Artificial Sequence: Synthetic oligonucleotide
ggtagtcttgacactcggtttattt

SEQ ID NO: 218 - DNA, 25 bp, Artificial Sequence
  Description of Artificial Sequence: Synthetic oligonucleotide
gtagtcttgacactcggtttatttg

SEQ ID NO: 219 - DNA, 25 bp, Artificial Sequence
  Description of Artificial Sequence: Synthetic oligonucleotide
tagtcttgacactcggtttatttgg

SEQ ID NO: 220 - DNA, 25 bp, Artificial Sequence
  Description of Artificial Sequence: Synthetic oligonucleotide
agtcttgacactcggtttatttgga

SEQ ID NO: 221 - DNA, 25 bp, Artificial Sequence
  Description of Artificial Sequence: Synthetic oligonucleotide
gtcttgacactcggtttatttggaa

SEQ ID NO: 222 - DNA, 25 bp, Artificial Sequence
  Description of Artificial Sequence: Synthetic oligonucleotide
tcttgacactcggtttatttggaaa

SEQ ID NO: 223 - DNA, 25 bp, Artificial Sequence
  Description of Artificial Sequence: Synthetic oligonucleotide
cttgacactcggtttatttggaaaa

SEQ ID NO: 224 - DNA, 25 bp, Artificial Sequence
  Description of Artificial Sequence: Synthetic oligonucleotide
ttgacactcggtttatttggaaaaa

SEQ ID NO: 225 - DNA, 25 bp, Artificial Sequence
  Description of Artificial Sequence: Synthetic oligonucleotide
tgacactcggtttatttggaaaaag

SEQ ID NO: 226 - DNA, 25 bp, Artificial Sequence
  Description of Artificial Sequence: Synthetic oligonucleotide
gacactcggtttatttggaaaaaga

SEQ ID NO: 227 - DNA, 25 bp, Artificial Sequence
  Description of Artificial Sequence: Synthetic oligonucleotide
acactcggtttatttggaaaaagac

SEQ ID NO: 228 - DNA, 25 bp, Artificial Sequence
  Description of Artificial Sequence: Synthetic oligonucleotide
cactcggtttatttggaaaaagaca

SEQ ID NO: 229 - DNA, 25 bp, Artificial Sequence
  Description of Artificial Sequence: Synthetic oligonucleotide
actcggtttatttggaaaaagacat

SEQ ID NO: 230 - DNA, 25 bp, Artificial Sequence
  Description of Artificial Sequence: Synthetic oligonucleotide
ctcggtttatttggaaaaagacata

SEQ ID NO: 231 - DNA, 25 bp, Artificial Sequence
  Description of Artificial Sequence: Synthetic oligonucleotide
tcggtttatttggaaaaagacatat

SEQ ID NO: 232 - DNA, 25 bp, Artificial Sequence
  Description of Artificial Sequence: Synthetic oligonucleotide
cggtttatttggaaaaagacatatt

SEQ ID NO: 233 - DNA, 25 bp, Artificial Sequence
  Description of Artificial Sequence: Synthetic oligonucleotide
ggtttatttggaaaaagacatattt

SEQ ID NO: 234 - DNA, 25 bp, Artificial Sequence
  Description of Artificial Sequence: Synthetic oligonucleotide
gtttatttggaaaaagacatattta

SEQ ID NO: 235 - DNA, 25 bp, Artificial Sequence
  Description of Artificial Sequence: Synthetic oligonucleotide
tttatttggaaaaagacatatttaa

SEQ ID NO: 236 - DNA, 25 bp, Artificial Sequence
  Description of Artificial Sequence: Synthetic oligonucleotide
ttatttggaaaaagacatatttaat

SEQ ID NO: 237 - DNA, 30 bp, Artificial Sequence
  Description of Artificial Sequence: Synthetic oligonucleotide
cggtagtcttgacactcggtttatttggaa

SEQ ID NO: 238 - DNA, 30 bp, Artificial Sequence
  Description of Artificial Sequence: Synthetic oligonucleotide
ggtagtcttgacactcggtttatttggaaa

SEQ ID NO: 239 - DNA, 30 bp, Artificial Sequence
  Description of Artificial Sequence: Synthetic oligonucleotide
gtagtcttgacactcggtttatttggaaaa

SEQ ID NO: 240 - DNA, 30 bp, Artificial Sequence
  Description of Artificial Sequence: Synthetic oligonucleotide
tagtcttgacactcggtttatttggaaaaa

SEQ ID NO: 241 - DNA, 30 bp, Artificial Sequence
  Description of Artificial Sequence: Synthetic oligonucleotide
agtcttgacactcggtttatttggaaaaag

SEQ ID NO: 242 - DNA, 30 bp, Artificial Sequence
  Description of Artificial Sequence: Synthetic oligonucleotide
gtcttgacactcggtttatttggaaaaaga

SEQ ID NO: 243 - DNA, 30 bp, Artificial Sequence
  Description of Artificial Sequence: Synthetic oligonucleotide
tcttgacactcggtttatttggaaaaagac

SEQ ID NO: 244 - DNA, 30 bp, Artificial Sequence
  Description of Artificial Sequence: Synthetic oligonucleotide
cttgacactcggtttatttggaaaaagaca

SEQ ID NO: 245 - DNA, 30 bp, Artificial Sequence
  Description of Artificial Sequence: Synthetic oligonucleotide
ttgacactcggtttatttggaaaaagacat

SEQ ID NO: 246 - DNA, 30 bp, Artificial Sequence
  Description of Artificial Sequence: Synthetic oligonucleotide
tgacactcggtttatttggaaaaagacata

SEQ ID NO: 247 - DNA, 30 bp, Artificial Sequence
  Description of Artificial Sequence: Synthetic oligonucleotide
gacactcggtttatttggaaaaagacatat

SEQ ID NO: 248 - DNA, 30 bp, Artificial Sequence
  Description of Artificial Sequence: Synthetic oligonucleotide
acactcggtttatttggaaaaagacatatt

SEQ ID NO: 249 - DNA, 30 bp, Artificial Sequence
  Description of Artificial Sequence: Synthetic oligonucleotide
cactcggtttatttggaaaaagacatattt

SEQ ID NO: 250 - DNA, 30 bp, Artificial Sequence
  Description of Artificial Sequence: Synthetic oligonucleotide
actcggtttatttggaaaaagacatattta

SEQ ID NO: 251 - DNA, 30 bp, Artificial Sequence
  Description of Artificial Sequence: Synthetic oligonucleotide
ctcggtttatttggaaaaagacatatttaa

SEQ ID NO: 252 - DNA, 30 bp, Artificial Sequence
  Description of Artificial Sequence: Synthetic oligonucleotide
tcggtttatttggaaaaagacatatttaat

SEQ ID NO: 253 - DNA, 25 bp, Artificial Sequence
  Description of Artificial Sequence: Synthetic oligonucleotide
gctgagtctgtttttaacattcatt

SEQ ID NO: 254 - DNA, 25 bp, Artificial Sequence
  Description of Artificial Sequence: Synthetic oligonucleotide
tgagtctgtttttaacattcattaa

SEQ ID NO: 255 - DNA, 25 bp, Artificial Sequence
  Description of Artificial Sequence: Synthetic oligonucleotide
agtctgtttttaacattcattaatc

SEQ ID NO: 256 - DNA, 25 bp, Artificial Sequence
  Description of Artificial Sequence: Synthetic oligonucleotide
tctgtttttaacattcattaatcca

SEQ ID NO: 257 - DNA, 24 bp, Artificial Sequence
  Description of Artificial Sequence: Synthetic oligonucleotide
aatttatacagaaaaaggtttatt

SEQ ID NO: 258 - DNA, 25 bp, Artificial Sequence
  Description of Artificial Sequence: Synthetic oligonucleotide
atttatacagaaaaaggtttatttg

SEQ ID NO: 259 - DNA, 25 bp, Artificial Sequence
  Description of Artificial Sequence: Synthetic oligonucleotide
ttatacagaaaaaggtttatttggc

SEQ ID NO: 260 - DNA, 24 bp, Artificial Sequence
  Description of Artificial Sequence: Synthetic oligonucleotide
tacagaaaaaggtttatttggctc

SEQ ID NO: 261 - DNA, 10 bp, Artificial Sequence
  Description of Artificial Sequence: Synthetic oligonucleotide
tgtgagccaa

SEQ ID NO: 262 - DNA, 10 bp, Artificial Sequence
  Description of Artificial Sequence: Synthetic oligonucleotide
gtgagccaaa

SEQ ID NO: 263 - DNA, 10 bp, Artificial Sequence
  Description of Artificial Sequence: Synthetic oligonucleotide
tgagccaaat

SEQ ID NO: 264 - DNA, 10 bp, Artificial Sequence
  Description of Artificial Sequence: Synthetic oligonucleotide
gagccaaata

SEQ ID NO: 265 - DNA, 10 bp, Artificial Sequence
  Description of Artificial Sequence: Synthetic oligonucleotide
agccaaataa

SEQ ID NO: 266 - DNA, 10 bp, Artificial Sequence
  Description of Artificial Sequence: Synthetic oligonucleotide
gccaaataaa

SEQ ID NO: 267 - DNA, 10 bp, Artificial Sequence
  Description of Artificial Sequence: Synthetic oligonucleotide
ccaaataaac

SEQ ID NO: 268 - DNA, 10 bp, Artificial Sequence
  Description of Artificial Sequence: Synthetic oligonucleotide
caaataaacc

SEQ ID NO: 269 - DNA, 10 bp, Artificial Sequence
  Description of Artificial Sequence: Synthetic oligonucleotide
aaataaacct

SEQ ID NO: 270 - DNA, 10 bp, Artificial Sequence
  Description of Artificial Sequence: Synthetic oligonucleotide
aataaacctt

SEQ ID NO: 271 - DNA, 10 bp, Artificial Sequence
  Description of Artificial Sequence: Synthetic oligonucleotide
ataaaccttt

SEQ ID NO: 272 - DNA, 10 bp, Artificial Sequence
  Description of Artificial Sequence: Synthetic oligonucleotide
taaacctttt

SEQ ID NO: 273 - DNA, 10 bp, Artificial Sequence
  Description of Artificial Sequence: Synthetic oligonucleotide
aaaccttttt

SEQ ID NO: 274 - DNA, 10 bp, Artificial Sequence
  Description of Artificial Sequence: Synthetic oligonucleotide
aacctttttc

SEQ ID NO: 275 - DNA, 15 bp, Artificial Sequence
  Description of Artificial Sequence: Synthetic oligonucleotide
agaactgtgagccaa

SEQ ID NO: 276 - DNA, 15 bp, Artificial Sequence
  Description of Artificial Sequence: Synthetic oligonucleotide
gaactgtgagccaaa

SEQ ID NO: 277 - DNA, 15 bp, Artificial Sequence
  Description of Artificial Sequence: Synthetic oligonucleotide
aactgtgagccaaat

SEQ ID NO: 278 - DNA, 15 bp, Artificial Sequence
  Description of Artificial Sequence: Synthetic oligonucleotide
actgtgagccaaata

SEQ ID NO: 279 - DNA, 15 bp, Artificial Sequence
  Description of Artificial Sequence: Synthetic oligonucleotide
ctgtgagccaaataa

SEQ ID NO: 280 - DNA, 15 bp, Artificial Sequence
  Description of Artificial Sequence: Synthetic oligonucleotide
tgtgagccaaataaa

SEQ ID NO: 281 - DNA, 15 bp, Artificial Sequence
  Description of Artificial Sequence: Synthetic oligonucleotide
gtgagccaaataaac

SEQ ID NO: 282 - DNA, 15 bp, Artificial Sequence
  Description of Artificial Sequence: Synthetic oligonucleotide
tgagccaaataaacc

SEQ ID NO: 283 - DNA, 15 bp, Artificial Sequence
  Description of Artificial Sequence: Synthetic oligonucleotide
gagccaaataaacct

SEQ ID NO: 284 - DNA, 15 bp, Artificial Sequence
  Description of Artificial Sequence: Synthetic oligonucleotide
agccaaataaacctt

SEQ ID NO: 285 - DNA, 15 bp, Artificial Sequence
  Description of Artificial Sequence: Synthetic oligonucleotide
gccaaataaaccttt

SEQ ID NO: 286 - DNA, 15 bp, Artificial Sequence
  Description of Artificial Sequence: Synthetic oligonucleotide
ccaaataaacctttt

SEQ ID NO: 287 - DNA, 15 bp, Artificial Sequence
  Description of Artificial Sequence: Synthetic oligonucleotide
caaataaaccttttt

SEQ ID NO: 288 - DNA, 15 bp, Artificial Sequence
  Description of Artificial Sequence: Synthetic oligonucleotide
aaataaacctttttc

SEQ ID NO: 289 - DNA, 15 bp, Artificial Sequence
  Description of Artificial Sequence: Synthetic oligonucleotide
aataaacctttttct

SEQ ID NO: 290 - DNA, 15 bp, Artificial Sequence
  Description of Artificial Sequence: Synthetic oligonucleotide
ataaacctttttctg

SEQ ID NO: 291 - DNA, 15 bp, Artificial Sequence
  Description of Artificial Sequence: Synthetic oligonucleotide
taaacctttttctgt

SEQ ID NO: 292 - DNA, 15 bp, Artificial Sequence
  Description of Artificial Sequence: Synthetic oligonucleotide
aaacctttttctgta

SEQ ID NO: 293 - DNA, 15 bp, Artificial Sequence
  Description of Artificial Sequence: Synthetic oligonucleotide
aacctttttctgtat

SEQ ID NO: 294 - DNA, 15 bp, Artificial Sequence
  Description of Artificial Sequence: Synthetic oligonucleotide
acctttttctgtata

SEQ ID NO: 295 - DNA, 20 bp, Artificial Sequence
  Description of Artificial Sequence: Synthetic oligonucleotide
ccatcagaactgtgagccaa

SEQ ID NO: 296 - DNA, 20 bp, Artificial Sequence
  Description of Artificial Sequence: Synthetic oligonucleotide
catcagaactgtgagccaaa

SEQ ID NO: 297 - DNA, 20 bp, Artificial Sequence
  Description of Artificial Sequence: Synthetic oligonucleotide
atcagaactgtgagccaaat

SEQ ID NO: 298 - DNA, 20 bp, Artificial Sequence
  Description of Artificial Sequence: Synthetic oligonucleotide
tcagaactgtgagccaaata

SEQ ID NO: 299 - DNA, 20 bp, Artificial Sequence
  Description of Artificial Sequence: Synthetic oligonucleotide
cagaactgtgagccaaataa

SEQ ID NO: 300 - DNA, 20 bp, Artificial Sequence
  Description of Artificial Sequence: Synthetic oligonucleotide
agaactgtgagccaaataaa

SEQ ID NO: 301 - DNA, 20 bp, Artificial Sequence
  Description of Artificial Sequence: Synthetic oligonucleotide
gaactgtgagccaaataaac

SEQ ID NO: 302 - DNA, 20 bp, Artificial Sequence
  Description of Artificial Sequence: Synthetic oligonucleotide
aactgtgagccaaataaacc

SEQ ID NO: 303 - DNA, 20 bp, Artificial Sequence
  Description of Artificial Sequence: Synthetic oligonucleotide
actgtgagccaaataaacct

SEQ ID NO: 304 - DNA, 20 bp, Artificial Sequence
  Description of Artificial Sequence: Synthetic oligonucleotide
ctgtgagccaaataaacctt

SEQ ID NO: 305 - DNA, 20 bp, Artificial Sequence
  Description of Artificial Sequence: Synthetic oligonucleotide
tgtgagccaaataaaccttt

SEQ ID NO: 306 - DNA, 20 bp, Artificial Sequence
  Description of Artificial Sequence: Synthetic oligonucleotide
gtgagccaaataaacctttt

SEQ ID NO: 307 - DNA, 20 bp, Artificial Sequence
  Description of Artificial Sequence: Synthetic oligonucleotide
tgagccaaataaaccttttt

SEQ ID NO: 308 - DNA, 20 bp, Artificial Sequence
  Description of Artificial Sequence: Synthetic oligonucleotide
gagccaaataaacctttttc

SEQ ID NO: 309 - DNA, 20 bp, Artificial Sequence
  Description of Artificial Sequence: Synthetic oligonucleotide
agccaaataaacctttttct

SEQ ID NO: 310 - DNA, 20 bp, Artificial Sequence
  Description of Artificial Sequence: Synthetic oligonucleotide
gccaaataaacctttttctg

SEQ ID NO: 311 - DNA, 20 bp, Artificial Sequence
  Description of Artificial Sequence: Synthetic oligonucleotide
ccaaataaacctttttctgt

SEQ ID NO: 312 - DNA, 20 bp, Artificial Sequence
  Description of Artificial Sequence: Synthetic oligonucleotide
caaataaacctttttctgta

SEQ ID NO: 313 - DNA, 20 bp, Artificial Sequence
  Description of Artificial Sequence: Synthetic oligonucleotide
aaataaacctttttctgtat

SEQ ID NO: 314 - DNA, 20 bp, Artificial Sequence
  Description of Artificial Sequence: Synthetic oligonucleotide
aataaacctttttctgtata

SEQ ID NO: 315 - DNA, 20 bp, Artificial Sequence
  Description of Artificial Sequence: Synthetic oligonucleotide
ataaacctttttctgtataa

SEQ ID NO: 316 - DNA, 20 bp, Artificial Sequence
  Description of Artificial Sequence: Synthetic oligonucleotide
taaacctttttctgtataaa

SEQ ID NO: 317 - DNA, 20 bp, Artificial Sequence
  Description of Artificial Sequence: Synthetic oligonucleotide
aaacctttttctgtataaat

SEQ ID NO: 318 - DNA, 20 bp, Artificial Sequence
  Description of Artificial Sequence: Synthetic oligonucleotide
aacctttttctgtataaatt

SEQ ID NO: 319 - DNA, 25 bp, Artificial Sequence
  Description of Artificial Sequence: Synthetic oligonucleotide
gccatcagaactgtgagccaaataa

SEQ ID NO: 320 - DNA, 25 bp, Artificial Sequence
  Description of Artificial Sequence: Synthetic oligonucleotide
ccatcagaactgtgagccaaataaa

SEQ ID NO: 321 - DNA, 25 bp, Artificial Sequence
  Description of Artificial Sequence: Synthetic oligonucleotide
catcagaactgtgagccaaataaac

SEQ ID NO: 322 - DNA, 25 bp, Artificial Sequence
  Description of Artificial Sequence: Synthetic oligonucleotide
atcagaactgtgagccaaataaacc

SEQ ID NO: 323 - DNA, 25 bp, Artificial Sequence
  Description of Artificial Sequence: Synthetic oligonucleotide
tcagaactgtgagccaaataaacct

SEQ ID NO: 324 - DNA, 25 bp, Artificial Sequence
  Description of Artificial Sequence: Synthetic oligonucleotide
cagaactgtgagccaaataaacctt

SEQ ID NO: 325 - DNA, 25 bp, Artificial Sequence
  Description of Artificial Sequence: Synthetic oligonucleotide
agaactgtgagccaaataaaccttt

SEQ ID NO: 326 - DNA, 25 bp, Artificial Sequence
  Description of Artificial Sequence: Synthetic oligonucleotide
gaactgtgagccaaataaacctttt

SEQ ID NO: 327 - DNA, 25 bp, Artificial Sequence
  Description of Artificial Sequence: Synthetic oligonucleotide
aactgtgagccaaataaaccttttt

SEQ ID NO: 328 - DNA, 25 bp, Artificial Sequence
  Description of Artificial Sequence: Synthetic oligonucleotide
actgtgagccaaataaacctttttc

SEQ ID NO: 329 - DNA, 25 bp, Artificial Sequence
  Description of Artificial Sequence: Synthetic oligonucleotide
ctgtgagccaaataaacctttttct

SEQ ID NO: 330 - DNA, 25 bp, Artificial Sequence
  Description of Artificial Sequence: Synthetic oligonucleotide
tgtgagccaaataaacctttttctg

SEQ ID NO: 331 - DNA, 25 bp, Artificial Sequence
  Description of Artificial Sequence: Synthetic oligonucleotide
gtgagccaaataaacctttttctgt

SEQ ID NO: 332 - DNA, 25 bp, Artificial Sequence
  Description of Artificial Sequence: Synthetic oligonucleotide
tgagccaaataaacctttttctgta

SEQ ID NO: 333 - DNA, 25 bp, Artificial Sequence
  Description of Artificial Sequence: Synthetic oligonucleotide
gagccaaataaacctttttctgtat

SEQ ID NO: 334 - DNA, 25 bp, Artificial Sequence
  Description of Artificial Sequence: Synthetic oligonucleotide
agccaaataaacctttttctgtata

SEQ ID NO: 335 - DNA, 25 bp, Artificial Sequence
  Description of Artificial Sequence: Synthetic oligonucleotide
gccaaataaacctttttctgtataa

SEQ ID NO: 336 - DNA, 25 bp, Artificial Sequence
  Description of Artificial Sequence: Synthetic oligonucleotide
ccaaataaacctttttctgtataaa

SEQ ID NO: 337 - DNA, 25 bp, Artificial Sequence
  Description of Artificial Sequence: Synthetic oligonucleotide
caaataaacctttttctgtataaat

SEQ ID NO: 338 - DNA, 25 bp, Artificial Sequence
  Description of Artificial Sequence: Synthetic oligonucleotide
aaataaacctttttctgtataaatt

SEQ ID NO: 339 - DNA, 25 bp, Artificial Sequence
  Description of Artificial Sequence: Synthetic oligonucleotide
aataaacctttttctgtataaatta

SEQ ID NO: 340 - DNA, 30 bp, Artificial Sequence
  Description of Artificial Sequence: Synthetic oligonucleotide
gccatcagaactgtgagccaaataaacctt

SEQ ID NO: 341 - DNA, 30 bp, Artificial Sequence
  Description of Artificial Sequence: Synthetic oligonucleotide
ccatcagaactgtgagccaaataaaccttt

SEQ ID NO: 342 - DNA, 30 bp, Artificial Sequence
  Description of Artificial Sequence: Synthetic oligonucleotide
catcagaactgtgagccaaataaacctttt

SEQ ID NO: 343 - DNA, 30 bp, Artificial Sequence
  Description of Artificial Sequence: Synthetic oligonucleotide
atcagaactgtgagccaaataaaccttttt

SEQ ID NO: 344 - DNA, 30 bp, Artificial Sequence
  Description of Artificial Sequence: Synthetic oligonucleotide
tcagaactgtgagccaaataaacctttttc

SEQ ID NO: 345 - DNA, 30 bp, Artificial Sequence
  Description of Artificial Sequence: Synthetic oligonucleotide
cagaactgtgagccaaataaacctttttct

SEQ ID NO: 346 - DNA, 30 bp, Artificial Sequence
  Description of Artificial Sequence: Synthetic oligonucleotide
agaactgtgagccaaataaacctttttctg

SEQ ID NO: 347 - DNA, 30 bp, Artificial Sequence
  Description of Artificial Sequence: Synthetic oligonucleotide
gaactgtgagccaaataaacctttttctgt

SEQ ID NO: 348 - DNA, 30 bp, Artificial Sequence
  Description of Artificial Sequence: Synthetic oligonucleotide
aactgtgagccaaataaacctttttctgta

SEQ ID NO: 349 - DNA, 30 bp, Artificial Sequence
  Description of Artificial Sequence: Synthetic oligonucleotide
actgtgagccaaataaacctttttctgtat

SEQ ID NO: 350 - DNA, 30 bp, Artificial Sequence
  Description of Artificial Sequence: Synthetic oligonucleotide
ctgtgagccaaataaacctttttctgtata

SEQ ID NO: 351 - DNA, 30 bp, Artificial Sequence
  Description of Artificial Sequence: Synthetic oligonucleotide
tgtgagccaaataaacctttttctgtataa

SEQ ID NO: 352 - DNA, 30 bp, Artificial Sequence
  Description of Artificial Sequence: Synthetic oligonucleotide
gtgagccaaataaacctttttctgtataaa

SEQ ID NO: 353 - DNA, 30 bp, Artificial Sequence
  Description of Artificial Sequence: Synthetic oligonucleotide
tgagccaaataaacctttttctgtataaat

SEQ ID NO: 354 - DNA, 30 bp, Artificial Sequence
  Description of Artificial Sequence: Synthetic oligonucleotide
gagccaaataaacctttttctgtataaatt

SEQ ID NO: 355 - DNA, 30 bp, Artificial Sequence
  Description of Artificial Sequence: Synthetic oligonucleotide
agccaaataaacctttttctgtataaatta

SEQ ID NO: 356 - DNA, 25 bp, Artificial Sequence
  Description of Artificial Sequence: Synthetic oligonucleotide
aatgaatgttaaaaacagactcagc

SEQ ID NO: 357 - DNA, 25 bp, Artificial Sequence
  Description of Artificial Sequence: Synthetic oligonucleotide
ttaatgaatgttaaaaacagactca

SEQ ID NO: 358 - DNA, 25 bp, Artificial Sequence
  Description of Artificial Sequence: Synthetic oligonucleotide
gattaatgaatgttaaaaacagact

SEQ ID NO: 359 - DNA, 25 bp, Artificial Sequence
  Description of Artificial Sequence: Synthetic oligonucleotide
tggattaatgaatgttaaaaacaga

SEQ ID NO: 360 - DNA, 25 bp, Artificial Sequence
  Description of Artificial Sequence: Synthetic oligonucleotide
aataaacctttttctgtataaatta

SEQ ID NO: 361 - DNA, 25 bp, Artificial Sequence
  Description of Artificial Sequence: Synthetic oligonucleotide
caaataaacctttttctgtataaat

SEQ ID NO: 362 - DNA, 25 bp, Artificial Sequence
  Description of Artificial Sequence: Synthetic oligonucleotide
gccaaataaacctttttctgtataa

SEQ ID NO: 363 - DNA, 24 bp, Artificial Sequence
  Description of Artificial Sequence: Synthetic oligonucleotide
gagccaaataaacctttttctgta

SEQ ID NO: 364 - DNA, 1710 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 365 - DNA, 1574 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 366 - DNA, 768 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 367 - DNA, 25 bp, Artificial Sequence
  Description of Artificial Sequence: Synthetic oligonucleotide
gggcattttaatatatctctgaact

SEQ ID NO: 368 - DNA, 29 bp, Artificial Sequence
  Description of Artificial Sequence: Synthetic oligonucleotide
ctataggatccacagggcattttaatatc

SEQ ID NO: 369 - DNA, 28 bp, Artificial Sequence
  Description of Artificial Sequence: Synthetic oligonucleotide
ggatccacagggaggaggcattttaata

SEQ ID NO: 370 - DNA, 30 bp, Artificial Sequence
  Description of Artificial Sequence: Synthetic oligonucleotide
tataggatccacagggaggaggcattttaa

SEQ ID NO: 371 - DNA, 25 bp, Artificial Sequence
  Description of Artificial Sequence: Synthetic oligonucleotide
catcacacaaaagatgcaaatcttc

SEQ ID NO: 372 - DNA, 30 bp, Artificial Sequence; identical to SEQ ID NO: 370
  Description of Artificial Sequence: Synthetic oligonucleotide

SEQ ID NO: 373 - DNA, 30 bp, Artificial Sequence
  Description of Artificial Sequence: Synthetic oligonucleotide
aggatccacagggagggggcattttaatat

SEQ ID NO: 374 - DNA, 30 bp, Artificial Sequence
  Description of Artificial Sequence: Synthetic oligonucleotide
taggatccacagggagggggcattttaata

SEQ ID NO: 375 - DNA, 30 bp, Artificial Sequence
  Description of Artificial Sequence: Synthetic oligonucleotide
aggatccacagggaggaggcattttaatat

SEQ ID NO: 376 - DNA, 30 bp, Artificial Sequence
  Description of Artificial Sequence: Synthetic oligonucleotide
taggatccacagggaggaggcattttaata

SEQ ID NO: 377 - DNA, 28 bp, Artificial Sequence
  Description of Artificial Sequence: Synthetic oligonucleotide
aggatccacagggaggaggcattttaat

SEQ ID NO: 378 - DNA, 25 bp, Artificial Sequence
  Description of Artificial Sequence: Synthetic oligonucleotide
gggcattttaatatatctctgaact

SEQ ID NO: 379 - DNA, 30 bp, Artificial Sequence; identical to SEQ ID NO: 370
  Description of Artificial Sequence: Synthetic oligonucleotide

SEQ ID NO: 380 - DNA, 30 bp, Artificial Sequence; identical to SEQ ID NO: 376
  Description of Artificial Sequence: Synthetic oligonucleotide

SEQ ID NO: 381 - DNA, 25 bp, Homo sapiens
agttcagagatatattaaaatgccc

SEQ ID NO: 382 - DNA, 29 bp, Homo sapiens
gatattaaaatgccctgtggatcctatag

SEQ ID NO: 383 - DNA, 28 bp, Homo sapiens
tattaaaatgcctcctccctgtggatcc

SEQ ID NO: 384 - DNA, 30 bp, Homo sapiens
ttaaaatgcctcctccctgtggatcctata

SEQ ID NO: 385 - DNA, 25 bp, Homo sapiens
gaagatttgcatcttttgtgtgatg

SEQ ID NO: 386 - DNA, 30 bp, Homo sapiens
ttaaaatgccccctccctgtggatcctata

SEQ ID NO: 387 - DNA, 30 bp, Homo sapiens
atattaaaatgccccctccctgtggatcct

SEQ ID NO: 388 - DNA, 30 bp, Homo sapiens
tattaaaatgccccctccctgtggatccta

SEQ ID NO: 389 - DNA, 30 bp, Homo sapiens; identical to SEQ ID NO: 387

SEQ ID NO: 390 - DNA, 30 bp, Homo sapiens; identical to SEQ ID NO: 388

SEQ ID NO: 391 - DNA, 28 bp, Homo sapiens
attaaaatgccccctccctgtggatcct

SEQ ID NO: 392 - DNA, 25 bp, Homo sapiens
agttcagagatatattaaaatgccc

SEQ ID NO: 393 - DNA, 29 bp, Homo sapiens
taaaatgcctcctccctgtggatcctata

SEQ ID NO: 394 - DNA, 30 bp, Homo sapiens
tattaaaatgcctcctccctgtggatccta

SEQ ID NO: 395 - DNA, 15 bp, Artificial Sequence
  Description of Artificial Sequence: Synthetic oligonucleotide
agcgtcggaaggtgg

SEQ ID NO: 396 - DNA, 16 bp, Artificial Sequence
  Description of Artificial Sequence: Synthetic oligonucleotide
ataggatccacaggga

SEQ ID NO: 397 - DNA, 14 bp, Artificial Sequence
  Description of Artificial Sequence: Synthetic oligonucleotide
agatcccctctgcc

SEQ ID NO: 398 - DNA, 15 bp, Artificial Sequence
  Description of Artificial Sequence: Synthetic oligonucleotide
cagcgtcggaaggtg

SEQ ID NO: 399 - DNA, 16 bp, Artificial Sequence
  Description of Artificial Sequence: Synthetic oligonucleotide
acagcgtcggaaggtc

SEQ ID NO: 400 - DNA, 16 bp, Artificial Sequence
  Description of Artificial Sequence: Synthetic oligonucleotide
gacagcgtcggaaggt

SEQ ID NO: 401 - DNA, 16 bp, Artificial Sequence
  Description of Artificial Sequence: Synthetic oligonucleotide
agacagcgtcggaagg

SEQ ID NO: 402 - DNA, 12 bp, Artificial Sequence
  Description of Artificial Sequence: Synthetic oligonucleotide
cgtcgcaaggtg

SEQ ID NO: 403 - DNA, 14 bp, Artificial Sequence
  Description of Artificial Sequence: Synthetic oligonucleotide
agcgtcggaaggtg

SEQ ID NO: 404 - DNA, 16 bp, Artificial Sequence
  Description of Artificial Sequence: Synthetic oligonucleotide
acagcgtcggaaggtg

SEQ ID NO: 405 - DNA, 20 bp, Artificial Sequence
  Description of Artificial Sequence: Synthetic oligonucleotide
tagacagcgtcggaaggtgg

SEQ ID NO: 406 - DNA, 20 bp, Artificial Sequence
  Description of Artificial Sequence: Synthetic oligonucleotide
ctagacagcgtcggaaggtg

SEQ ID NO: 407 - DNA, 20 bp, Artificial Sequence
  Description of Artificial Sequence: Synthetic oligonucleotide
cctagacagcgtcggaaggt

SEQ ID NO: 408 - DNA, 25 bp, Artificial Sequence
  Description of Artificial Sequence: Synthetic oligonucleotide
gtcctaggtggggacagacaataaa

SEQ ID NO: 409 - DNA, 25 bp, Artificial Sequence
  Description of Artificial Sequence: Synthetic oligonucleotide
gggtcctaggtggggacagacaata

SEQ ID NO: 410 - DNA, 25 bp, Artificial Sequence
  Description of Artificial Sequence: Synthetic oligonucleotide
gggggtcctaggtggggacagacaa

SEQ ID NO: 411 - DNA, 25 bp, Artificial Sequence
  Description of Artificial Sequence: Synthetic oligonucleotide
gtgggggtcctaggtggggacagac

SEQ ID NO: 412 - DNA, 25 bp, Artificial Sequence
  Description of Artificial Sequence: Synthetic oligonucleotide
gggtgggggtcctaggtggggacag

SEQ ID NO: 413 - DNA, 25 bp, Artificial Sequence
  Description of Artificial Sequence: Synthetic oligonucleotide
gggggtgggggtcctaggtggggac

SEQ ID NO: 414 - DNA, 25 bp, Artificial Sequence
  Description of Artificial Sequence: Synthetic oligonucleotide
tcgggggtgggggtcctaggtgggg

SEQ ID NO: 415 - DNA, 25 bp, Artificial Sequence
  Description of Artificial Sequence: Synthetic oligonucleotide
ggtcgggggtgggggtcctaggtgg

SEQ ID NO: 416 - DNA, 25 bp, Artificial Sequence
  Description of Artificial Sequence: Synthetic oligonucleotide
agggtcgggggtgggggtcctaggt

SEQ ID NO: 417 - DNA, 25 bp, Artificial Sequence
  Description of Artificial Sequence: Synthetic oligonucleotide
cgagggtcgggggtgggggtcctag

SEQ ID NO: 418 - DNA, 25 bp, Artificial Sequence
  Description of Artificial Sequence: Synthetic oligonucleotide
cgcgagggtcgggggtgggggtcct

SEQ ID NO: 419 - DNA, 25 bp, Artificial Sequence
  Description of Artificial Sequence: Synthetic oligonucleotide
ttcgcgagggtcgggggtgggggtc

SEQ ID NO: 420 - DNA, 25 bp, Artificial Sequence
  Description of Artificial Sequence: Synthetic oligonucleotide
tattcgcgagggtcgggggtggggg

SEQ ID NO: 421 - DNA, 25 bp, Artificial Sequence
  Description of Artificial Sequence: Synthetic oligonucleotide
tttattcgcgagggtcgggggtggg

SEQ ID NO: 422 - DNA, 25 bp, Artificial Sequence
  Description of Artificial Sequence: Synthetic oligonucleotide
cttttattcgcgagggtcgggggtg

SEQ ID NO: 423 - DNA, 25 bp, Artificial Sequence
  Description of Artificial Sequence: Synthetic oligonucleotide
gccttttattcgcgagggtcggggg

SEQ ID NO: 424 - DNA, 25 bp, Artificial Sequence
  Description of Artificial Sequence: Synthetic oligonucleotide
gggccttttattcgcgagggtcggg

SEQ ID NO: 425 - DNA, 25 bp, Artificial Sequence
  Description of Artificial Sequence: Synthetic oligonucleotide
gagggccttttattcgcgagggtcg

SEQ ID NO: 426 - DNA, 25 bp, Artificial Sequence
  Description of Artificial Sequence: Synthetic oligonucleotide
tggagggccttttattcgcgagggt

SEQ ID NO: 427 - DNA, 25 bp, Artificial Sequence
  Description of Artificial Sequence: Synthetic oligonucleotide
gatggagggccttttattcgcgagg

SEQ ID NO: 428 - DNA, 25 bp, Artificial Sequence
  Description of Artificial Sequence: Synthetic oligonucleotide
cagatggagggccttttattcgcga

SEQ ID NO: 429 - DNA, 25 bp, Artificial Sequence
  Description of Artificial Sequence: Synthetic oligonucleotide
ggcagatggagggccttttattcgc

SEQ ID NO: 430 - DNA, 25 bp, Artificial Sequence
  Description of Artificial Sequence: Synthetic oligonucleotide
tgggcagatggagggccttttattc

SEQ ID NO: 431 - DNA, 25 bp, Artificial Sequence
  Description of Artificial Sequence: Synthetic oligonucleotide
tttgggcagatggagggccttttat

SEQ ID NO: 432 - DNA, 25 bp, Artificial Sequence
  Description of Artificial Sequence: Synthetic oligonucleotide
gctttgggcagatggagggcctttt

SEQ ID NO: 433 - DNA, 25 bp, Artificial Sequence
  Description of Artificial Sequence: Synthetic oligonucleotide
gagctttgggcagatggagggcctt

SEQ ID NO: 434 - DNA, 25 bp, Artificial Sequence
  Description of Artificial Sequence: Synthetic oligonucleotide
cagagctttgggcagatggagggcc

SEQ ID NO: 435 - DNA, 25 bp, Artificial Sequence
  Description of Artificial Sequence: Synthetic oligonucleotide
tccagagctttgggcagatggaggg

SEQ ID NO: 436 - DNA, 25 bp, Artificial Sequence
  Description of Artificial Sequence: Synthetic oligonucleotide
agtccagagctttgggcagatggag

SEQ ID NO: 437 - DNA, 25 bp, Artificial Sequence
  Description of Artificial Sequence: Synthetic oligonucleotide
ggagtccagagctttgggcagatgg

SEQ ID NO: 438 - DNA, 25 bp, Artificial Sequence
  Description of Artificial Sequence: Synthetic oligonucleotide
gtggagtccagagctttgggcagat

SEQ ID NO: 439 - DNA, 25 bp, Artificial Sequence
  Description of Artificial Sequence: Synthetic oligonucleotide
ctgtggagtccagagctttgggcag

SEQ ID NO: 440 - DNA, 25 bp, Artificial Sequence
  Description of Artificial Sequence: Synthetic oligonucleotide
cactgtggagtccagagctttgggc

SEQ ID NO: 441 - DNA, 25 bp, Artificial Sequence
  Description of Artificial Sequence: Synthetic oligonucleotide
gacactgtggagtccagagctttgg

SEQ ID NO: 442 - DNA, 25 bp, Artificial Sequence
  Description of Artificial Sequence: Synthetic oligonucleotide
cggacactgtggagtccagagcttt

SEQ ID NO: 443 - DNA, 25 bp, Artificial Sequence
  Description of Artificial Sequence: Synthetic oligonucleotide
cgcggacactgtggagtccagagct

SEQ ID NO: 444 - DNA, 25 bp, Artificial Sequence
  Description of Artificial Sequence: Synthetic oligonucleotide
accgcggacactgtggagtccagag

SEQ ID NO: 445 - DNA, 25 bp, Artificial Sequence
  Description of Artificial Sequence: Synthetic oligonucleotide
aaaccgcggacactgtggagtccag

SEQ ID NO: 446 - DNA, 25 bp, Artificial Sequence
  Description of Artificial Sequence: Synthetic oligonucleotide
gcaaaccgcggacactgtggagtcc

SEQ ID NO: 447 - DNA, 25 bp, Artificial Sequence
  Description of Artificial Sequence: Synthetic oligonucleotide
acgcaaaccgcggacactgtggagt

SEQ ID NO: 448 - DNA, 25 bp, Artificial Sequence
  Description of Artificial Sequence: Synthetic oligonucleotide
caacgcaaaccgcggacactgtgga

SEQ ID NO: 449 - DNA, 25 bp, Homo sapiens
tttattgtctgtccccacctaggac

SEQ ID NO: 450 - DNA, 25 bp, Homo sapiens
tattgtctgtccccacctaggaccc

SEQ ID NO: 451 - DNA, 25 bp, Homo sapiens
ttgtctgtccccacctaggaccccc

SEQ ID NO: 452 - DNA, 25 bp, Homo sapiens
gtctgtccccacctaggacccccac

SEQ ID NO: 453 - DNA, 25 bp, Homo sapiens
ctgtccccacctaggacccccaccc

SEQ ID NO: 454 - DNA, 25 bp, Homo sapiens
gtccccacctaggacccccaccccc

SEQ ID NO: 455 - DNA, 25 bp, Homo sapiens
ccccacctaggacccccacccccga

SEQ ID NO: 456 - DNA, 25 bp, Homo sapiens
ccacctaggacccccacccccgacc

SEQ ID NO: 457 - DNA, 25 bp, Homo sapiens
acctaggacccccacccccgaccct

SEQ ID NO: 458 - DNA, 25 bp, Homo sapiens
ctaggacccccacccccgaccctcg

SEQ ID NO: 459 - DNA, 25 bp, Homo sapiens
aggacccccacccccgaccctcgcg

SEQ ID NO: 460 - DNA, 25 bp, Homo sapiens
gacccccacccccgaccctcgcgaa

SEQ ID NO: 461 - DNA, 25 bp, Homo sapiens
cccccacccccgaccctcgcgaata

SEQ ID NO: 462 - DNA, 25 bp, Homo sapiens
cccacccccgaccctcgcgaataaa

SEQ ID NO: 463 - DNA, 25 bp, Homo sapiens
cacccccgaccctcgcgaataaaag

SEQ ID NO: 464 - DNA, 25 bp, Homo sapiens
cccccgaccctcgcgaataaaaggc

SEQ ID NO: 465 - DNA, 25 bp, Homo sapiens
cccgaccctcgcgaataaaaggccc

SEQ ID NO: 466 - DNA, 25 bp, Homo sapiens
cgaccctcgcgaataaaaggccctc

SEQ ID NO: 467 - DNA, 25 bp, Homo sapiens
accctcgcgaataaaaggccctcca

SEQ ID NO: 468 - DNA, 25 bp, Homo sapiens
cctcgcgaataaaaggccctccatc

SEQ ID NO: 469 - DNA, 25 bp, Homo sapiens
tcgcgaataaaaggccctccatctg

SEQ ID NO: 470 - DNA, 25 bp, Homo sapiens
gcgaataaaaggccctccatctgcc

SEQ ID NO: 471 - DNA, 25 bp, Homo sapiens
gaataaaaggccctccatctgccca

SEQ ID NO: 472 - DNA, 25 bp, Homo sapiens
ataaaaggccctccatctgcccaaa

SEQ ID NO: 473 - DNA, 25 bp, Homo sapiens
aaaaggccctccatctgcccaaagc

SEQ ID NO: 474 - DNA, 25 bp, Homo sapiens
aaggccctccatctgcccaaagctc

SEQ ID NO: 475 - DNA, 25 bp, Homo sapiens
ggccctccatctgcccaaagctctg

SEQ ID NO: 476 - DNA, 25 bp, Homo sapiens
ccctccatctgcccaaagctctgga

SEQ ID NO: 477 - DNA, 25 bp, Homo sapiens
ctccatctgcccaaagctctggact

SEQ ID NO: 478 - DNA, 25 bp, Homo sapiens
ccatctgcccaaagctctggactcc

SEQ ID NO: 479 - DNA, 25 bp, Homo sapiens
atctgcccaaagctctggactccac

SEQ ID NO: 480 - DNA, 25 bp, Homo sapiens
ctgcccaaagctctggactccacag

SEQ ID NO: 481 - DNA, 25 bp, Homo sapiens
gcccaaagctctggactccacagtg

SEQ ID NO: 482 - DNA, 25 bp, Homo sapiens
ccaaagctctggactccacagtgtc

SEQ ID NO: 483 - DNA, 25 bp, Homo sapiens
aaagctctggactccacagtgtccg

SEQ ID NO: 484 - DNA, 25 bp, Homo sapiens
agctctggactccacagtgtccgcg

SEQ ID NO: 485 - DNA, 25 bp, Homo sapiens
ctctggactccacagtgtccgcggt

SEQ ID NO: 486 - DNA, 25 bp, Homo sapiens
ctggactccacagtgtccgcggttt

SEQ ID NO: 487 - DNA, 25 bp, Homo sapiens
ggactccacagtgtccgcggtttgc

SEQ ID NO: 488 - DNA, 25 bp, Homo sapiens
actccacagtgtccgcggtttgcgt

SEQ ID NO: 489 - DNA, 25 bp, Homo sapiens
tccacagtgtccgcggtttgcgttg

SEQ ID NO: 490 - DNA, 20 bp, Artificial Sequence
  Description of Artificial Sequence: Synthetic oligonucleotide
acagacaataaataccgagg

SEQ ID NO: 491 - DNA, 45 bp, Homo sapiens
gccatcagaactgtgagccaaataaacctttttctgtataaatta

SEQ ID NO: 492 - DNA, 62 bp, Homo sapiens
acatctcctggatgattagttcagagatatattaaaatgccccctccctgtggatcctatag

SEQ ID NO: 493 - DNA, 47 bp, Homo sapiens
gatgattagttcagagatatattaaaatgccccctccctgtggatcc

SEQ ID NO: 494 - DNA, 62 bp, Homo sapiens; identical to SEQ ID NO: 492

SEQ ID NO: 495 - DNA, 60 bp, Homo sapiens
atctcctggatgattagttcagagatatattaaaatgccccctccctgtggatcctatag

SEQ ID NO: 496 - DNA, 45 bp, Homo sapiens
tgattagttcagagatatattaaaatgccccctccctgtggatcc

SEQ ID NO: 497 - RNA, 250 nt, Artificial Sequence
  Description of Artificial Sequence: Synthetic polynucleotide
  misc_feature (1)..(250): This sequence may encompass 150-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 498 - RNA, 90 nt, Artificial Sequence
  Description of Artificial Sequence: Synthetic oligonucleotide
  misc_feature (1)..(90): This sequence may encompass 55-90 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 499 - RNA, 17 nt, Artificial Sequence
  Description of Artificial Sequence: Synthetic oligonucleotide
caaaaaaaaaaaaaaaa

SEQ ID NO: 500 - protein, 41 aa, Artificial Sequence
  Description of Artificial Sequence: Synthetic polypeptide
RMRKFKNKGKDTAELRRRRVEVSVELRKAKKDEQILKRRNV

SEQ ID NO: 501 - protein, 7 aa, Artificial Sequence
  Description of Artificial Sequence: Synthetic peptide
PKKKRKV